(19) United States
(12) Patent Application Publication
(10) Pub. No.: US 2023/0192669 A1
(43) Pub. Date: Jun. 22, 2023

(54) COMPOUND, ORGANIC ELECTROLUMINESCENCE DEVICE AND ELECTRONIC APPARATUS

(71) Applicant: Shaanxi Lighte Optoelectronics Material Co., Ltd., Xi'an (CN)

(72) Inventors: Yiyi ZHENG, Xi'an (CN); Tiantian MA, Xi'an (CN)

(21) Appl. No.: 17/928,869

(22) PCT Filed: Mar. 26, 2021
(86) PCT No.: PCT/CN2021/083413

§ 371 Date: Nov. 30, 2022

(30) Foreign Application Priority Data
Jul. 20, 2020 (CN) 202010699979.1
Aug. 19, 2020 (CN) 202010835565.7

(51) Int. Cl. C07D 405/10; H10K 50/11; H10K 85/60; C07D 405/14; C07D 409/10; C07D 495/04; C07D 307/91; C07D 471/04; C07D 491/048; C07F 7/08; H10K 85/40; C07D 405/04; C07D 239/26; C07D 239/70; C07D 251/24; C07D 413/10; C07D 417/10; C07C 13/64

(52) U.S. Cl. CPC C07D 405/10; H10K 50/11; H10K 85/6574; H10K 85/654; C07D 405/14; C07D 409/10; H10K 85/6576; C07D 495/04; H10K 85/657; C07D 307/91; C07D 471/04; H10K 85/6572; H10K 85/615; C07D 491/048; C07F 7/0812; H10K 85/40; C07D 405/04; C07D 239/26; C07D 239/70; C07D 251/24; C07D 413/10; C07D 417/10; H10K 85/622; C07C 13/64; C07B 2200/05

(57) ABSTRACT

The present disclosure provides an organic compound, an organic electroluminescence device and an electronic apparatus. A chemical structure of the organic compound in the present disclosure contains an adamantane spirofluorene ring and a dibenzo five-membered ring. The organic compound has an excellent energy transfer function and can be used as a luminescent layer material in an electroluminescent device, thus improving an efficiency of the organic electroluminescence device and increasing a service life of the organic electroluminescence device.

### CROSS REFERENCE TO RELATED APPLICATIONS

The present application claims the priority of Chinese patent application No. CN202010699979.1 filed on Jul. 20, 2020, and the contents disclosed by the Chinese patent applications are hereby incorporated by reference in their entirety as a part of the present application. The present application claims the priority of Chinese patent application No. CN202010835565.7 filed on Aug. 19, 2020, and the contents of the Chinese patent applications are hereby incorporated by reference in their entirety as a part of the present application.

### TECHNICAL FIELD

The present disclosure belongs to the technical field of organic electroluminescence materials, and specifically relates to a compound, and an organic electroluminescence device and electronic apparatus using the same.

### BACKGROUND

An organic electroluminescence device, such as an organic light-emitting diode (OLED), usually includes a cathode and an anode which are disposed oppositely, and a functional layer disposed between the cathode and the anode. The functional layer is composed of a plurality of organic or inorganic film layers, and generally includes an organic luminescent layer, a hole transport layer located between the organic luminescent layer and the anode, and an electron transport layer located between the organic luminescent layer and the cathode. When a voltage is applied to the cathode and the anode, an electric field is generated between the cathode and the anode. Under an effect of the electric field, electrons on the cathode side move towards the electroluminescent layer and holes on the anode side also move towards the electroluminescent layer, the electrons and the holes combine at the electroluminescent layer to form excitons, and the excitons are in an excited state to release energy outwards, so that the electroluminescence layer emits light outwards.

In the prior art, CN104039778A and the like disclose materials that can prepare a luminescent layer in the organic electroluminescence device. However, the current organic electroluminescence material still has problems of short luminescent service life and low luminescent efficiency. Hence, there is a need to continue to develop a novel material to further improve the service life and efficiency performance of the organic electroluminescence device.

### SUMMARY

The present disclosure aims to provide an organic electroluminescence material with an excellent performance, which can be used as a luminescent layer in an organic electroluminescence device.

In order to achieve the above objective, the present disclosure provides a compound. A structural formula of the compound is shown in a chemical formula 1:

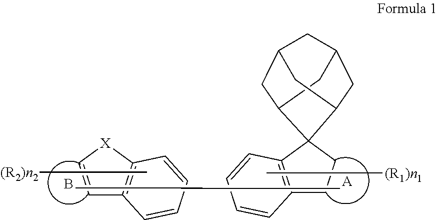

where a ring A and a ring B are each independently an aromatic ring with 6 to 14 carbon atoms;

X is selected from O, S, Si(R₃R₄) or C(R₃R₄);

each R₁ is the same as or different from each other, and is independently selected from: deuterium, a halogen group, cyano, alkyl with 1 to 12 carbon atoms, haloalkyl with 1 to 12 carbon atoms, alkoxy with 1 to 12 carbon atoms, cycloalkyl with 3 to 12 carbon atoms, heterocyclyl with 3 to 12 carbon atoms, alkylsilyl with 3 to 12 carbon atoms, arylsilyl with 6 to 18 carbon atoms, alkylthio with 1 to 12 carbon atoms, aryloxy with 6 to 18 carbon atoms, arylthio with 6 to 18 carbon atoms, aralkoxy with 7 to 18 carbon atoms or

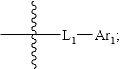

Ar₁ is selected from substituted or unsubstituted aryl with 6 to 40 carbon atoms, or substituted or unsubstituted heteroaryl with 3 to 40 carbon atoms;

L₁ is selected from a single bond, substituted or unsubstituted arylene with 6 to 30 carbon atoms, or substituted or unsubstituted heteroarylene with 3 to 30 carbon atoms;

substituents in Ar₁ and L₁ are the same as or different from each other, and are each independently selected from the group consisting of deuterium, cyano, a halogen group, nitro, a group U, alkoxy with 1 to 12 carbon atoms, haloalkyl with 1 to 12 carbon atoms, alkyl with 1 to 12 carbon atoms, cycloalkyl with 3 to 12 carbon atoms, heterocyclyl with 3 to 12 carbon atoms, alkylsilyl with 3 to 12 carbon atoms, arylsilyl with 6 to 18 carbon atoms, alkylthio with 1 to 12 carbon atoms, aryloxy with 6 to 18 carbon atoms, aralkyl with 7 to 18 carbon atoms, and aryloxy with 6 to 18 carbon atoms; the group U is selected from substituted or unsubstituted aryl with 6 to 30 carbon atoms, or substituted or unsubstituted heteroaryl with 3 to 30 carbon atoms, and substituents in the aryl and the heteroaryl are selected from the group consisting of deuterium, cyano, a halogen group, alkyl with 1 to 12 carbon atoms, alkoxy with 1 to 12 carbon atoms, cycloalkyl with 3 to 12 carbon atoms, haloalkyl with 1 to 12 carbon atoms, alkylsilyl with 3 to 12 carbon atoms, aryl with 6 to 20 carbon atoms, and heteroaryl with 3 to 18 carbon atoms;

or, in each L₁ and each Ar₁, when there are two substituents on a same atom, optionally, the two substituents are connected to each other to form a 5- to 18-membered aliphatic ring or a 5- to 18-membered aromatic ring together with the atoms to which they are jointly connected;

each R₂ is the same as or different from each other, and is independently selected from deuterium, a halogen group, cyano, alkyl with 1 to 12 carbon atoms, haloalkyl with 1 to 12 carbon atoms, alkoxy with 1 to 12 carbon atoms, cycloalkyl with 3 to 12 carbon atoms, heterocyclyl with 3 to 12 carbon atoms, alkylsilyl with 3 to 12 carbon atoms, arylsilyl with 6 to 18 carbon atoms, alkylthio with 1 to 12 carbon atoms, aryloxy with 6 to 18 carbon atoms, arylthio with 6 to 18 carbon atoms, aralkyl with 7 to 18 carbon atoms or

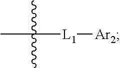

L₂ is selected from a single bond, substituted or unsubstituted arylene with 6 to 30 carbon atoms or

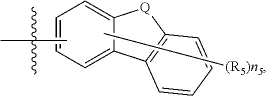

and the L₂ is not anthrylene;

Ar₂ is selected from substituted or unsubstituted aryl with 6 to 30 carbon atoms or

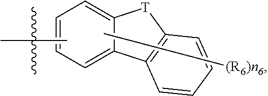

and the Ar₂ is not anthryl;

where Q and T are each independently selected from O, S, C(R₇R₈) or Si(R₇R₈);

R₃, R₄, R₇ and R₈ are the same as or different from each other, and are each independently selected from hydrogen, substituted or unsubstituted alkyl with 1 to 12 carbon atoms, substituted or unsubstituted haloalkyl with 1 to 12 carbon atoms, substituted or unsubstituted aryl with 6 to 18 carbon atoms, and substituted or unsubstituted heteroaryl with 3 to 18 carbon atoms, and substitution in the R₃, the R₄, the R₇ and the R₈ means that the R₃, the R₄, the R₇ and the R₈ are substituted by deuterium, a halogen group, cyano, or alkyl with 1 to 4 carbon atoms;

or the R₇ and the R₈ are connected to each other together with the atom to which they are jointly connected to form a 5- to 13-membered aliphatic ring or a 5- to 13-membered aromatic ring;

or the R₃ and the R₄ are connected to each other together with the atom to which they are jointly connected to form a 5- to 13-membered aliphatic ring or a 5- to 13-membered aromatic ring;

R₅ and R₆ are the same as or different from each other, and are each independently selected from hydrogen, deuterium, a halogen group, cyano, alkyl with 1 to 12 carbon atoms, haloalkyl with 1 to 12 carbon atoms, alkoxy with 1 to 12 carbon atoms, cycloalkyl with 3 to 12 carbon atoms, heterocyclyl with 3 to 12 carbon atoms, alkylsilyl with 3 to 12 carbon atoms, arylsilyl with 6 to 18 carbon atoms, alkylthio with 1 to 12 carbon atoms, aryl with 6 to 18 carbon atoms, dibenzothienyl or dibenzofuranyl; and

n₁ represents the number of R₁, n₂ represents the number of R₂, n₅ represents the number of R₅, n₆ represents the number of R₆, and n₁, n₂, n₅ and n₆ are each independently selected from 0, 1, 2, 3, 4, 5, 6, 7 or 8; when n₁ is greater than 1, any two R₁ are the same or different; when n₂ is greater than 1, any two R₂ are the same or different; when n₅ is greater than 1, any two R₅ are the same or different; and when n₆ is greater than 1, any two R₆ are the same or different.

According to a second aspect of the present disclosure, an organic electroluminescence device is provided. The organic electroluminescence device includes an anode and a cathode which are disposed oppositely, and a functional layer disposed between the anode and the cathode. The functional layer contains the above compound.

According to a third aspect of the present disclosure, an electronic apparatus is provided. The electronic apparatus includes the organic electroluminescence device of the present disclosure.

The structure of the compound of the present disclosure is a structure formed by connecting adamantyl spirofluorene as a main structure and a dibenzo five-membered ring through a single bond; the macromolecular structure has strong rigidity, and a freely rotating δ bond makes a certain twist angle be formed between two ring planes, so that the compound of the present disclosure has a high first triplet energy level and a suitable HOMO energy level distribution. So the compound of the present disclosure may serve as a host material to be applied to an organic electroluminescence layer to improve an efficiency performance of the organic electroluminescence device. Spiro combination of adamantyl and fluorenyl can greatly increase an electron cloud density of large planar conjugated structures through a hyperconjugation effect, enhance a hole mobility of the compound, and is conducive to promote transport balance of holes and electrons in the luminescent layer. Further, a recombination rate of the electrons and the holes in the organic luminescent layer can be increased, and transport of the electrons through the organic luminescent layer to the hole transport layer can be reduced or avoided, so that a material of the hole transport layer can be effectively protected from the impact of the electrons, and a service life of the organic electroluminescence device can be increased. The adamantyl spiro-combined to the fluorenyl has a large steric volume and strong rigidity, so it can reduce an interaction force between the large planar conjugated structures, reduce intermolecular π-π stacking, and adjust an intermolecular stacking degree, so as to make the compound have a more stable amorphous state during film formation, improve a film-forming property of the compound, and further increase the service life of the organic electroluminescence device.

In the compound of the present disclosure, the dibenzo five-membered ring and adamantane spirofluorenyl are connected through a δ bond, and the dibenzo five-membered ring has a strong energy transfer effect, so when applied to the host material of the luminescent layer, it can effectively promote energy transfer between a host and a dopant of the luminescent layer, reduce energy loss and further improve the luminescence efficiency.

Other features and advantages of the present disclosure will be illustrated in detail in the subsequent detailed description part.

### BRIEF DESCRIPTION OF THE DRAWINGS

The accompanying drawings are used to provide a further understanding of the present disclosure, constitute a part of the specification, are used to explain the present disclosure together with the specific implementations below, but do not constitute a limitation to the present disclosure. In the accompanying drawings:

FIG. 1 is a schematic structural diagram of an organic electroluminescence device according to one embodiment of the present disclosure.

FIG. 2 is a schematic structural diagram of an electronic apparatus according to one embodiment of the present disclosure.

### DESCRIPTION OF REFERENCE NUMERALS OF MAIN DEVICES IN THE FIGURES IS AS FOLLOWS

100, anode; 200, cathode; 310, hole injection layer; 321, hole transport layer; 322, electron blocking layer; 330, organic luminescent layer; 340, electron transport layer; 350, electron injection layer; and 400, electronic apparatus.

### DETAILED DESCRIPTION

The specific embodiments of the present disclosure are illustrated in detail below with reference to the accompanying drawings. It should be understood that the specific embodiments described here are merely used to illustrate and explain the present disclosure and not to limit the present disclosure.

In the figures, thicknesses of regions and layers may be exaggerated for clarity. The same reference numerals in the accompanying drawings denote the same or similar structures, and thus their detailed descriptions will be omitted.

In the present disclosure,

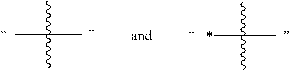

have the same meaning, and both refer to a position bound with other substituents or binding positions.

In the present disclosure, in L₁, L₂, R₁, R₂, R₅, R₆, Ar₁ and Ar₂, the number of carbon atoms in the substituted aryl or heteroaryl refers to the total number of carbon atoms in the aryl or the heteroaryl and the substituent on the aryl or the heteroaryl. For example, the substituted aryl with 18 carbon atoms refers to the total number of carbon atoms in the aryl and the substituent being 18. For example, 2,4-diphenyl-1,3,5-triazinyl belongs to the substituted heteroaryl with 15 carbon atoms.

In this specification, two expressions “substituted or unsubstituted aryl with 6 to 40 carbon atoms” and “substituted or unsubstituted aryl having 6 to 40 carbon atoms” have the same meaning, and both refer to the total number of carbon atoms in the aryl and the substituent on the aryl being 6 to 40. Similarly, in this specification, two expressions “substituted or unsubstituted heteroaryl with 3 to 30 carbon atoms” and “substituted or unsubstituted heteroaryl having 3 to 30 carbon atoms” have the same meaning, and both refer to the total number of carbon atoms in the heteroaryl and the substituent on the heteroaryl being 3 to 30.

In the present disclosure, when no specific definition is provided otherwise, “hetero” refers to a functional group including at least one heteroatom such as B, N, O, S, Se, Si or P and remaining atoms being carbon and hydrogen. The unsubstituted alkyl may be a “saturated alkyl group” without any double bonds or triple bonds.

The description modes “each . . . is independently”, “. . . is respectively and independently” and “. . . is independently selected from” used in the present disclosure can be interchanged, and should be understood in a broad sense, which means that in different groups, specific options expressed between the same symbols do not influence each other, or means that in the same group, specific options expressed between the same symbols do not influence each other. For example, the meaning of description of “in

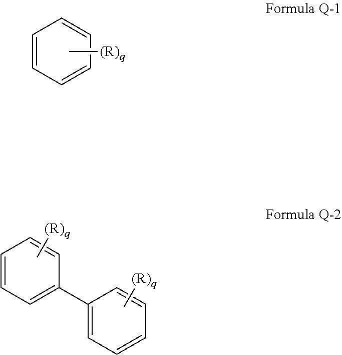

where each q is independently 0, 1, 2 or 3, and each R” is independently selected from hydrogen, fluorine and chlorine” is as follows: the formula Q-1 represents that q substituents R″ exist on a benzene ring, each R″ may be the same or different, and options of each R″ do not influence each other; and the formula Q-2 represents that each benzene ring of biphenyl has q substituents R″, the number q of the substituents R″ on the two benzene rings may be the same or different, each R″ may be the same or different, and options of each R″ do not influence each other.

In the present disclosure, “optional” or “optionally” means that the subsequently described event or environment may, but does not have to occur, and the description includes an occasion where the event or environment occurs or does not occur. For example, “a heterocyclic group optionally substituted with alkyl” means that alkyl may, but does not have to, be present, and the description includes both a situation where the heterocyclic group is substituted with the alkyl and a situation where the heterocyclic group is not substituted with the alkyl. “Two substituents connected to a same atom are connected to each other to form a saturated or unsaturated 5- to 18-membered aliphatic ring or a 5- to 18-membered aromatic ring together with the atoms to which they are jointly connected” means that the two substituents connected to the same atom may form a ring, but do not have to form a ring, including both a situation where the two substituents are connected to each other to form a saturated or unsaturated 5- to 18-membered aliphatic ring or a 5- to 18-membered aromatic ring, and a situation where the two substituents exist independently of each other.

In the present disclosure, the term “substituted or unsubstituted” means no substituent or being substituted with one or more substituents. The substituents include, but are not limited to, deuterium, a halogen group (F, Cl, and Br), cyano, alkyl, alkenyl, alkynyl, haloalkyl, aryl, heteroaryl, aryloxy, arylthio, cycloalkyl, heterocyclyl, trialkylsilyl, alkyl, cycloalkyl, alkoxy and alkylthio.

In the present disclosure, “alkyl” may include straight chain alkyl or branched chain alkyl. The alkyl may have 1 to 12 carbon atoms. In the present disclosure, a numerical range such as “1 to 12” refers to each integer in the given range. For example, “1 to 12 carbon atoms” means the alkyl capable of containing 1 carbon atom, 2 carbon atoms, 3 carbon atoms, 4 carbon atoms, 5 carbon atoms, 6 carbon atoms, 7 carbon atoms, 8 carbon atoms, 9 carbon atoms, 10 carbon atoms, 11 carbon atoms, and 12 carbon atoms. The alkyl may further be medium-sized alkyl with 1 to 10 carbon atoms. The alkyl may further be lower alkyl with 1 to 6 carbon atoms. In some other embodiments, the alkyl group contains 1 to 4 carbon atoms. In further other embodiments, the alkyl group contains 1 to 3 carbon atoms. The alkyl group may be optionally substituted with one or more substituents described in the present disclosure. Examples of the alkyl group include, but are not limited to, methyl (Me, —CH₃), ethyl (Et, —CH₂CH₃), n-propyl (n-Pr, —CH₂CH₂CH₃), isopropyl (i-Pr, —CH(CH₃)₂), n-butyl (n-Bu, —CH₂CH₂CH₂CH₃), isobutyl (i-Bu, —CH₂CH(CH₃)₂), sec-butyl (s-Bu, —CH(CH₃)CH₂CH₃), tert-butyl (t-Bu, —C(CH₃)₃), and the like. In addition, the alkyl may be substituted or unsubstituted.

In the present disclosure, alkylsilyl refers to

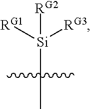

where R^{G1}, R^{G2} and R^{G3} are each independently alkyl, and specific examples of the alkylsilyl include, but are not limited to, trimethylsilyl, triethylsilyl, tert-butyldimethylsilyl and propyldimethylsilyl.

In the present disclosure, arylsilyl refers to

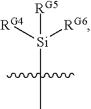

where R^{G4}, R^{G5} and R^{G6} are each independently aryl, and specific examples of the arylsilyl include, but are not limited to, triphenylsilyl, diphenylsilyl, phenylsilyl and the like, but not limited to these.

In the present disclosure, a halogen group serving as a substituent is, for example, fluorine, chlorine, bromine or iodine.

In the present disclosure, “alkoxy” means that an alkyl group is connected to the rest part of the molecule through an oxygen atom, where the alkyl group has the meaning as described in the present disclosure. Unless otherwise specified, the alkoxy group contains 1 to 12 carbon atoms. In one embodiment, the alkoxy group contains 1 to 6 carbon atoms. In another embodiment, the alkoxy group contains 1 to 4 carbon atoms. In further another embodiment, the alkoxy group contains 1 to 3 carbon atoms. The alkoxy group may be optionally substituted with one or more substituents described in the present disclosure.

Examples of the alkoxy group include, but are not limited to, methoxyl (MeO, —OCH₃), ethoxy (EtO, —OCH₂CH₃), 1-propoxy (n-PrO, n-propoxy, —OCH₂CH₂CH₃), 2-propoxy (i-PrO, i-propoxy, —OCH(CH₃)₂), 1-butoxy (n-BuO, n-butoxy, —OCH₂CH₂CH₂CH₃), 2-methyl-1-propoxy (i-BuO, i-butoxy, —OCH₂CH(CH₃)₂), 2-butoxy (s-BuO, s-butoxy, —OCH(CH₃)CH₂CH₃), 2-methyl propoxy (t-BuO, t-butoxy, —OC(CH₃)₃), and the like.

In the present disclosure, “alkylthio” means that an alkyl group is connected to the rest part of the molecule through a sulfur atom, where the alkyl group has the meaning as described in the present disclosure. Unless otherwise specified, the alkylthio group contains 1 to 12 carbon atoms. In one embodiment, the alkylthio group contains 1 to 6 carbon atoms. In another embodiment, the alkylthio group contains 1 to 4 carbon atoms. In further another embodiment, the alkylthio group contains 1 to 3 carbon atoms. The alkylthio group may be optionally substituted with one or more substituents described in the present disclosure. Examples of the alkylthio group include, but are not limited to, methylthio (MeS, —SCH₃), ethylthio (EtS, —SCH₂CH₃), 1-propylthio (n-PrS, n-propylthio, —SCH₂CH₂CH₃), 2-propylthio (i-PrS, i-propylthio, —SCH(CH₃)₂), and the like.

In the present disclosure, “haloalkyl” or “haloalkoxy” means an alkyl or alkoxy group substituted with one or more halogen atoms, where the alkyl and alkoxy groups have the meaning described in the present disclosure, and such examples contain, but are not limited to, trifluoromethyl, trifluoromethoxy and the like. In one embodiment, haloalkyl with 1 to 6 carbon atoms contains fluorine-substituted C₁-C₆ alkyl. In another embodiment, C₁-C₄ haloalkyl contains fluorine-substituted C₁-C₄ alkyl. In further another embodiment, C₁-C₂ haloalkyl contains fluorine substituted C₁-C₂ alkyl.

In the present disclosure, cycloalkyl refers to group obtained by removing a hydrogen atom from a monocyclic or polycyclic saturated cyclic hydrocarbon, and “cycloalkyl” may have one or more connecting points connected to the rest part of the molecule. In some embodiments, cycloalkyl is a ring system containing 3 to 12 cyclic carbon atoms. In some other embodiments, cycloalkyl is a ring system containing 5 to 10 cyclic carbon atoms. In further other embodiments, cycloalkyl is a ring system containing 5 to 7 cyclic carbon atoms. In further other embodiments, cycloalkyl is a ring system containing 3 to 6 cyclic carbon atoms. For example, “cycloalkyl with 3 to 12 carbon atoms” means cycloalkyl capable of containing 3 carbon atoms, 4 carbon atoms, 5 carbon atoms, 6 carbon atoms, 7 carbon atoms, 8 carbon atoms, 9 carbon atoms, 10 carbon atoms, 11 carbon atoms, and 12 carbon atoms. The cycloalkyl group may be independently unsubstituted or substituted with one or more substituents described in the present disclosure. As its non-limiting examples, cycloalkyl has cyclopropyl, cyclobutyl, cyclopentyl, cyclohexyl, norbornyl, adamantyl, and the like.

In the present disclosure, “heterocycle” and “heterocyclyl” may be used interchangeably, and both refer to a monocyclic, bicyclic or tricyclic system, where one or more atoms on the ring may be independently and optionally replaced with a heteroatom, and the ring may be fully saturated or contain one or more unsaturated bonds, but is never aromatic, with merely one connecting point connected to other molecules. Hydrogen atoms on one or more rings are independently and optionally substituted with one or more substituents described in the present disclosure. In some embodiments,“heterocycle”, “heterocyclyl”, “heteroalicyclic” or “heterocyclic” groups are a 3- to 7-membered monocyclic ring (1 to 6 carbon atoms and 1, 2 or 3 heteroatoms selected from N, O, P or S, where S or P is optionally substituted with one or more oxygen atoms to obtain groups like SO, SO₂, PO and PO₂, when the ring is a three-membered ring, there is merely one heteroatom), or a 7- to 10-membered bicyclic ring (4 to 9 carbon atoms and 1, 2 or 3 heteroatoms selected from N, O, P or S, where S or P is optionally substituted with one or more oxygen atoms to obtain groups like SO, SO₂, PO and PO₂). The heterocyclyl may be a carbon group or a nitrogen group, and a —CH₂— group may be optionally replaced by —C(═O)—. Sulfur atoms of the ring may be optionally oxidized to S-oxides. Nitrogen atoms of the ring may be optionally oxidized into N-oxygen compounds.

Examples of heterocycle include, but are not limited to, pyrrolidinyl, tetrahydrofuranyl, dihydrofuranyl, tetrahydrothienyl, tetrahydropyranyl, dihydropyranyl, tetrahydrothiopyranyl, piperidinyl, morpholinyl, thiomorpholinyl, thioxanyl, piperazinyl, homopiperazinyl, azetidinyl, oxetanyl, thietanyl, homopiperidinyl, glycidyl, azepanyl, oxepanyl, thiazepanyl, oxazepanyl, diazepanyl, thiazepanyl, 2-pyrrolinyl, 3-pyrrolinyl, indoline, 2H-pyranyl, 4H-pyranyl, dioxanyl, 1,3-dioxopentyl, pyrazolinyl, dithianyl, dithiolanyl, dihydrothienyl, pyrazolidinyl imidazolinyl, imidazolidinyl and 1,2,3,4-tetrahydroisoquinolyl.

In the present disclosure, the aryl refers to an optional functional group or substituent derived from an aromatic carbocyclic ring. The aryl may be monocyclic aryl or polycyclic aryl. In other words, aryl may be monocyclic aryl, fused-ring aryl, two or more monocyclic aryl connected in a conjugated mode through carbon-carbon bonds, monocyclic aryl and fused-ring aryl which are connected in a conjugated mode through carbon-carbon bonds, and two or more fused-ring aryl connected in a conjugated mode through carbon-carbon bonds. That is, two or more aromatic groups connected in a conjugated mode through carbon-carbon bonds may also be regarded as aryl of the present disclosure. The fused-ring aryl refers to two or more rings in which the two carbon atoms in the ring system are shared by two adjacent rings, where at least one of the rings is aromatic, for example the other rings may be cycloalkyl, cycloalkenyl, or aryl. For example, in the present disclosure, biphenyl, terphenyl and the like are aryl. Examples of aryl may include, but are not limited to, phenyl, naphthyl, fluorenyl, anthryl, phenanthryl, biphenyl, terphenyl, tetraphenyl, pentaphenyl, benzo[9,10]phenanthryl, pyrenyl, benzofluoranthenyl, chrysenyl, and the like. In this specification, the number of carbon atoms in the aryl may be selected from 6, 10, 12, 13, 14, 15, 16, 17, 18, 20, 25 or 30. In some embodiments, aryl is aryl with 6 to 30 carbon atoms. In some other embodiments, aryl is aryl with 6 to 15 carbon atoms. In some other embodiments, aryl is aryl with 6 to 18 carbon atoms. In some other implementations, aryl is aryl with 6 to 20 carbon atoms.

In the present disclosure, the substituted aryl may be one or two or more hydrogen atoms in the aryl substituted by groups such as a deuterium atom, a halogen group, cyano(—CN), aryl, heteroaryl, trialkylsilyl, alkyl, cycloalkyl, alkoxy, alkylthio, haloalkyl, aryloxy, arylthio, silyl, alkylamino, aryl and heterocyclyl. Specific examples of heteroaryl-substituted aryl include, but are not limited to, phenyl substituted with dibenzofuranyl, phenyl substituted with dibenzothienyl, phenyl substituted with pyridyl, and the like. It should be understood that the number of carbon atoms in the substituted aryl refers to the total number of carbon atoms in the aryl and the substituent on the aryl. For example, the substituted aryl with 18 carbon atoms refers to the total number of carbon atoms in the aryl and its substituent being 18.

In the present disclosure, fluorenyl serving as the aryl may be substituted, and the two substituents may be combined with each other to form a spiro structure. Specific examples include but are not limited to the following structures:

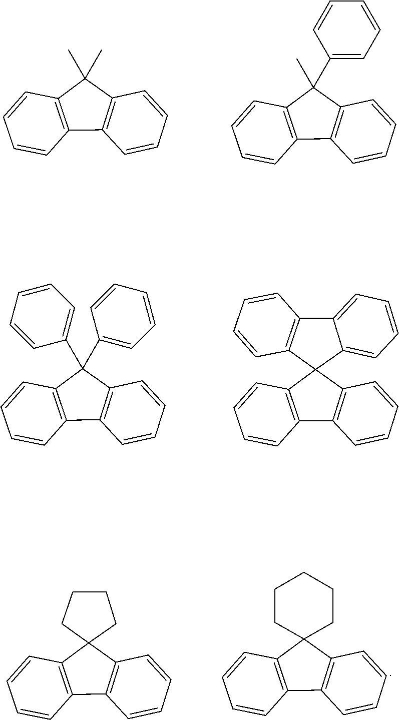

In the present disclosure, arylene is a bivalent group, and other than that, the above description about the aryl may be applied.

In the present disclosure, heteroaryl refers to a monocyclic or polycyclic system containing 1, 2, 3, 4, 5, or 6 heteroatoms independently selected from O, N, P, Si, Se, B or S in the ring, where at least one ring system is aromatic. Each ring system in the heteroaryl contains a ring consisting of 5 to 7 ring atoms, and one or more attachment points are connected with the rest part of the molecule. The heteroaryl may be monocyclic heteroaryl or polycyclic heteroaryl. In other words, the heteroaryl may be a single aromatic ring system or a plurality of aromatic ring systems connected in a conjugated mode through carbon-carbon bonds, and any aromatic ring system is an aromatic monocyclic ring or an aromatic fused ring. The fused-ring heteroaryl refers to two or more rings in which the two atoms in the ring system are shared by two adjacent rings, where at least one of the rings is aromatic, for example, the other rings may be cycloalkyl, heteroaryl, cycloalkenyl, or aryl.

Illustratively, the heteroaryl may include thienyl, furyl, pyrrolyl, imidazolyl, thiazolyl, oxazolyl, isothiazolyl, oxadiazolyl, triazolyl, azolyl, furazanyl, pyridyl, bipyridyl, phenanthridinyl, pyrimidinyl, triazinyl, acridinyl, pyridazinyl, pyrazinyl, quinolyl, quinazolinyl, quinoxalinyl, phenoxazinyl, phthalazinyl, pyridopyrimidyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuryl, phenanthrolinyl, isoxazolyl, thiadiazolyl, benzothiazolyl, phenothiazinyl, silafluorenyl, dibenzofuranyl, N-arylcarbazolyl (e.g., N-phenylcarbazolyl), N-heteroarylcarbazolyl (e.g., N-pyridylcarbazolyl), N-alkylcarbazolyl (e.g., N-methylcarbazolyl), and the like, but is not limited to this. Where thienyl, furyl, phenanthrolinyl and the like are heteroaryl of the single aromatic ring system, and N-arylcarbazolyl and N-heteroarylcarbazolyl are heteroaryl of a plurality of ring systems connected in a conjugated mode through carbon-carbon bonds.

In the present disclosure, the substituted heteroaryl may be one or two or more hydrogen atoms in the heteroaryl substituted by groups such as a deuterium atom, a halogen group, cyano, aryl, heteroaryl, trialkylsilyl, alkyl, cycloalkyl, alkoxy and alkylthio and the like. Specific examples of heteroaryl substituted with aryl includes, but are not limited to, dibenzofuranyl substituted with phenyl, dibenzothienyl substituted with phenyl, pyridyl substituted with phenyl, and the like. It should be understood that the number of carbon atoms in the substituted heteroaryl refers to the total number of carbon atoms in the heteroaryl and the substituent on the heteroaryl. For example, the substituted heteroaryl with 14 carbon atoms refers to the total number of carbon atoms in the heteroaryl and the substituent being 14.

In the present disclosure, the number of carbon atoms of the heteroaryl may be selected from 3, 4, 5, 6, 7, 8, 9, 10, 11, 12, 13, 14, 15, 16, 17, 18, 20, 21, 22, 23, 24, 25, 26, 27, 28, 29 or 30. In some embodiments, heteroaryl is heteroaryl with 3 to 12 carbon atoms. In some other embodiments, aryl is aryl with 3 to 15 carbon atoms. In some other embodiments, aryl is aryl with 5 to 12 carbon atoms.

In this specification, heteroarylene is a bivalent group, and other than that, the above description about the heteroaryl may be applied.

In the present disclosure, heteroaryl with 3 to n ring-forming carbon atoms refers to the number of carbon atoms located on the heteroaromatic ring in the heteroaryl being 3 to n, and the carbon atoms in the substituent on the heteroaryl are not counted.

In the present disclosure, explanation for aryl may be applied to arylene, explanation for heteroaryl may also be applied to heteroarylene, explanation for alkyl may be applied to alkylene, and explanation for cycloalkyl may be applied to cycloalkylene.

In the present disclosure, a ring system formed by n atoms is an n-membered ring. For example, phenyl is 6-membered aryl. The 6- to 13-membered aromatic ring refers to a benzene ring, an indene ring, a naphthalene ring and the like.

“Ring” in the present disclosure contains a saturated ring and an unsaturated ring; the saturated ring is cycloalkyl and heterocycloalkyl, and the unsaturated ring is cycloalkenyl, heterocycloalkenyl, aryl and heteroaryl.

In the present disclosure, an unpositioned connecting bond refers to a single bond

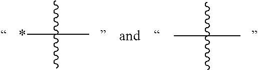

extending from the ring system, which means that one end of the connecting bond may be connected with any position in the ring system through which the bond penetrates, and the other end of the connecting bond is connected with the rest part of a compound molecule. For example, as shown in the following formula (X), naphthyl represented by the formula (X) is connected with other positions of the molecule through two unpositioned connecting bonds penetrating through a dicyclic ring, and its represented meaning includes any one possible connecting mode as shown in formulae (X-1) to (X-10).

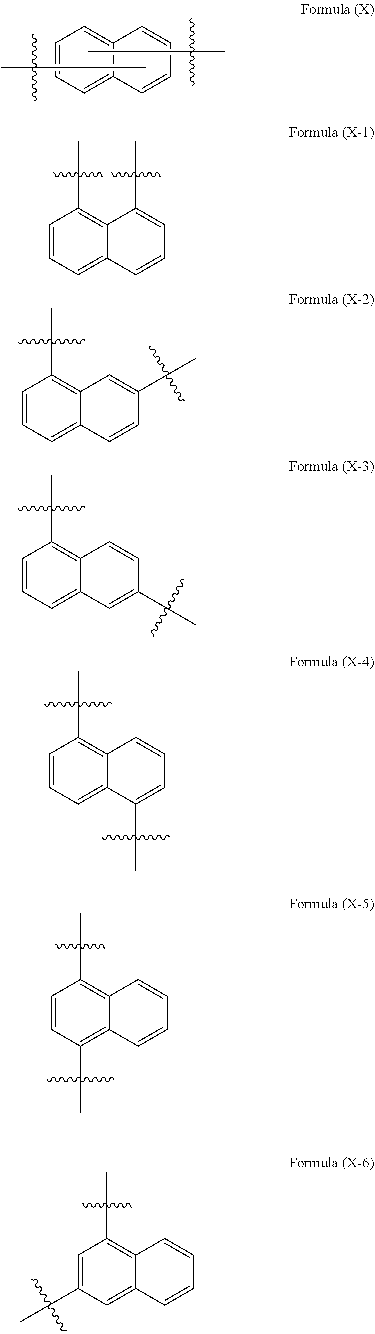

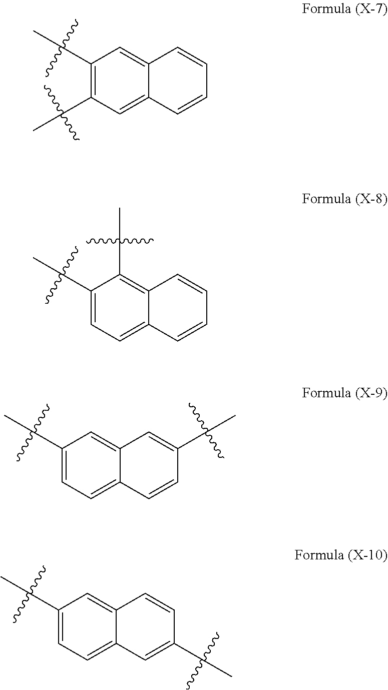 [SMILES: *c1cc2ccccc2cc1* ; *c1ccc2ccccc2c1* ; *c1ccc2ccc(*)cc2c1 ; *c1ccc2cc(*)ccc2c1]

For example, as shown in the following formula (X′), phenanthryl represented by the formula (X′) is connected with other positions of the molecule through one unpositioned connecting bond extending from the center of a benzene ring on one side, and its represented meaning includes any one possible connecting mode as shown in formulae (X′-1) to (X′-4).

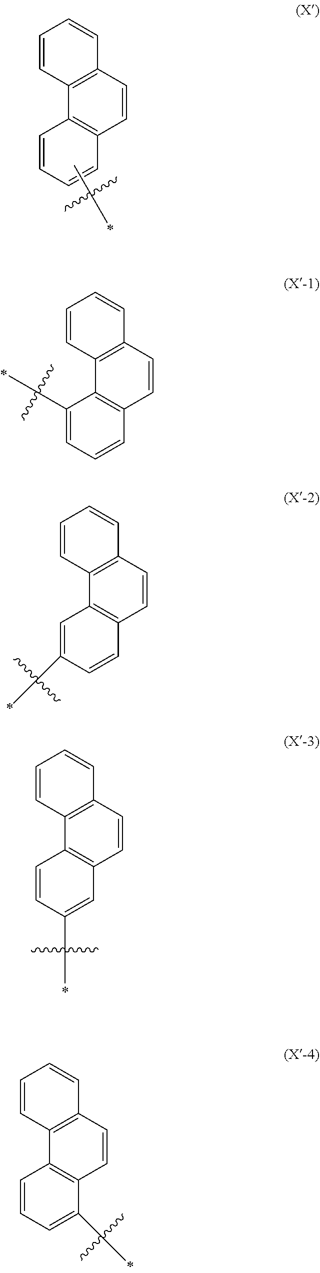

An unpositioned substituent in the present disclosure, refers to a substituent connected through a single bond extending from the center of the ring system, which means that the substituent may be connected at any possible position of the ring system. For example, as shown in the following formula (Y), a substituent R group represented by the formula (Y) is connected with a quinoline ring through one unpositioned connecting bond, and its represented meaning includes any one possible connecting mode as shown in formulae (Y-1) to (Y-7).

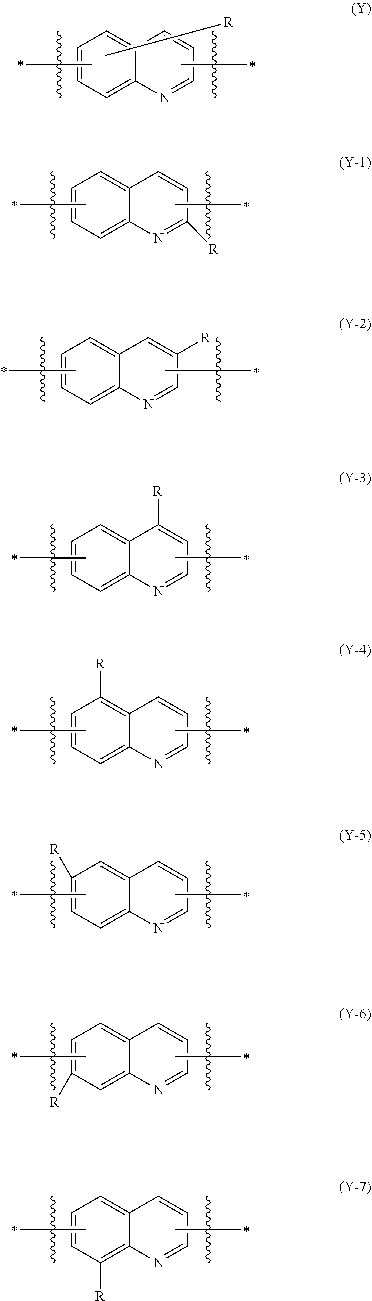

The present disclosure provides a compound. A structural formula of the compound is shown in a chemical formula 1:

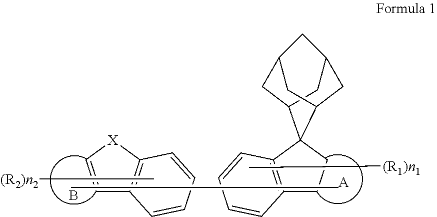

where a ring A and a ring B are each independently an aromatic ring with 6 to 14 carbon atoms;

X is selected from O, S, Si(R₃R₄) or C(R₃R₄);

each R₁ is the same as or different from each other, and is independently selected from: deuterium, a halogen group, cyano, alkyl with 1 to 12 carbon atoms, haloalkyl with 1 to 12 carbon atoms, alkoxy with 1 to 12 carbon atoms, cycloalkyl with 3 to 12 carbon atoms, heterocyclyl with 3 to 12 carbon atoms, alkylsilyl with 3 to 12 carbon atoms, arylsilyl with 6 to 18 carbon atoms, alkylthio with 1 to 12 carbon atoms, aryloxy with 6 to 18 carbon atoms, arylthio with 6 to 18 carbon atoms, aralkoxy with 7 to 18 carbon atoms or

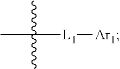

Ar₁ is selected from substituted or unsubstituted aryl with 6 to 40 carbon atoms, or substituted or unsubstituted heteroaryl with 3 to 40 carbon atoms;

L₁ is selected from a single bond, substituted or unsubstituted arylene with 6 to 30 carbon atoms, or substituted or unsubstituted heteroarylene with 3 to 30 carbon atoms;

substituents in Ar₁ and L₁ are the same as or different from each other, and are each independently selected from the group consisting of: deuterium, cyano, a halogen group, nitro, a group U, alkoxy with 1 to 12 carbon atoms, haloalkyl with 1 to12 carbon atoms, alkyl with 1 to 12 carbon atoms, cycloalkyl with 3 to 12 carbon atoms, heterocyclyl with 3 to 12 carbon atoms, alkylsilyl with 3 to 12 carbon atoms, arylsilyl with 6 to 18 carbon atoms, alkylthio with 1 to 12 carbon atoms, aryloxy with 6 to 18 carbon atoms, aralkyl with 7 to 18 carbon atoms, or aryloxy with 6 to 18 carbon atoms, the group U is selected from substituted or unsubstituted aryl with 6 to 30 carbon atoms, or substituted or unsubstituted heteroaryl with 3 to 30 carbon atoms, and substituents in the aryl and the heteroaryl (namely substituents in the group U) are selected from the group consisting of deuterium, cyano, a halogen group, alkyl with 1 to 12 carbon atoms, alkoxy with 1 to 12 carbon atoms, cycloalkyl with 3 to 12 carbon atoms, haloalkyl with 1 to 12 carbon atoms, alkylsilyl with 3 to 12 carbon atoms, aryl with 6 to 20 carbon atoms, and heteroaryl with 3 to 18 carbon atoms;

or, in each L₁ and each Ar₁, when there are two substituents on a same atom, optionally, the two substituents are connected to each other to form a 5- to 18-membered aliphatic ring or a 5- to 18-membered aromatic ring together with the atoms to which they are jointly connected;

each R₂ is the same as or different from each other, and is independently selected from deuterium, a halogen group, cyano, alkyl with 1 to 12 carbon atoms, haloalkyl with 1 to 12 carbon atoms, alkoxy with 1 to 12 carbon atoms, cycloalkyl with 3 to 12 carbon atoms, heterocyclyl with 3 to 12 carbon atoms, alkylsilyl with 3 to 12 carbon atoms, arylsilyl with 6 to 18 carbon atoms, alkylthio with 1 to 12 carbon atoms, aryloxy with 6 to 18 carbon atoms, arylthio with 6 to 18 carbon atoms, aralkyl with 7 to 18 carbon atoms or

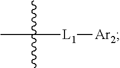

L₂ is selected from a single bond, substituted or unsubstituted arylene with 3 to 30 carbon atoms or

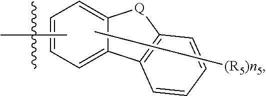

and the L₂ is not anthrylene;

Ar₂ is selected from substituted or unsubstituted aryl with 3 to 30 carbon atoms or

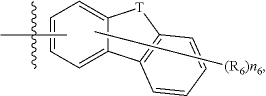

and the Ar₂ is not anthryl;

where Q and T are each independently selected from O, S, C(R₇R₈) or Si(R₇R₈);

R₃, R₄, R₇ and R₈ are the same as or different from each other, and are each independently selected from hydrogen, substituted or unsubstituted alkyl with 1 to 12 carbon atoms, substituted or unsubstituted haloalkyl with 1 to 12 carbon atoms, substituted or unsubstituted aryl with 6 to 18 carbon atoms, and substituted or unsubstituted heteroaryl with 3 to 18 carbon atoms, and substitution in the R₃, the R₄, the R₇ and the R₈ means that the R₃, the R₄, the R₇ and the R₈ are substituted by deuterium, a halogen group, cyano, or alkyl with 1 to 4 carbon atoms;

or the R₇ and the R₈ are connected to each other to form a 5- to 13-membered aliphatic ring or a 5- to 13-membered aromatic ring together with the atoms to which they are jointly connected;

or the R₃ and the R₄ are connected to each other to form a 5- to 13-membered aliphatic ring or a 5- to 13-membered aromatic ring together with the atoms to which they are jointly connected;

R₅ and R₆ are the same as or different from each other, and are each independently selected from hydrogen, deuterium, a halogen group, cyano, alkyl with 1 to 12 carbon atoms, haloalkyl with 1 to 12 carbon atoms, alkoxy with 1 to 12 carbon atoms, cycloalkyl with 3 to 12 carbon atoms, heterocyclyl with 3 to 12 carbon atoms, alkylsilyl with 3 to 12 carbon atoms, arylsilyl with 6 to 18 carbon atoms, alkylthio with 1 to 12 carbon atoms, aryl with 6 to 18 carbon atoms, dibenzothienyl or dibenzofuranyl; and

n₁ represents the number of R₁, n₂ represents the number of R₂, n₅ represents the number of R₅, n₆ represents the number of R₆, and n₁, n₂, n₅ and n₆ are each independently selected from 0, 1, 2, 3, 4, 5, 6, 7 or 8; when n₁ is greater than 1, any two R₁ are the same or different; when n₂ is greater than 1, any two R₂ are the same or different; when n₅ is greater than 1, any two R₅ are the same or different; and when n₆ is greater than 1, any two R₆ are the same or different.

In the present disclosure, the ring A refers to

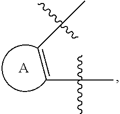

and the ring B refers to

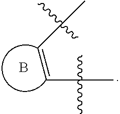

For example, in the compound

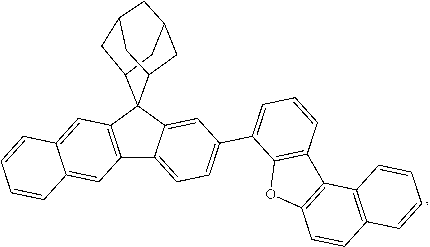 [SMILES: c1ccc2cc3c(cc2c1)-c1ccc(-c2cccc4c2oc2ccc5ccccc5c24)cc1C31C2CC3CC(C2)CC1C3]

the ring A is a naphthalene ring, and the number of the substituent R₁ is 0; and the ring B is a naphthalene ring, X is oxygen, and the number of the substituent R₂ is 0. It may be understood that the ring B at least includes one benzene ring structure, which makes the compound of the present disclosure at least include one dibenzo five-membered fused ring structure.

Since the ring A or the ring B in the compound is of the fused ring structure, a large planar conjugated structure of the compound is larger, the rigidity is stronger, and the electron cloud density is higher, which makes the compound have a stronger hole transport ability, can further increase a recombination rate of electrons and holes in an organic luminescent layer, and reduce or avoid the electrons from penetrating through the organic luminescent layer and transferring to the hole transport layer, so that a material of a hole transport layer can be effectively protected from the impact of the electrons, and luminescence service life of the organic electroluminescence device can be increased.

A structure of the compound of the present disclosure is a structure formed by connecting adamantyl spirofluorene as a main structure and a dibenzo five-membered ring through a single bond; the macromolecular structure has strong rigidity, and a freely rotating δ bond makes a certain twist angle be formed between two ring planes, so that the compound of the present disclosure has a high first triplet energy level and a suitable HOMO energy level distribution, and the dibenzo five-membered fused ring has an excellent energy transfer effect and thus can effectively promote the energy transfer between host and dopant materials of the luminescent layer. So the compound of the present disclosure may serve as the host material of the luminescent layer to be applied to an organic electroluminescence layer to improve an efficiency performance of the organic electroluminescence device. Spiro combination of adamantyl and fluorenyl can greatly increase the electron cloud density of the large planar conjugated structures through a hyperconjugation effect, enhance a hole mobility of the compound, and is conducive to promote transport balance of the holes and the electrons in the luminescent layer. Further, the recombination rate of the electrons and the holes in the organic luminescent layer can be increased, and transport of the electrons through the organic luminescent layer to the hole transport layer can be reduced or avoided, so that a material of the hole transport layer can be effectively protected from the impact of the electrons, and the service life of the organic electroluminescence device can be increased. The adamantyl spiro-combined to the fluorenyl has a large steric volume and strong rigidity, so it can reduce an interaction force between the large planar conjugated structures, reduce intermolecular π-π stacking, and adjust an intermolecular stacking degree, so as to make the compound have a more stable amorphous state during film formation, improve a film-forming property of the compound, and further increase the service life of the organic electroluminescence device.

In the compound of the present disclosure, the dibenzo five-membered ring and adamantyl spirofluorenyl are connected through a δ bond, and the dibenzo five-membered ring has a strong energy transfer effect, so when applied to the host material of the luminescent layer, it can effectively promote energy transfer between a host and a dopant of the luminescent layer, reduce energy loss and further improve the luminescence efficiency.

In an optional embodiment of the present disclosure, in the formula (1), n₁+n₂=1.

In an optional embodiment of the present disclosure, in the formula (1), n₁ is 1, n₂ is 0, and R₁ is

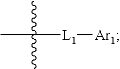

or, n₁ is 0, n₂ is 1, and R₂ is

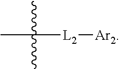

In an optional embodiment of the present disclosure, X is selected from O, S, Si(R₃R₄) or C(R₃R₄), the R₃ and the R₄ are the same, and the R₃ and the R₄ are selected from methyl or phenyl; or the R₃ and the R₄ are connected to each other to form a cyclopentane ring, a cyclohexane ring or a fluorene ring together with the atoms to which they are commonly connected.

In an optional embodiment of the present disclosure, Q and T are each independently selected from O, S, C(R₇R₈) or Si(R₇R₈), the R₇ and the R₈ are the same, and the R₇ and the R₈ are selected from methyl or phenyl; or the R₇ and the R₈ are connected to each other to form a cyclopentane ring, a cyclohexane ring or a fluorene ring together with the atoms to which they are jointly connected.

In some embodiments, the structural formula of the compound of the present disclosure is shown in any one of formulae (f-1) to (f-16):

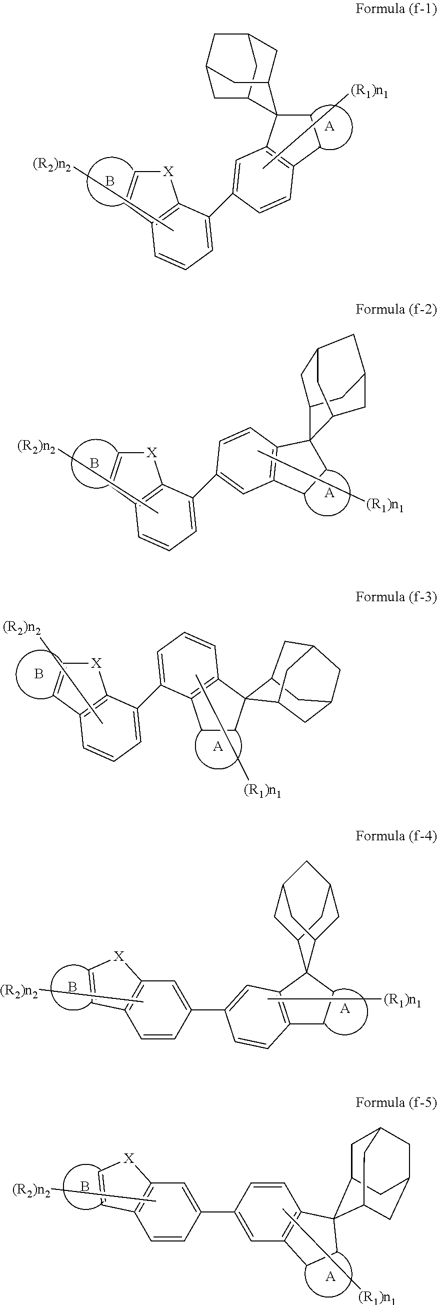

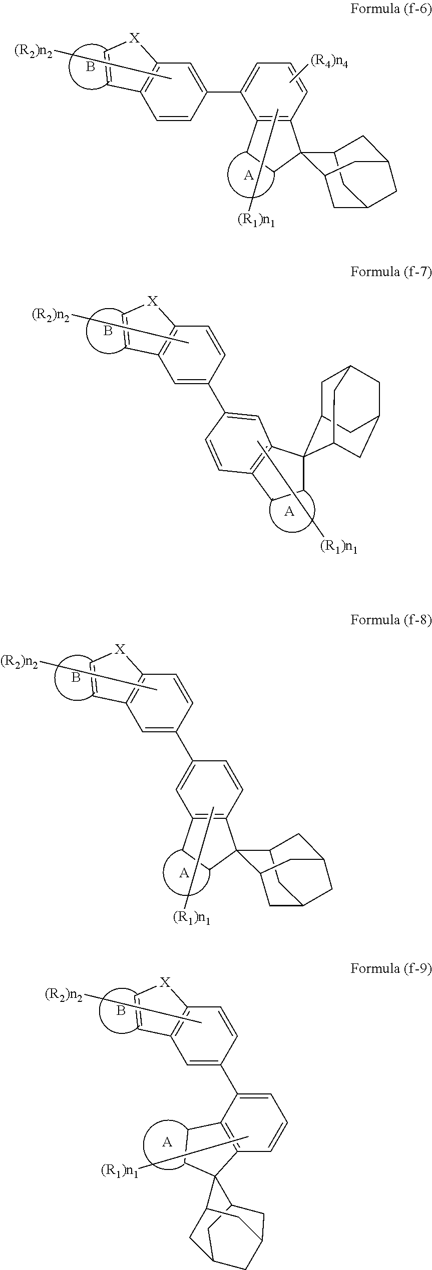

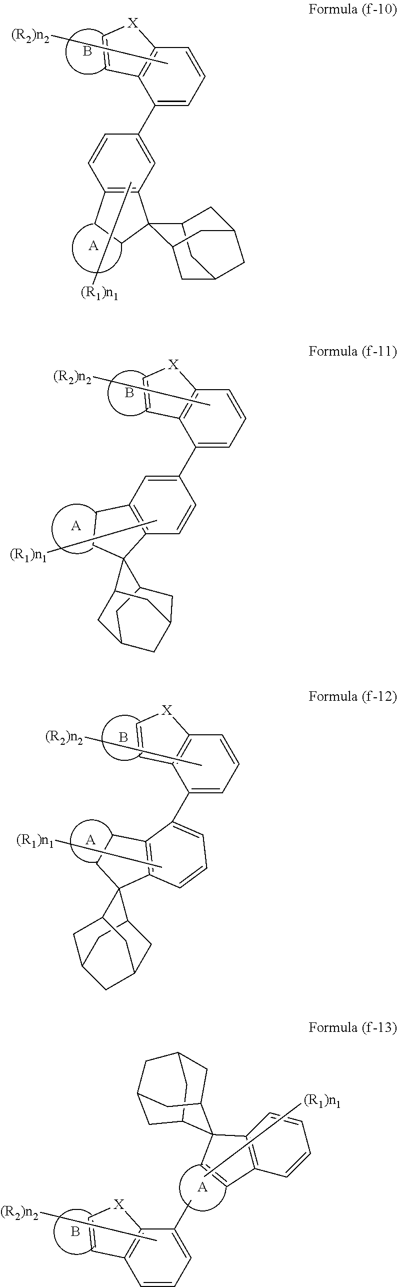

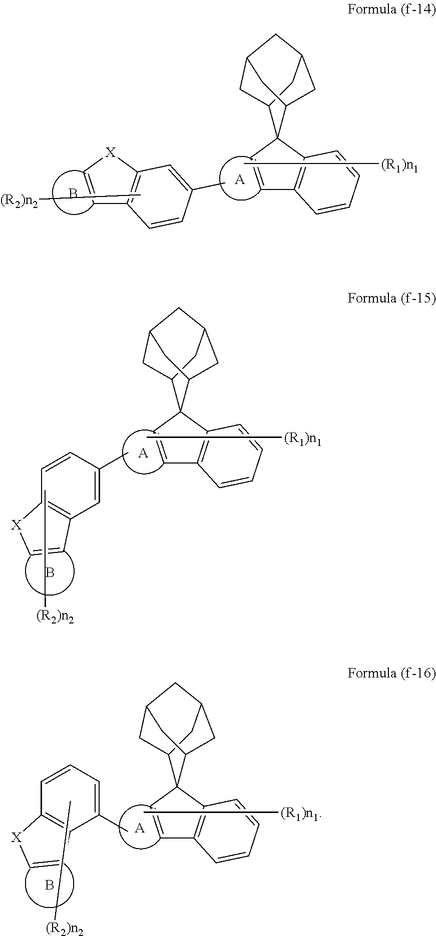

In an optional embodiment of the present disclosure, the ring A and the ring B in the formula (1) are each independently a benzene ring, a naphthalene ring, an anthracene ring or a phenanthrene ring.

In an optional embodiment of the present disclosure,

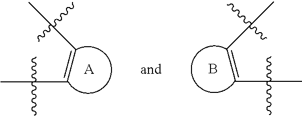

in the formula (1) are each independently selected from the following structures:

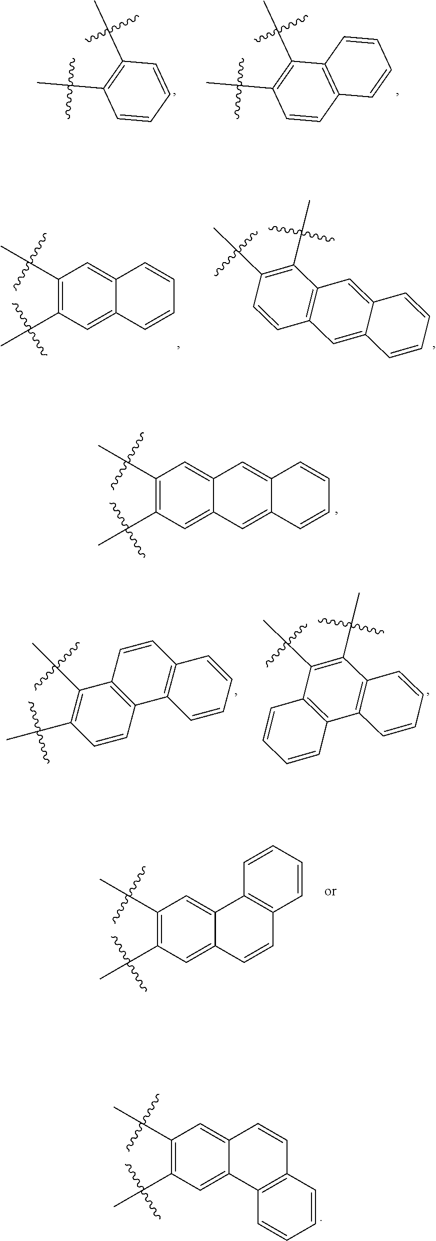

Further, the ring A is selected from

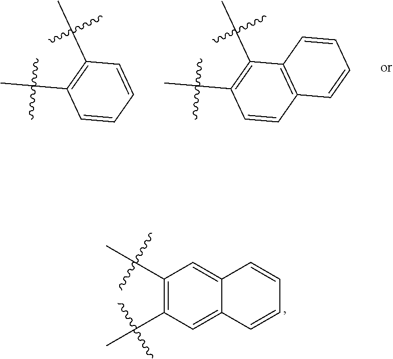 [SMILES: *c1ccccc1* ; *c1ccc2ccccc2c1* ; *c1cc2ccccc2cc1*]

and the ring B is selected from

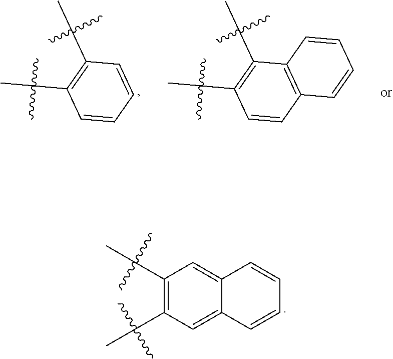 [SMILES: *c1ccccc1* ; *c1ccc2ccccc2c1* ; *c1cc2ccccc2cc1*]

In an optional embodiment of the present disclosure, the structural formula of the compound of the present disclosure is shown in any one of formulae (q-1) to (q-12):

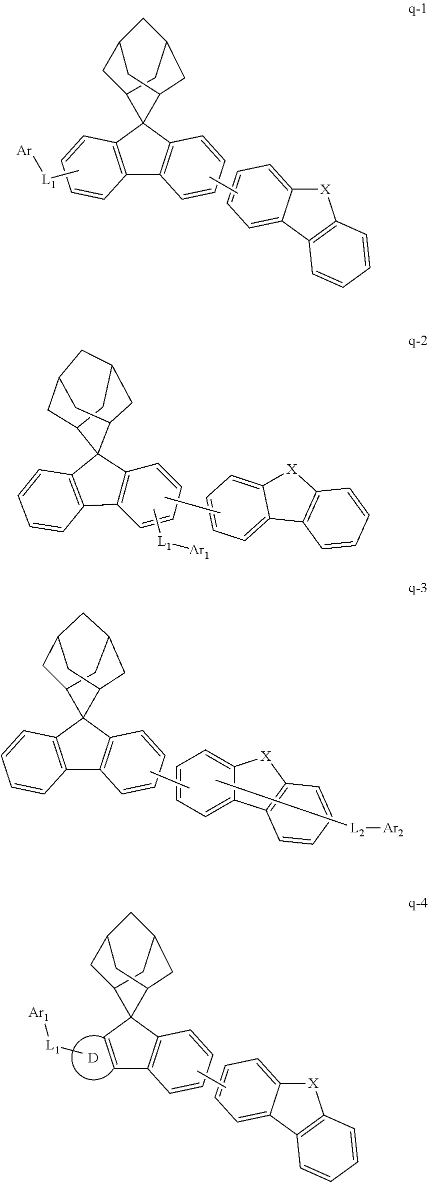

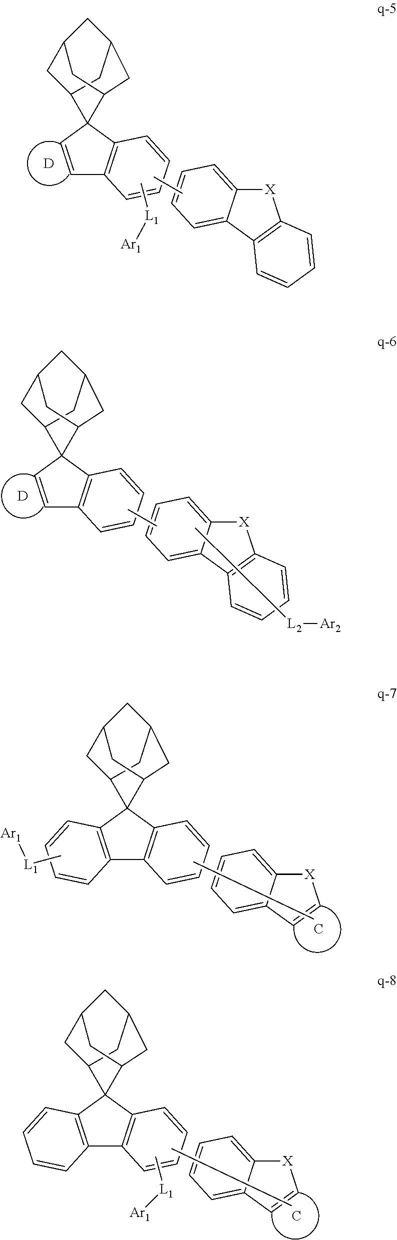

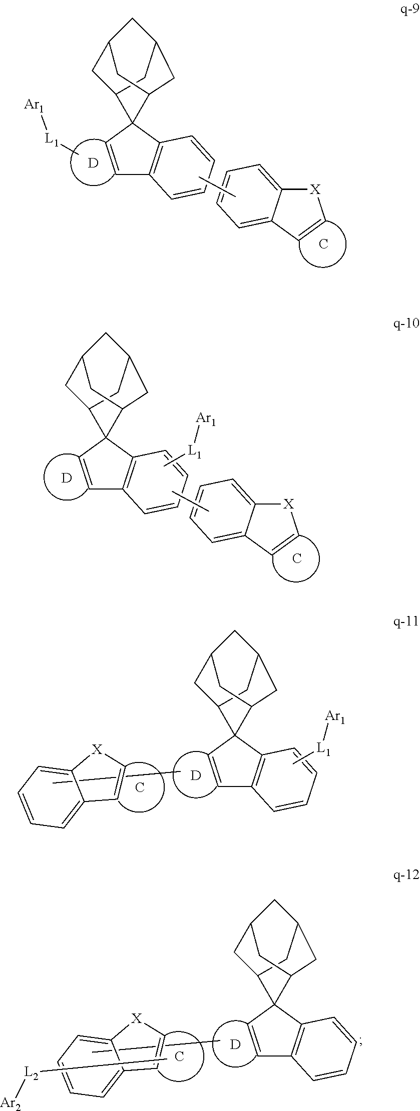

where the ring C and the ring D are naphthalene rings, and X, L₁, L₂, Ar₁ and Ar₂ are defined as described in the specification.

In an optional embodiment of the present disclosure, the L₁ is selected from a single bond, substituted or unsubstituted arylene with 6 to 20 carbon atoms, and substituted or unsubstituted heteroarylene with 3 to 20 carbon atoms.

In an optional embodiment of the present disclosure, the L₁ is selected from a single bond, substituted or unsubstituted arylene with 6 to 18 carbon atoms, and substituted or unsubstituted heteroarylene with 4 to 18 carbon atoms.

Further, in an optional embodiment of the present disclosure, substituents in the L₁ are the same as or different from each other, and are each independently selected from the group consisting of deuterium, fluorine, chlorine, bromine, cyano, alkyl with 1 to 4 carbon atoms, haloalkyl with 1 to 4 carbon atoms, trialkylsilyl with 3 to 9 carbon atoms, cycloalkyl with 5 to 7 carbon atoms, aryl with 6 to 15 carbon atoms, and heteroaryl with 3 to 12 carbon atoms.

Further, in an optional embodiment of the present disclosure, the substituents in the L₁ are the same as or different from each other, and are each independently selected from the group consisting of deuterium, fluorine, chlorine, cyano, methyl, ethyl, isopropyl, n-propyl, tert-butyl, methoxyl, ethoxy, trifluoromethyl, trimethylsilyl, phenyl and naphthyl.

In an optional embodiment of the present disclosure, the L₁ is selected from one of a single bond, substituted or unsubstituted phenylene, substituted or unsubstituted biphenylene, substituted or unsubstituted terphenylene, substituted or unsubstituted naphthylene, substituted or unsubstituted fluorenylene, substituted or unsubstituted phenanthrylene, substituted or unsubstituted anthrylene, substituted or unsubstituted pyrenylene, spiro[cyclopentane-1,9′-fluorenylene], spiro[cyclohexane-1,9′-fluorenylene], substituted or unsubstituted pyridylene, substituted or unsubstituted pyrimidylene, substituted or unsubstituted 9,9-dimethyl-9H-9-silafluorenylene, substituted or unsubstituted dibenzofurylene, substituted or unsubstituted dibenzothienylene, substituted or unsubstituted quinolylene, substituted or unsubstituted isoquinolylene, substituted or unsubstituted carbazolylene, substituted or unsubstituted phenanthrolinylene, and substituted or unsubstituted spirobifluorenylene, or is a subunit group formed by connecting two or three of the above subunits through a single bond; and substituents in the L₁ are the same as or different from each other, and are each independently selected from the group consisting of deuterium, fluorine, chlorine, cyano, methyl, ethyl, isopropyl, n-propyl, tert-butyl, methoxy, ethoxy, trifluoromethyl, trimethylsilyl, phenyl and naphthyl.

In an optional embodiment of the present disclosure, the L₁ is selected from a single bond, or is selected from the group consisting of groups shown in chemical formulae (j-1) to (j-12);

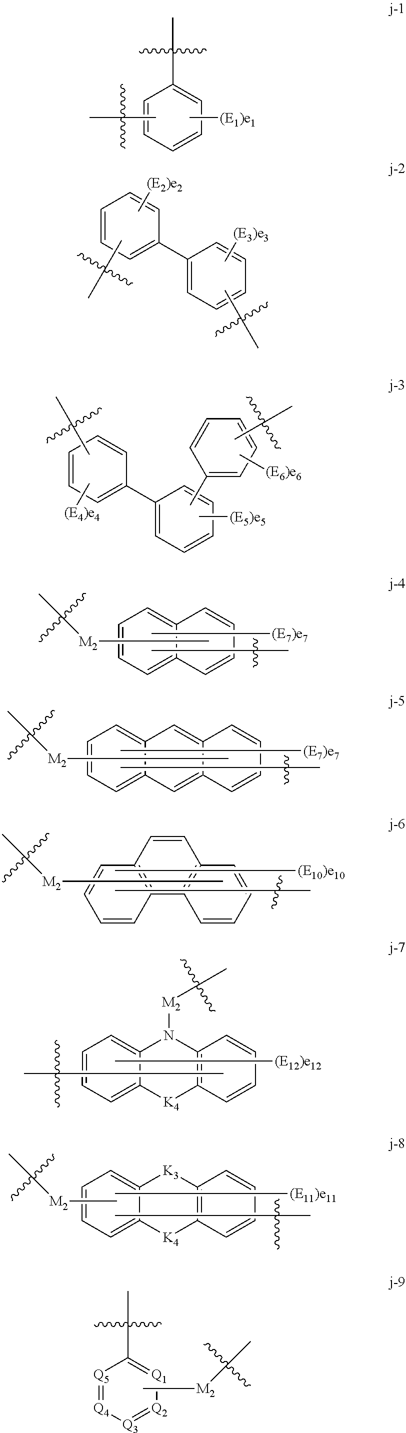

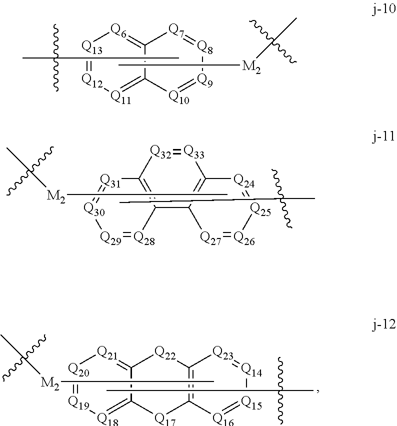

where M₂ is selected from a single bond or

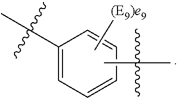

Q₁ to Q₅ are each independently selected from N or C(J₅), and at least one of Q₁ to Q₅ is selected from N; and when two or more of Q₁ to Q₅ are selected from C(J₅), any two J₅ are the same or different.

Q₆ to Q₁₃ are each independently selected from N or C(J₆), and at least one of Q₆ to Q₁₃ is selected from N; and when two or more of Q₆ to Q₁₃ are selected from C(J₆), any two J₆ are the same or different.

Q₁₄ to Q₂₃ are each independently selected from N, C or C(J₇), and at least one of Q₁₄ to Q₂₃ is selected from N; and when two or more of Q₁₄ to Q₂₃ are selected from C(J₇), any two J₇ are the same or different.

Q₂₄ to Q₃₃ are each independently selected from N, C or C(J₈), and at least one of Q₂₄ to Q₃₃ is selected from N; and when two or more of Q₂₄ to Q₃₃ are selected from C(J₈), any two J₈ are the same or different.

E₁ to E₁₂ and J₅ to J₈ are each selected from: hydrogen, deuterium, fluorine, chlorine, bromine, cyano, heteroaryl with 3 to 18 carbon atoms, aryl with 6 to 20 carbon atoms, trialkylsilyl with 3 to 9 carbon atoms, arylsilyl with 8 to 12 carbon atoms, alkyl with 1 to 10 carbon atoms, haloalkyl with 1 to 10 carbon atoms, cycloalkyl with 3 to 10 carbon atoms, heterocycloalkyl with 2 to 10 carbon atoms, alkoxy with 1 to 10 carbon atoms, alkylthio with 1 to 10 carbon atoms, aryloxy with 6 to 12 carbon atoms, or arylthio with 6 to 12 carbon atoms.

e₁ to e₁₂ are represented by eᵣ, E₁ to E₁₄ are represented by Eᵣ, r is a variable and represents any integer from 1 to 12, and eᵣ represents the number of a substituent Eᵣ; when r is selected from 1, 2, 3, 4, 5, 6 or 9, eᵣ is selected from 1, 2, 3 or 4; when r is selected from 7 or 11, eᵣ is selected from 1, 2, 3, 4, 5 or 6; when r is 12, eᵣ is selected from 1, 2, 3, 4, 5, 6 or 7; when r is selected from 8 or 10, eᵣ is selected from 1, 2, 3, 4, 5, 6, 7 or 8; and when eᵣ is greater than 1, any two Eᵣ are the same or different.

K₃ is selected from O, S, Se, N(E₁₅), C(E₁₆E₁₇) or Si(E₁₃E₁₄), where each of E₁₃, E₁₄, E₁₆ and E₁₇ is independently selected from: hydrogen, aryl with 6 to 20 carbon atoms, heteroaryl with 3 to 20 carbon atoms, and alkyl with 1 to 10 carbon atoms.

Or optionally, the E₁₆ and the E₁₇ are connected to each other to form a 5- to 13-membered aliphatic ring or a 5- to 13-membered aromatic ring together with the atoms to which they are commonly connected.

Or optionally, the E₁₃ and the E₁₄ are connected to each other to form a 5- to 13-membered aliphatic ring or a 5- to 13-membered aromatic ring together with the atoms to which they are jointly connected.

Each K₄ is independently selected from a single bond, O, S, Se, N(E₂₀), C(E₂₁E₂₂) or Si(E₁₈E₁₉), where each of E₂₀, E₂₁, E₂₂, E₁₈ and E₁₉ is independently selected from: hydrogen, aryl with 6 to 20 carbon atoms, heteroaryl with 3 to 20 carbon atoms, or alkyl with 1 to 10 carbon atoms.

Or optionally, the E₂₁ and the E₂₂ are connected to each other to form a 5- to 13-membered aliphatic ring or a 5- to 13-membered aromatic ring together with the atoms to which they are jointly connected.

Or optionally, the E₁₈ and the E₁₉ are connected to each other to form a 5- to 13-membered aliphatic ring or a 5- to 13-membered aromatic ring together with the atoms to which they are jointly connected.

For example, when L₁ is

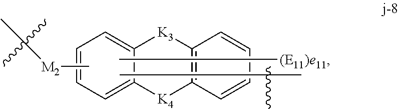

M₂ and K₄ are single bonds, each E₁₁ is hydrogen, and K₃ is C(E₁₆E₁₇), optionally, a situation that E₁₆ and E₁₇ connected to the same atom are connected to each other form a saturated or unsaturated 5- to 13-membered aliphatic ring together with the atoms to which they are jointly connected means that E₁₆ and E₁₇ may be connected to each other to form one 5- to 13-membered ring, or they may exist independently of each other; when E₁₆ and E₁₇ form the aliphatic ring, the number of atoms on the ring may be a 5-membered ring, for example

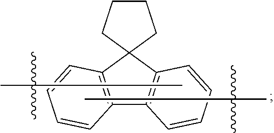

may also be a 6-membered ring, for example

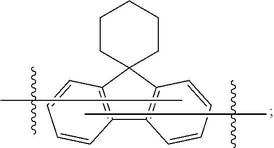

may further be a 10-membered ring, for example

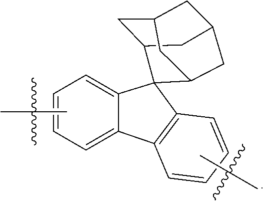

Certainly, the number of atoms on the ring formed by the interconnection of E₁₆ and E₁₇ may further be other values, which will not be listed one by one here. Meanwhile, the ring formed by the interconnection of E₁₆ and E₁₇ may further be an aromatic ring, such as a 13-membered aromatic ring,

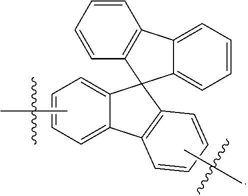

Optionally, the meaning that E₁₃ and E₁₄ are connected to each other to form a 5- to 13-membered aliphatic ring or aromatic ring together with the atoms to which they are jointly connected is the same as that of E₁₆ and E₁₇. Optionally, the meaning that E₂₁ and E₂₂ are connected to each other to form a 5- to 13-membered aliphatic ring or a 5- to 13-membered aromatic ring together with the atoms to which they are jointly connected is the same as that of E₁₆ and E₁₇. Optionally, the meaning that E₁₈ and E₁₉ are connected to each other to form a 5- to 13-membered aliphatic ring or a 5- to 13-membered aromatic ring together with the atoms to which they are jointly connected is the same as that of E₁₆ and E₁₇.

In an optional embodiment of the present disclosure, the L₁ is selected from a single bond, or substituted or unsubstituted W₁, where the unsubstituted W₁ is selected from the group consisting of the following groups:

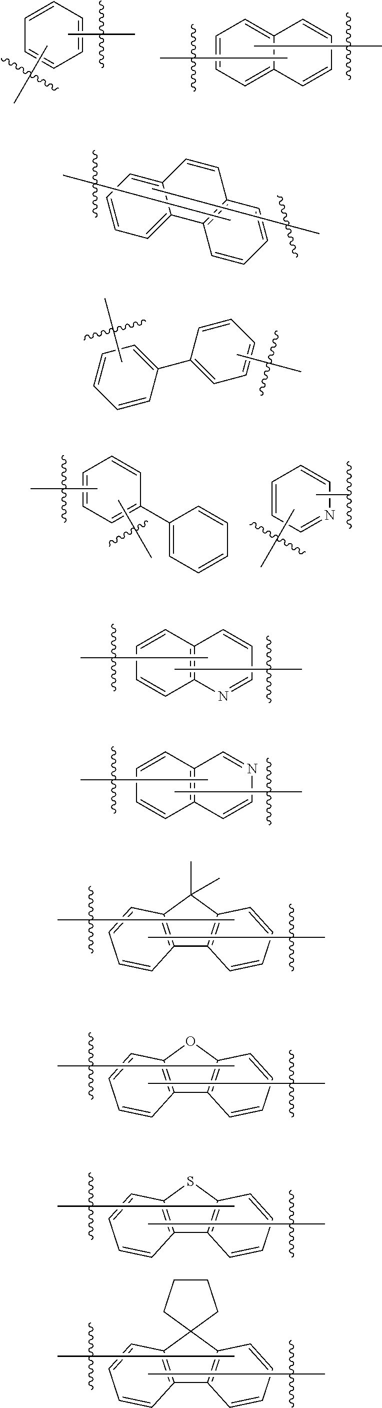

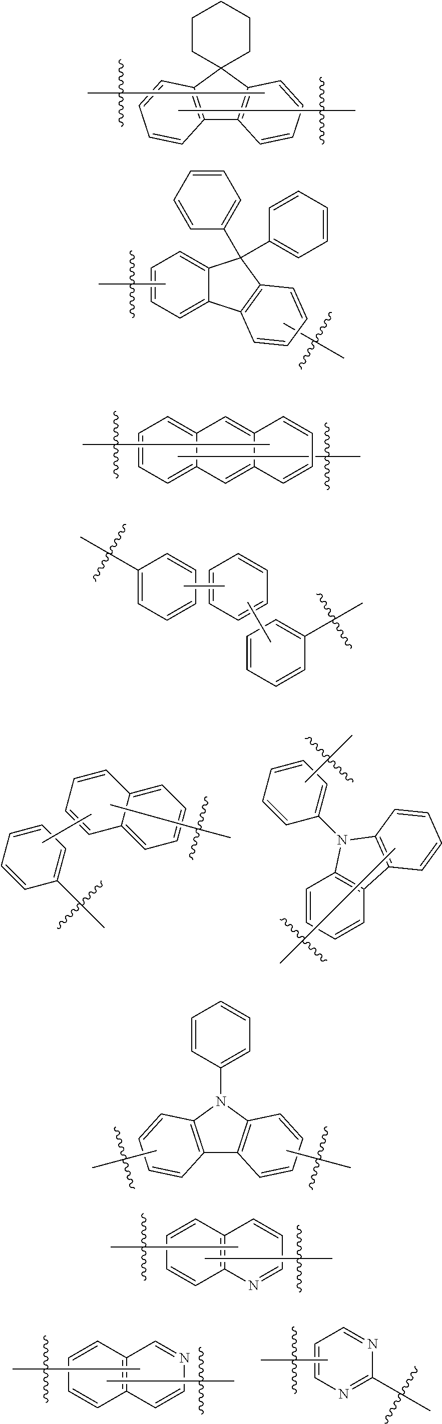

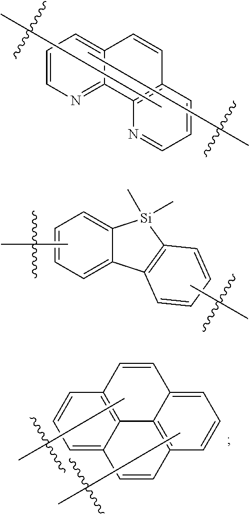

where the substituted W₁ is a group formed by substituting the unsubstituted W₁ with one or more substituents selected from deuterium, fluorine, chlorine, bromine, cyano, alkyl with 1 to 4 carbon atoms, haloalkyl with 1 to 4 carbon atoms, trialkylsilyl with 3 to 9 carbon atoms, cycloalkyl with 5 to 7 carbon atoms, aryl with 6 to 15 carbon atoms, or heteroaryl with 3 to 12 carbon atoms, and when there are a plurality of substituents on the substituted W₁, any two substituents are the same or different.

Further, in an optional embodiment of the present disclosure, the substituted W₁ is a group formed by substituting the unsubstituted W₁ with one or more substituents selected from deuterium, fluorine, chlorine, cyano, methyl, ethyl, isopropyl, n-propyl, tert-butyl, methoxyl, ethoxy, trifluoromethyl, trimethylsilyl, phenyl and naphthyl. In the present disclosure, “a plurality of” substituents mean more than one, which may be 2, 3, 4, 5, 6, 7 or 8.

In an optional embodiment of the present disclosure, the L₁ is selected from a single bond or the group consisting of the following groups:

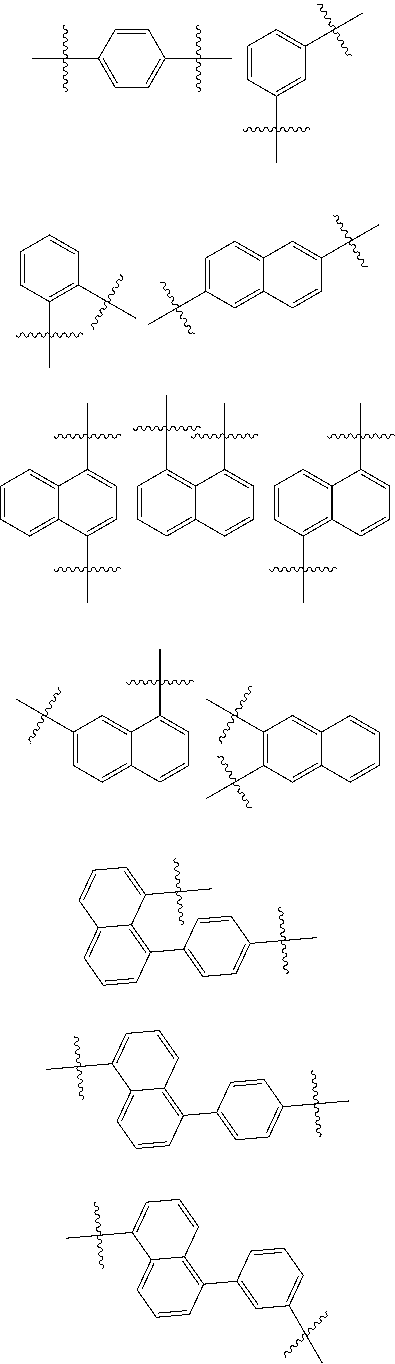

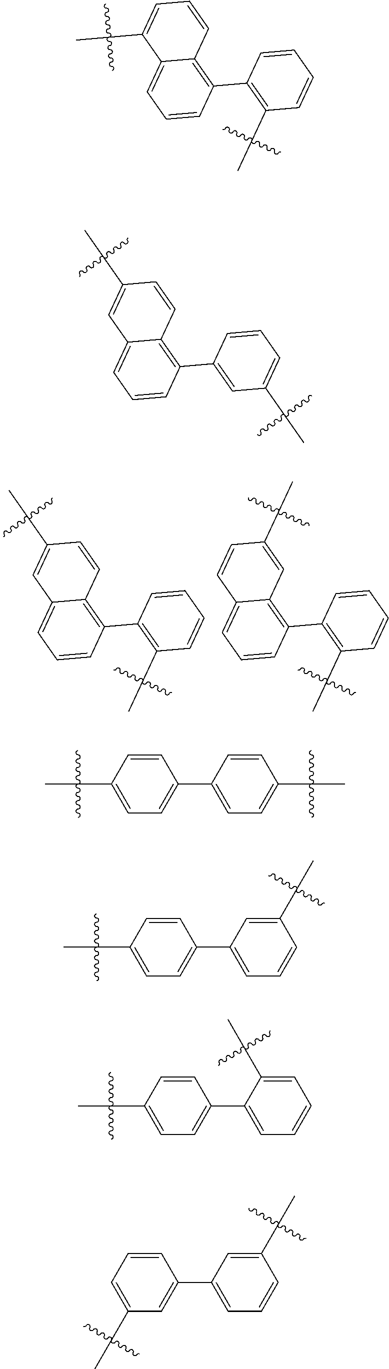

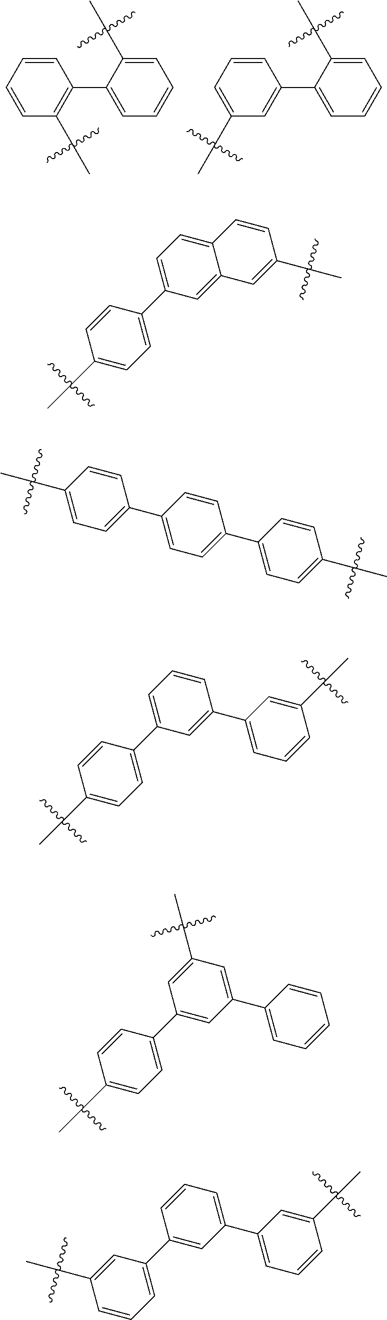

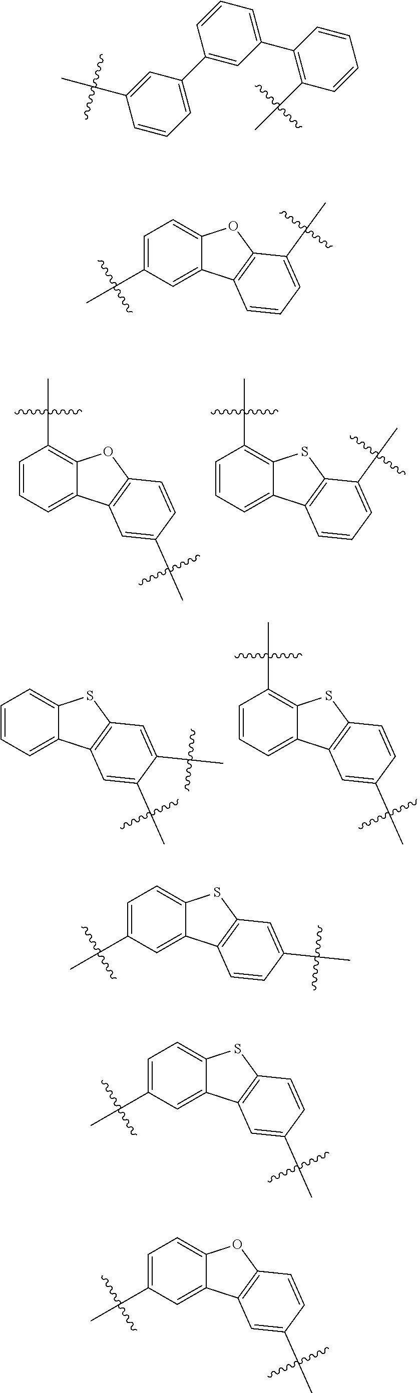

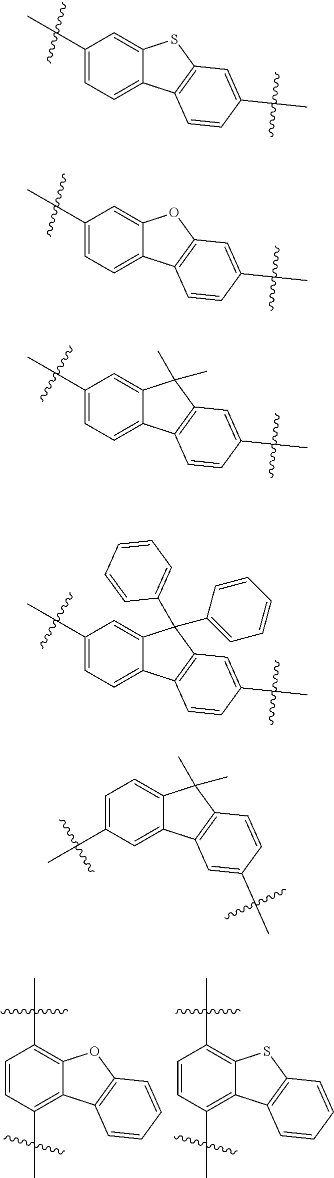

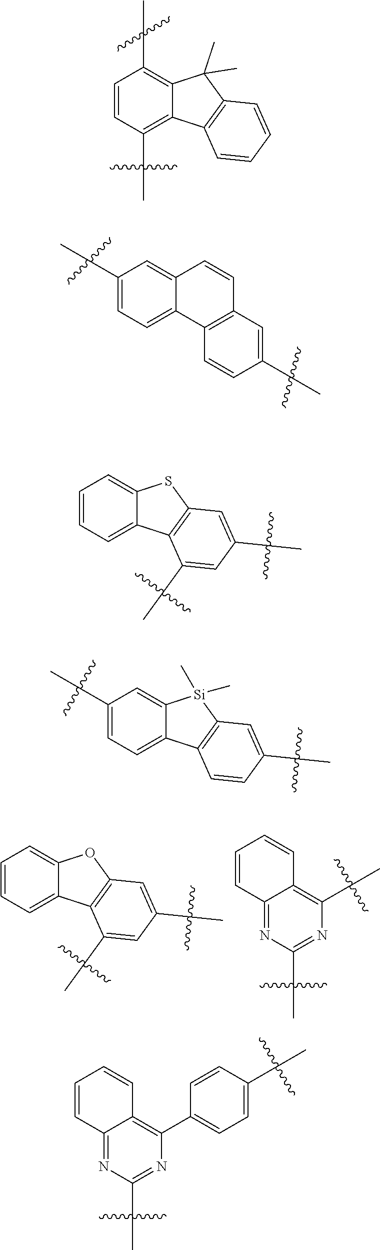

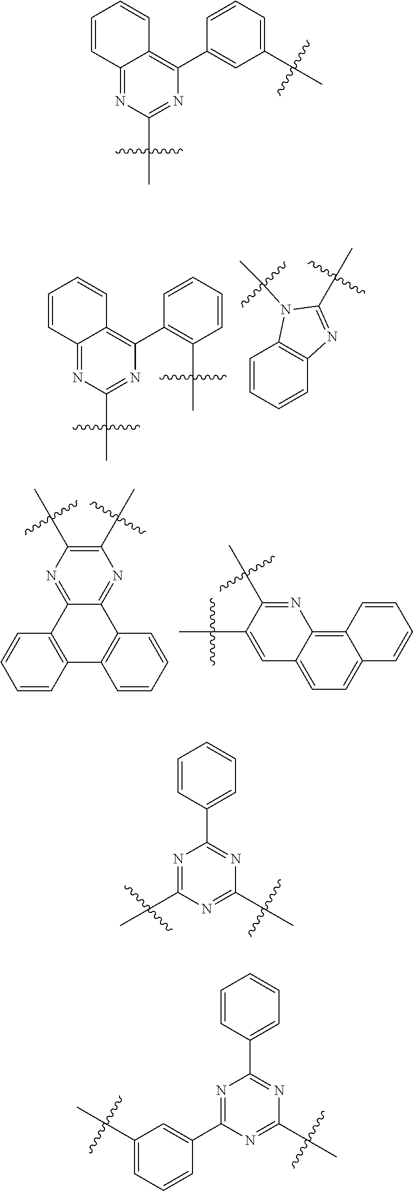

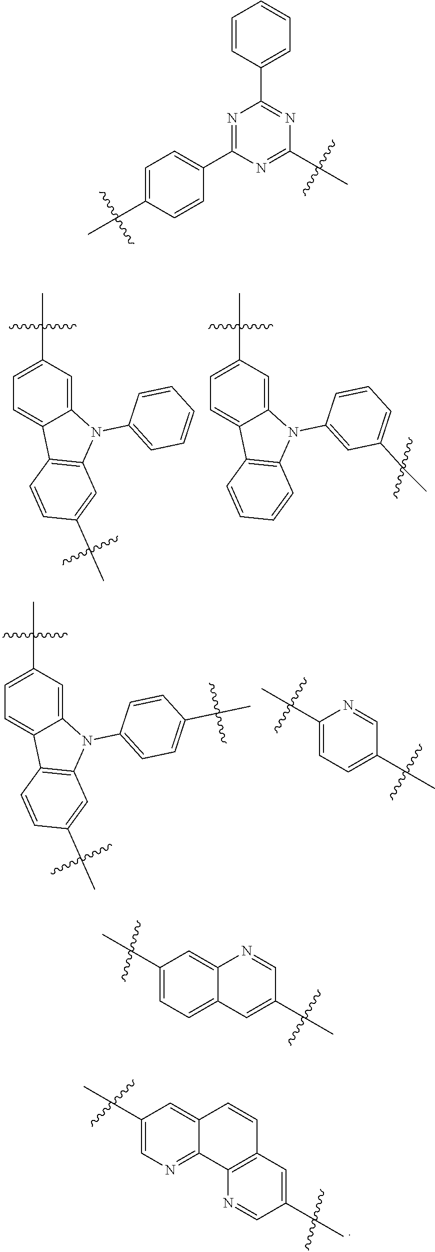

L₁ is not limited to the above groups.

In some embodiments of the present disclosure, Ar₁ is selected from the group consisting of groups shown in formulae (i-1) to (i-18);

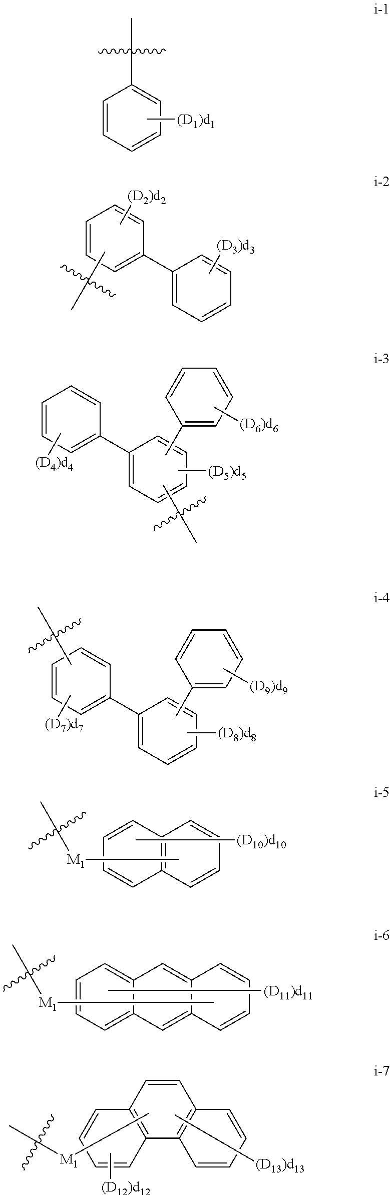

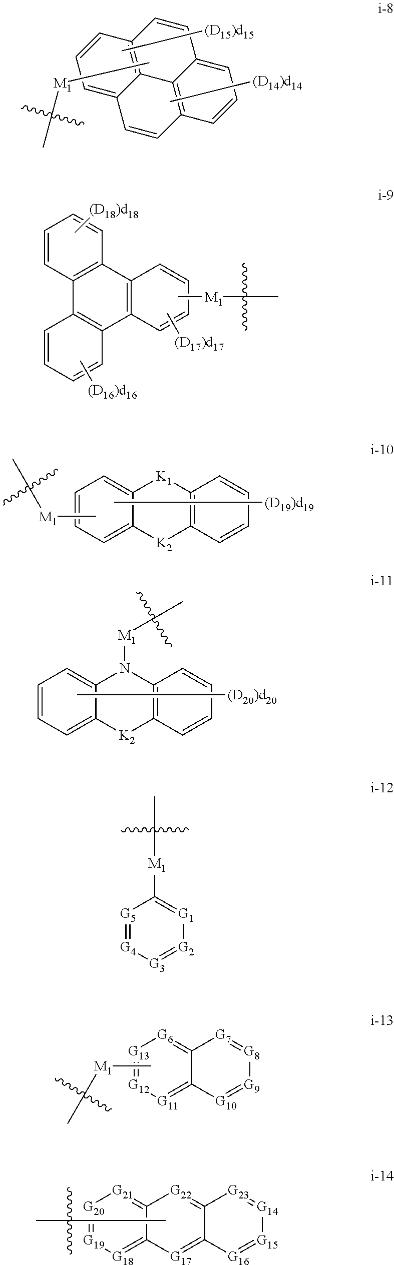

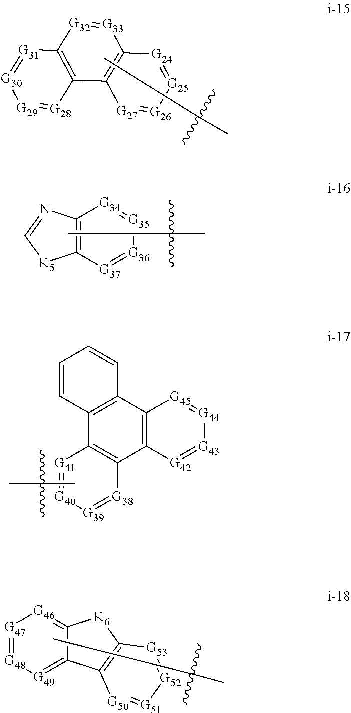

where M₁ is selected from a single bond or

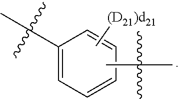

G₁ to G₅ are each independently selected from N or C(F₁), and at least one of G₁ to G₅ is selected from N; and when two or more of G₁ to G₅ are selected from C(F₁), any two F₁ are the same or different.

G₆ to G₁₃ are each independently selected from N or C(F₂), and at least one of G₆ to G₁₃ is selected from N; and when two or more of G₆ to G₁₃ are selected from C(F₂), any two F₂ are the same or different.

G₁₄ to G₂₃ are each independently selected from N or C(F₃), and at least one of G₁₄ to G₂₃ is selected from N; and when two or more of G₁₄ to G₂₃ are selected from C(F₃), any two F₃ are the same or different; or optionally, the adjacent two F₃ are connected to each other to form a 5- to 10-membered aromatic ring or a 5- to 10-membered heteroaromatic ring.

G₂₄ to G₃₃ are each independently selected from N or C(F₄), and at least one of G₂₄ to G₃₃ is selected from N; and when two or more of G₂₄ to G₃₃ are selected from C(F₄), any two F₄ are the same or different; or optionally, the adjacent two F₄ are connected to each other to form a 5- to 10-membered aromatic ring or a 5- to 10-membered heteroaromatic ring.

G₃₄ to G₃₇ are each independently selected from N or C(F₅), and when two or more of G₃₄ to G₃₇ are selected from C(F₅), any two F₅ are the same or different; or optionally, the adjacent two F₅ are connected to each other to form a 5- to 10-membered aromatic ring or a 5- to 10-membered heteroaromatic ring.

G₃₈ to G₄₅ are each independently selected from N or C(F₆), and at least one of G₃₈ to G₄₅ is selected from N; and when two or more of G₃₈ to G₄₅ are selected from C(F₆), any two F₆ are the same or different.

G₄₆ to G₅₃ are each independently selected from N or C(F₇), and at least one of G₄₆ to G₅₃ is selected from N; and when two or more of G₄₆ to G₅₃ are selected from C(F₇), any two F₇ are the same or different; or optionally, the adjacent two F₇ are connected to each other to form a 5- to 10-membered aromatic ring or a 5- to 10-membered heteroaromatic ring.

D₁ is selected from hydrogen, deuterium, fluorine, chlorine, bromine, cyano, trialkylsilyl with 3 to 12 carbon atoms, alkyl with 1 to 10 carbon atoms, haloalkyl with 1 to 10 carbon atoms, cycloalkyl with 3 to 10 carbon atoms, alkoxy with 1 to 10 carbon atoms, or alkylthio with 1 to 10 carbon atoms.

D₂ to D₉ and D₂₁ are each independently selected from: hydrogen, deuterium, fluorine, chlorine, bromine, cyano, trialkylsilyl with 3 to 12 carbon atoms, alkyl with 1 to 10 carbon atoms, haloalkyl with 1 to 10 carbon atoms, cycloalkyl with 3 to 10 carbon atoms, alkoxy with 1 to 10 carbon atoms, alkylthio with 1 to 10 carbon atoms, aryl with 3 to 20 carbon atoms, or heteroaryl with 3 to 18 carbon atoms.

D₁₀ to D₂₀ and F₁ to F₇ are each independently selected from: hydrogen, deuterium, fluorine, chlorine, bromine, cyano, trialkylsilyl with 3 to 12 carbon atoms, alkyl with 1 to 10 carbon atoms, haloalkyl with 1 to 10 carbon atoms, cycloalkyl with 3 to 10 carbon atoms, alkoxy with 1 to 10 carbon atoms, alkylthio with 1 to 10 carbon atoms, alkylsilyl with 3 to 9 carbon atoms, triphenylsilyl or a group B; the group B is selected from substituted or unsubstituted aryl with 6 to 25 carbon atoms, or substituted or unsubstituted heteroaryl with 3 to 18 carbon atoms; and substituents in the aryl or the heteroaryl are each independently selected from the group consisting of deuterium, fluorine, chlorine, cyano, alkyl with 1 to 6 carbon atoms, alkoxy with 1 to 6 carbon atoms, cycloalkyl with 3 to 7 atoms, aryl with 6 to 15 carbon atoms, heteroaryl with 3 to 12 carbon atoms, alkoxy with 1 to 4 carbon atoms, haloalkyl with 1 to 4 carbon atoms and alkylsilyl with 3 to 9 carbon atoms.

d₁ or d₂₁ are represented by dₖ, D₁ or D₂₁ are represented by Dₖ, k is a variable and represents any integer from 1 to 21, and dₖ represents the number of a substituent Dₖ; where when k is selected from 5 or 17, dₖ is selected from 1, 2 or 3; when k is selected from 2, 7, 8, 12, 15, 16, 18 or 21, dₖ is selected from 1, 2, 3 or 4; when k is selected from 1, 3, 4, 6, 9 or 14, dₖ is selected from 1, 2, 3, 4 or 5; when k is 13, dₖ is selected from 1, 2, 3, 4, 5 or 6; when k is selected from 10 or 19, dₖ is selected from 1, 2, 3, 4, 5, 6 or 7; when k is 20, dₖ is selected from 1, 2, 3, 4, 5, 6, 7 or 8; when k is 11, dₖ is selected from 1, 2, 3, 4, 5, 6, 7, 8 or 9; and when dₖ is greater than 1, any two Dₖ are the same or different.

K₁ and K₆ are each independently selected from O, S, N(D₂₂), C(D₂₃D₂₄) or Si(D₂₈D₂₉), where each of D₂₂, D₂₃, D₂₄, D₂₈ and D₂₉ is independently selected from: aryl with 6 to 18 carbon atoms, heteroaryl with 3 to 18 carbon atoms, alkyl with 1 to 10 carbon atoms or cycloalkyl with 3 to 10 carbon atoms.

Or optionally, the D₂₃ and the D₂₄ are connected to each other to form a 5- to 14-membered aliphatic ring or a 5- to 14-membered aromatic ring together with the atoms to which they are jointly connected.

Or optionally, the D₂₈ and the D₂₉ are connected to each other to form a 5- to 14-membered aliphatic ring or a 5- to 14-membered aromatic ring together with the atoms to which they are jointly connected.

K₂ is selected from a single bond, O, S, N(D₂₅), C(D₂₆D₂₇) or Si(D₃₀D₃₁), where each of D₂₅, D₂₆, D₂₇, D₃₀ and D₃₁ is independently selected from: aryl with 6 to 18 carbon atoms, heteroaryl with 3 to 18 carbon atoms, alkyl with 1 to 10 carbon atoms or cycloalkyl with 3 to 10 carbon atoms.

Or optionally, the D₂₆ and the D₂₇ are connected to each other to form a 5- to 14-membered aliphatic ring or a 5- to 14-membered aromatic ring together with the atoms to which they are jointly connected.

Or optionally, the D₃₀ and the D₃₁ are connected to each other to form a 5- to 14-membered aliphatic ring or a 5- to 14-membered aromatic ring together with the atoms to which they are jointly connected.

K₅ is selected from O, S, Se, N(D₃₂), and C(D₃₃D₃₄), where each of D₃₂, D₃₃ and D₃₄ is each independently selected from: aryl with 6 to 18 carbon atoms, heteroaryl with 3 to 18 carbon atoms, alkyl with 1 to 10 carbon atoms or cycloalkyl with 3 to 10 carbon atoms.

In the formula i-16, “optionally, the adjacent two F₅ are connected to each other to form the 5- to 10-membered aromatic ring or a 5- to 10-membered heteroaromatic ring” means that when any two adjacent cyclic atoms, G₃₄ and G₃₅ or G₃₅ and G₃₆ or G₃₆ and G₃₇ are both C(F₅), the adjacent two F₅ may exist independently of each other, or may be connected to each other to form a fused aromatic ring or heterocyclic atom together with the cyclic atom to which they are connected. For example, when in i-16

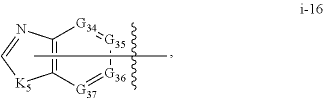

K₅ is O, G₃₄ and G₃₅ are both CH, G₃₆ and G₃₇ are both C(F₅) and they form a 6-membered aromatic ring, that is, the formula i-16 is

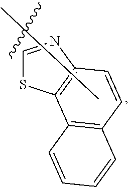

ring formation of the adjacent F₅ may further be to form other aromatic rings or heteroaromatic rings, which will not be listed one by one here. Optionally, the meaning that the adjacent two F₃, F₄ or F₇ are connected to each other to form the ring is the same as this, which will not be listed one by one.

“Optionally, the D₂₃ and the D₂₄ are connected to each other to form the 5- to 14-membered aliphatic ring or the 5- to 14-membered aromatic ring together with the atoms to which they are jointly connected” means that D₂₃ and D₂₄ may be connected to each other to form a ring, or may also exist independently of each other. For example, in the formula

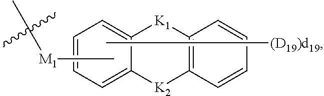

when M₁ is a single bond, K₂ is a single bond, each D₁₉ is hydrogen, and K₁ is C(D₂₃D₂₄), and when D₂₃ and D₂₄ form the ring, the ring may be a 5-membered aliphatic ring, for example

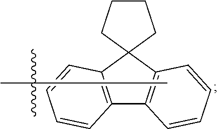

may also be a 6-membered aliphatic ring, for example

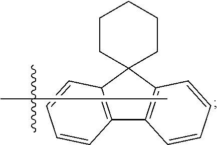

may also be a 13-membered aromatic ring, for example

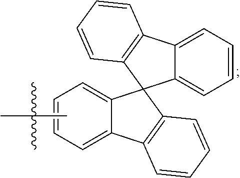

and may further be a 14-membered heteroaromatic ring, for example

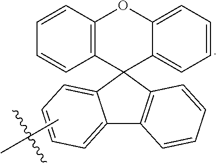

Certainly, the number of carbon atoms on the ring formed by the interconnection of D₂₃ and D₂₄ may further be other values, which will not be listed one by one here. Optionally, the meaning that D₂₆ and D₂₇ are connected to each other to form a 5- to 14-membered aliphatic ring or aromatic ring together with the atoms to which they are jointly connected is the same as that of D₂₃ and D₂₄. Optionally, the meaning that D₂₈ and D₂₉, D₂₆ and D₂₇, and D₃₀ and D₃₁ are connected to each other to form the 5- to 14-membered aliphatic ring or aromatic ring together with the atoms to which they are jointly connected is the same as that of D₂₃ and D₂₄.

Optionally, at least two of G₁ to G₅ are selected from N.

Optionally, at least two of G₆ to G₁₃ are selected from N.

Optionally, at least two of G₁₄ to G₂₃ are selected from N.

Optionally, at least two of G₂₄ to G₃₃ are selected from N.

Optionally, at least two of G₃₈ to G₄₅ are selected from N.

Optionally, at least two of G₄₆ to G₅₃ are selected from N.

In some specific embodiments of the present disclosure, Ar₁ is selected from substituted or unsubstituted aryl with 6, 10, 12, 14, 15, 16, 18, 20 and 25 carbon atoms, and substituted or unsubstituted heteroaryl with 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 and 36. In some embodiments of the present disclosure, An is selected from substituted or unsubstituted aryl with 6 to 18 carbon atoms, or substituted or unsubstituted heteroaryl with 15 to 26 carbon atoms.

Optionally, in some embodiments of the present disclosure, substituents in Ar₁ are one or more, are the same as or different from each other, and are each independently selected from deuterium, fluorine, chlorine, cyano, aryl with 6 to 20 carbon atoms, substituted or unsubstituted heteroaryl with 3 to 18 carbon atoms, alkyl with 1 to 6 carbon atoms, haloalkyl with 1 to 6 carbon atoms, alkoxy with 1 to 6 carbon atoms, alkylthio with 1 to 6 carbon atoms, cycloalkyl with 5 to 10 carbon atoms, trialkylsilyl with 3 to 9 carbon atoms, aryloxy with 6 to 20 carbon atoms, arylthio with 6 to 20 carbon atoms, or arylsilyl with 6 to 18 carbon atoms.

In some embodiments of the present disclosure, the An is selected from a substituted or unsubstituted group W₃, where the unsubstituted group W₃ is selected from the group consisting of the following groups:

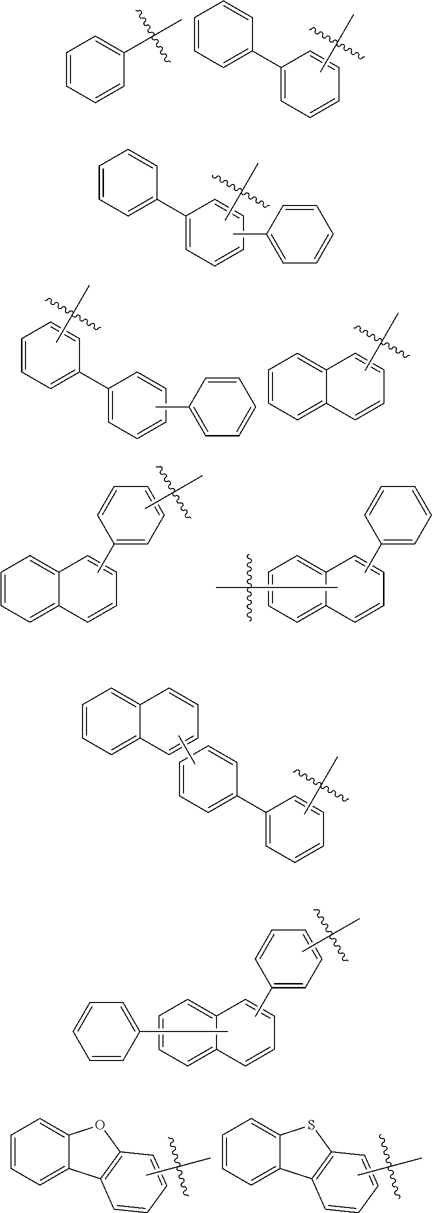

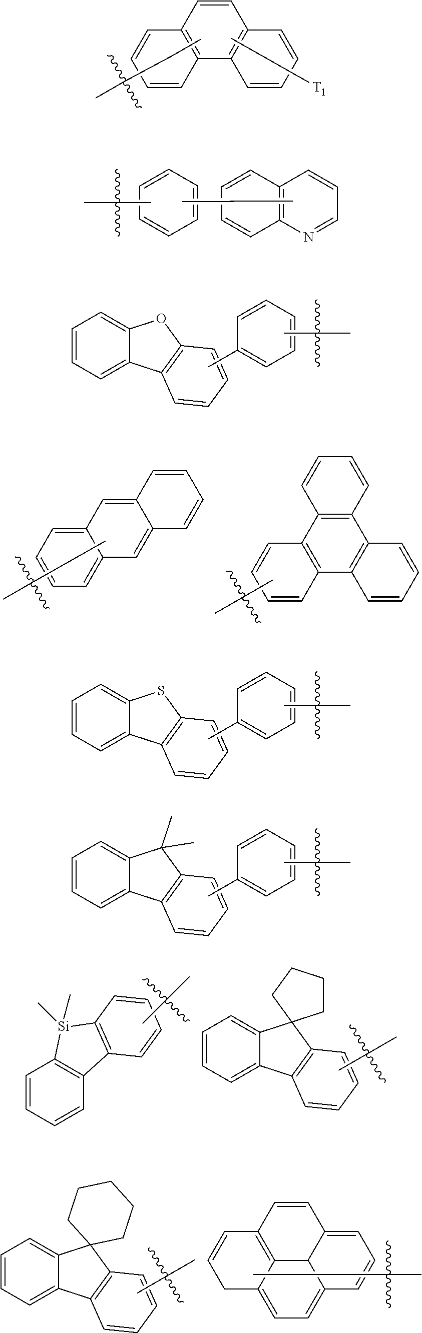

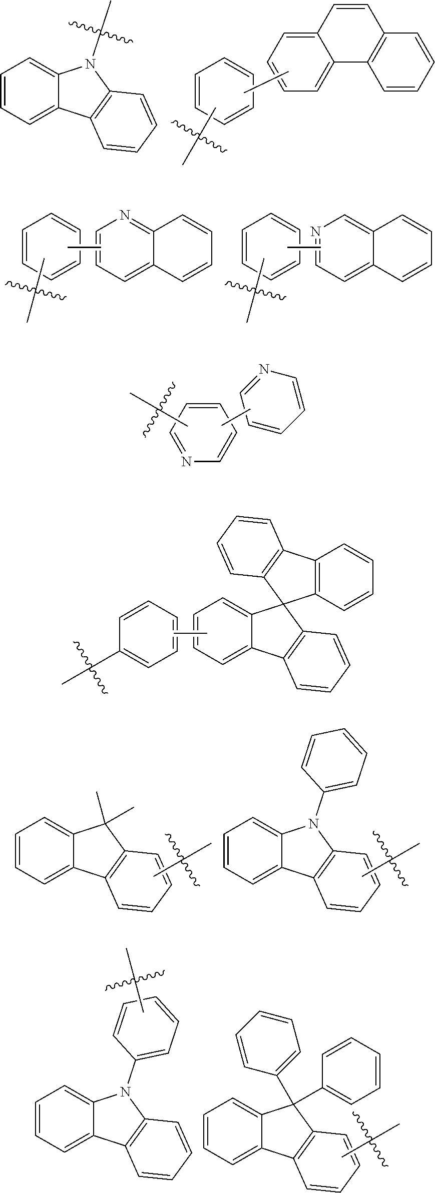

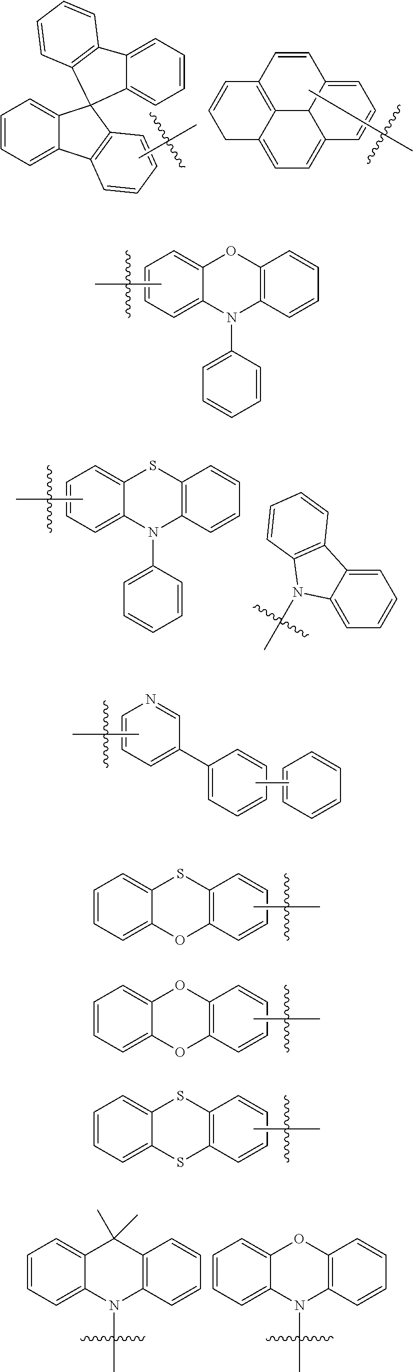

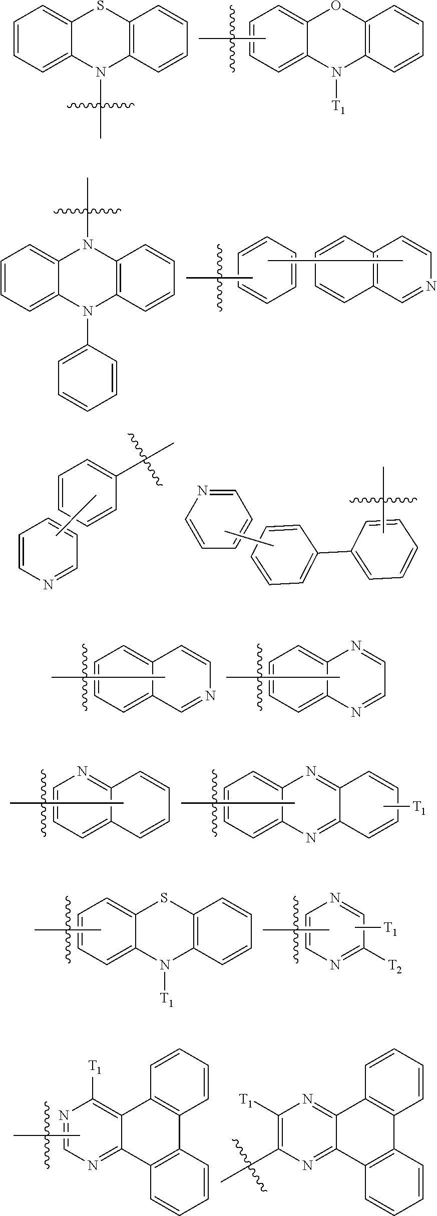

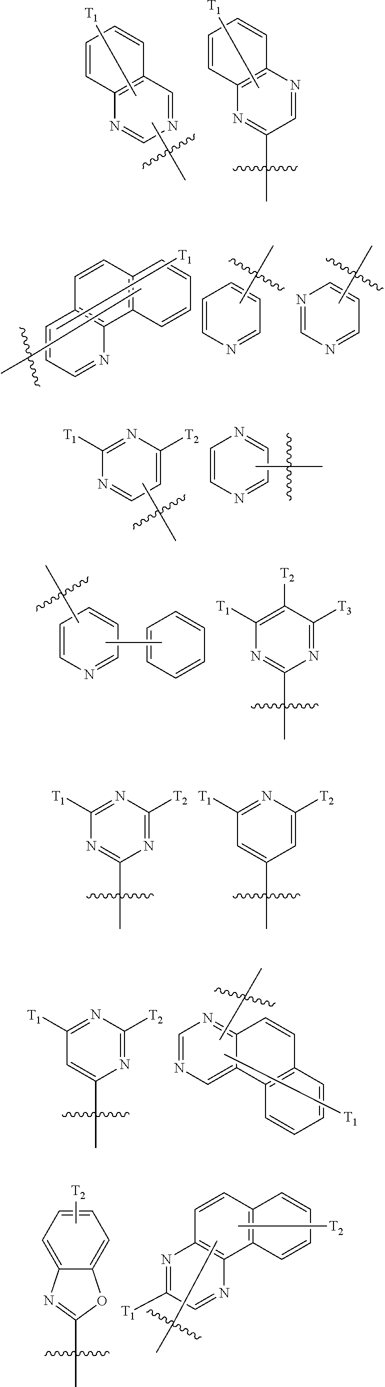

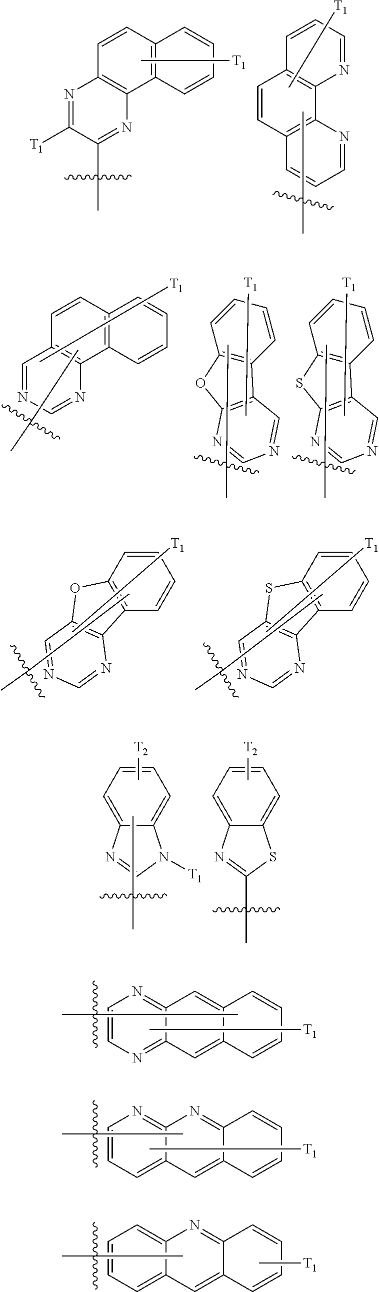

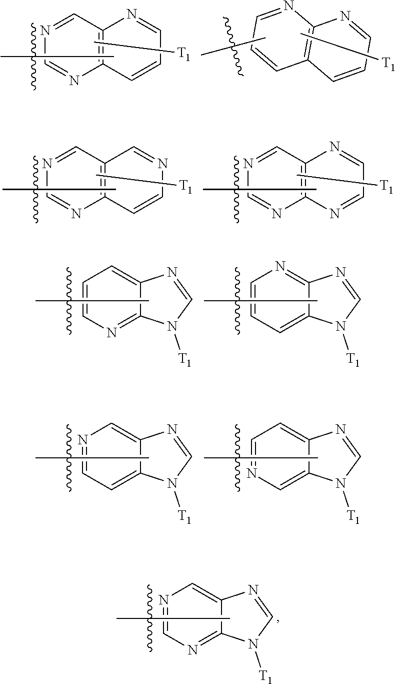

where each T₁, each T₂ and each T₃ are the same as or different from each other, and are each independently selected from hydrogen, aryl with 6 to 20 carbon atoms, and heteroaryl with 3 to 20 carbon atoms; and any two T₁ are the same or different, any two T₂ are the same or different, and any two T₃ are the same or different; and

where the substituted group W₃ is a group formed by substituting unsubstituted group W₃ with one or more substituents selected from deuterium, fluorine, chlorine, cyano, alkyl with 1 to 4 carbon atoms, alkoxy with 1 to 4 carbon atoms, cycloalkyl with 3 to 7 carbon atoms, aryl with 6 to 15 carbon atoms, heteroaryl with 3 to 18 carbon atoms, alkylthio with 1 to 4 carbon atoms, haloalkyl with 1 to 4 carbon atoms, or alkylsilyl with 3 to 9 carbon atoms; and when there are a plurality of substituents on the W₃, any two substituents are the same or different.

It should be noted that “substituted group W₃” means that the unsubstituted group W₃ is substituted with one or more substituents, and the substituents may replace hydrogen atoms at any position in the above unsubstituted group W₃, for example, it may also be any hydrogen atom in T₁, T₂ and T₃.

It should further be noted that, in the above group W₃, when the T₁, the T₂ or the T₃ is hydrogen, an unpositioned connecting bond may also be connected to group W₃ instead of the T₁, the T₂ or the T₃. For example,

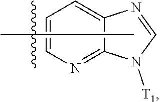

when the T₁ is hydrogen,

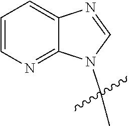 [SMILES: *n1cnc2cccnc21]

may also be represented.

Further, each T₁, each T₂ and each T₃ are the same as or different from each other, and are each independently selected from: hydrogen, phenyl, naphthyl, anthryl, phenanthryl, diphenyl, terphenyl, fluorenyl, dibenzothienyl, dibenzofuranyl, N-phenylcarbazolyl, carbazol-9-yl-phenyl, pyridyl, pyrimidinyl, pyrazinyl, quinolyl, isoquinolyl, quinazolinyl, benzoxazine, triphenylene or phenanthrolinyl; the substituted group W₃ is a group formed by substituting the unsubstituted group W₃ with one or more substituents selected from: fluorine, deuterium, cyano, trifluoromethyl, trimethylsilyl, methyl, ethyl, isopropyl, tert-butyl, methoxy, ethoxy, isopropoxy, cyclopentyl, cyclohexyl, phenyl, biphenyl, naphthyl, fluorenyl, 9,9-dimethylfluorenyl, pyridyl, pyrimidinyl, quinolyl, isoquinolyl, carbazolyl, N-phenylcarbazolyl, dibenzofuranyl, or dibenzothienyl; and when there are a plurality of substituents on the W₃, any two substituents are the same or different.

In some embodiments of the present disclosure, An may be selected from electron-deficient heteroaryl (also known as electron-poor heteroaryl), and heteroatoms on it can reduce the electron cloud density of a conjugated system of the heteroaryl as a whole instead of improving the electron cloud density of the conjugated system of the heteroaryl. For example, lone pair electrons on the heteroatoms do not participate in the conjugated system, and the electron cloud density of the conjugated system is reduced due to the strong electronegativity of the heteroatoms. For example, the electron-deficient heteroaryl may include, but is not limited to, pyridyl, pyrimidinyl, sym-triazinyl, quinolyl, isoquinolyl, benzopyrazolyl, benzimidazolyl, quinoxalinyl, phenanthrolyl and the like. In this way, the Ar₁ may form an electron transport core group of the compound, so that the compound can effectively achieve electron transport, and can effectively balance a transport rate of the electrons and the holes in the organic luminescent layer. In this way, the compound can serve as a bipolar host material of a organic luminescent layer to simultaneously transport the electrons and the holes, and may also serve as an electron-type host material of an organic luminescent layer to be combined with a hole-type host material of a organic luminescent layer. In this type of compound, the dibenzo five-membered ring with a hole transport ability and the electron-deficient heteroaryl (Ar₁) with an electron transport ability are respectively connected with a core structure of adamantyl spirofluorene through single bonds, which is more conducive to balance between the electron transport performance and the hole transport performance of the compound. In addition, the compound connected in this connection mode has a high T₁ energy level, has improved tolerance to excitons, and is more suitable as the host material of the luminescent layer.

In some more specific embodiments of the present disclosure, the Ar₁ is selected from the following structures:

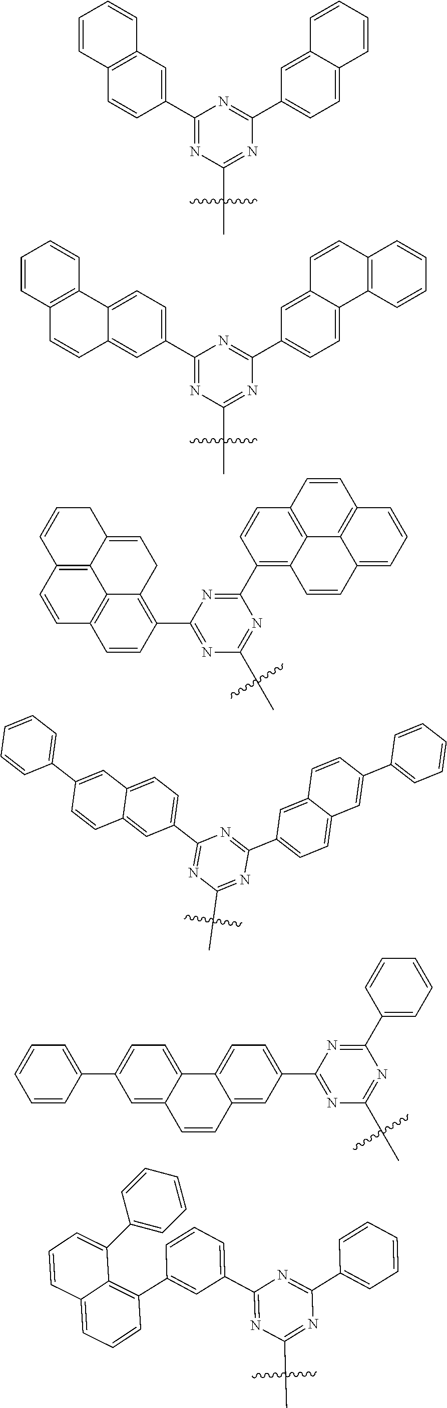

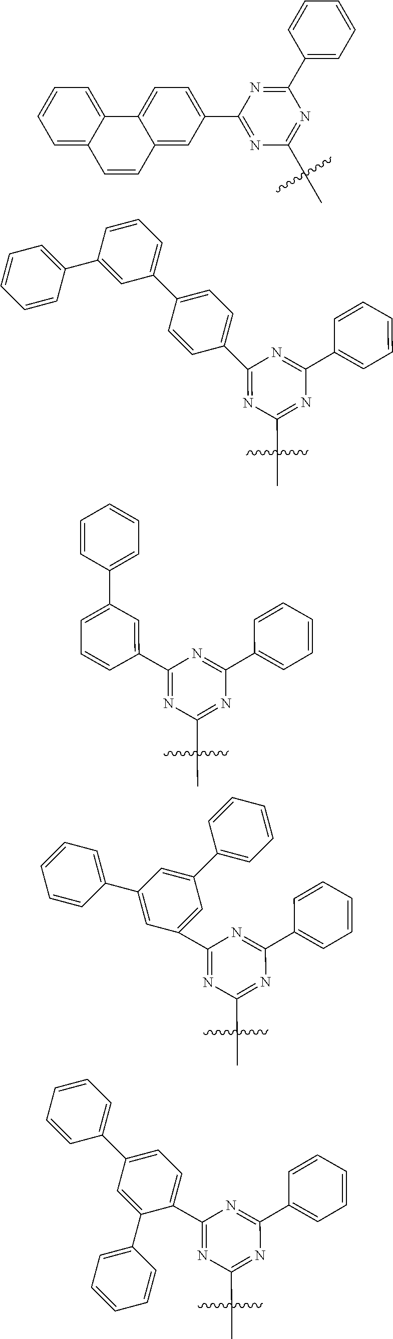

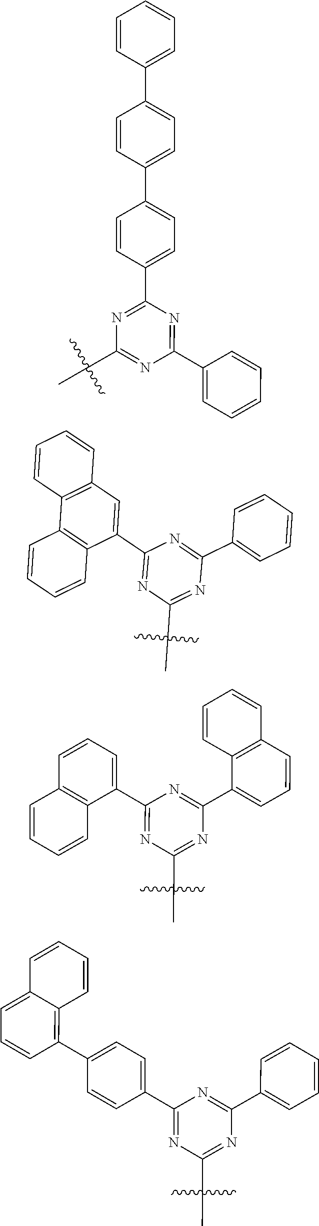 [SMILES: *c1nc(-c2ccccc2)nc(-c2ccc(-c3ccc(-c4ccccc4)cc3)cc2)n1 ; *c1nc(-c2ccccc2)nc(-c2cc3ccccc3c3ccccc23)n1 ; *c1nc(-c2cccc3ccccc23)nc(-c2cccc3ccccc23)n1 ; *c1nc(-c2ccccc2)nc(-c2ccc(-c3cccc4ccccc34)cc2)n1]

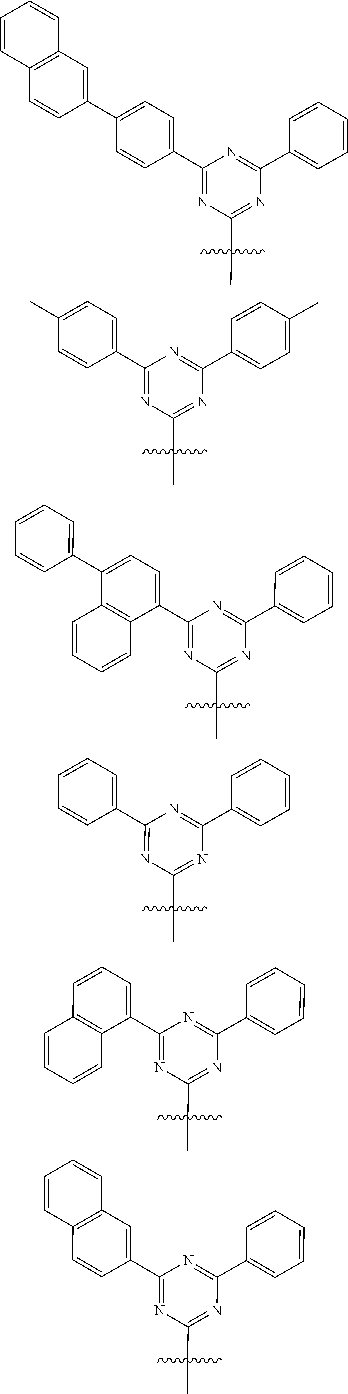

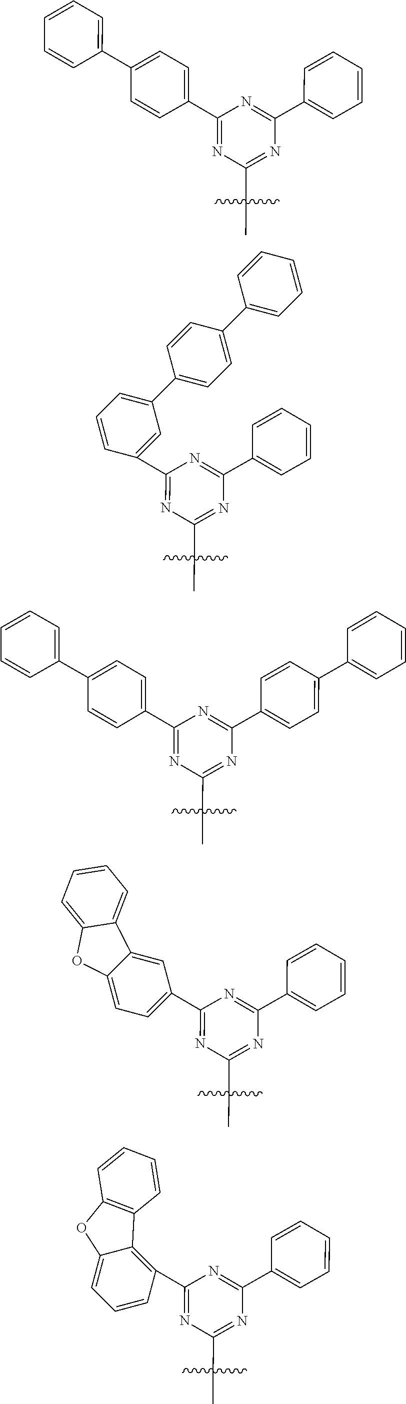 [SMILES: *c1nc(-c2ccccc2)nc(-c2ccc(-c3ccccc3)cc2)n1 ; *c1nc(-c2ccccc2)nc(-c2cccc(-c3ccc(-c4ccccc4)cc3)c2)n1 ; *c1nc(-c2ccc(-c3ccccc3)cc2)nc(-c2ccc(-c3ccccc3)cc2)n1 ; *c1nc(-c2ccccc2)nc(-c2ccc3oc4ccccc4c3c2)n1 ; *c1nc(-c2ccccc2)nc(-c2cccc3oc4ccccc4c23)n1]

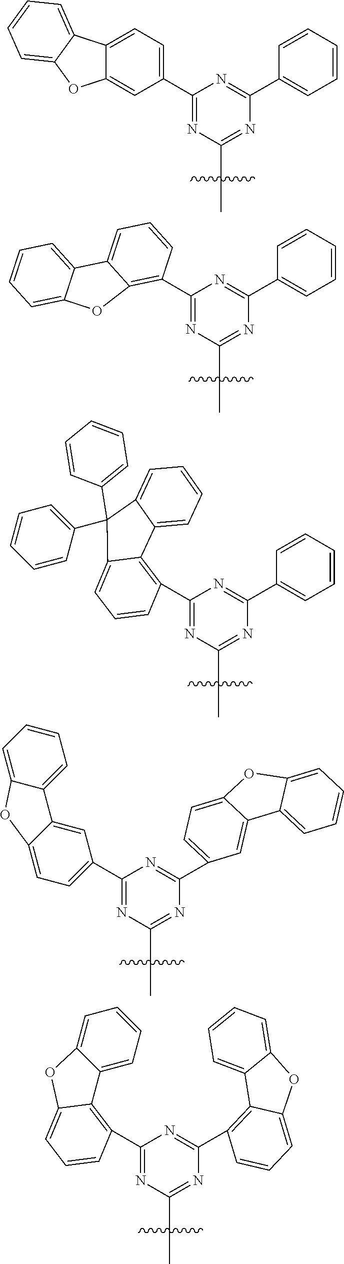 [SMILES: *c1nc(-c2ccccc2)nc(-c2ccc3c(c2)oc2ccccc23)n1 ; *c1nc(-c2ccccc2)nc(-c2cccc3c2oc2ccccc23)n1 ; *c1nc(-c2ccccc2)nc(-c2cccc3c2-c2ccccc2C3(c2ccccc2)c2ccccc2)n1 ; *c1nc(-c2ccc3oc4ccccc4c3c2)nc(-c2ccc3oc4ccccc4c3c2)n1 ; *c1nc(-c2cccc3oc4ccccc4c23)nc(-c2cccc3oc4ccccc4c23)n1]

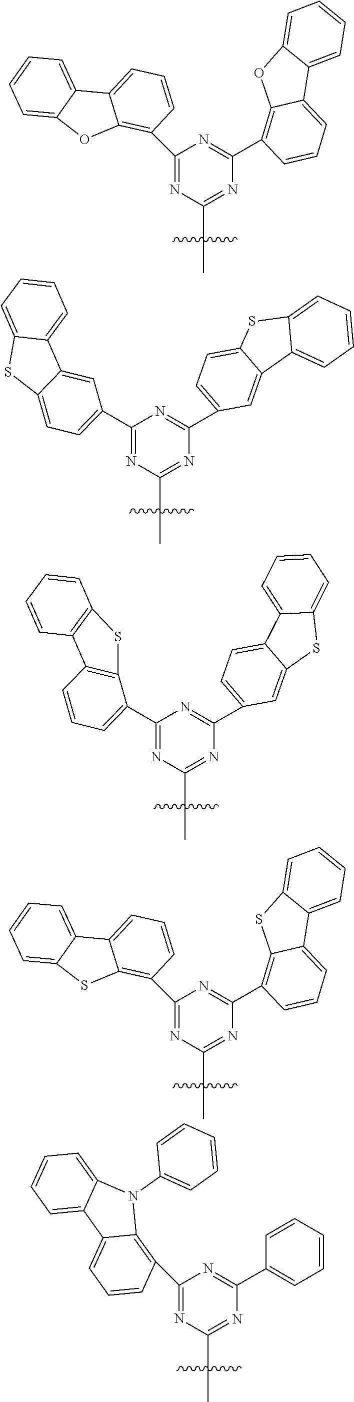

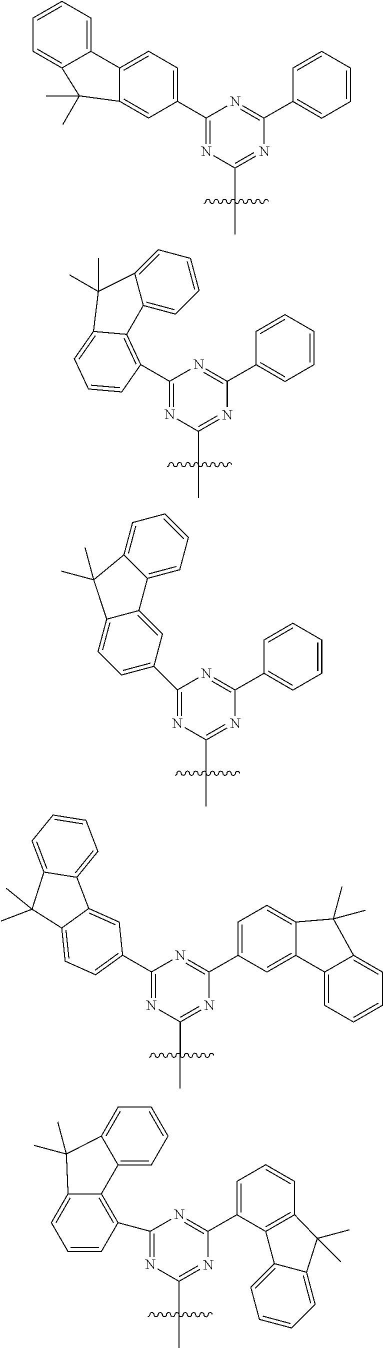

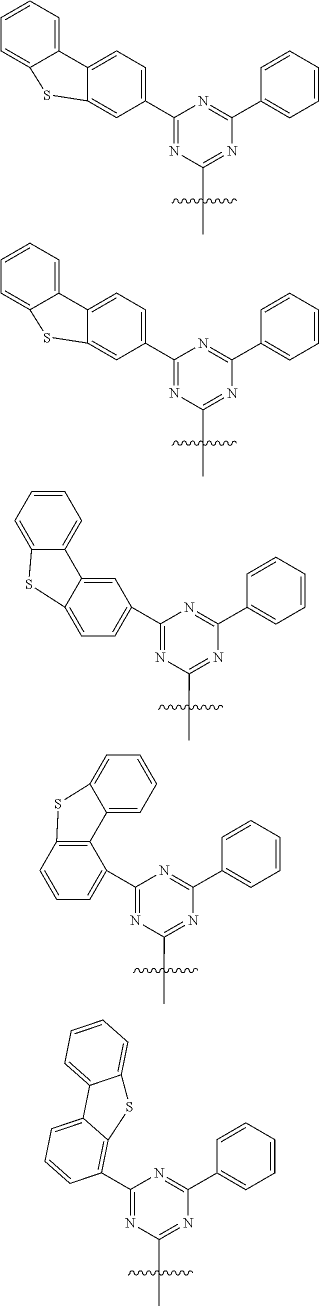 [SMILES: *c1nc(-c2ccccc2)nc(-c2ccc3c(c2)sc2ccccc23)n1 ; *c1nc(-c2ccccc2)nc(-c2ccc3c(c2)sc2ccccc23)n1 ; *c1nc(-c2ccccc2)nc(-c2ccc3sc4ccccc4c3c2)n1 ; *c1nc(-c2ccccc2)nc(-c2cccc3sc4ccccc4c23)n1 ; *c1nc(-c2ccccc2)nc(-c2cccc3c2sc2ccccc23)n1]

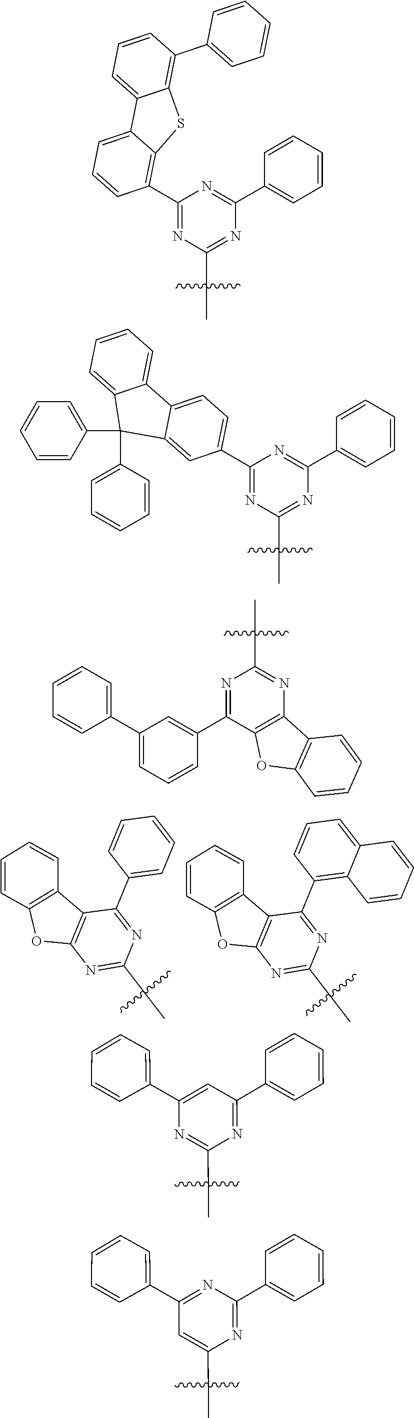

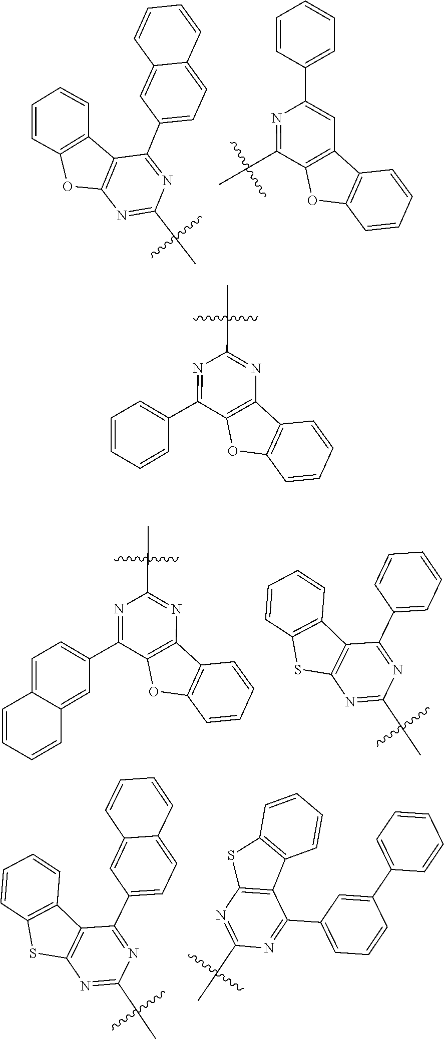

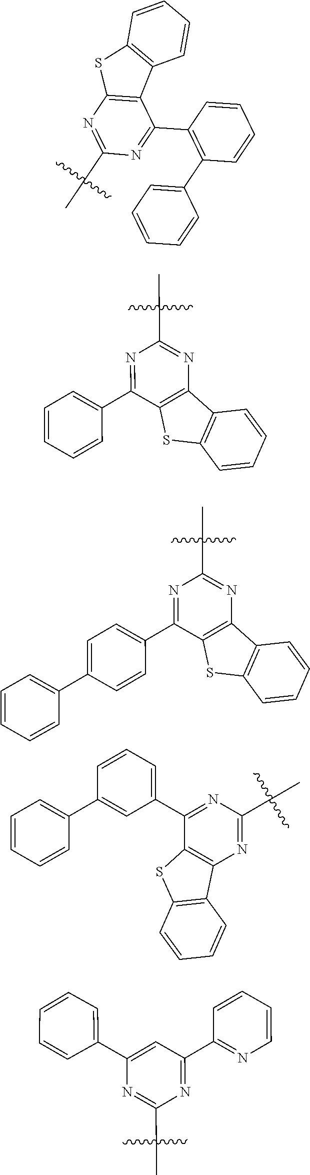

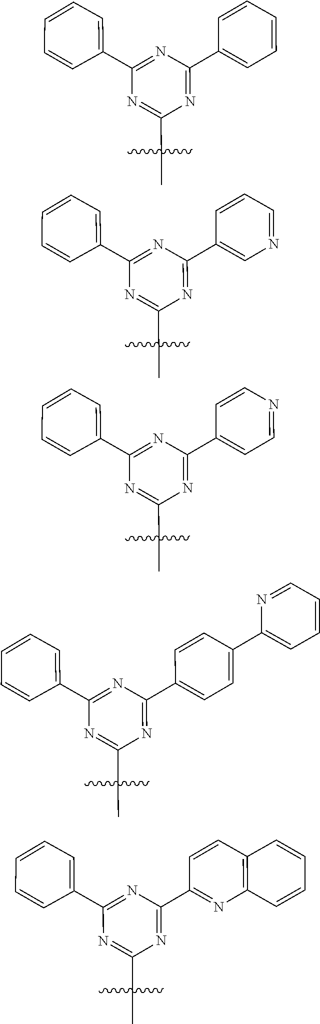 [SMILES: *c1nc(-c2ccccc2)nc(-c2ccccc2)n1 ; *c1nc(-c2ccccc2)nc(-c2cccnc2)n1 ; *c1nc(-c2ccccc2)nc(-c2ccncc2)n1 ; *c1nc(-c2ccccc2)nc(-c2ccc(-c3ccccn3)cc2)n1 ; *c1nc(-c2ccccc2)nc(-c2ccc3ccccc3n2)n1]

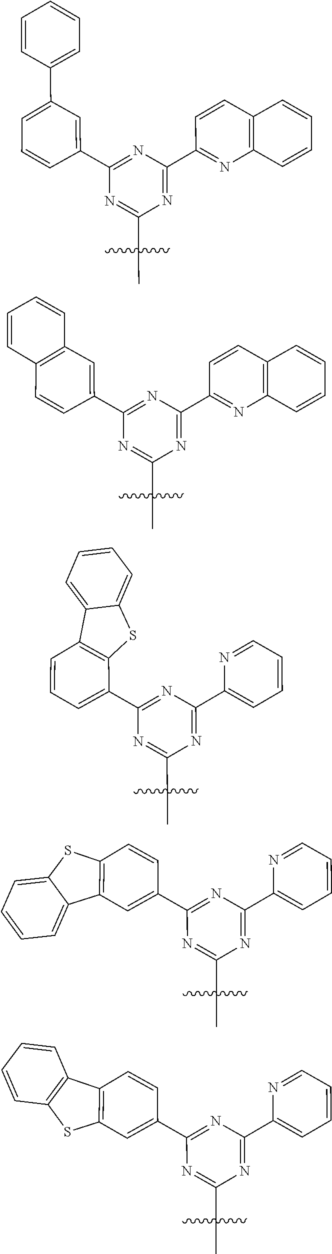 [SMILES: *c1nc(-c2cccc(-c3ccccc3)c2)nc(-c2ccc3ccccc3n2)n1 ; *c1nc(-c2ccc3ccccc3c2)nc(-c2ccc3ccccc3n2)n1 ; *c1nc(-c2ccccn2)nc(-c2cccc3c2sc2ccccc23)n1 ; *c1nc(-c2ccc3sc4ccccc4c3c2)nc(-c2ccccn2)n1 ; *c1nc(-c2ccc3c(c2)sc2ccccc23)nc(-c2ccccn2)n1]

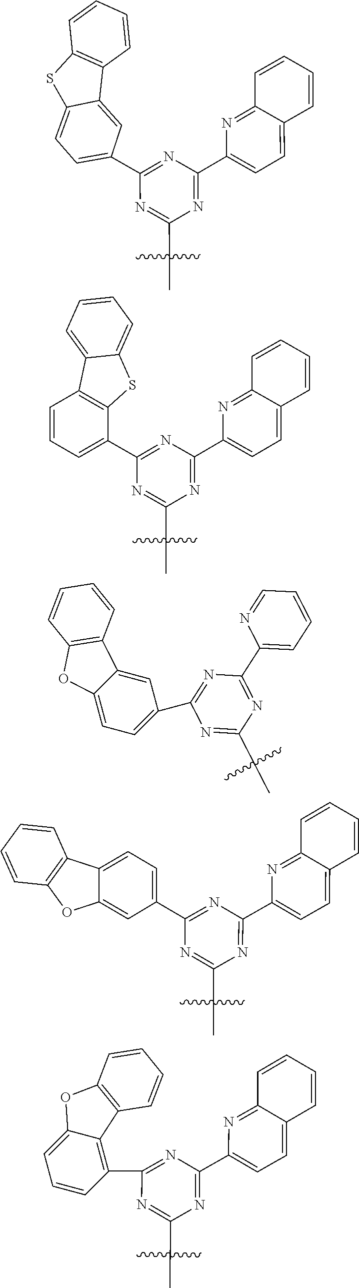 [SMILES: *c1nc(-c2ccc3sc4ccccc4c3c2)nc(-c2ccc3ccccc3n2)n1 ; *c1nc(-c2ccc3ccccc3n2)nc(-c2cccc3c2sc2ccccc23)n1 ; *c1nc(-c2ccc3oc4ccccc4c3c2)nc(-c2ccccn2)n1 ; *c1nc(-c2ccc3c(c2)oc2ccccc23)nc(-c2ccc3ccccc3n2)n1 ; *c1nc(-c2ccc3ccccc3n2)nc(-c2cccc3oc4ccccc4c23)n1]

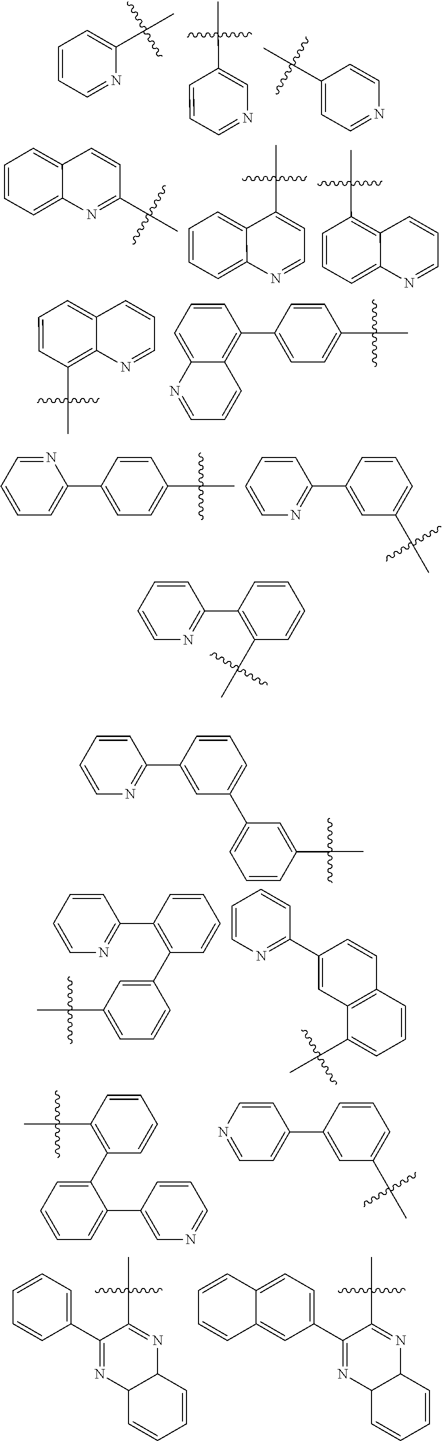

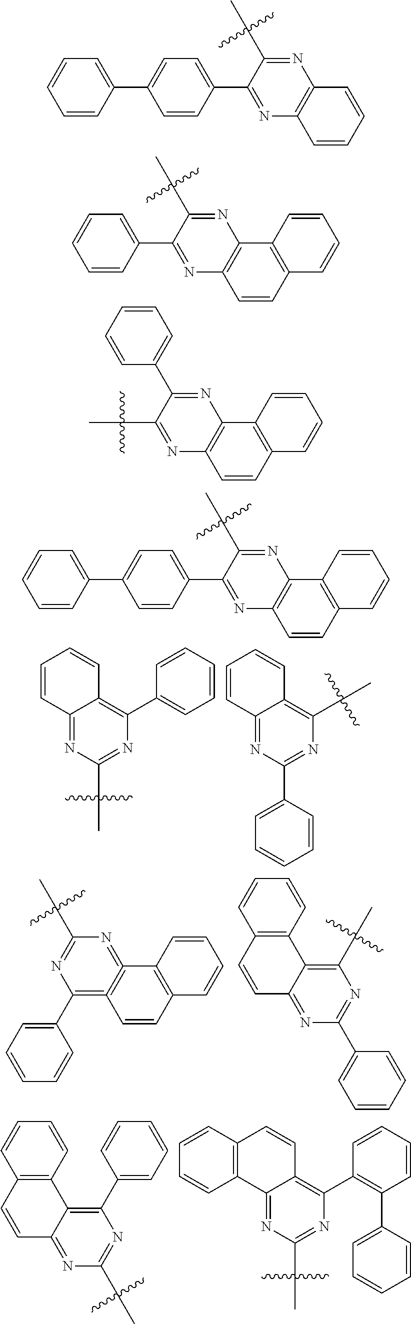

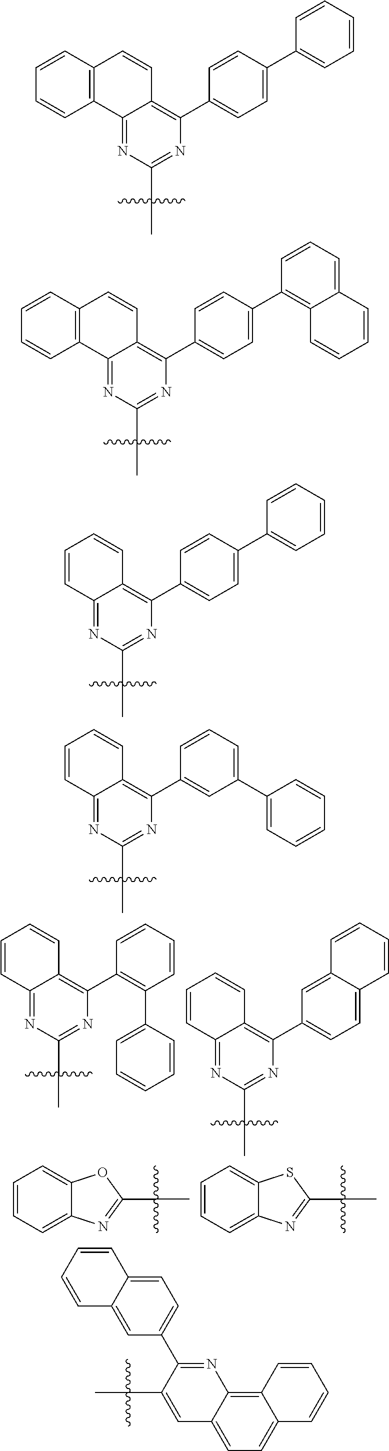

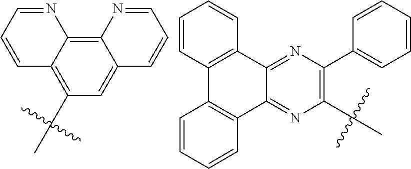

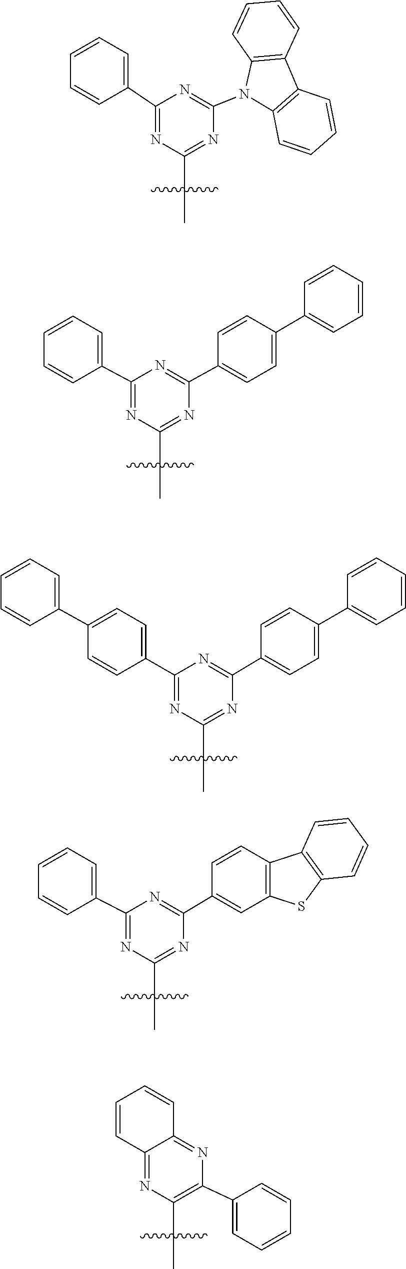 [SMILES: *c1nc(-c2ccccc2)nc(-n2c3ccccc3c3ccccc32)n1 ; *c1nc(-c2ccccc2)nc(-c2ccc(-c3ccccc3)cc2)n1 ; *c1nc(-c2ccc(-c3ccccc3)cc2)nc(-c2ccc(-c3ccccc3)cc2)n1 ; *c1nc(-c2ccccc2)nc(-c2ccc3c(c2)sc2ccccc23)n1 ; *c1nc2ccccc2nc1-c1ccccc1]

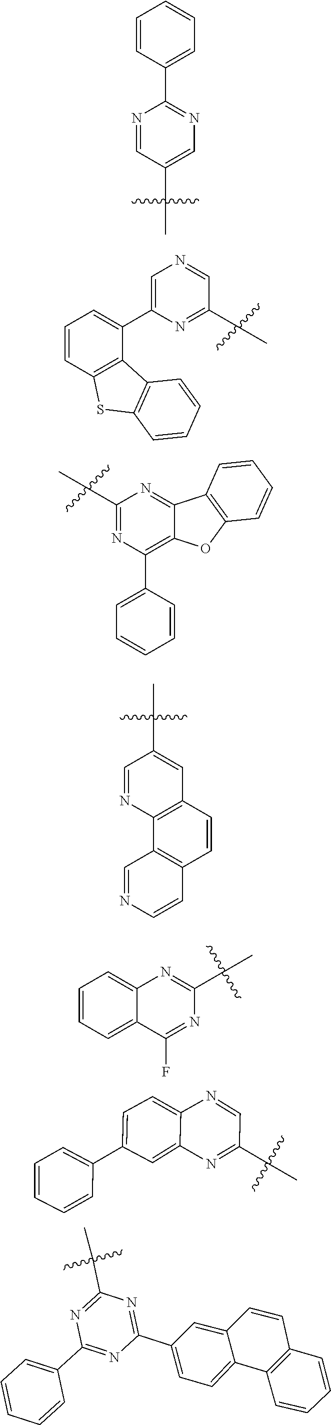

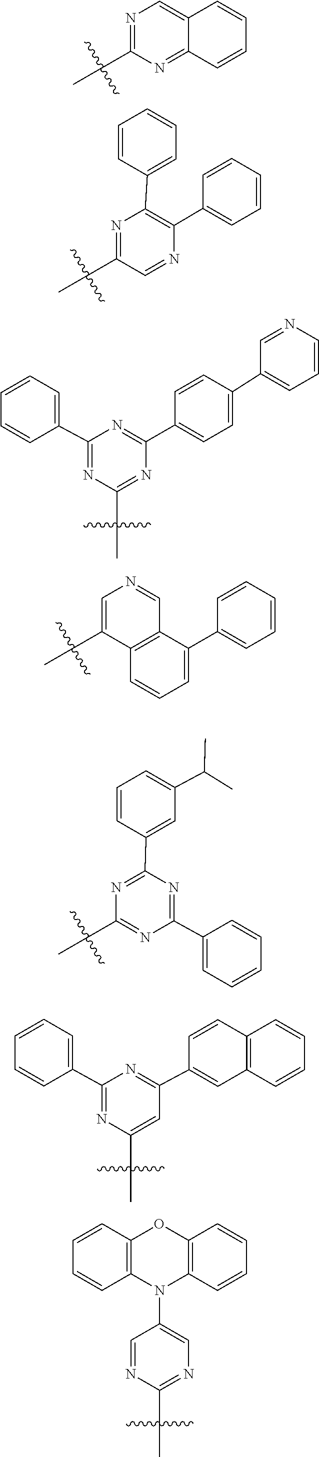

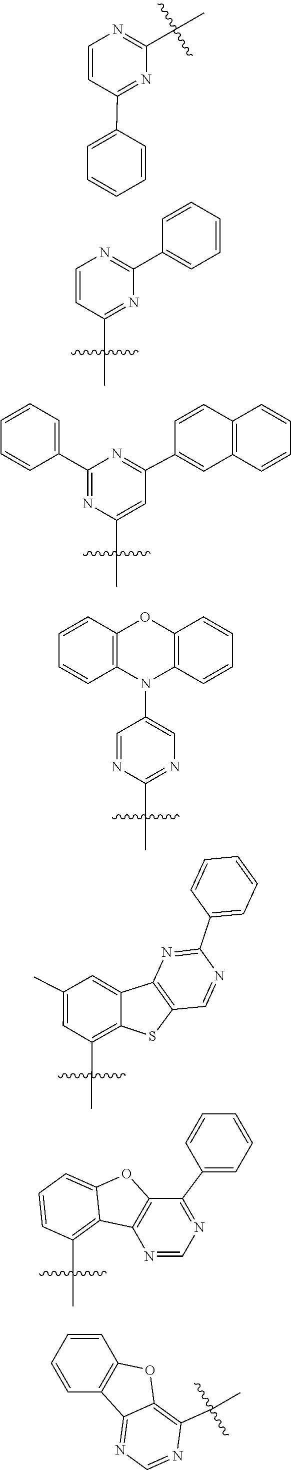

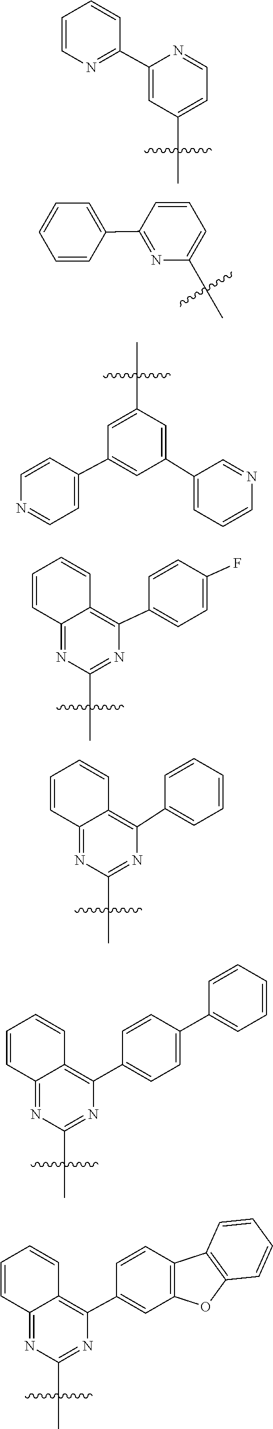

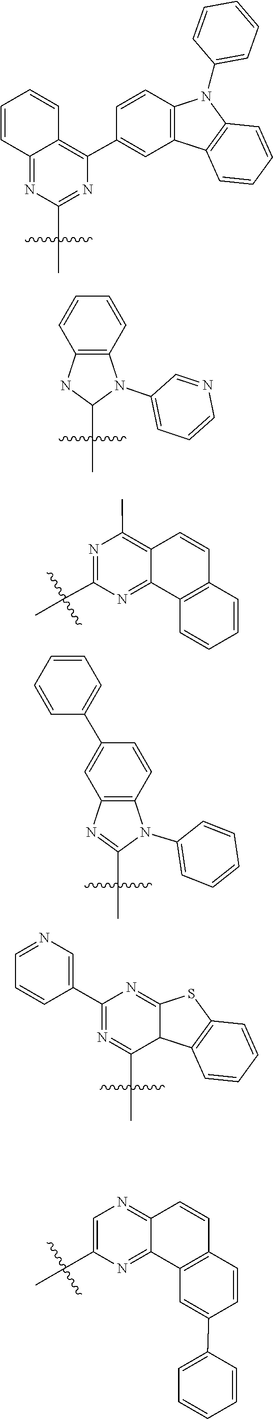

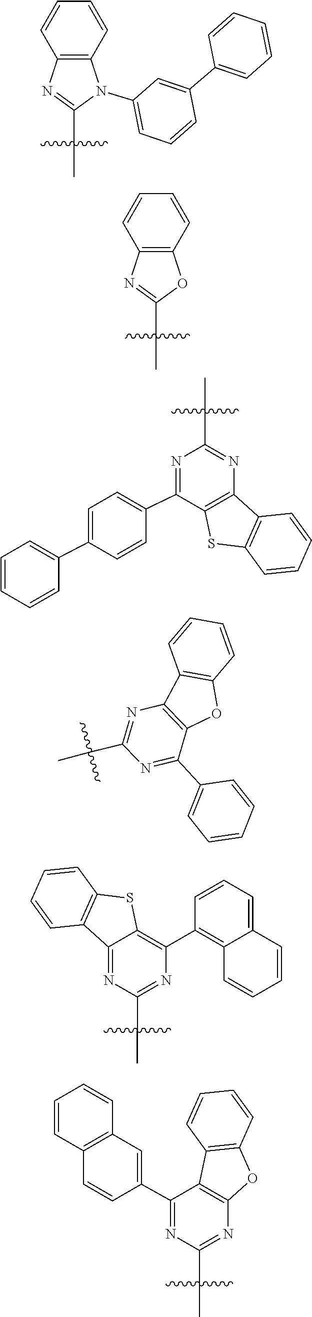

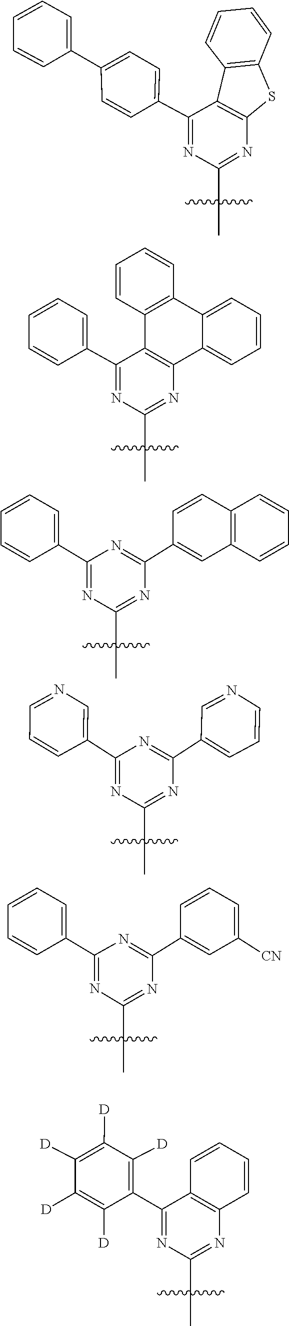

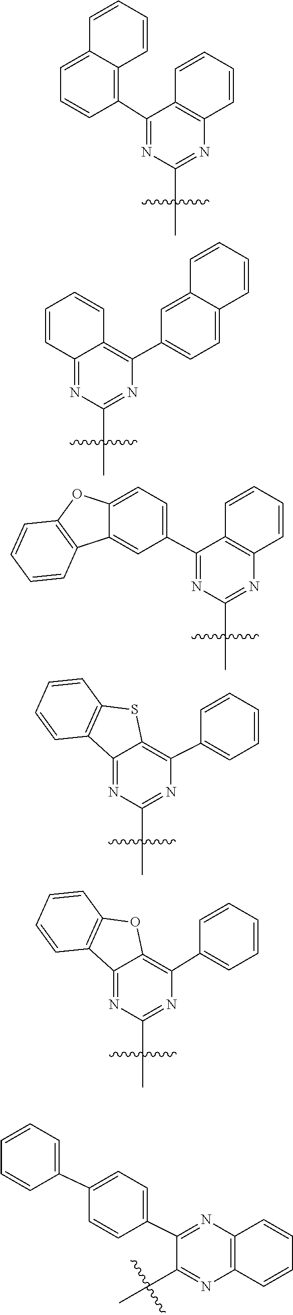 [SMILES: *c1nc(-c2cccc3ccccc23)c2ccccc2n1 ; *c1nc(-c2ccc3ccccc3c2)c2ccccc2n1 ; *c1nc(-c2ccc3oc4ccccc4c3c2)c2ccccc2n1 ; *c1nc(-c2ccccc2)c2sc3ccccc3c2n1 ; *c1nc(-c2ccccc2)c2oc3ccccc3c2n1 ; *c1nc2ccccc2nc1-c1ccc(-c2ccccc2)cc1]

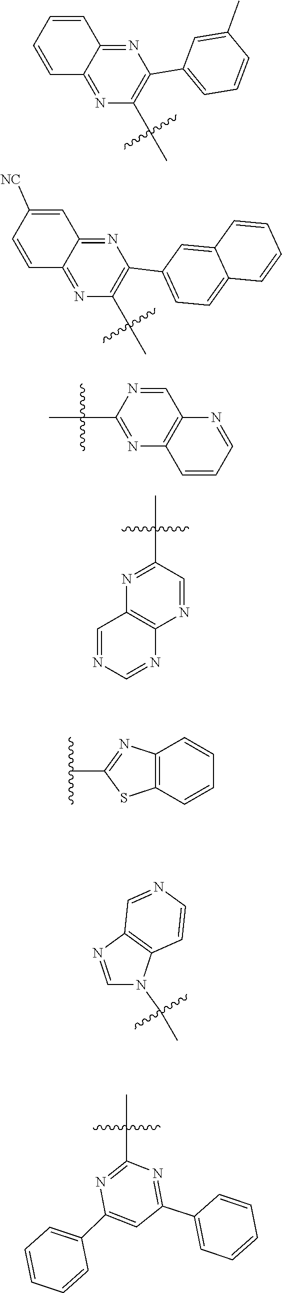

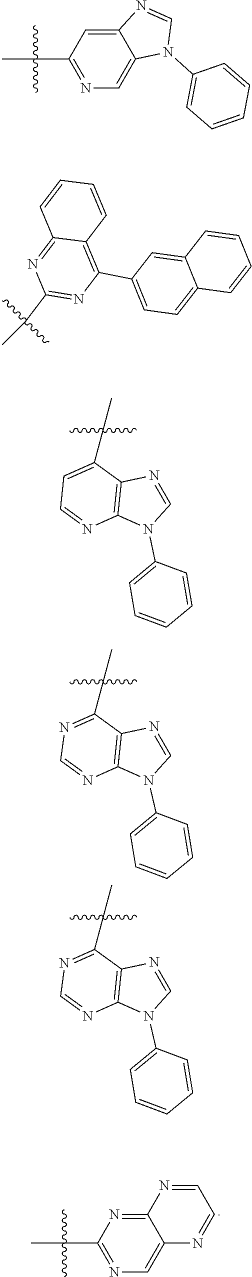 [SMILES: *c1cc2ncn(-c3ccccc3)c2cn1 ; *c1nc(-c2ccc3ccccc3c2)c2ccccc2n1 ; *c1ccnc2c1ncn2-c1ccccc1 ; *c1ncnc2c1ncn2-c1ccccc1 ; *c1ncnc2c1ncn2-c1ccccc1 ; *c1ncc2nccnc2n1]

In some other embodiments of the present disclosure, when R₂ is not

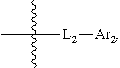

the Ar₁ may further be selected from aryl or electron-rich heteroaryl. These electron-rich aromatic groups can increase the electron cloud density of the conjugated system as a whole, and can adjust an HOMO energy level of the compound, so the compound will have the better hole transport ability. Furthermore, dibenzofuranyl and dibenzothienyl further have an excellent energy transfer effect, when the compound serves as the host of the luminescent layer, energy loss can be reduced, and a device efficiency can be improved.

In some more specific embodiments of the present disclosure, the Ar₁ is selected from the following structures:

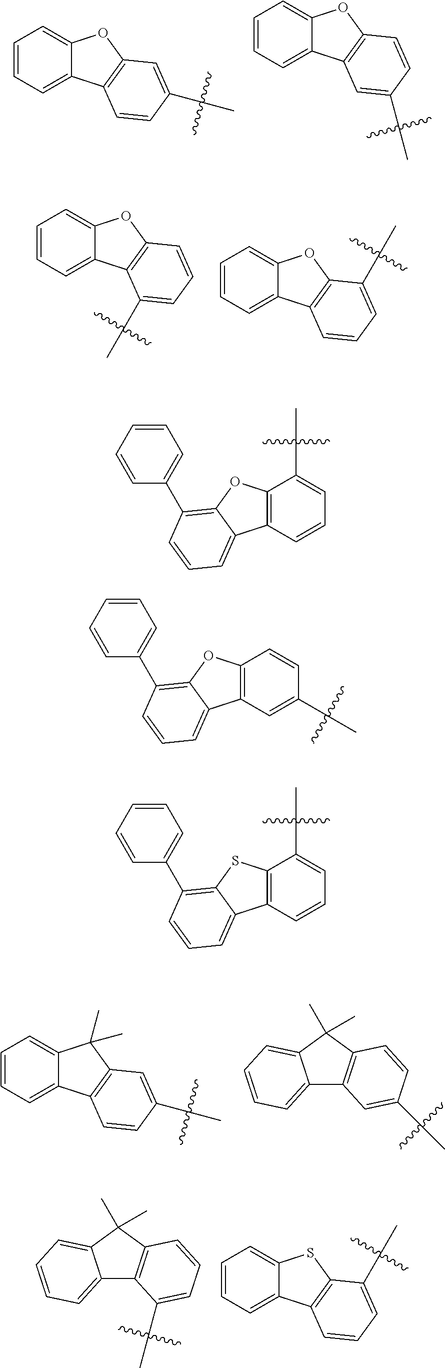

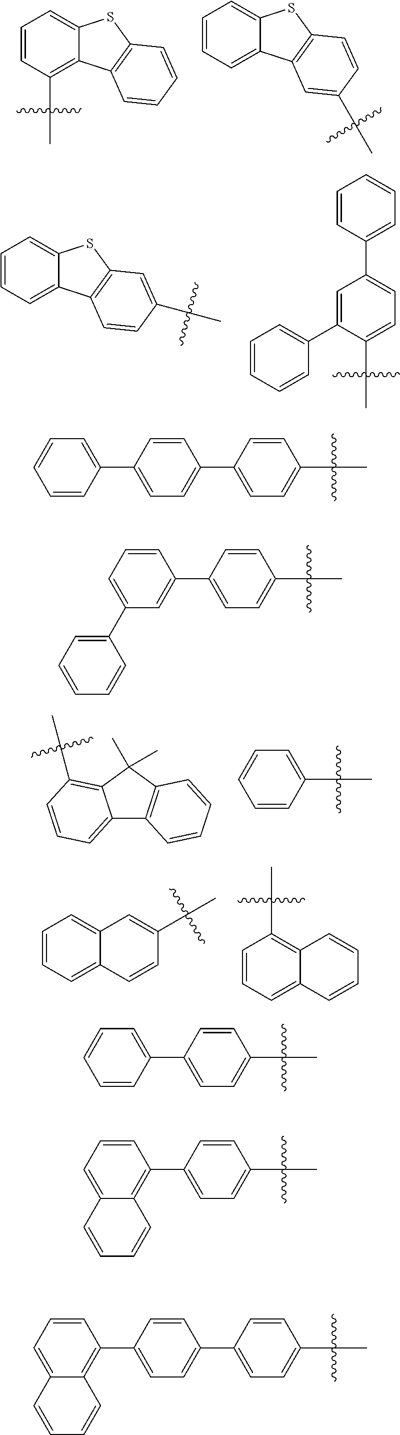

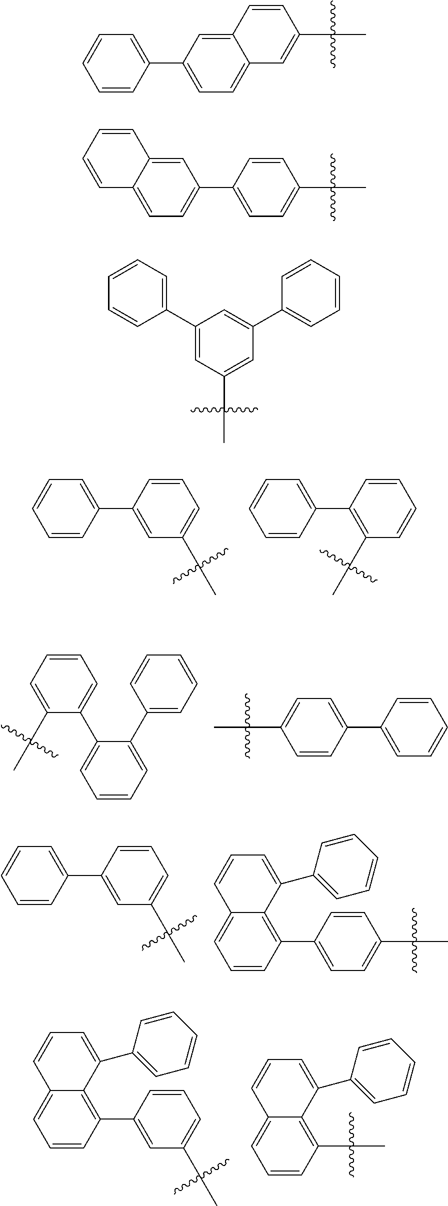

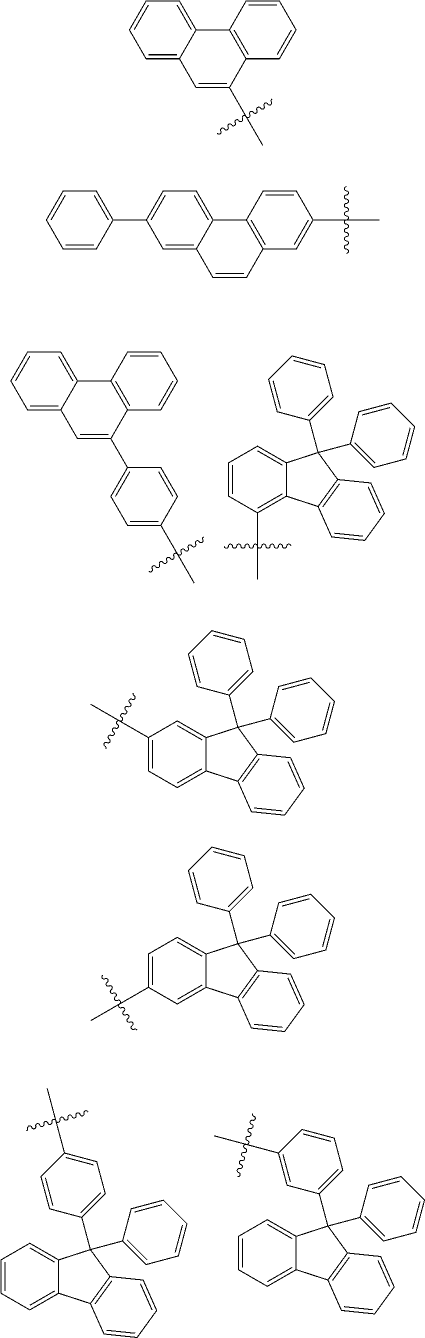

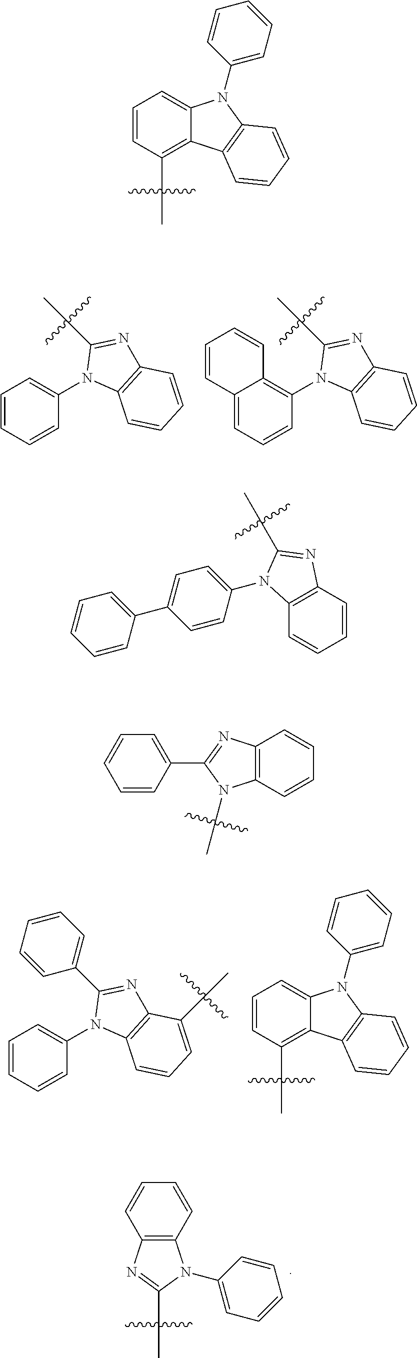

In some embodiments of the present disclosure, L₂ is selected from a single bond, substituted or unsubstituted arylene with 6 to 18 carbon atoms or

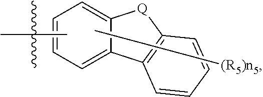

and the L₂ is not anthrylene.

In an optional embodiment of the present disclosure, Q is selected from O, S, C(R₇R₈) or Si(R₇R₈), the R₇ and the R₈ are the same, and the R₇ and the R₈ are selected from methyl or phenyl; or the R₇ and R₈ are connected to each other to form a cyclopentane ring, a cyclohexane ring or a fluorene ring together with the atoms to which they are jointly connected. Each R₅ is independently selected from deuterium, fluorine, chlorine, cyano, methyl, ethyl, isopropyl, n-propyl, tert-butyl, methoxyl, ethoxy, trifluoromethyl, trimethylsilyl, phenyl or naphthyl.

In some embodiments of the present disclosure, L₂ is selected from a single bond, substituted or unsubstituted arylene with 6 to 18 carbon atoms, substituted or unsubstituted dibenzothienylene, substituted or unsubstituted dibenzofurylene, substituted or unsubstituted dimethylfluorenylene, substituted or unsubstituted 9,9-dimethyl-9H-9-silafluorenylene, and the L₂ is not anthrylene.

Further, in some more specific embodiments, substituents in the L₂ are the same as or different from each other, and each independently selected from the groups formed by being substituted by deuterium, fluorine, chlorine, cyano, alkyl with 1 to 4 carbon atoms, alkoxy with 1 to 4 carbon atoms, cycloalkyl with 3 to 7 carbon atoms, aryl with 6 to 15 carbon atoms, heteroaryl with 5 to 18 carbon atoms, alkylthio with 1 to 4 carbon atoms, haloalkyl with 1 to 4 carbon atoms, and alkylsilyl with 3 to 9 carbon atoms; and when there are a plurality of substituents in the L₂, any two substituents are the same or different.

In some embodiments of the present disclosure, the L₂ is selected from one of a single bond, substituted or unsubstituted phenylene, substituted or unsubstituted biphenylene, substituted or unsubstituted terphenylene, substituted or unsubstituted naphthylene, substituted or unsubstituted 9,9-dimethylfluorenylene, substituted or unsubstituted phenanthrylene, spiro[cyclopentane-1,9′-fluorenylene], spiro[cyclohexane-1,9′-fluorenylene], substituted or unsubstituted 9,9-dimethyl-9H-9-silafluorenylene, substituted or unsubstituted dibenzofurylene, substituted or unsubstituted dibenzothienylene, and substituted or unsubstituted spirobifluorenylene, or is a subunit group formed by connecting two or three of the above subunits through a single bond; and substituents in the L₂ are the same as or different from each other, and are each independently selected from the group consisting of deuterium, fluorine, chlorine, cyano, methyl, ethyl, isopropyl, n-propyl, tert-butyl, methoxy, ethoxy, trifluoromethyl, trimethylsilyl, phenyl and naphthyl.

In some embodiments of the present disclosure, the L₂ is selected from a single bond, or substituted or unsubstituted W₂, where the unsubstituted W₂ is selected from the group consisting of the following groups:

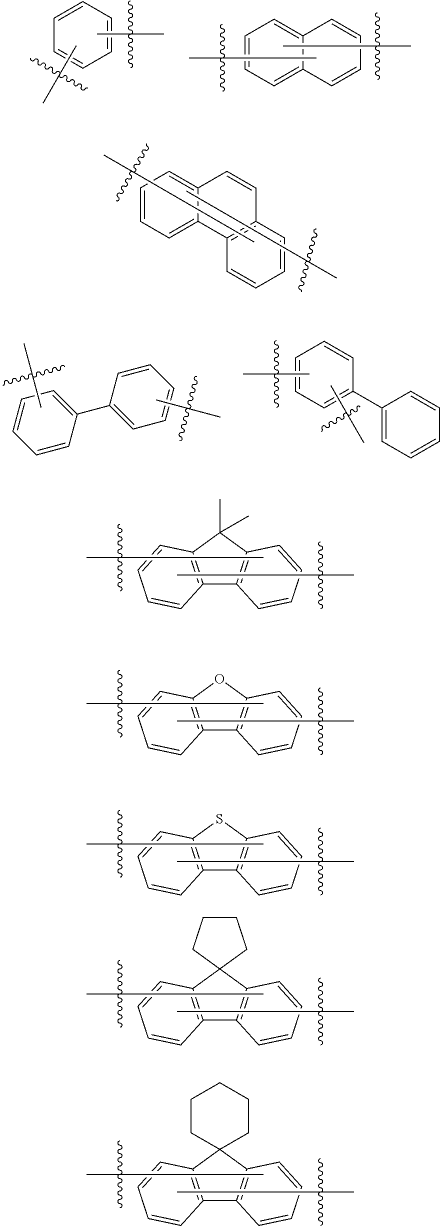

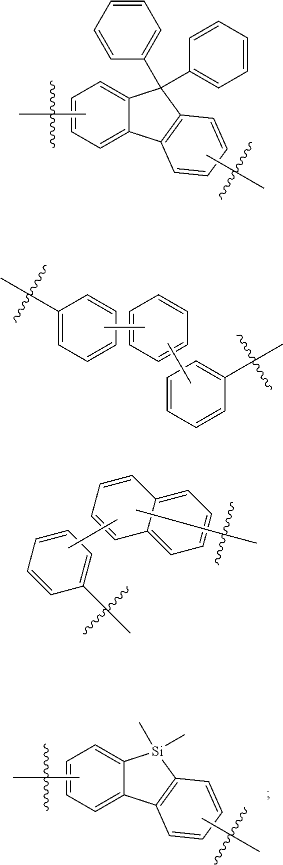

where the substituted W₂ is a group formed by substituting the unsubstituted W₂ with one or more substituents selected from deuterium, fluorine, chlorine, bromine, cyano, alkyl with 1 to 4 carbon atoms, haloalkyl with 1 to 4 carbon atoms, trialkylsilyl with 3 to 9 carbon atoms, cycloalkyl with 5 to 7 carbon atoms, and aryl with 6 to 15 carbon atoms, and when there are a plurality of substituents on the substituted W₂, any two substituents are the same or different.

Further, in an optional embodiment of the present disclosure, the substituted W₂ is a group formed by substituting the unsubstituted W₂ with one or more groups selected from deuterium, fluorine, chlorine, cyano, methyl, ethyl, isopropyl, n-propyl, tert-butyl, methoxyl, ethoxy, trifluoromethyl, trimethylsilyl, phenyl or naphthyl.

In some embodiments of the present disclosure, L₂ is selected from the following structures:

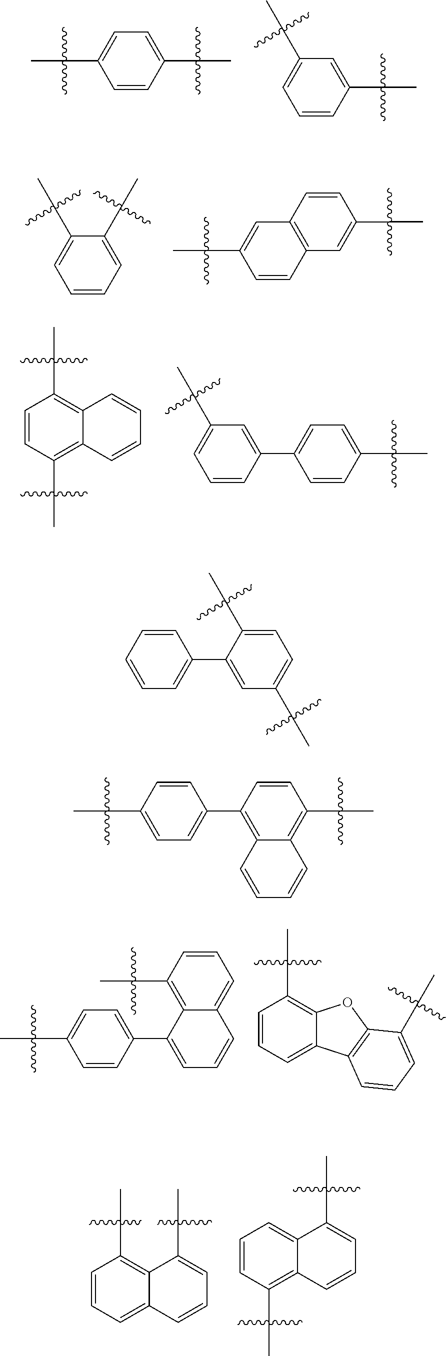

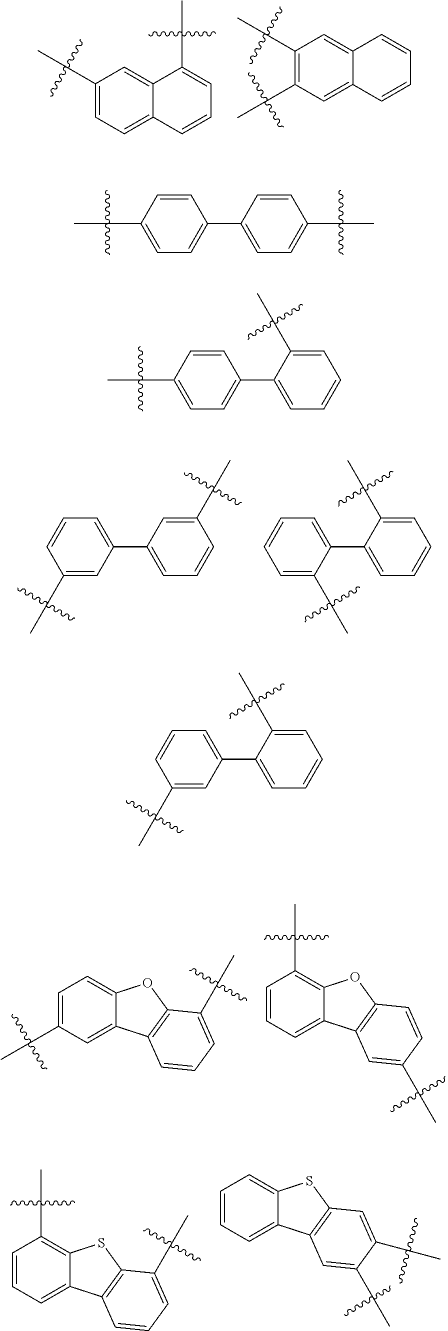

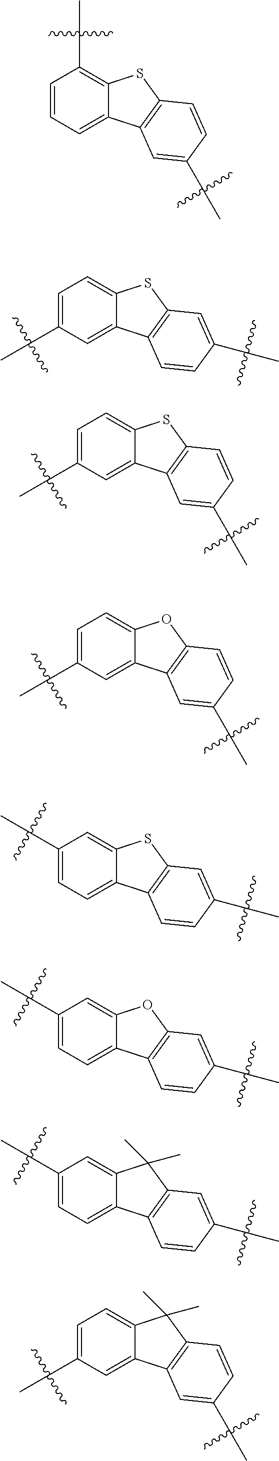

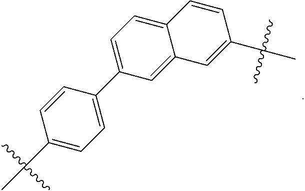 [SMILES: *c1ccc(-c2ccc3ccc(*)cc3c2)cc1]

The L₂ is not limited to the above groups.

In some embodiments of the present disclosure, Ar₂ is selected from substituted or unsubstituted arylene with 6 to 25 carbon atoms or

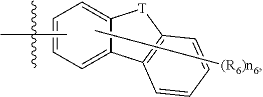

and the Ar₂ is not anthrylene.

In an optional embodiment of the present disclosure, T is selected from O, S, C(R₇R₈) or Si(R₇R₈), the R₇ and the R₈ are the same, and the R₇ and the R₈ are selected from methyl or phenyl; or the R₇ and R₈ are connected to each other to form a cyclopentane ring, a cyclohexane ring or a fluorene ring together with the atoms to which they are jointly connected. Each R₆ is independently selected from deuterium, fluorine, chlorine, cyano, methyl, ethyl, isopropyl, n-propyl, tert-butyl, methoxyl, trifluoromethyl, trimethylsilyl, phenyl, fluorenyl, phenyl substituted with cyano, phenyl substituted with fluorine, naphthyl, dibenzothienyl, dibenzofuranyl, cyclohexyl, or trimethylsilyl.

In some embodiments of the present disclosure, Ar₂ is selected from substituted or unsubstituted aryl with 6, 10, 12, 14, 15, 16, 18, 20 or 25 carbon atoms, substituted or unsubstituted dibenzothienyl, substituted or unsubstituted dibenzofuranyl, and substituted or unsubstituted 9,9-dimethyl-9H-9-silafluorenyl, and the Ar₂ is not anthryl.

Further, in some embodiments of the present disclosure, substituents in the Ar₂ are the same as or different from each other, and are each independently selected from the groups formed by being substituted by deuterium; fluorine; chlorine; cyano; alkyl with 1 to 4 carbon atoms; alkoxy with 1 to 4 carbon atoms; cycloalkyl with 3 to 7 carbon atoms; aryl with 6 to 15 carbon atoms which can be optionally substituted by 0, 1, 2 or 3 substituents selected from deuterium, fluorine, cyano or methyl; heteroaryl with 5 to 18 carbon atoms which can be optionally substituted by 0, 1, 2 or 3 substituents selected from deuterium, fluorine, cyano, or methyl; alkylthio with 1 to 4 carbon atoms; haloalkyl with 1 to 4 carbon atoms; and alkylsilyl with 3 to 9 carbon atoms; and when there are a plurality of substituents in the Ar₂, any two substituents are the same or different.

In some embodiments of the present disclosure, the Ar₂ is selected from one of substituted or unsubstituted phenyl, substituted or unsubstituted biphenyl, substituted or unsubstituted terphenyl, substituted or unsubstituted naphthyl, substituted or unsubstituted fluorenyl, substituted or unsubstituted silafluorenyl, substituted or unsubstituted dibenzofuranyl, substituted or unsubstituted dibenzothienyl, substituted or unsubstituted phenanthryl, substituted or unsubstituted pyrenyl, substituted or unsubstituted perylenyl, substituted or unsubstituted triphenylene, substituted or unsubstituted spirobifluorenyl, spiro[cyclopentane-1,9′-fluorenyl], and spiro[cyclohexane-1,9′-fluorenyl], or is a group formed by connecting two or three of the above groups through a single bond; and substituents in the Ar₂ are the same as or different from each other, and are each independently selected from the group consisting of deuterium, fluorine, chlorine, cyano, methyl, ethyl, isopropyl, n-propyl, tert-butyl, methoxy, trifluoromethyl, trimethylsilyl, phenyl, fluorenyl, cyano substituted phenyl, fluorine substituted phenyl, naphthyl, dibenzothienyl, dibenzofuranyl, cyclohexyl and trimethylsilyl, and when there are a plurality of substituents in the Ar₂, the substituents are the same as or different from each other.

In some embodiments of the present disclosure, the Ar₂ is selected from a substituted or unsubstituted group W₄, where the unsubstituted group W₄ is selected from the group consisting of the following groups:

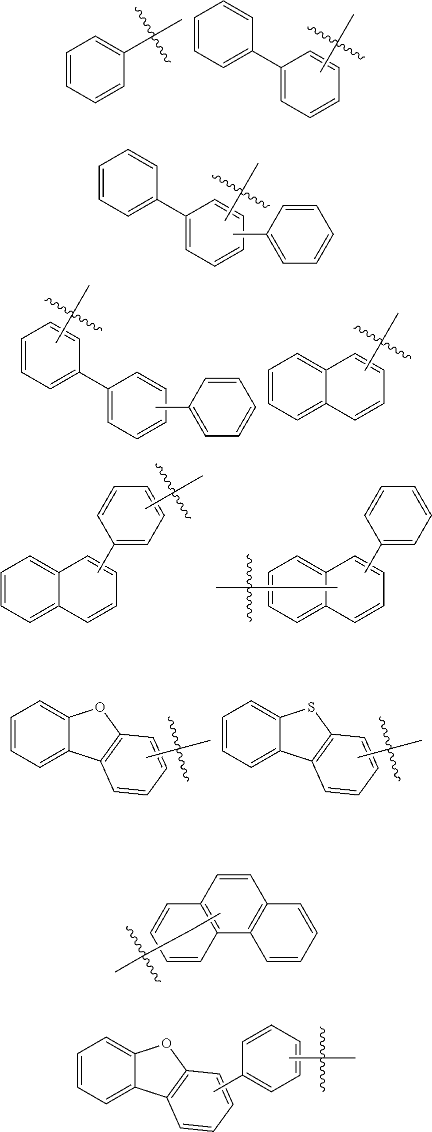

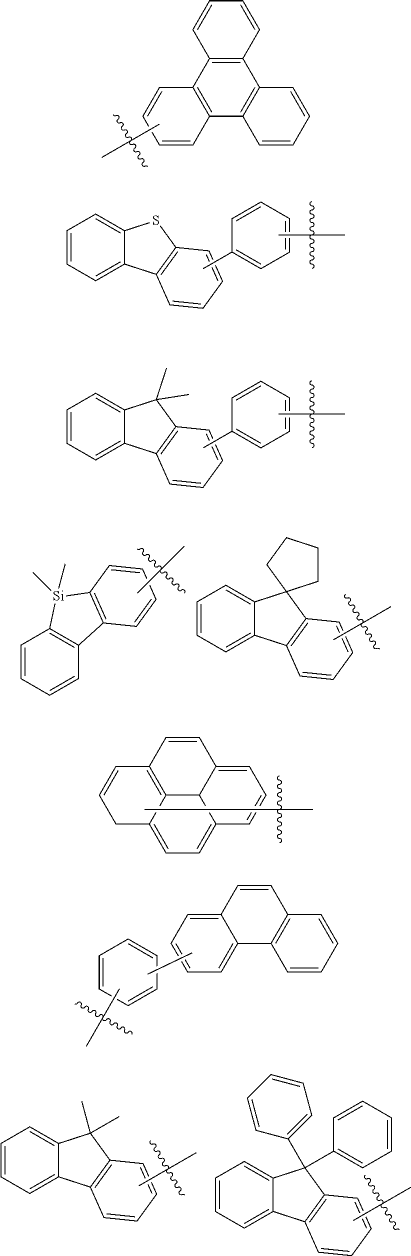

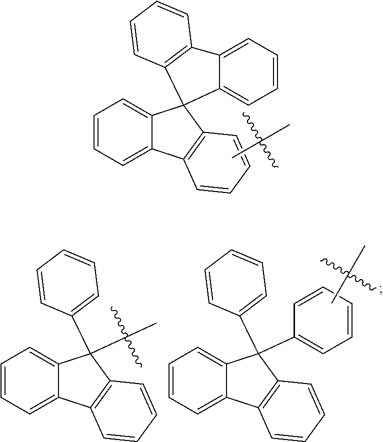

where the substituted group W₄ is a group formed by substituting the unsubstituted group W₄ with one or more substituents selected from deuterium, fluorine, chlorine, cyano, alkyl with 1 to 4 carbon atoms, alkoxy with 1 to 4 carbon atoms, cycloalkyl with 3 to 7 carbon atoms, aryl with 6 to 14 carbon atoms, heteroaryl with 3 to 12 carbon atoms, alkylthio with 1 to 4 carbon atoms, haloalkyl with 1 to 4 carbon atoms, or alkylsilyl with 3 to 9 carbon atoms; and when there are a plurality of substituents on the W_{4,} any two substituents are the same or different.

Further, the substituted group W₄ is a group formed by substituting the unsubstituted group W₄ with one or more substituents selected from deuterium, fluorine, chlorine, cyano, methyl, ethyl, isopropyl, n-propyl, tert-butyl, methoxyl, trifluoromethyl, trimethylsilyl, phenyl, fluorenyl, phenyl substituted with cyano, phenyl substituted with fluorine, naphthyl, dibenzothienyl, dibenzofuranyl, cyclohexyl and trimethylsilyl.

In some embodiments of the present disclosure, each R₁ is the same as or different from each other, and is each independently selected from: deuterium, fluorine, chlorine, cyano, alkyl with 1 to 4 carbon atoms, haloalkyl with 1 to 4 carbon atoms, alkoxy with 1 to 4 carbon atoms, cycloalkyl with 5 to 10 carbon atoms, heterocyclyl with 3 to 7 carbon atoms, trimethylsilyl, triphenylsilyl, alkylthio with 1 to 4 carbon atoms or

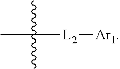

More specifically, in some embodiments of the present disclosure, each R₁ is the same as or different from each other, and is each independently selected from: fluorine, deuterium, cyano, trifluoromethyl, trimethylsilyl, methyl, ethyl, isopropyl, tert-butyl, methoxyl, ethoxy, isopropoxy, trifluoromethyl, cyclopentyl, cyclohexyl, phenyl, triphenylsilyl or

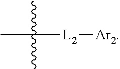

In some embodiments of the present disclosure, each R₂ is the same as or different from each other, and is each independently selected from: deuterium, fluorine, chlorine, cyano, alkyl with 1 to 4 carbon atoms, haloalkyl with 1 to 4 carbon atoms, alkoxy with 1 to 4 carbon atoms, cycloalkyl with 3 to 7 carbon atoms, alkylsilyl with 3 to 8 carbon atoms, triphenylsilyl, alkylthio with 1 to 4 carbon atoms or

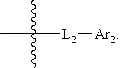

In some embodiments of the present disclosure, each R₂ is the same as or different from each other, and is each independently selected from: fluorine, deuterium, cyano, trifluoromethyl, trimethylsilyl, methyl, ethyl, isopropyl, tert-butyl, methoxyl, ethoxy, isopropoxy, trifluoromethyl, cyclopentyl, cyclohexyl, phenyl, triphenylsilyl or

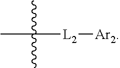

In some embodiments of the present disclosure, Ar₂ is selected from aryl or electron-rich dibenzofuranyl, and dibenzothienyl. These electron-rich aromatic groups as a whole can increase the electron cloud density of the conjugated system, for example, lone pair electrons on the oxygen atoms and the sulfur atoms can participate in the conjugated system to increase the electron cloud density of the conjugated system of the heteroaryl. Since the aryl and the electron-rich dibenzofuranyl and dibenzothienyl can effectively enhance the electron cloud density of the compound and can adjust the HOMO energy level of the compound, the compound will have better hole transport ability, and dibenzofuranyl and dibenzothienyl further have the excellent energy transfer effect. In this way, the compound can serve as the hole-type host material of the organic luminescent layer, and cooperate with the electron-type host material of the organic luminescent layer to form the host material of the organic luminescent layer together.

In some embodiments of the present disclosure, Ar₂ is selected from the following structures:

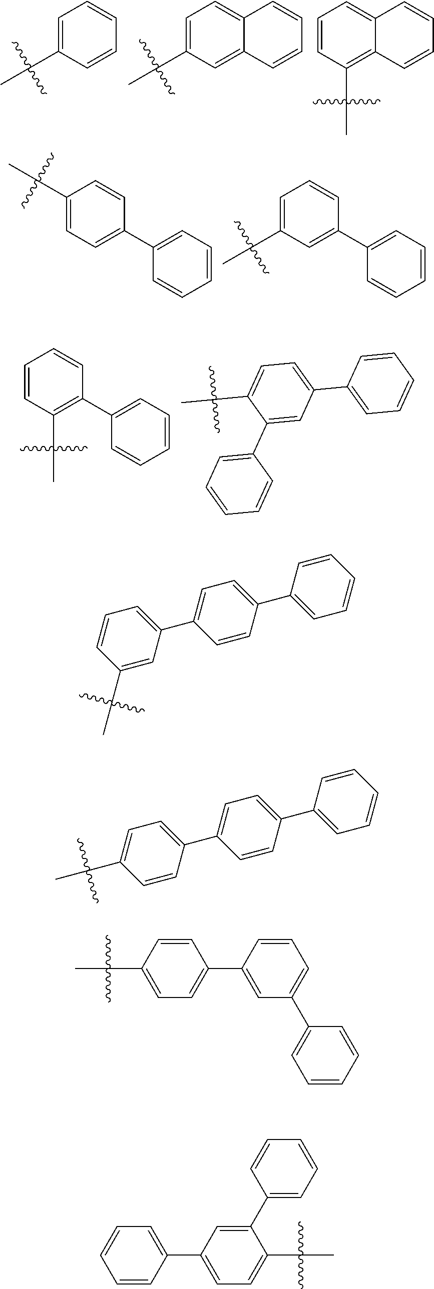

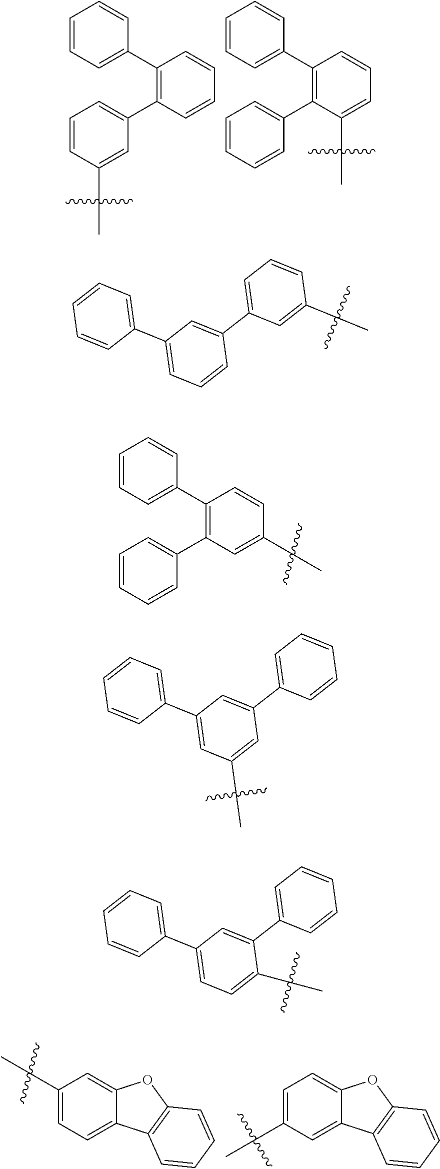

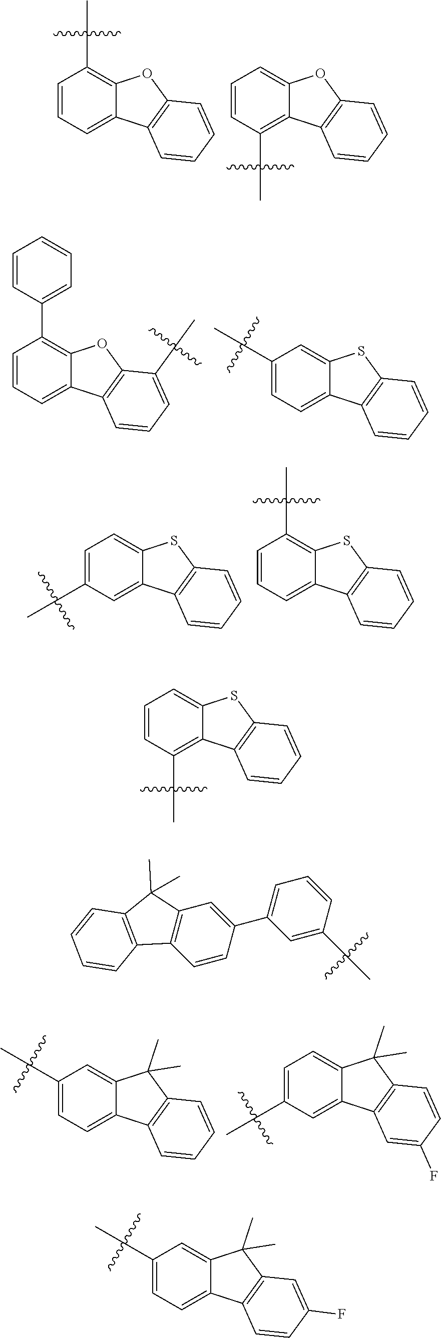

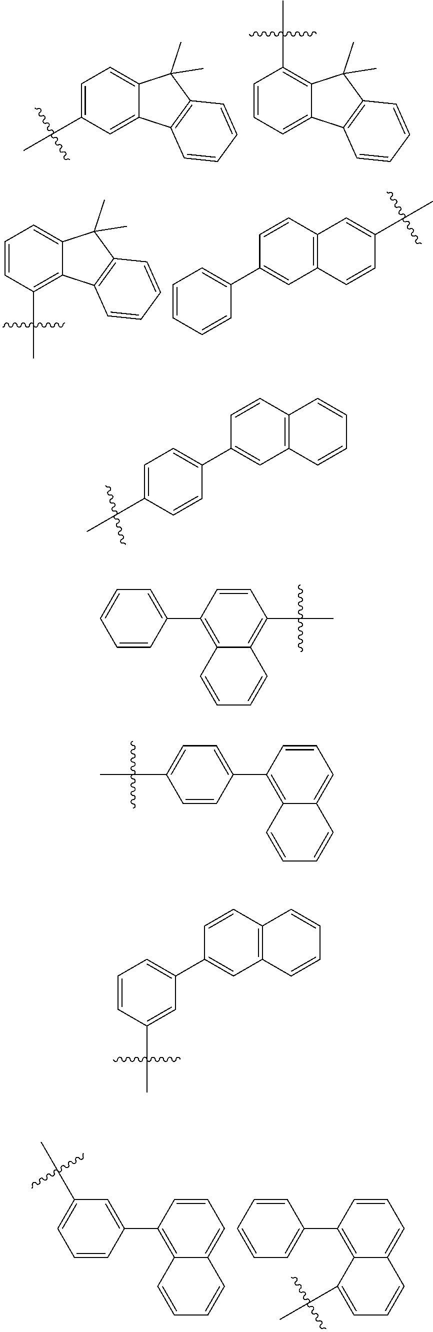

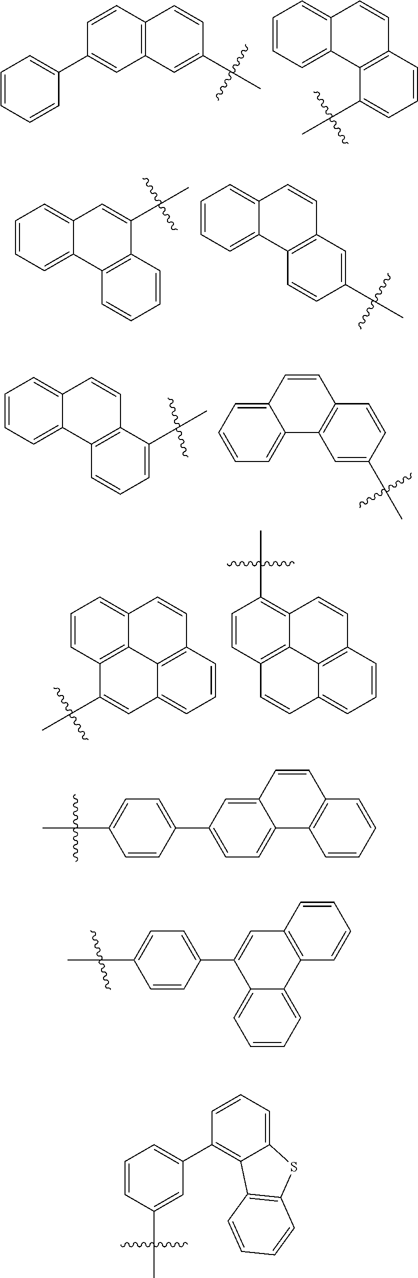

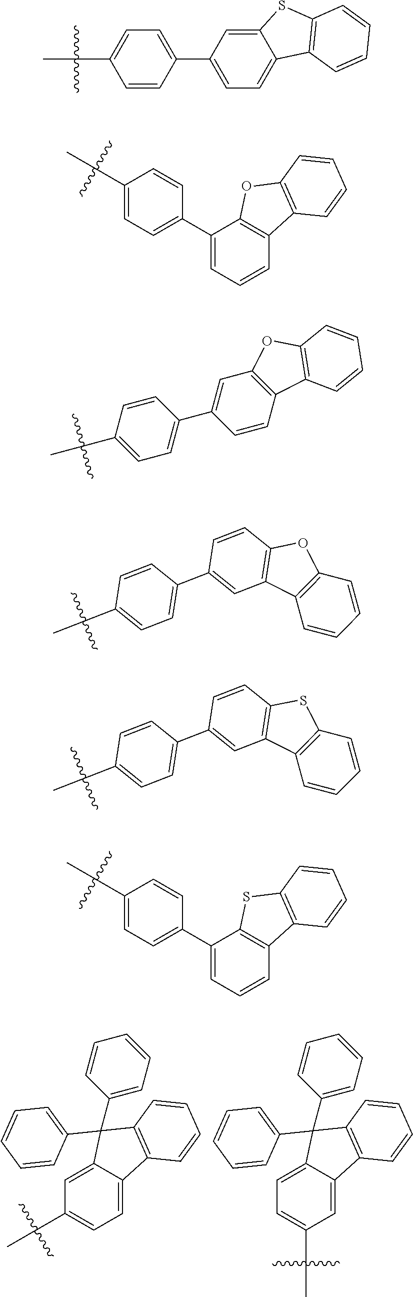

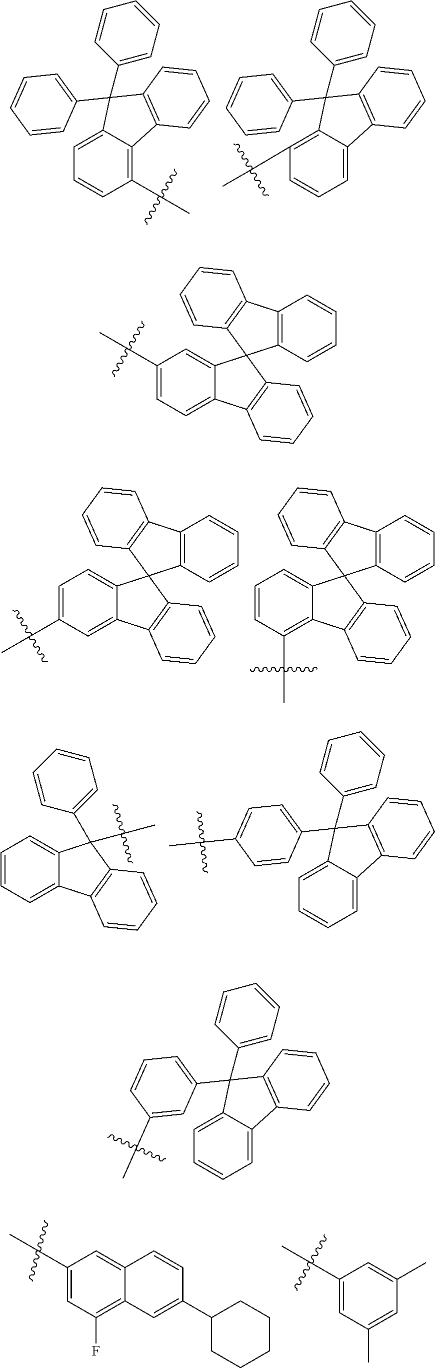

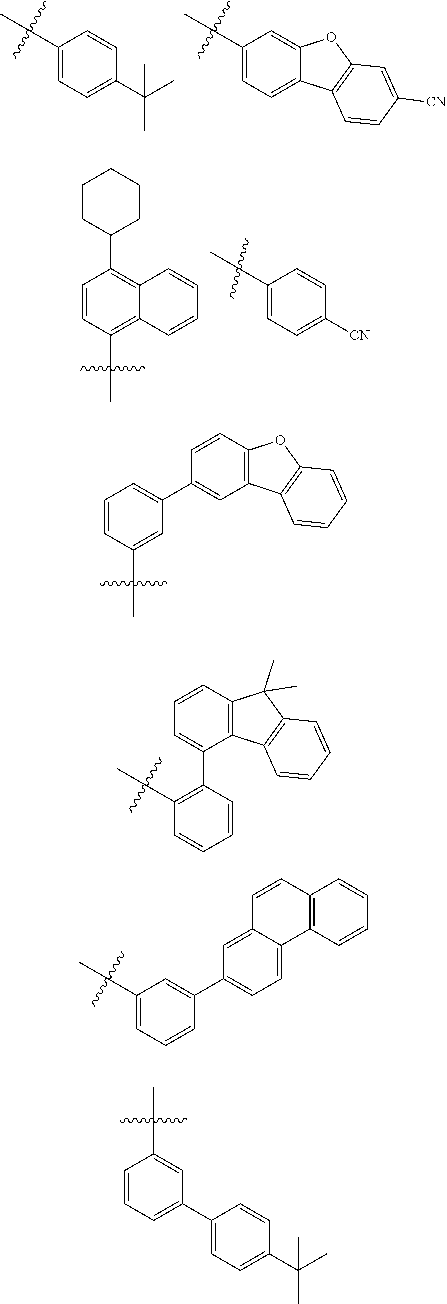

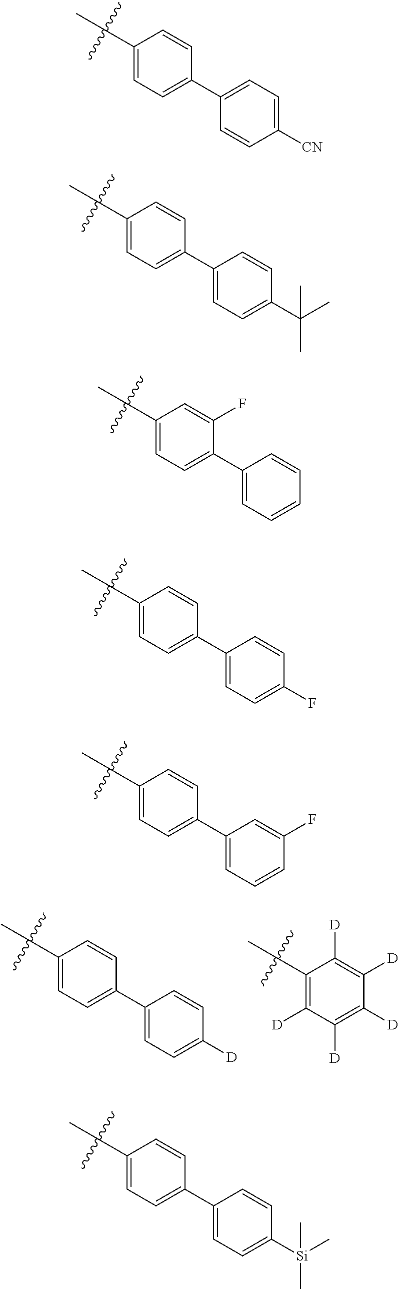

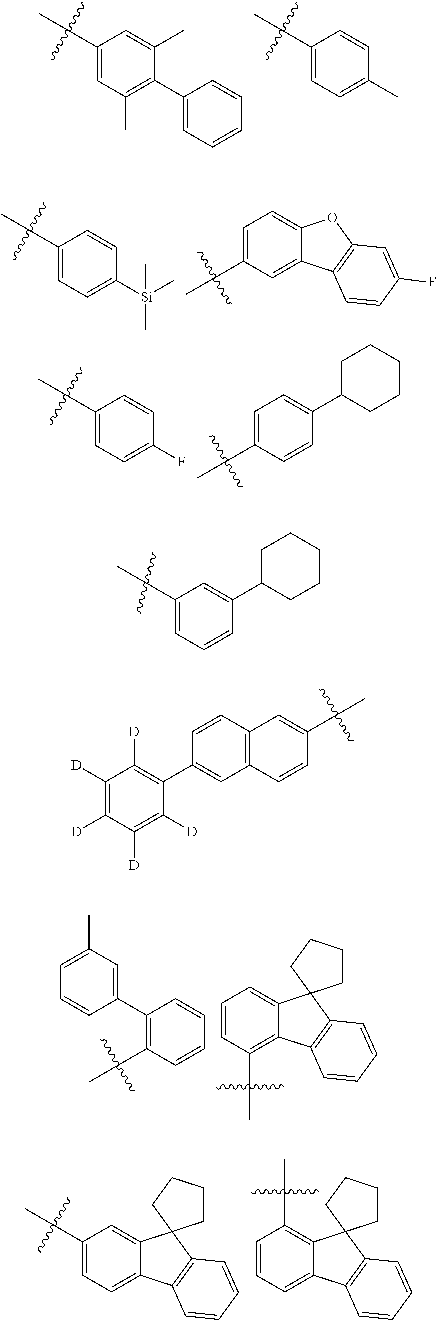

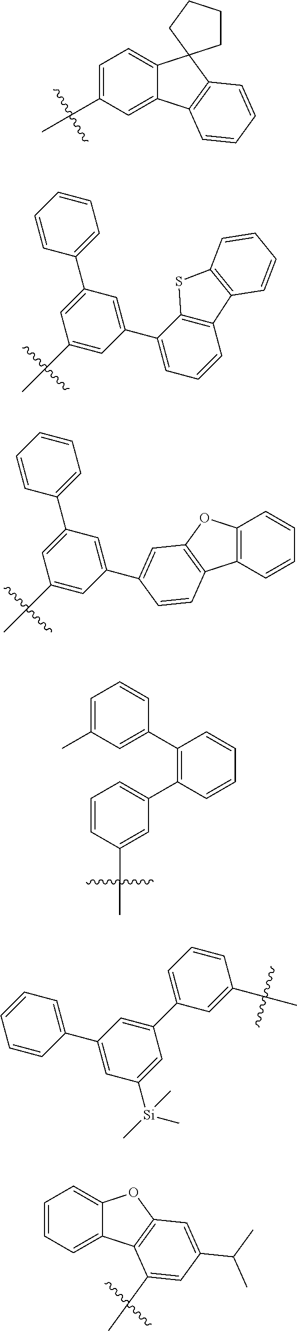 [SMILES: *c1ccc2c(c1)-c1ccccc1C21CCCC1 ; *c1cc(-c2ccccc2)cc(-c2cccc3c2sc2ccccc23)c1 ; *c1cc(-c2ccccc2)cc(-c2ccc3c(c2)oc2ccccc23)c1 ; *c1cccc(-c2ccccc2-c2cccc(C)c2)c1 ; *c1cccc(-c2cc(-c3ccccc3)cc([Si](C)(C)C)c2)c1 ; *c1cc(C(C)C)cc2oc3ccccc3c12]

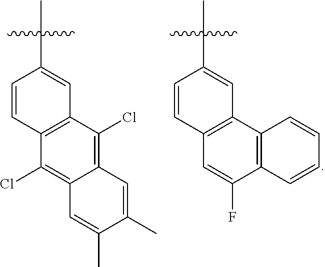 [SMILES: *c1ccc2c(Cl)c3cc(C)c(C)cc3c(Cl)c2c1 ; *c1ccc2cc(F)c3ccccc3c2c1]

The Ar₂ is not limited to the above groups.

Optionally, the compound is selected from the group consisting of the following compounds:

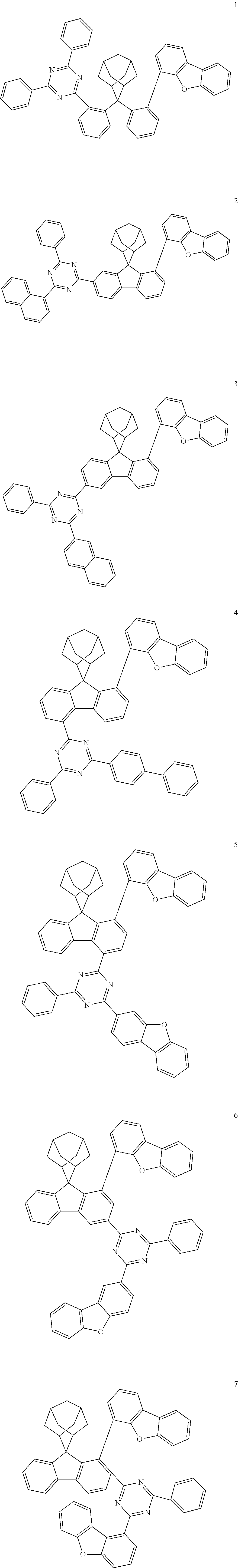

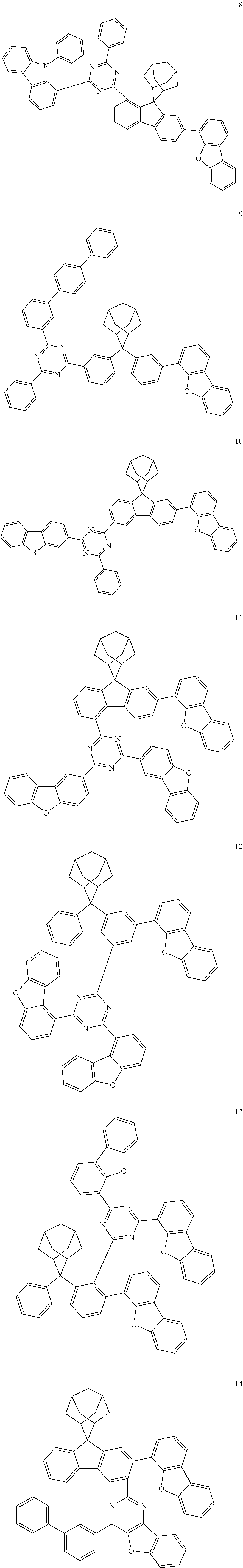

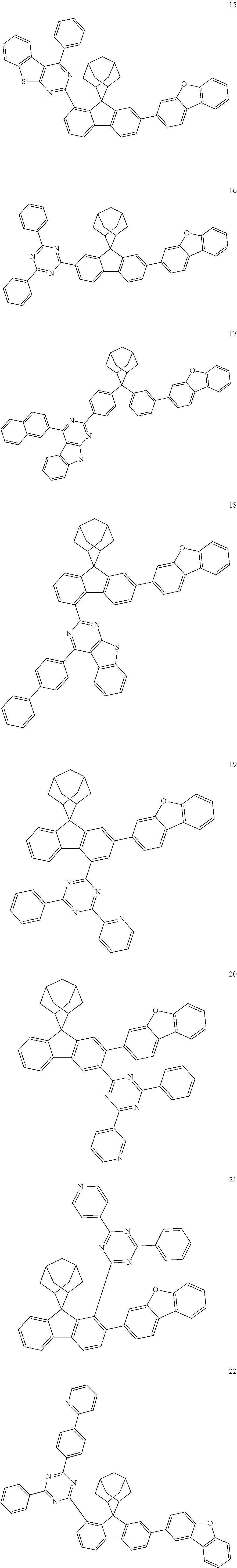

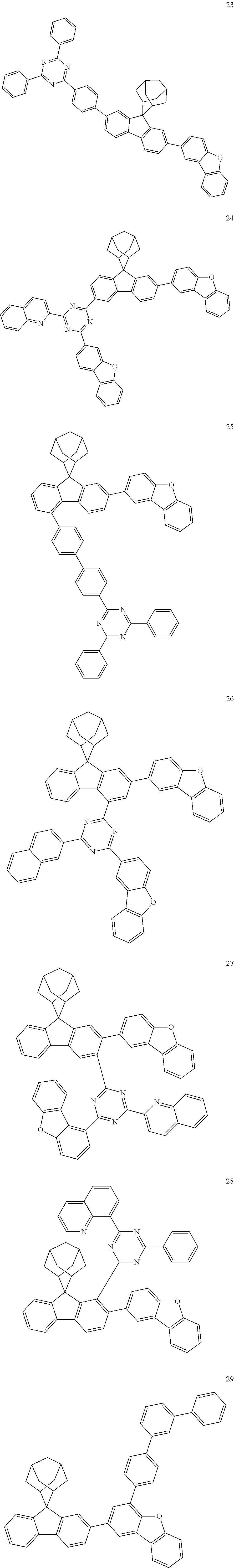

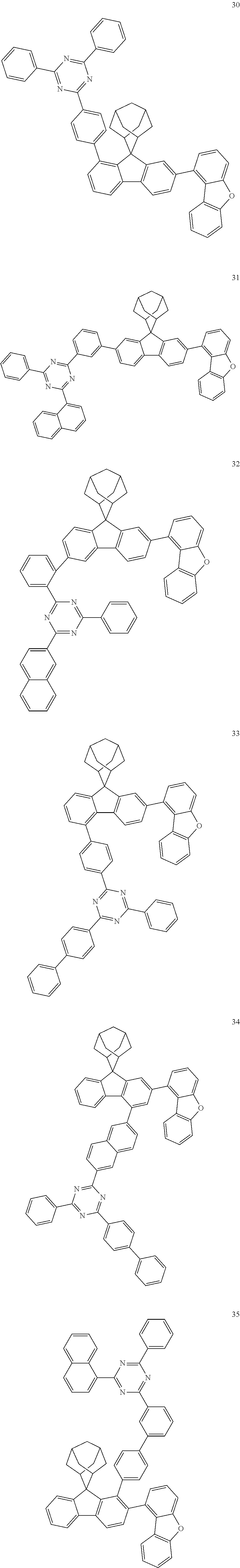

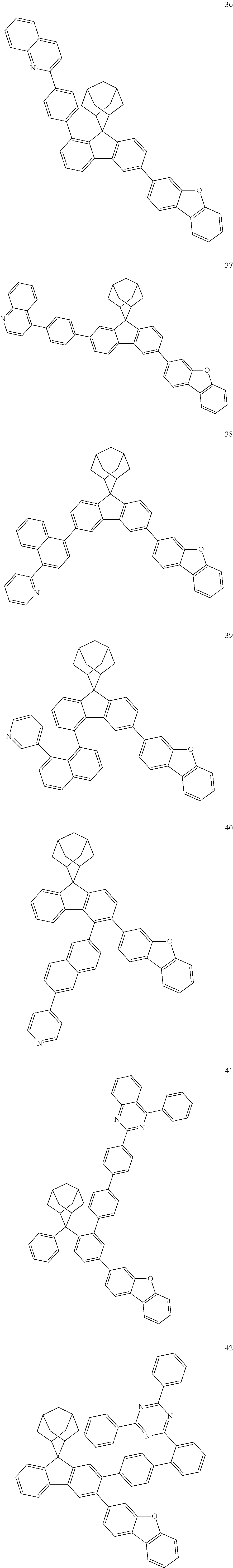

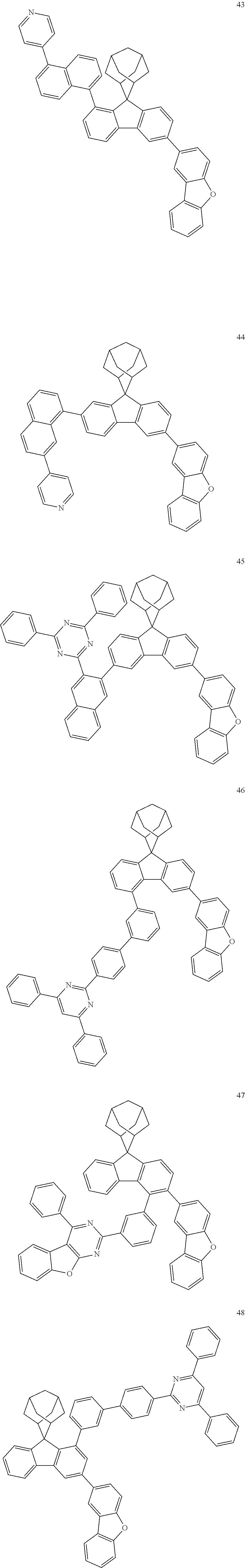

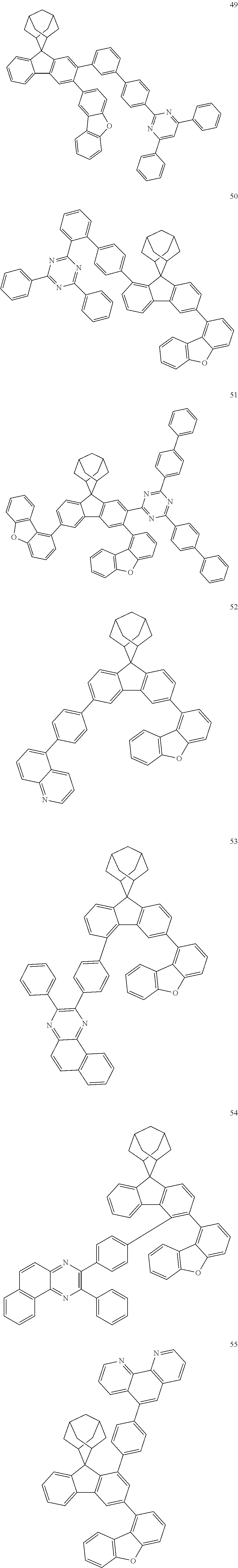

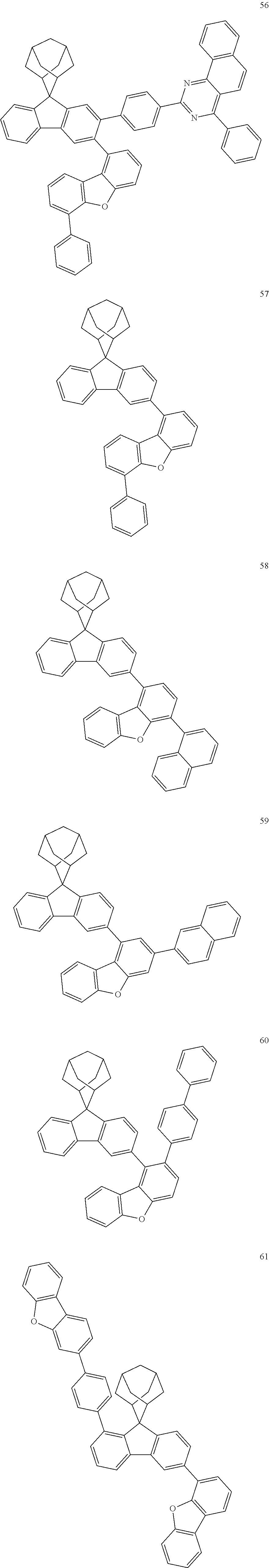

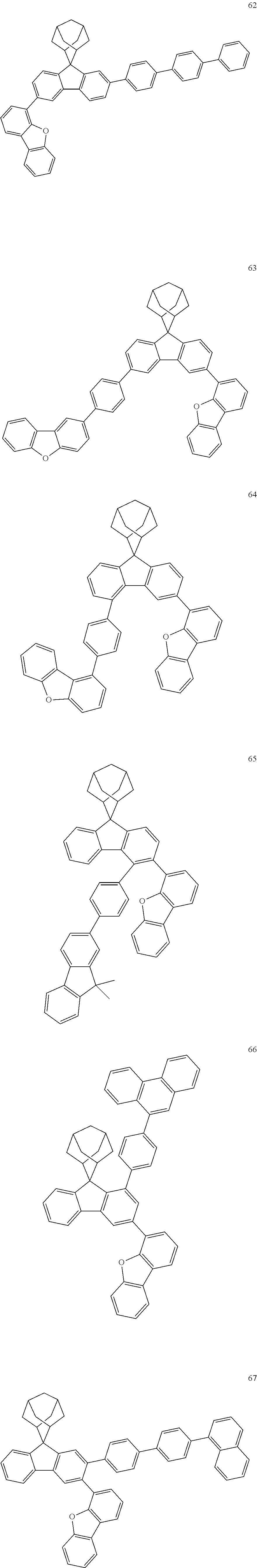

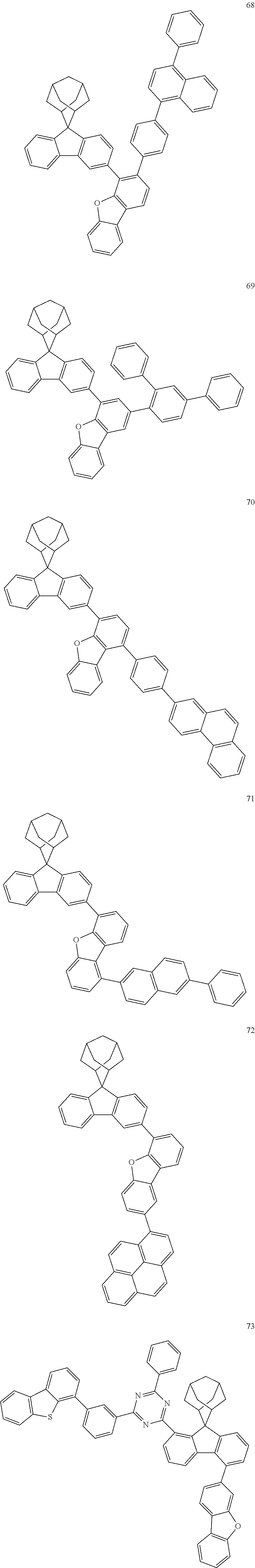

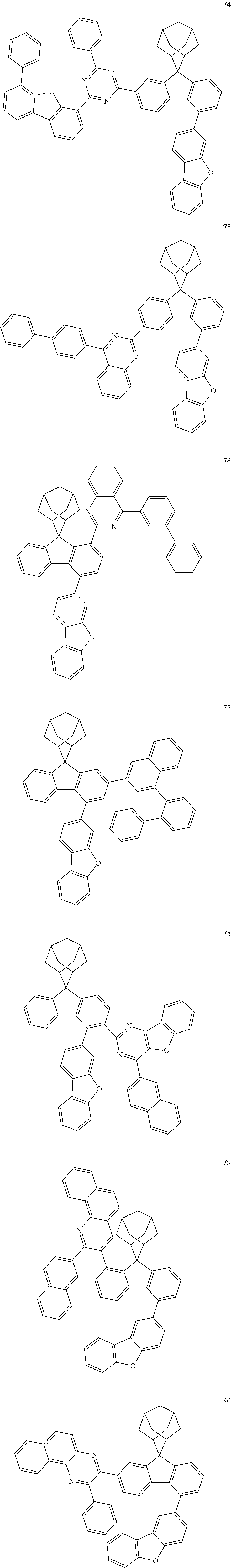

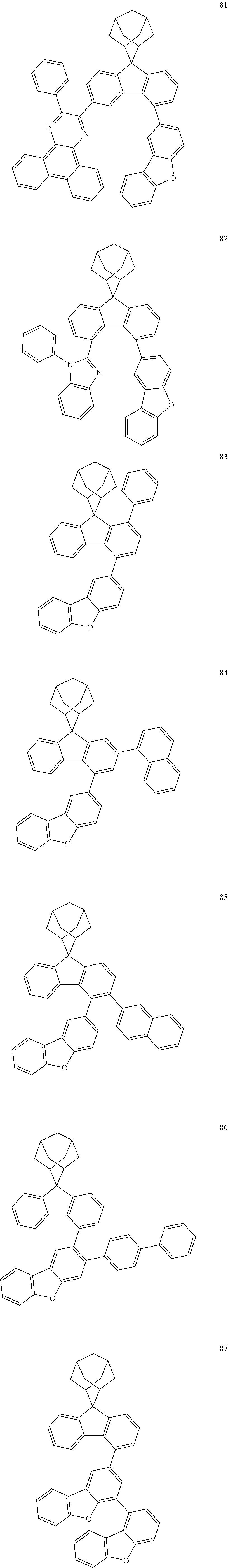

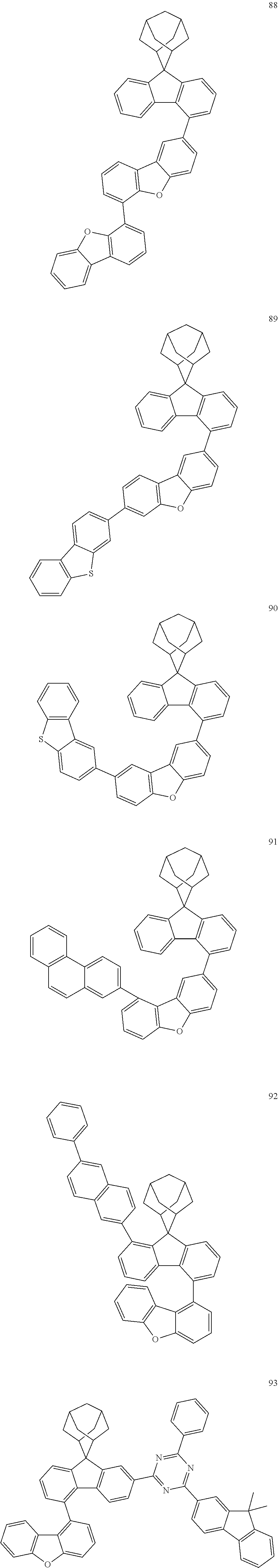

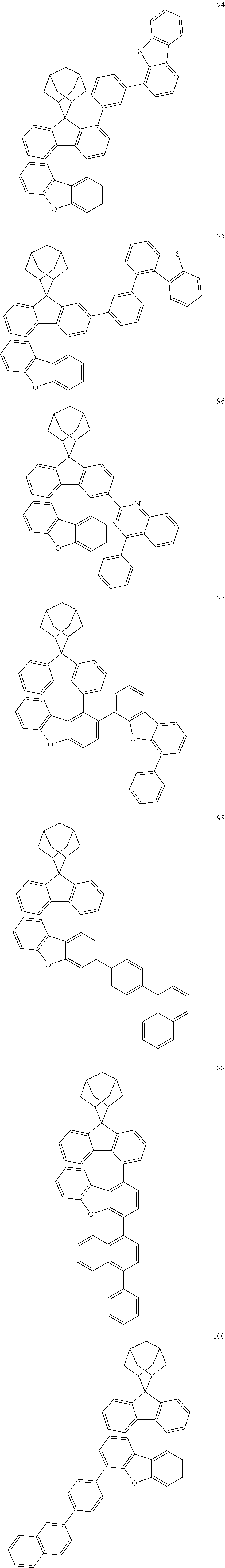

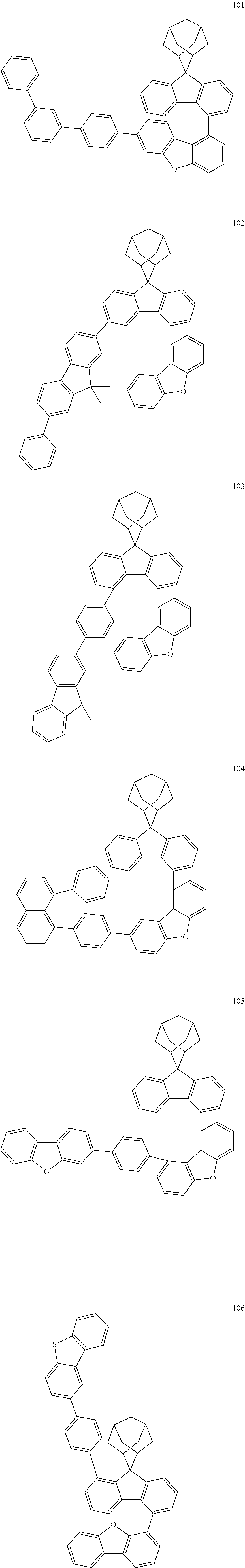

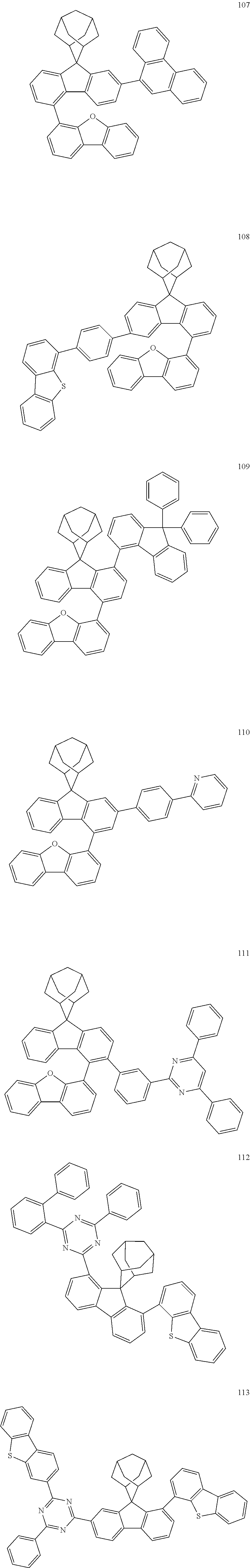

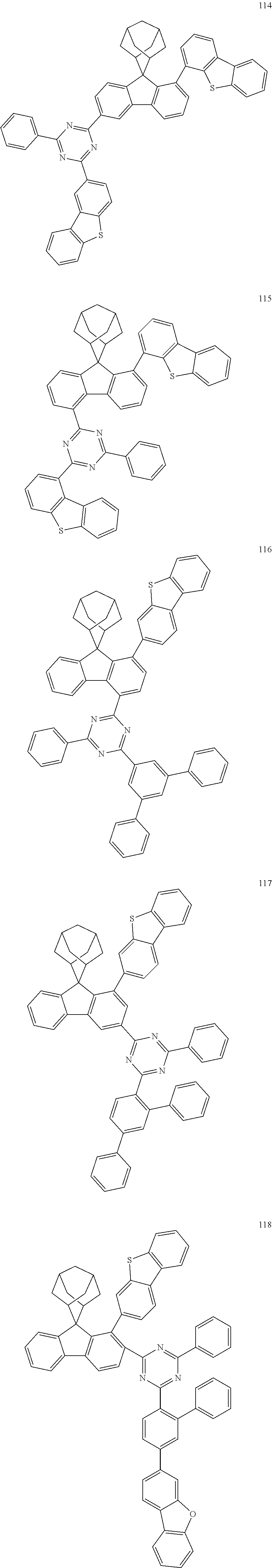

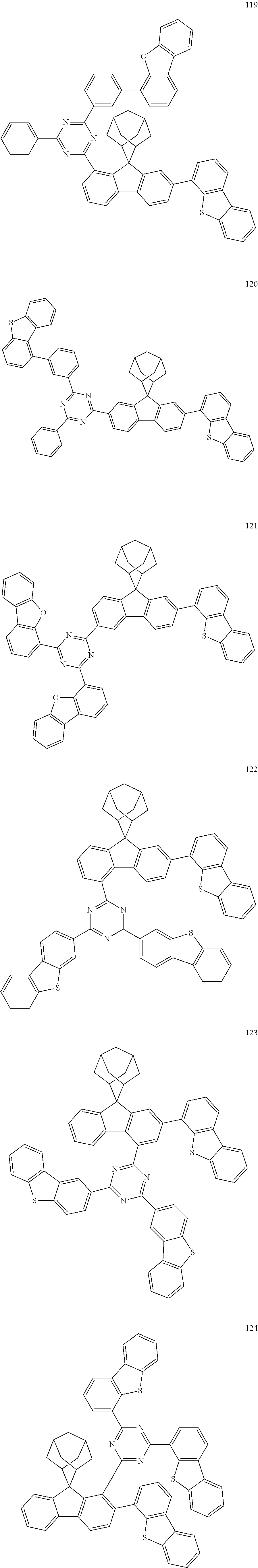

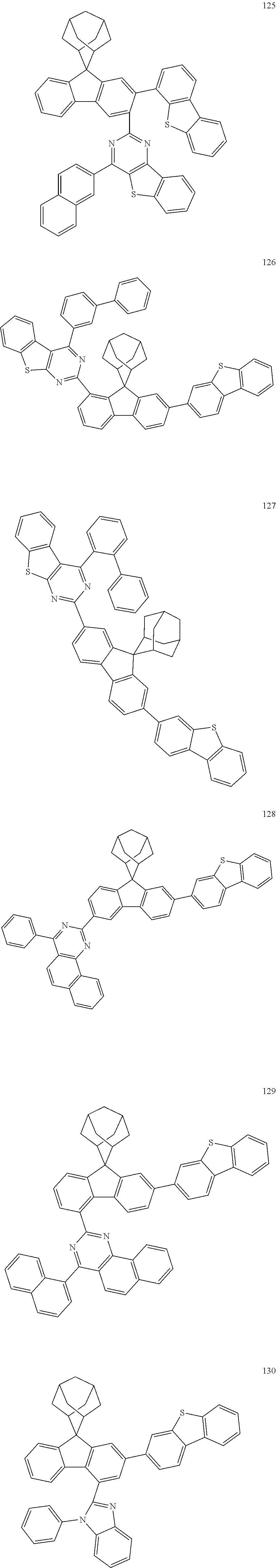

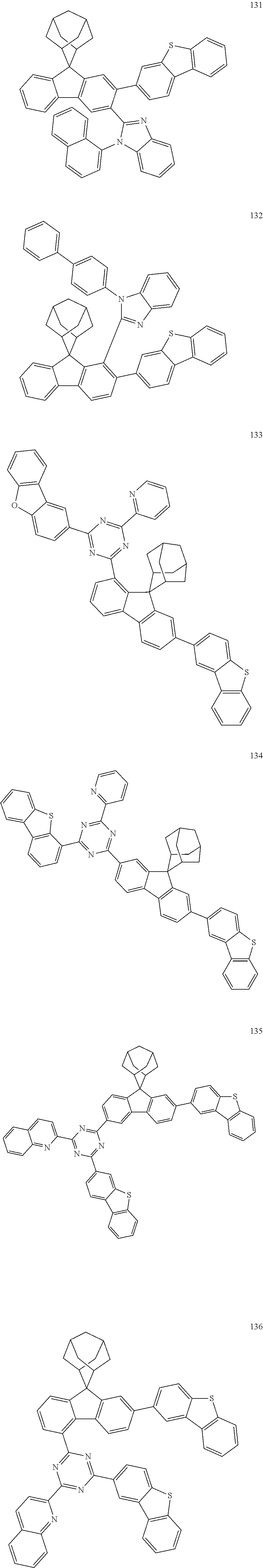

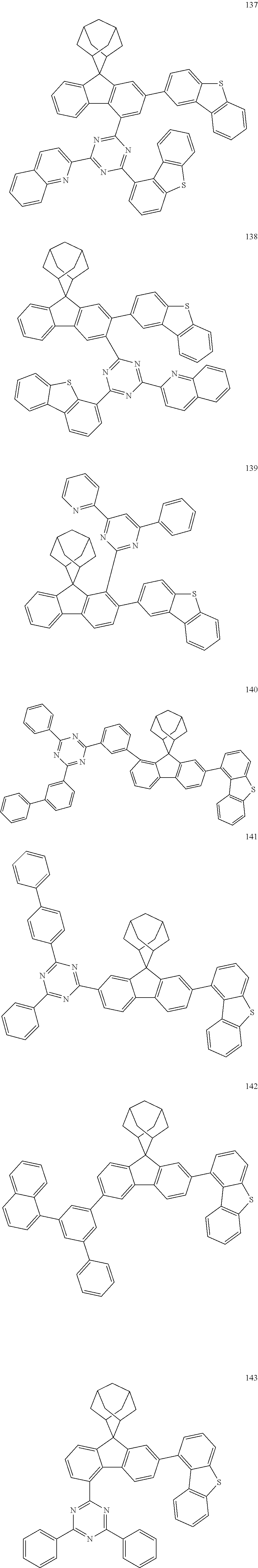

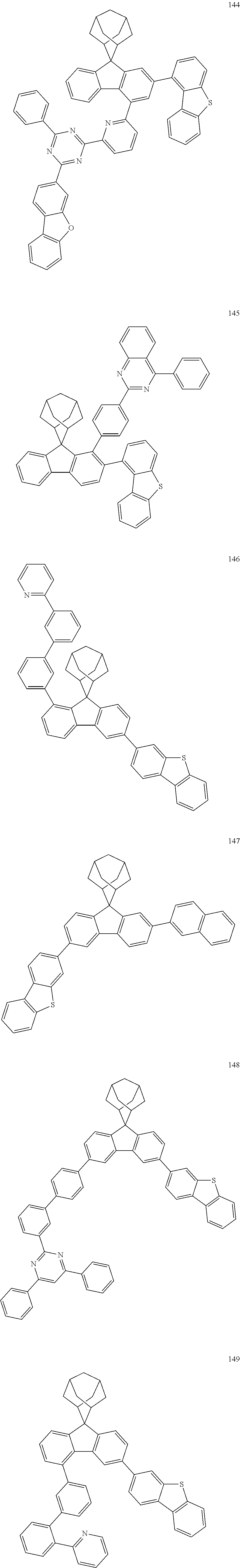

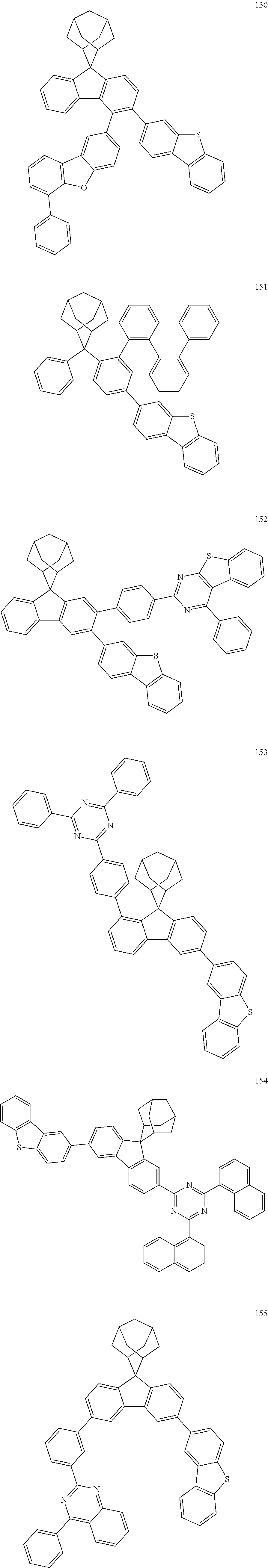

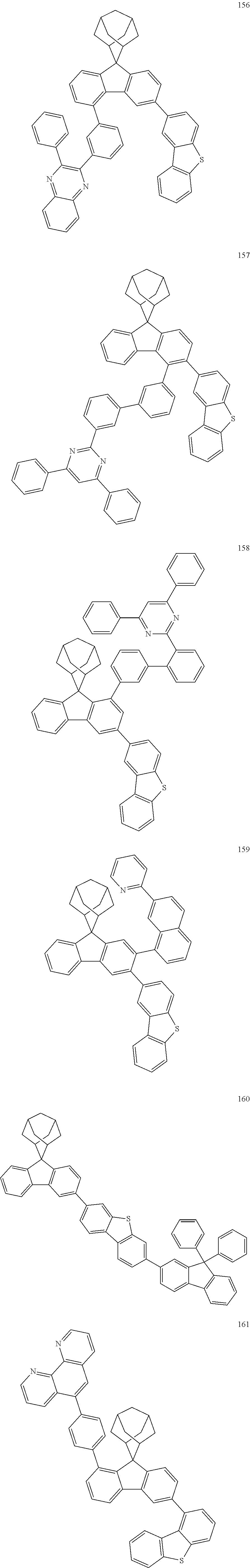

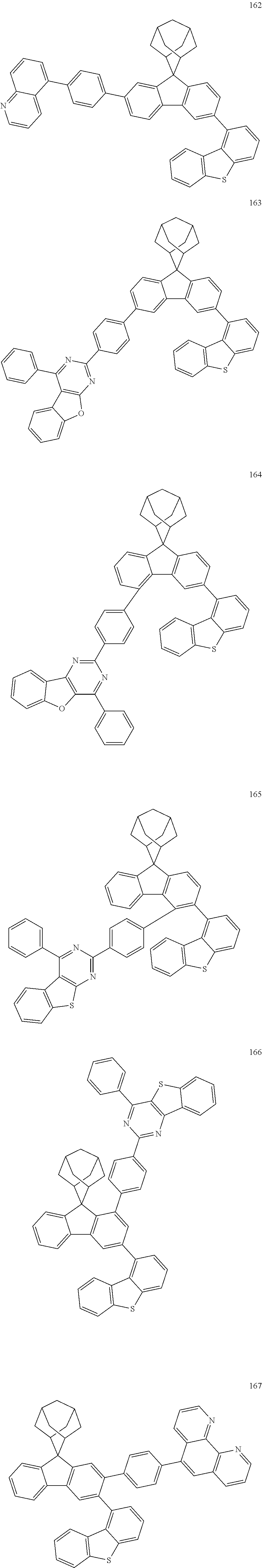

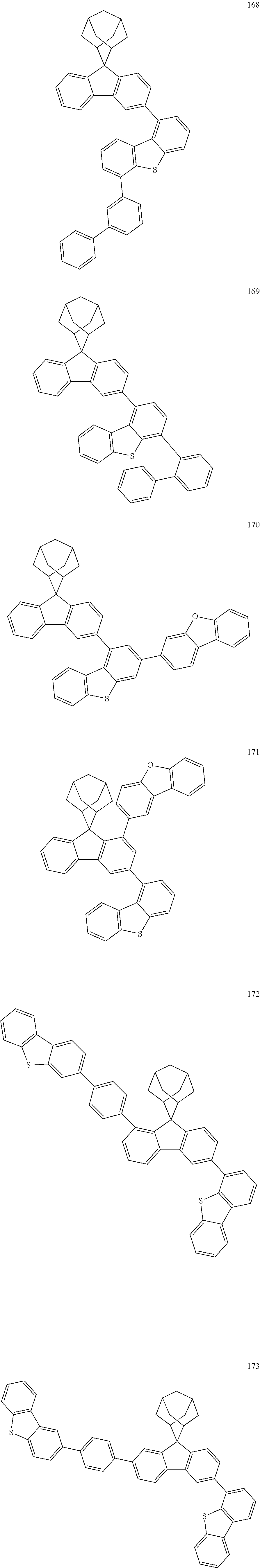

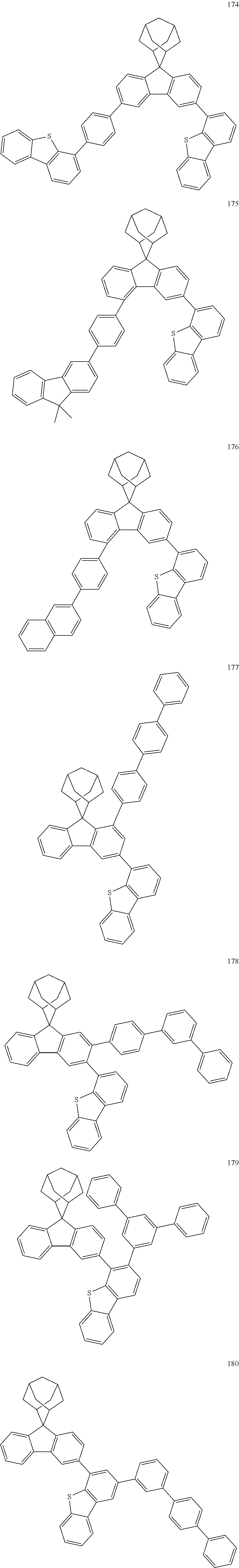

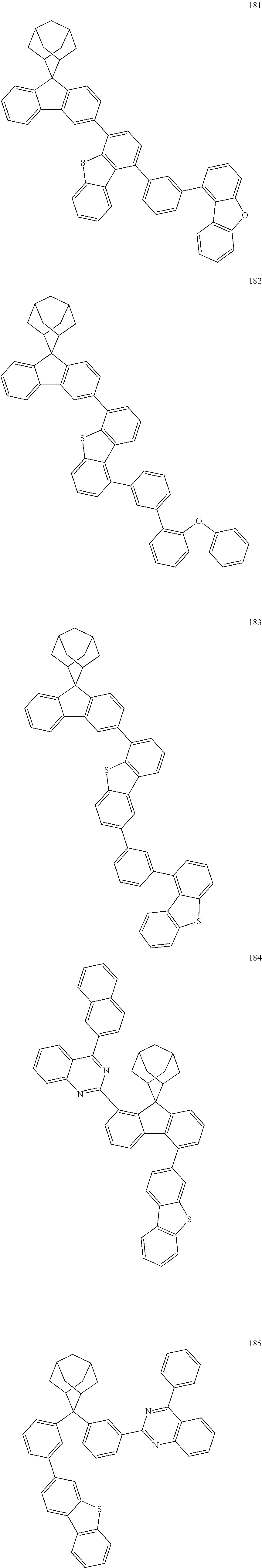 [SMILES: c1cc(-c2cccc3oc4ccccc4c23)cc(-c2ccc(-c3ccc4c(c3)-c3ccccc3C43C4CC5CC(C4)CC3C5)c3sc4ccccc4c23)c1 ; c1cc(-c2cccc3c2oc2ccccc23)cc(-c2cccc3sc4c(-c5ccc6c(c5)-c5ccccc5C65C6CC7CC(C6)CC5C7)cccc4c23)c1 ; c1cc(-c2ccc3sc4c(-c5ccc6c(c5)-c5ccccc5C65C6CC7CC(C6)CC5C7)cccc4c3c2)cc(-c2cccc3sc4ccccc4c23)c1 ; c1cc(-c2nc(-c3ccc4ccccc4c3)c3ccccc3n2)c2c(c1)-c1c(-c3ccc4c(c3)sc3ccccc34)cccc1C21C2CC3CC(C2)CC1C3 ; c1ccc(-c2nc(-c3ccc4c(c3)C3(c5cccc(-c6ccc7c(c6)sc6ccccc67)c5-4)C4CC5CC(C4)CC3C5)nc3ccccc23)cc1]

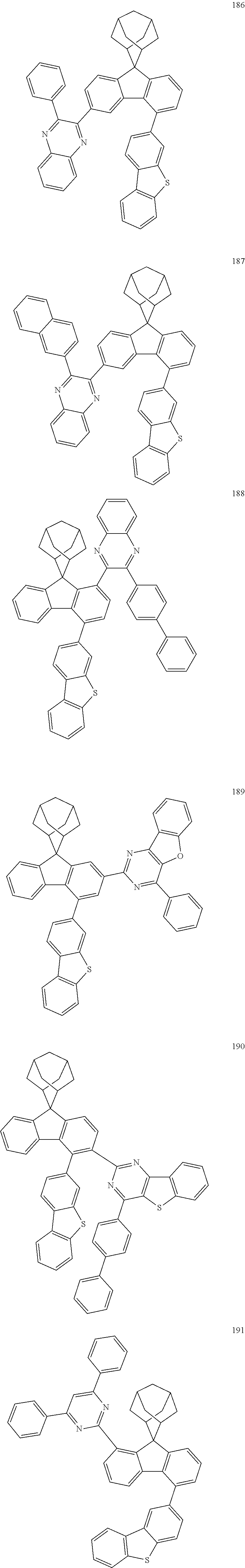

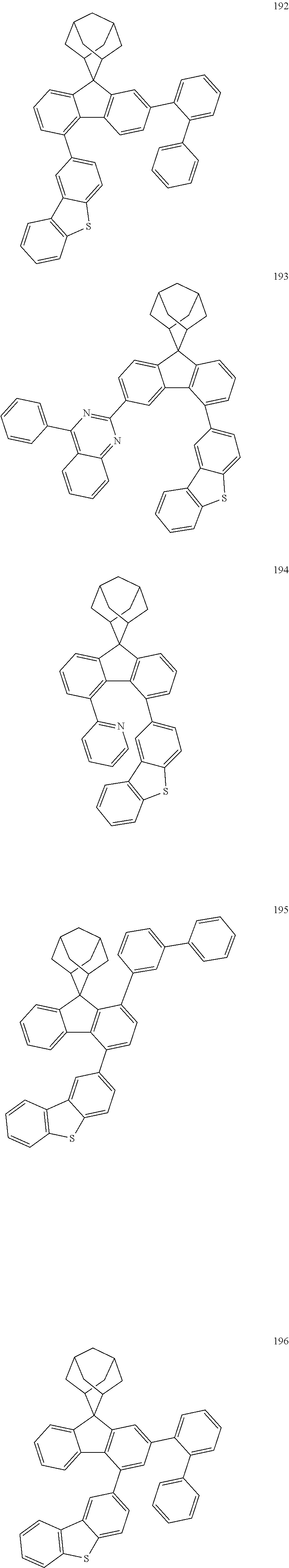 [SMILES: c1ccc(-c2ccccc2-c2ccc3c(c2)C2(c4cccc(-c5ccc6sc7ccccc7c6c5)c4-3)C3CC4CC(C3)CC2C4)cc1 ; c1ccc(-c2nc(-c3ccc4c(c3)-c3c(-c5ccc6sc7ccccc7c6c5)cccc3C43C4CC5CC(C4)CC3C5)nc3ccccc23)cc1 ; c1ccc(-c2cccc3c2-c2c(-c4ccc5sc6ccccc6c5c4)cccc2C32C3CC4CC(C3)CC2C4)nc1 ; c1ccc(-c2cccc(-c3ccc(-c4ccc5sc6ccccc6c5c4)c4c3C3(c5ccccc5-4)C4CC5CC(C4)CC3C5)c2)cc1 ; c1ccc(-c2ccccc2-c2cc(-c3ccc4sc5ccccc5c4c3)c3c(c2)C2(c4ccccc4-3)C3CC4CC(C3)CC2C4)cc1]

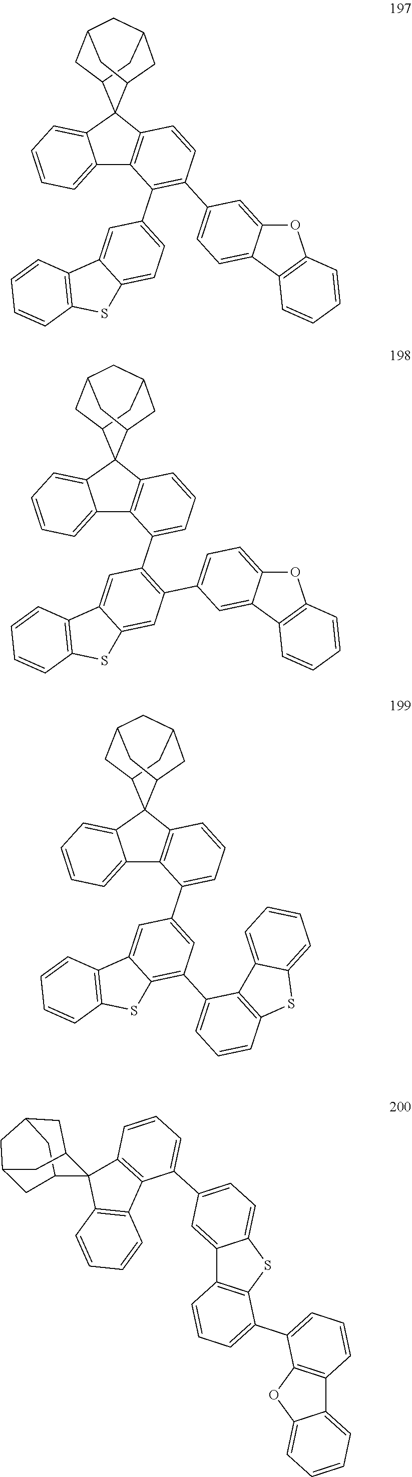 [SMILES: c1ccc2c(c1)-c1c(ccc(-c3ccc4c(c3)oc3ccccc34)c1-c1ccc3sc4ccccc4c3c1)C21C2CC3CC(C2)CC1C3 ; c1ccc2c(c1)-c1c(-c3cc4c(cc3-c3ccc5oc6ccccc6c5c3)sc3ccccc34)cccc1C21C2CC3CC(C2)CC1C3 ; c1ccc2c(c1)-c1c(-c3cc(-c4cccc5sc6ccccc6c45)c4sc5ccccc5c4c3)cccc1C21C2CC3CC(C2)CC1C3 ; c1ccc2c(c1)-c1c(-c3ccc4sc5c(-c6cccc7c6oc6ccccc67)cccc5c4c3)cccc1C21C2CC3CC(C2)CC1C3]

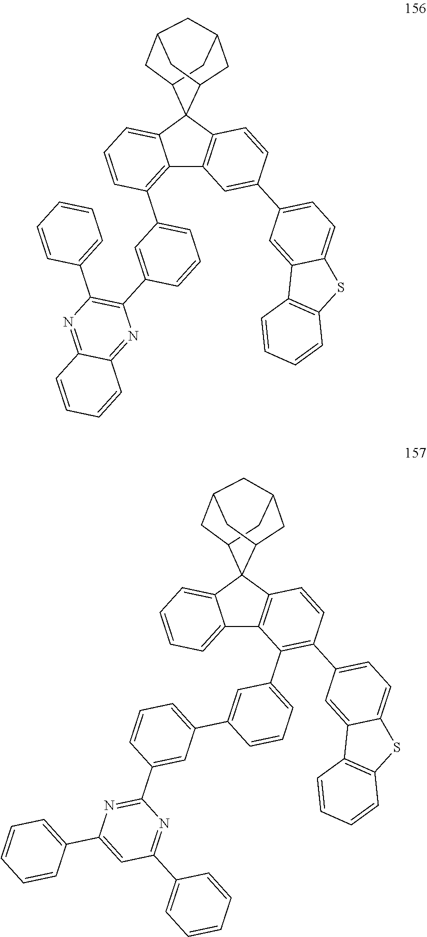 [SMILES: c1ccc(-c2nc3ccccc3nc2-c2cccc(-c3cccc4c3-c3cc(-c5ccc6sc7ccccc7c6c5)ccc3C43C4CC5CC(C4)CC3C5)c2)cc1 ; c1ccc(-c2cc(-c3ccccc3)nc(-c3cccc(-c4cccc(-c5c(-c6ccc7sc8ccccc8c7c6)ccc6c5-c5ccccc5C65C6CC7CC(C6)CC5C7)c4)c3)n2)cc1]

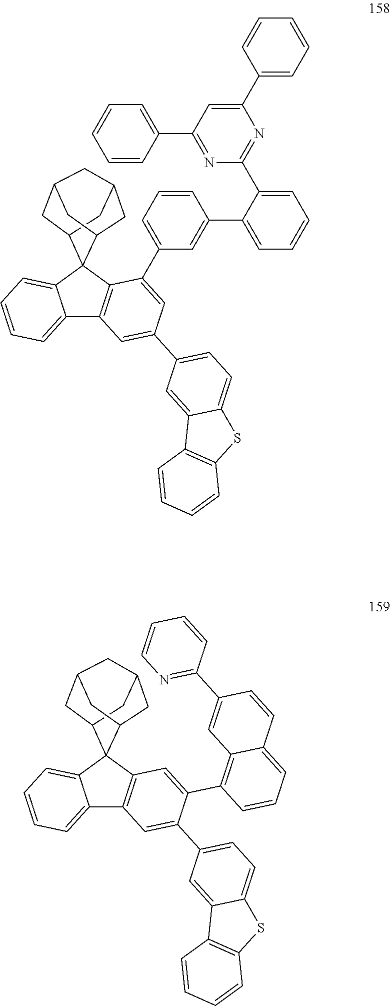 [SMILES: c1ccc(-c2cc(-c3ccccc3)nc(-c3ccccc3-c3cccc(-c4cc(-c5ccc6sc7ccccc7c6c5)cc5c4C4(c6ccccc6-5)C5CC6CC(C5)CC4C6)c3)n2)cc1 ; c1ccc(-c2ccc3cccc(-c4cc5c(cc4-c4ccc6sc7ccccc7c6c4)-c4ccccc4C54C5CC6CC(C5)CC4C6)c3c2)nc1]

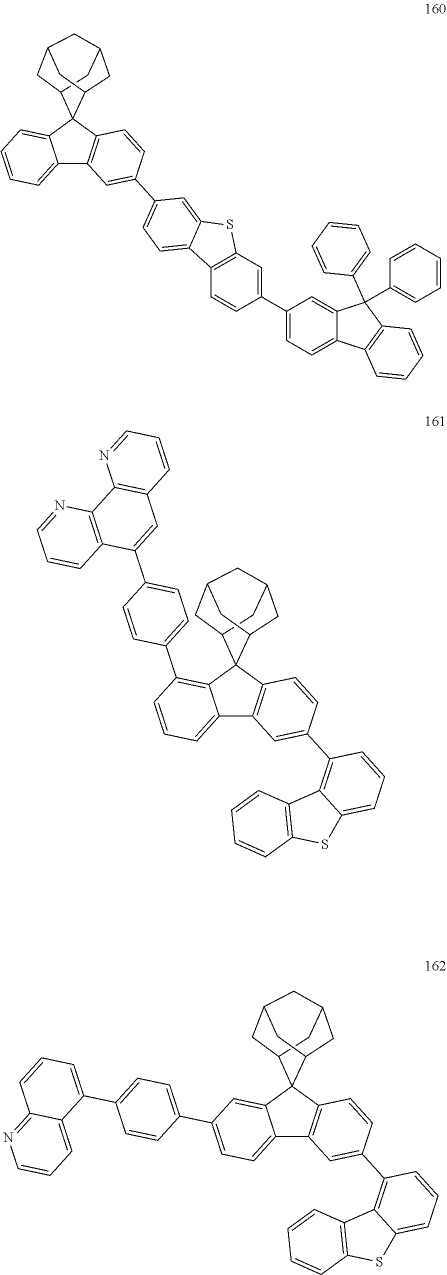 [SMILES: c1ccc(C2(c3ccccc3)c3ccccc3-c3ccc(-c4ccc5c(c4)sc4cc(-c6ccc7c(c6)-c6ccccc6C76C7CC8CC(C7)CC6C8)ccc45)cc32)cc1 ; c1cc(-c2ccc(-c3cc4cccnc4c4ncccc34)cc2)c2c(c1)-c1cc(-c3cccc4sc5ccccc5c34)ccc1C21C2CC3CC(C2)CC1C3 ; c1cc(-c2ccc(-c3ccc4c(c3)C3(c5ccc(-c6cccc7sc8ccccc8c67)cc5-4)C4CC5CC(C4)CC3C5)cc2)c2cccnc2c1]

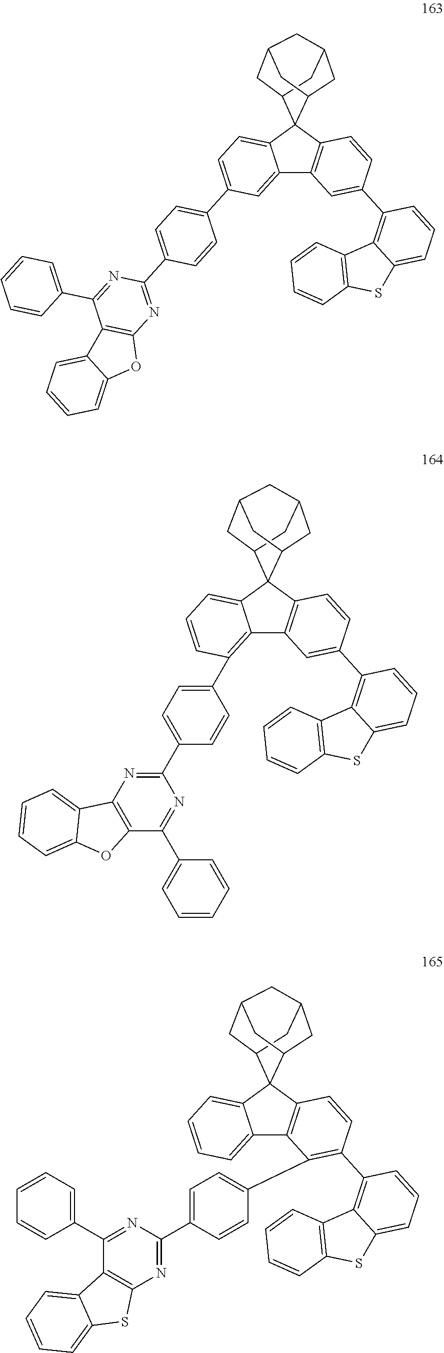 [SMILES: c1ccc(-c2nc(-c3ccc(-c4ccc5c(c4)-c4cc(-c6cccc7sc8ccccc8c67)ccc4C54C5CC6CC(C5)CC4C6)cc3)nc3oc4ccccc4c23)cc1 ; c1ccc(-c2nc(-c3ccc(-c4cccc5c4-c4cc(-c6cccc7sc8ccccc8c67)ccc4C54C5CC6CC(C5)CC4C6)cc3)nc3c2oc2ccccc23)cc1 ; c1ccc(-c2nc(-c3ccc(-c4c(-c5cccc6sc7ccccc7c56)ccc5c4-c4ccccc4C54C5CC6CC(C5)CC4C6)cc3)nc3sc4ccccc4c23)cc1]

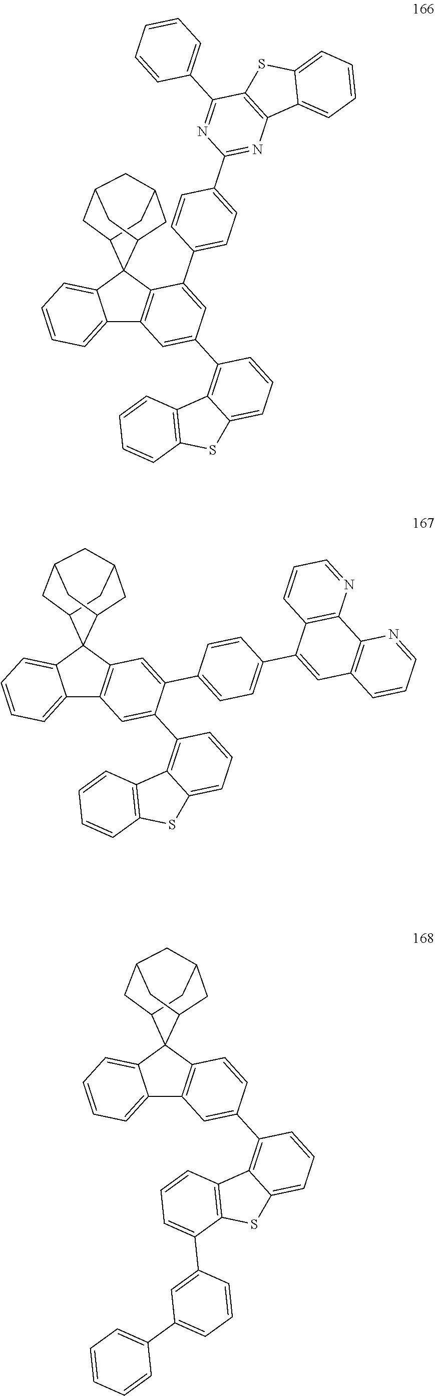 [SMILES: c1ccc(-c2nc(-c3ccc(-c4cc(-c5cccc6sc7ccccc7c56)cc5c4C4(c6ccccc6-5)C5CC6CC(C5)CC4C6)cc3)nc3c2sc2ccccc23)cc1 ; c1ccc2c(c1)-c1cc(-c3cccc4sc5ccccc5c34)c(-c3ccc(-c4cc5cccnc5c5ncccc45)cc3)cc1C21C2CC3CC(C2)CC1C3 ; c1ccc(-c2cccc(-c3cccc4c3sc3cccc(-c5ccc6c(c5)-c5ccccc5C65C6CC7CC(C6)CC5C7)c34)c2)cc1]

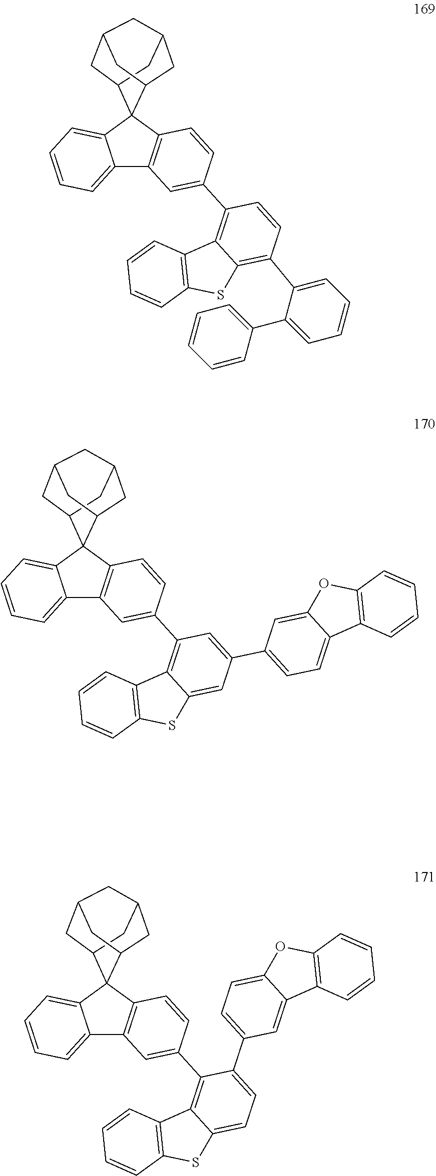 [SMILES: c1ccc(-c2ccccc2-c2ccc(-c3ccc4c(c3)-c3ccccc3C43C4CC5CC(C4)CC3C5)c3c2sc2ccccc23)cc1 ; c1ccc2c(c1)-c1cc(-c3cc(-c4ccc5c(c4)oc4ccccc45)cc4sc5ccccc5c34)ccc1C21C2CC3CC(C2)CC1C3 ; c1ccc2c(c1)-c1cc(-c3c(-c4ccc5oc6ccccc6c5c4)ccc4sc5ccccc5c34)ccc1C21C2CC3CC(C2)CC1C3]

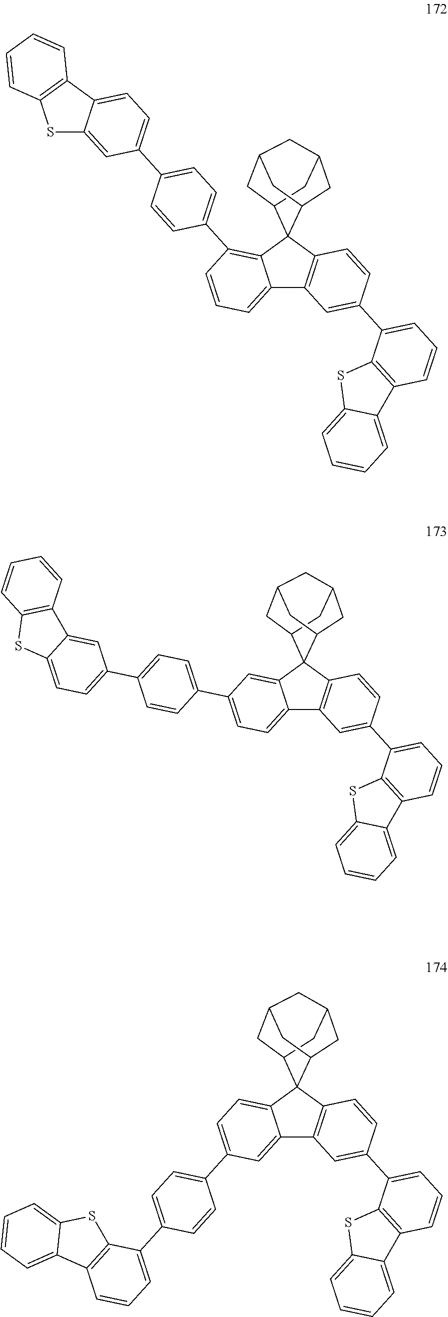 [SMILES: c1cc(-c2ccc(-c3ccc4c(c3)sc3ccccc34)cc2)c2c(c1)-c1cc(-c3cccc4c3sc3ccccc34)ccc1C21C2CC3CC(C2)CC1C3 ; c1ccc2c(c1)sc1ccc(-c3ccc(-c4ccc5c(c4)C4(c6ccc(-c7cccc8c7sc7ccccc78)cc6-5)C5CC6CC(C5)CC4C6)cc3)cc12 ; c1ccc2c(c1)sc1c(-c3ccc(-c4ccc5c(c4)-c4cc(-c6cccc7c6sc6ccccc67)ccc4C54C5CC6CC(C5)CC4C6)cc3)cccc12]

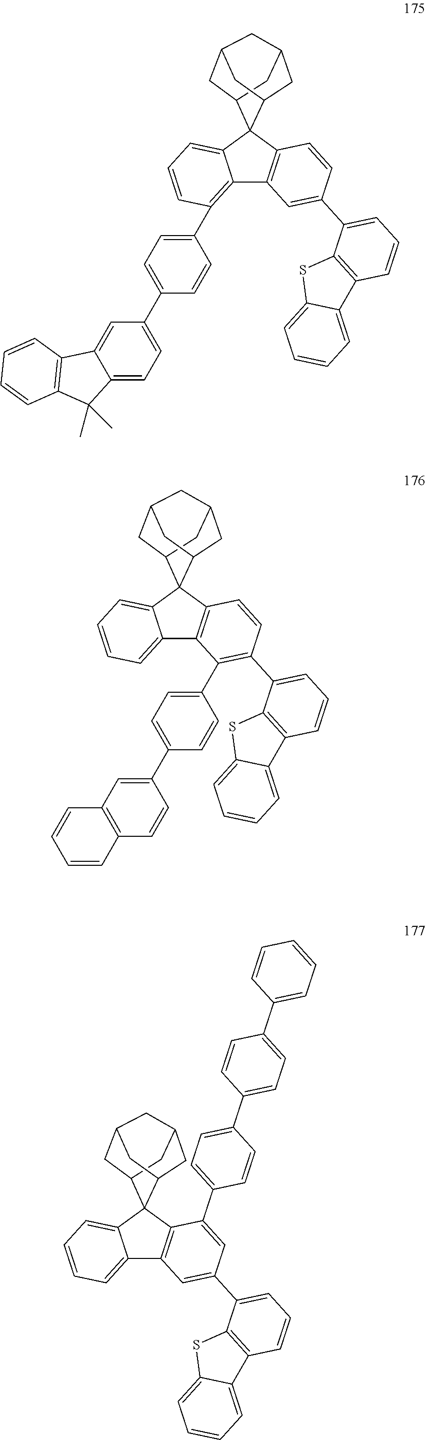 [SMILES: CC1(C)c2ccccc2-c2cc(-c3ccc(-c4cccc5c4-c4cc(-c6cccc7c6sc6ccccc67)ccc4C54C5CC6CC(C5)CC4C6)cc3)ccc21 ; c1ccc2c(c1)-c1c(ccc(-c3cccc4c3sc3ccccc34)c1-c1ccc(-c3ccc4ccccc4c3)cc1)C21C2CC3CC(C2)CC1C3 ; c1ccc(-c2ccc(-c3ccc(-c4cc(-c5cccc6c5sc5ccccc56)cc5c4C4(c6ccccc6-5)C5CC6CC(C5)CC4C6)cc3)cc2)cc1]

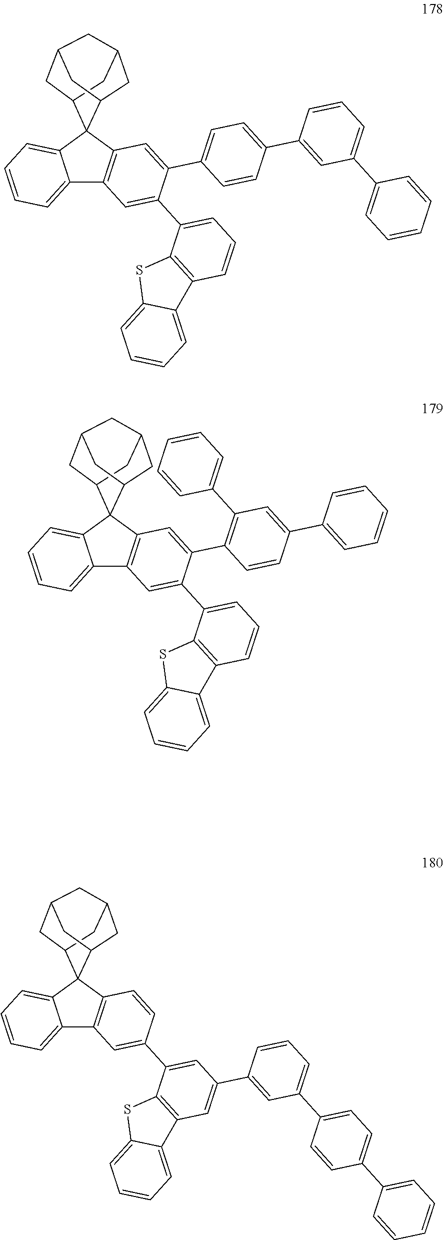 [SMILES: c1ccc(-c2cccc(-c3ccc(-c4cc5c(cc4-c4cccc6c4sc4ccccc46)-c4ccccc4C54C5CC6CC(C5)CC4C6)cc3)c2)cc1 ; c1ccc(-c2ccc(-c3cc4c(cc3-c3cccc5c3sc3ccccc35)-c3ccccc3C43C4CC5CC(C4)CC3C5)c(-c3ccccc3)c2)cc1 ; c1ccc(-c2ccc(-c3cccc(-c4cc(-c5ccc6c(c5)-c5ccccc5C65C6CC7CC(C6)CC5C7)c5sc6ccccc6c5c4)c3)cc2)cc1]

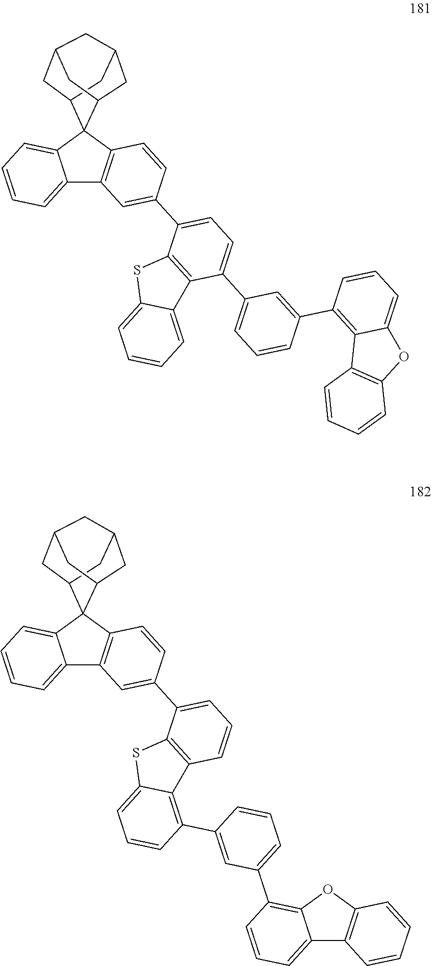 [SMILES: c1cc(-c2cccc3oc4ccccc4c23)cc(-c2ccc(-c3ccc4c(c3)-c3ccccc3C43C4CC5CC(C4)CC3C5)c3sc4ccccc4c23)c1 ; c1cc(-c2cccc3c2oc2ccccc23)cc(-c2cccc3sc4c(-c5ccc6c(c5)-c5ccccc5C65C6CC7CC(C6)CC5C7)cccc4c23)c1]

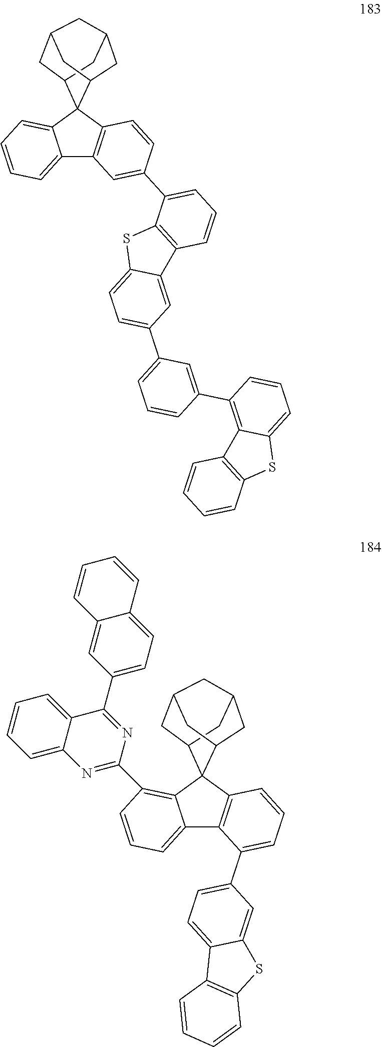 [SMILES: c1cc(-c2ccc3sc4c(-c5ccc6c(c5)-c5ccccc5C65C6CC7CC(C6)CC5C7)cccc4c3c2)cc(-c2cccc3sc4ccccc4c23)c1 ; c1cc(-c2nc(-c3ccc4ccccc4c3)c3ccccc3n2)c2c(c1)-c1c(-c3ccc4c(c3)sc3ccccc34)cccc1C21C2CC3CC(C2)CC1C3]

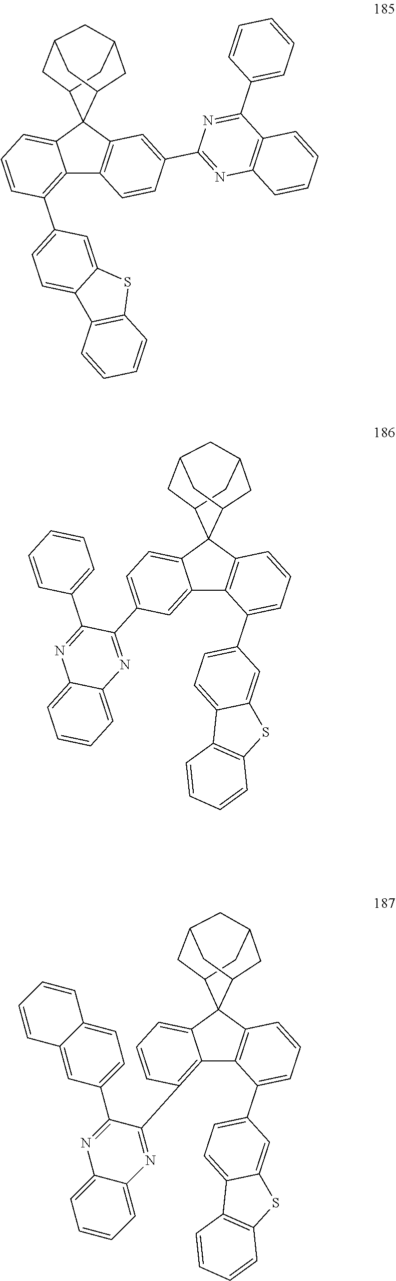 [SMILES: c1ccc(-c2nc(-c3ccc4c(c3)C3(c5cccc(-c6ccc7c(c6)sc6ccccc67)c5-4)C4CC5CC(C4)CC3C5)nc3ccccc23)cc1 ; c1ccc(-c2nc3ccccc3nc2-c2ccc3c(c2)-c2c(-c4ccc5c(c4)sc4ccccc45)cccc2C32C3CC4CC(C3)CC2C4)cc1 ; c1cc(-c2ccc3c(c2)sc2ccccc23)c2c(c1)C1(c3cccc(-c4nc5ccccc5nc4-c4ccc5ccccc5c4)c3-2)C2CC3CC(C2)CC1C3]

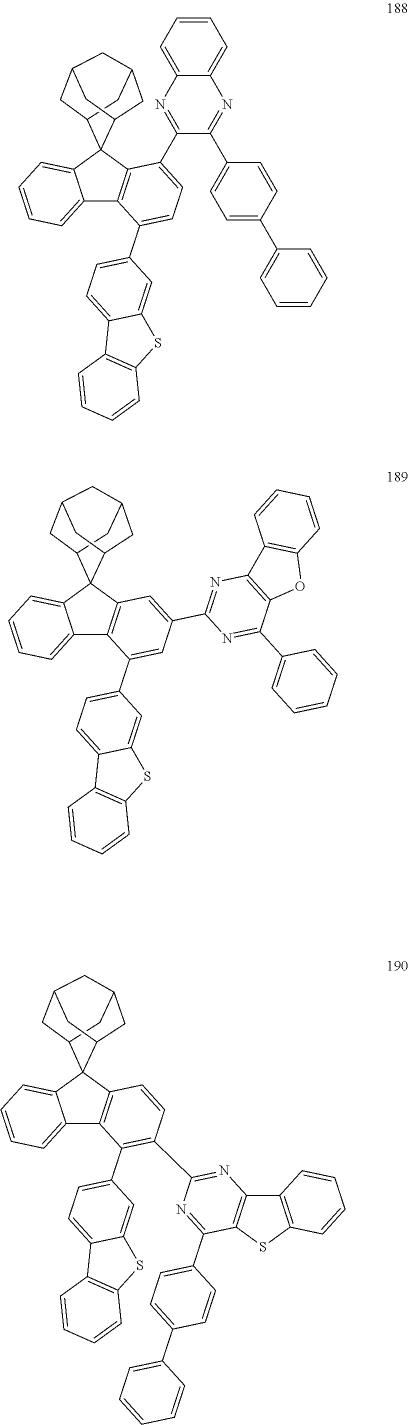 [SMILES: c1ccc(-c2ccc(-c3nc4ccccc4nc3-c3ccc(-c4ccc5c(c4)sc4ccccc45)c4c3C3(c5ccccc5-4)C4CC5CC(C4)CC3C5)cc2)cc1 ; c1ccc(-c2nc(-c3cc(-c4ccc5c(c4)sc4ccccc45)c4c(c3)C3(c5ccccc5-4)C4CC5CC(C4)CC3C5)nc3c2oc2ccccc23)cc1 ; c1ccc(-c2ccc(-c3nc(-c4ccc5c(c4-c4ccc6c(c4)sc4ccccc46)-c4ccccc4C54C5CC6CC(C5)CC4C6)nc4c3sc3ccccc34)cc2)cc1]

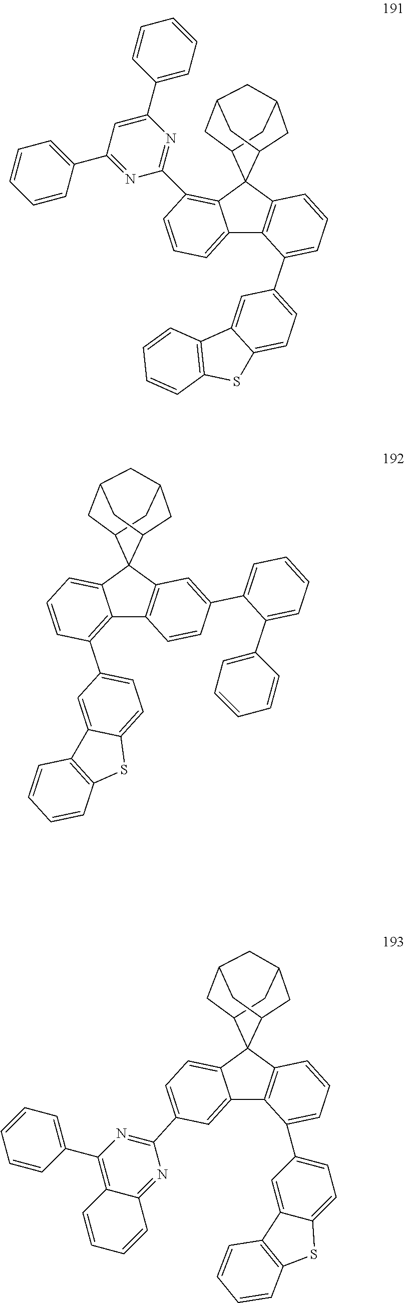 [SMILES: c1ccc(-c2cc(-c3ccccc3)nc(-c3cccc4c3C3(c5cccc(-c6ccc7sc8ccccc8c7c6)c5-4)C4CC5CC(C4)CC3C5)n2)cc1 ; c1ccc(-c2ccccc2-c2ccc3c(c2)C2(c4cccc(-c5ccc6sc7ccccc7c6c5)c4-3)C3CC4CC(C3)CC2C4)cc1 ; c1ccc(-c2nc(-c3ccc4c(c3)-c3c(-c5ccc6sc7ccccc7c6c5)cccc3C43C4CC5CC(C4)CC3C5)nc3ccccc23)cc1]

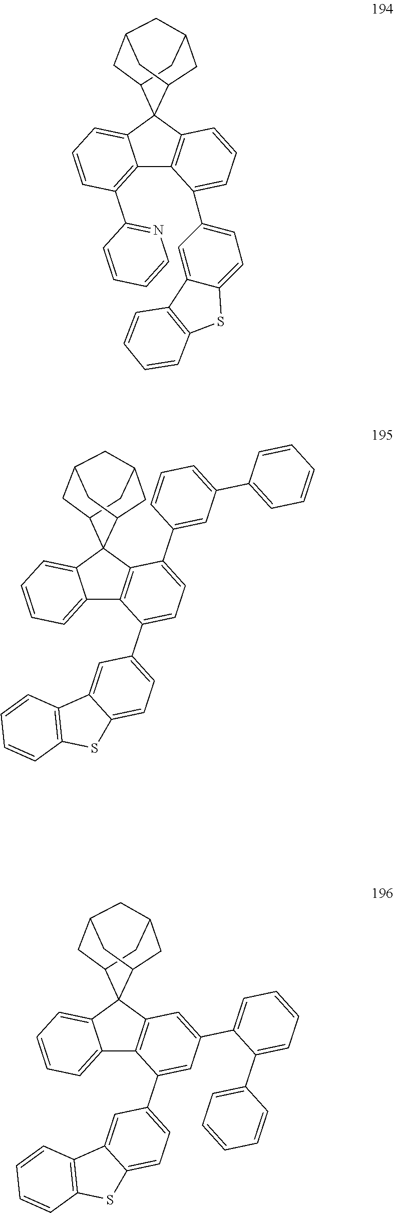 [SMILES: c1ccc(-c2cccc3c2-c2c(-c4ccc5sc6ccccc6c5c4)cccc2C32C3CC4CC(C3)CC2C4)nc1 ; c1ccc(-c2cccc(-c3ccc(-c4ccc5sc6ccccc6c5c4)c4c3C3(c5ccccc5-4)C4CC5CC(C4)CC3C5)c2)cc1 ; c1ccc(-c2ccccc2-c2cc(-c3ccc4sc5ccccc5c4c3)c3c(c2)C2(c4ccccc4-3)C3CC4CC(C3)CC2C4)cc1]

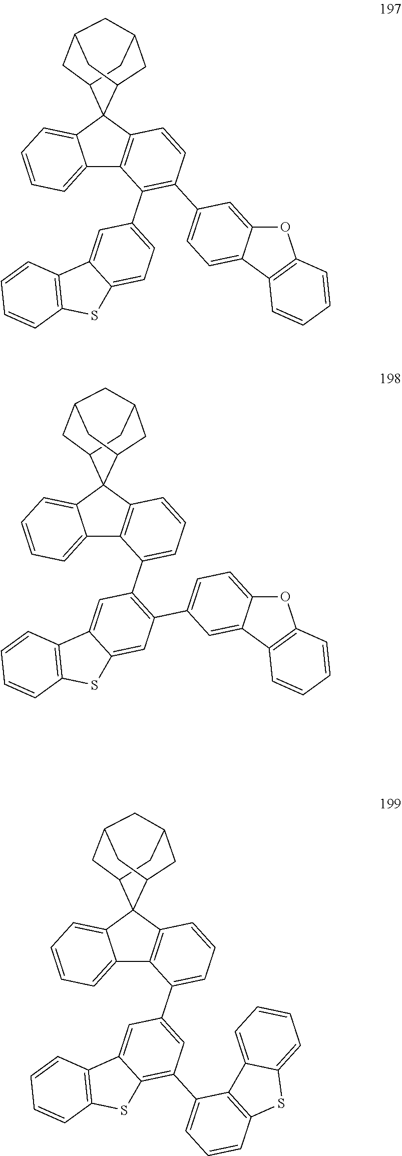 [SMILES: c1ccc2c(c1)-c1c(ccc(-c3ccc4c(c3)oc3ccccc34)c1-c1ccc3sc4ccccc4c3c1)C21C2CC3CC(C2)CC1C3 ; c1ccc2c(c1)-c1c(-c3cc4c(cc3-c3ccc5oc6ccccc6c5c3)sc3ccccc34)cccc1C21C2CC3CC(C2)CC1C3 ; c1ccc2c(c1)-c1c(-c3cc(-c4cccc5sc6ccccc6c45)c4sc5ccccc5c4c3)cccc1C21C2CC3CC(C2)CC1C3]

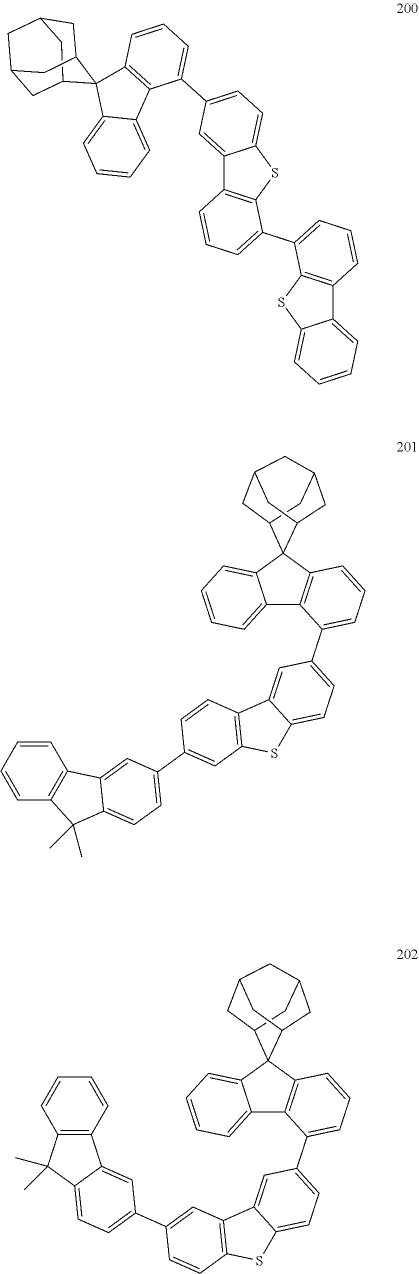 [SMILES: c1ccc2c(c1)-c1c(-c3ccc4sc5c(-c6cccc7c6sc6ccccc67)cccc5c4c3)cccc1C21C2CC3CC(C2)CC1C3 ; CC1(C)c2ccccc2-c2cc(-c3ccc4c(c3)sc3ccc(-c5cccc6c5-c5ccccc5C65C6CC7CC(C6)CC5C7)cc34)ccc21 ; CC1(C)c2ccccc2-c2cc(-c3ccc4sc5ccc(-c6cccc7c6-c6ccccc6C76C7CC8CC(C7)CC6C8)cc5c4c3)ccc21]

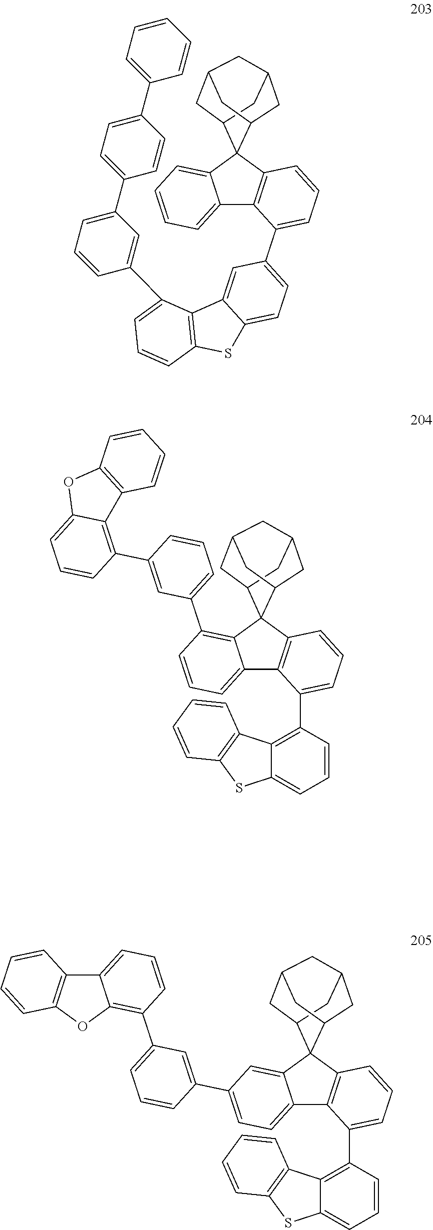 [SMILES: c1ccc(-c2ccc(-c3cccc(-c4cccc5sc6ccc(-c7cccc8c7-c7ccccc7C87C8CC9CC(C8)CC7C9)cc6c45)c3)cc2)cc1 ; c1cc(-c2cccc3c2C2(c4cccc(-c5cccc6sc7ccccc7c56)c4-3)C3CC4CC(C3)CC2C4)cc(-c2cccc3oc4ccccc4c23)c1 ; c1cc(-c2ccc3c(c2)C2(c4cccc(-c5cccc6sc7ccccc7c56)c4-3)C3CC4CC(C3)CC2C4)cc(-c2cccc3c2oc2ccccc23)c1]

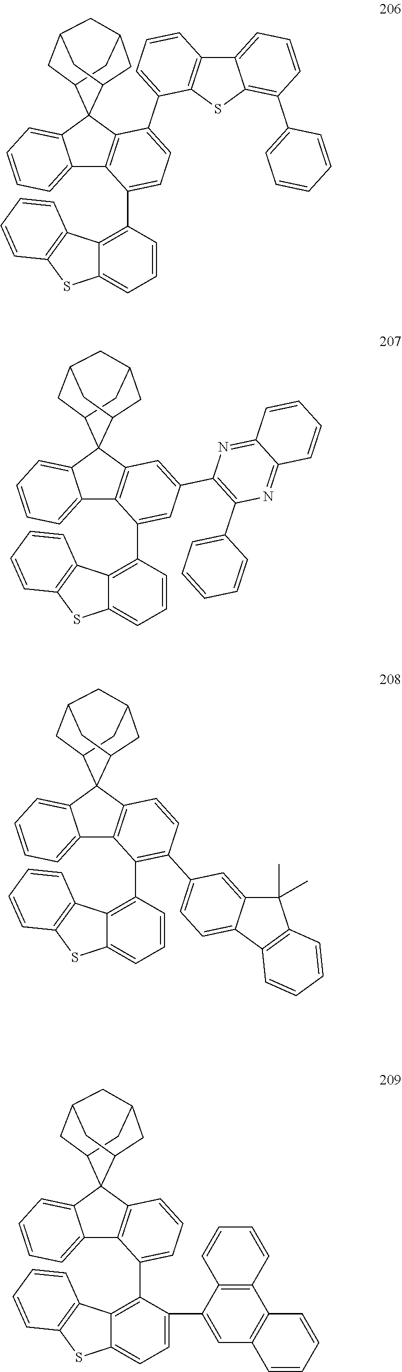 [SMILES: c1ccc(-c2cccc3c2sc2c(-c4ccc(-c5cccc6sc7ccccc7c56)c5c4C4(c6ccccc6-5)C5CC6CC(C5)CC4C6)cccc23)cc1 ; c1ccc(-c2nc3ccccc3nc2-c2cc(-c3cccc4sc5ccccc5c34)c3c(c2)C2(c4ccccc4-3)C3CC4CC(C3)CC2C4)cc1 ; CC1(C)c2ccccc2-c2ccc(-c3ccc4c(c3-c3cccc5sc6ccccc6c35)-c3ccccc3C43C4CC5CC(C4)CC3C5)cc21 ; c1ccc2c(c1)-c1c(-c3c(-c4cc5ccccc5c5ccccc45)ccc4sc5ccccc5c34)cccc1C21C2CC3CC(C2)CC1C3]

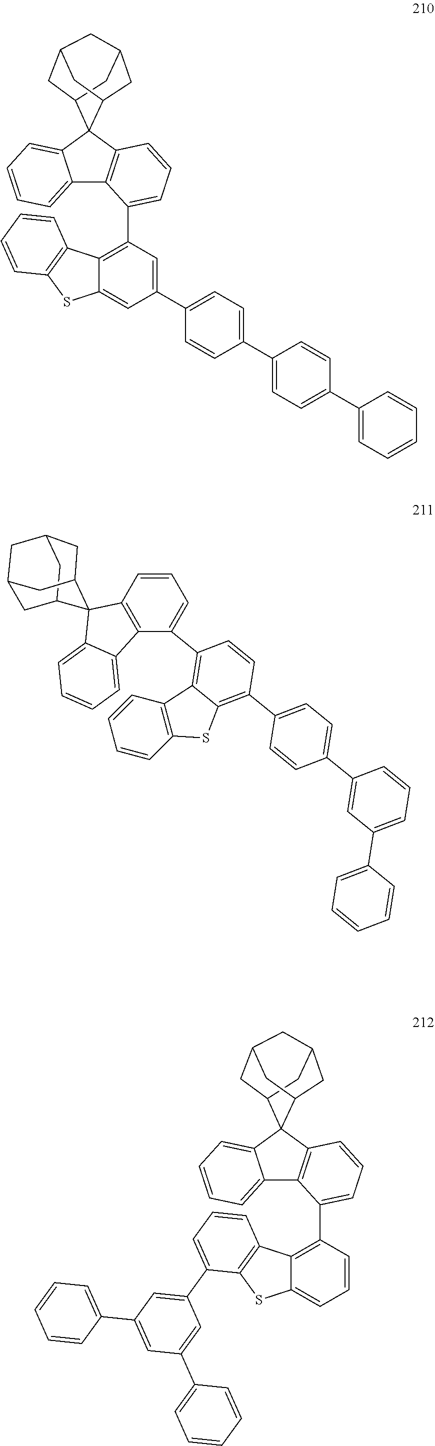 [SMILES: c1ccc(-c2ccc(-c3ccc(-c4cc(-c5cccc6c5-c5ccccc5C65C6CC7CC(C6)CC5C7)c5c(c4)sc4ccccc45)cc3)cc2)cc1 ; c1ccc(-c2cccc(-c3ccc(-c4ccc(-c5cccc6c5-c5ccccc5C65C6CC7CC(C6)CC5C7)c5c4sc4ccccc45)cc3)c2)cc1 ; c1ccc(-c2cc(-c3ccccc3)cc(-c3cccc4c3sc3cccc(-c5cccc6c5-c5ccccc5C65C6CC7CC(C6)CC5C7)c34)c2)cc1]

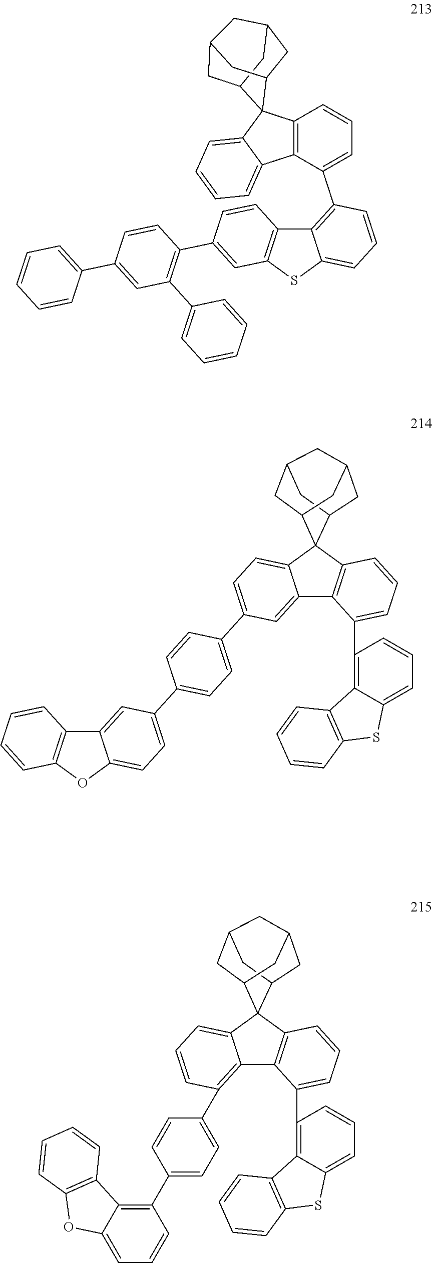 [SMILES: c1ccc(-c2ccc(-c3ccc4c(c3)sc3cccc(-c5cccc6c5-c5ccccc5C65C6CC7CC(C6)CC5C7)c34)c(-c3ccccc3)c2)cc1 ; c1cc(-c2cccc3sc4ccccc4c23)c2c(c1)C1(c3ccc(-c4ccc(-c5ccc6oc7ccccc7c6c5)cc4)cc3-2)C2CC3CC(C2)CC1C3 ; c1cc(-c2ccc(-c3cccc4oc5ccccc5c34)cc2)c2c(c1)C1(c3cccc(-c4cccc5sc6ccccc6c45)c3-2)C2CC3CC(C2)CC1C3]

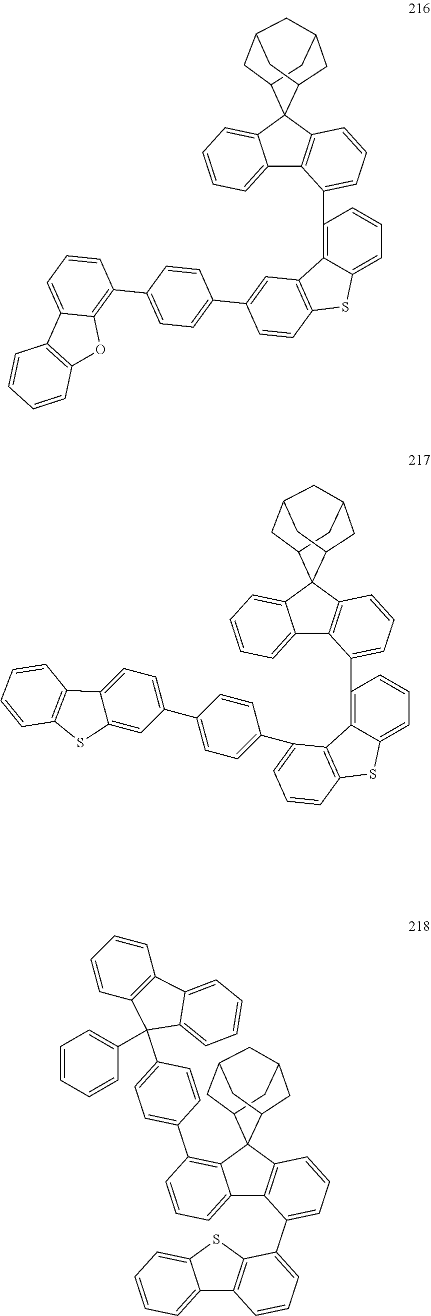 [SMILES: c1ccc2c(c1)-c1c(-c3cccc4sc5ccc(-c6ccc(-c7cccc8c7oc7ccccc78)cc6)cc5c34)cccc1C21C2CC3CC(C2)CC1C3 ; c1ccc2c(c1)-c1c(-c3cccc4sc5cccc(-c6ccc(-c7ccc8c(c7)sc7ccccc78)cc6)c5c34)cccc1C21C2CC3CC(C2)CC1C3 ; c1ccc(C2(c3ccc(-c4cccc5c4C4(c6cccc(-c7cccc8c7sc7ccccc78)c6-5)C5CC6CC(C5)CC4C6)cc3)c3ccccc3-c3ccccc32)cc1]

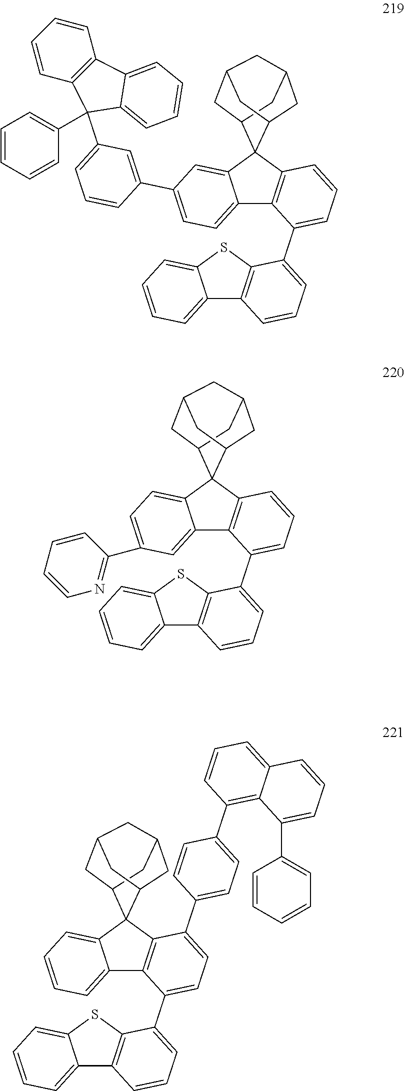 [SMILES: c1ccc(C2(c3cccc(-c4ccc5c(c4)C4(c6cccc(-c7cccc8c7sc7ccccc78)c6-5)C5CC6CC(C5)CC4C6)c3)c3ccccc3-c3ccccc32)cc1 ; c1ccc(-c2ccc3c(c2)-c2c(-c4cccc5c4sc4ccccc45)cccc2C32C3CC4CC(C3)CC2C4)nc1 ; c1ccc(-c2cccc3cccc(-c4ccc(-c5ccc(-c6cccc7c6sc6ccccc67)c6c5C5(c7ccccc7-6)C6CC7CC(C6)CC5C7)cc4)c23)cc1]

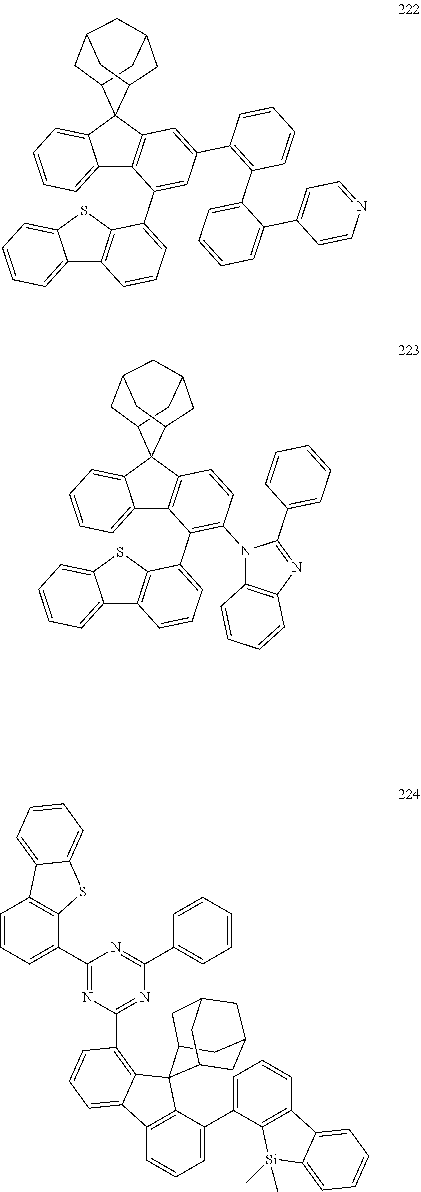 [SMILES: c1ccc(-c2ccccc2-c2cc(-c3cccc4c3sc3ccccc34)c3c(c2)C2(c4ccccc4-3)C3CC4CC(C3)CC2C4)c(-c2ccncc2)c1 ; c1ccc(-c2nc3ccccc3n2-c2ccc3c(c2-c2cccc4c2sc2ccccc24)-c2ccccc2C32C3CC4CC(C3)CC2C4)cc1 ; C[Si]1(C)c2ccccc2-c2cccc(-c3cccc4c3C3(c5c(-c6nc(-c7ccccc7)nc(-c7cccc8c7sc7ccccc78)n6)cccc5-4)C4CC5CC(C4)CC3C5)c21]

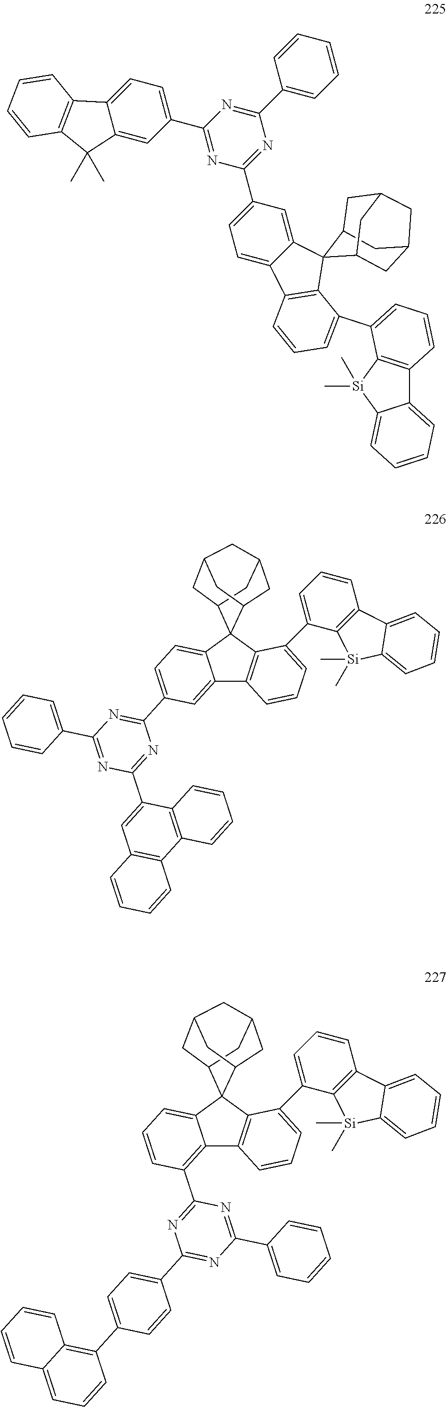 [SMILES: CC1(C)c2ccccc2-c2ccc(-c3nc(-c4ccccc4)nc(-c4ccc5c(c4)C4(c6c-5cccc6-c5cccc6c5[Si](C)(C)c5ccccc5-6)C5CC6CC(C5)CC4C6)n3)cc21 ; C[Si]1(C)c2ccccc2-c2cccc(-c3cccc4c3C3(c5ccc(-c6nc(-c7ccccc7)nc(-c7cc8ccccc8c8ccccc78)n6)cc5-4)C4CC5CC(C4)CC3C5)c21 ; C[Si]1(C)c2ccccc2-c2cccc(-c3cccc4c3C3(c5cccc(-c6nc(-c7ccccc7)nc(-c7ccc(-c8cccc9ccccc89)cc7)n6)c5-4)C4CC5CC(C4)CC3C5)c21]

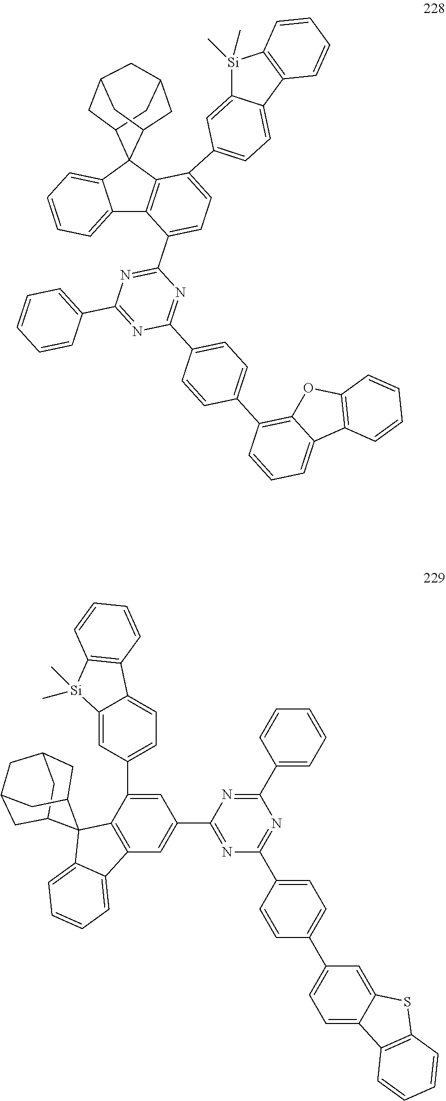 [SMILES: C[Si]1(C)c2ccccc2-c2ccc(-c3ccc(-c4nc(-c5ccccc5)nc(-c5ccc(-c6cccc7c6oc6ccccc67)cc5)n4)c4c3C3(c5ccccc5-4)C4CC5CC(C4)CC3C5)cc21 ; C[Si]1(C)c2ccccc2-c2ccc(-c3cc(-c4nc(-c5ccccc5)nc(-c5ccc(-c6ccc7c(c6)sc6ccccc67)cc5)n4)cc4c3C3(c5ccccc5-4)C4CC5CC(C4)CC3C5)cc21]

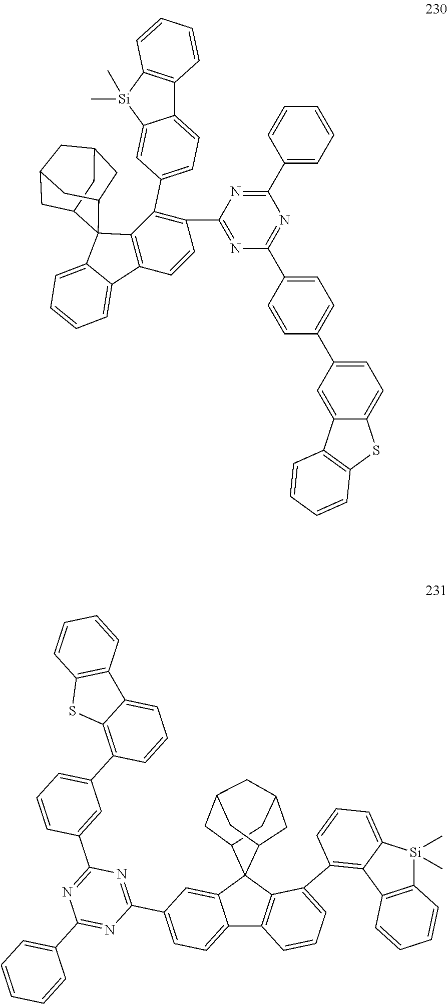 [SMILES: C[Si]1(C)c2ccccc2-c2ccc(-c3c(-c4nc(-c5ccccc5)nc(-c5ccc(-c6ccc7sc8ccccc8c7c6)cc5)n4)ccc4c3C3(c5ccccc5-4)C4CC5CC(C4)CC3C5)cc21 ; C[Si]1(C)c2ccccc2-c2c(-c3cccc4c3C3(c5cc(-c6nc(-c7ccccc7)nc(-c7cccc(-c8cccc9c8sc8ccccc89)c7)n6)ccc5-4)C4CC5CC(C4)CC3C5)cccc21]

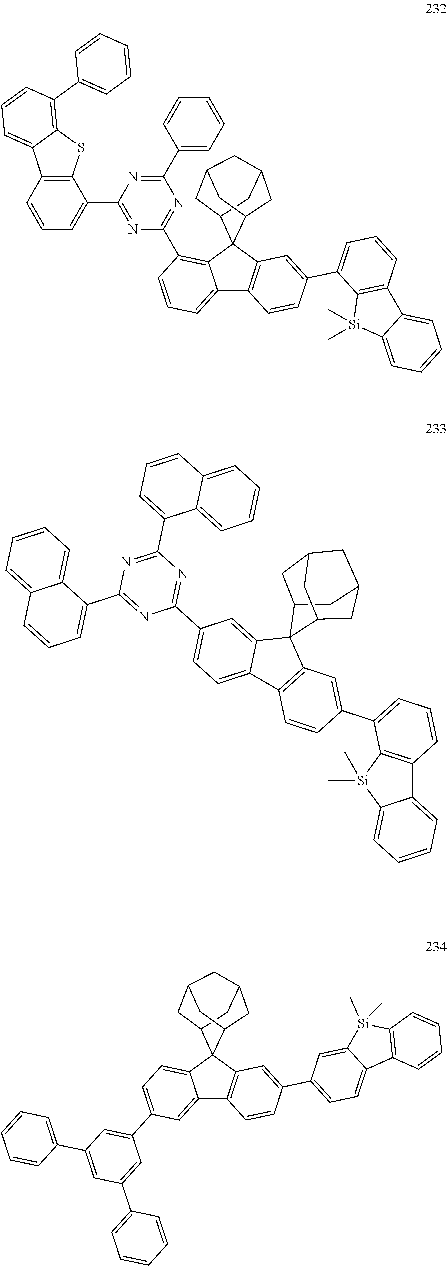 [SMILES: C[Si]1(C)c2ccccc2-c2cccc(-c3ccc4c(c3)C3(c5c(-c6nc(-c7ccccc7)nc(-c7cccc8c7sc7c(-c9ccccc9)cccc78)n6)cccc5-4)C4CC5CC(C4)CC3C5)c21 ; C[Si]1(C)c2ccccc2-c2cccc(-c3ccc4c(c3)C3(c5cc(-c6nc(-c7cccc8ccccc78)nc(-c7cccc8ccccc78)n6)ccc5-4)C4CC5CC(C4)CC3C5)c21 ; C[Si]1(C)c2ccccc2-c2ccc(-c3ccc4c(c3)C3(c5ccc(-c6cc(-c7ccccc7)cc(-c7ccccc7)c6)cc5-4)C4CC5CC(C4)CC3C5)cc21]

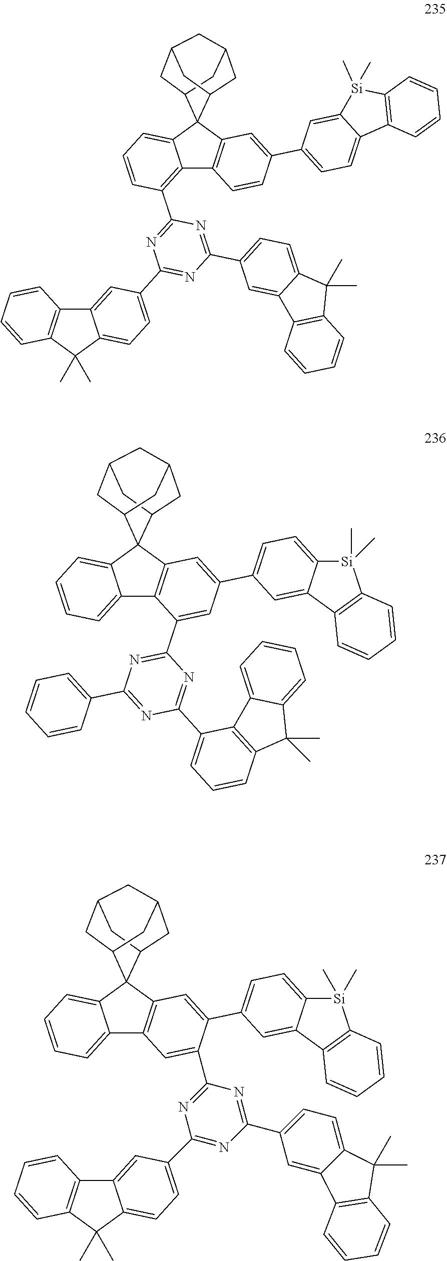 [SMILES: CC1(C)c2ccccc2-c2cc(-c3nc(-c4ccc5c(c4)-c4ccccc4C5(C)C)nc(-c4cccc5c4-c4ccc(-c6ccc7c(c6)[Si](C)(C)c6ccccc6-7)cc4C54C5CC6CC(C5)CC4C6)n3)ccc21 ; CC1(C)c2ccccc2-c2c(-c3nc(-c4ccccc4)nc(-c4cc(-c5ccc6c(c5)-c5ccccc5[Si]6(C)C)cc5c4-c4ccccc4C54C5CC6CC(C5)CC4C6)n3)cccc21 ; CC1(C)c2ccccc2-c2cc(-c3nc(-c4ccc5c(c4)-c4ccccc4C5(C)C)nc(-c4cc5c(cc4-c4ccc6c(c4)-c4ccccc4[Si]6(C)C)C4(c6ccccc6-5)C5CC6CC(C5)CC4C6)n3)ccc21]

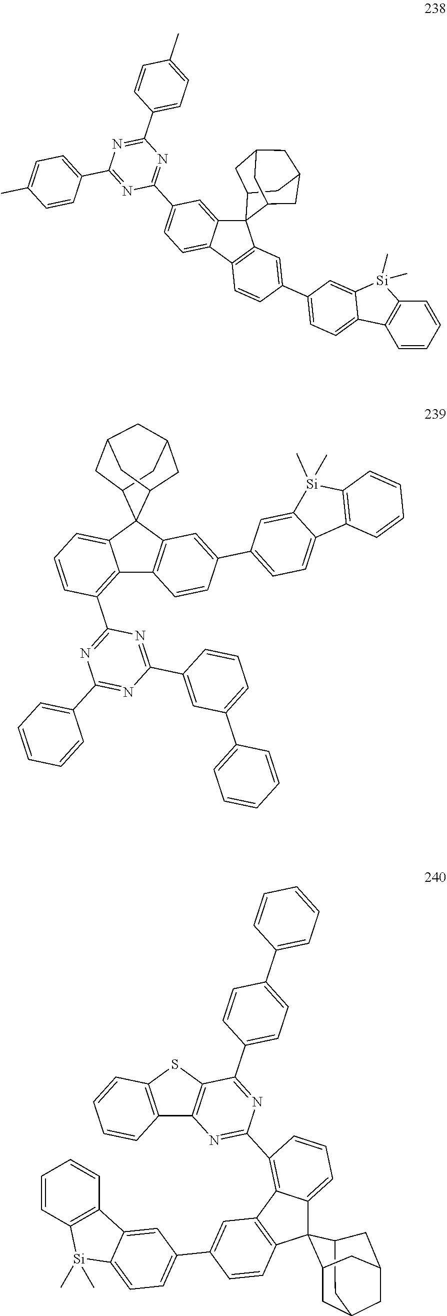 [SMILES: Cc1ccc(-c2nc(-c3ccc(C)cc3)nc(-c3ccc4c(c3)C3(c5cc(-c6ccc7c(c6)[Si](C)(C)c6ccccc6-7)ccc5-4)C4CC5CC(C4)CC3C5)n2)cc1 ; C[Si]1(C)c2ccccc2-c2ccc(-c3ccc4c(c3)C3(c5cccc(-c6nc(-c7ccccc7)nc(-c7cccc(-c8ccccc8)c7)n6)c5-4)C4CC5CC(C4)CC3C5)cc21 ; C[Si]1(C)c2ccccc2-c2cc(-c3ccc4c(c3)-c3c(-c5nc(-c6ccc(-c7ccccc7)cc6)c6sc7ccccc7c6n5)cccc3C43C4CC5CC(C4)CC3C5)ccc21]

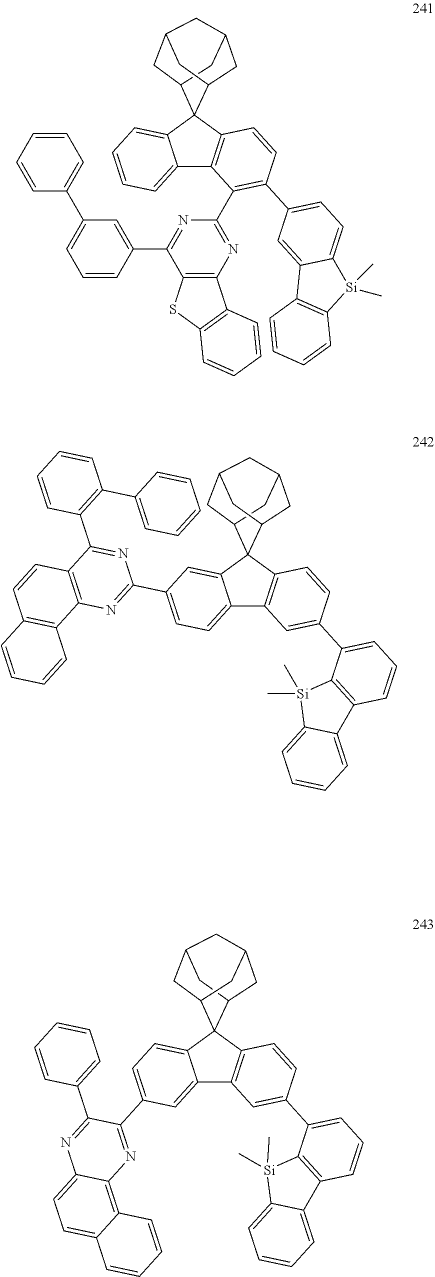 [SMILES: C[Si]1(C)c2ccccc2-c2cc(-c3ccc4c(c3-c3nc(-c5cccc(-c6ccccc6)c5)c5sc6ccccc6c5n3)-c3ccccc3C43C4CC5CC(C4)CC3C5)ccc21 ; C[Si]1(C)c2ccccc2-c2cccc(-c3ccc4c(c3)-c3ccc(-c5nc(-c6ccccc6-c6ccccc6)c6ccc7ccccc7c6n5)cc3C43C4CC5CC(C4)CC3C5)c21 ; C[Si]1(C)c2ccccc2-c2cccc(-c3ccc4c(c3)-c3cc(-c5nc6c(ccc7ccccc76)nc5-c5ccccc5)ccc3C43C4CC5CC(C4)CC3C5)c21]

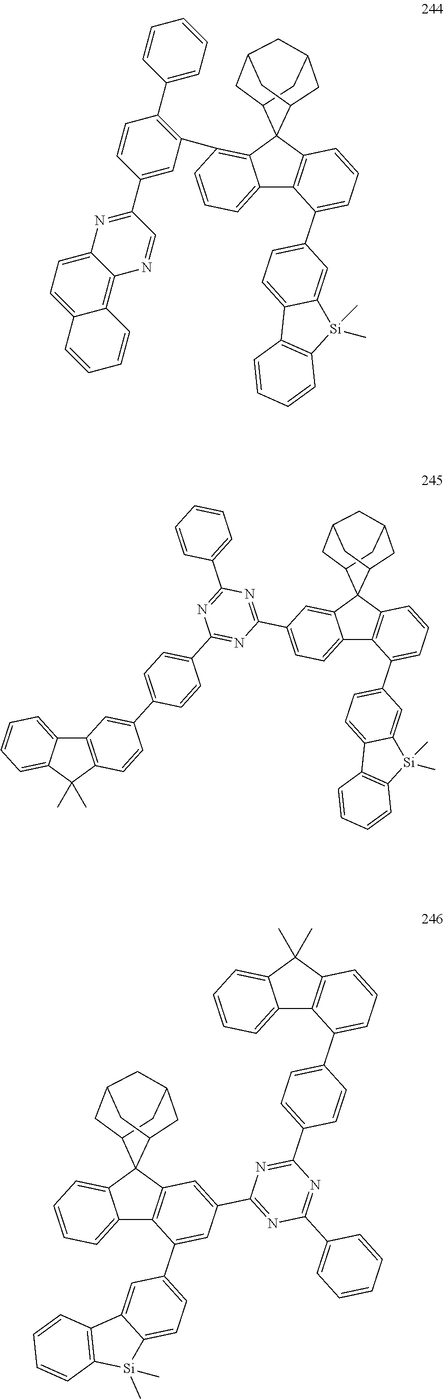 [SMILES: C[Si]1(C)c2ccccc2-c2ccc(-c3cccc4c3-c3cccc(-c5cc(-c6cnc7c(ccc8ccccc87)n6)ccc5-c5ccccc5)c3C43C4CC5CC(C4)CC3C5)cc21 ; CC1(C)c2ccccc2-c2cc(-c3ccc(-c4nc(-c5ccccc5)nc(-c5ccc6c(c5)C5(c7cccc(-c8ccc9c(c8)[Si](C)(C)c8ccccc8-9)c7-6)C6CC7CC(C6)CC5C7)n4)cc3)ccc21 ; CC1(C)c2ccccc2-c2c(-c3ccc(-c4nc(-c5ccccc5)nc(-c5cc(-c6ccc7c(c6)-c6ccccc6[Si]7(C)C)c6c(c5)C5(c7ccccc7-6)C6CC7CC(C6)CC5C7)n4)cc3)cccc21]

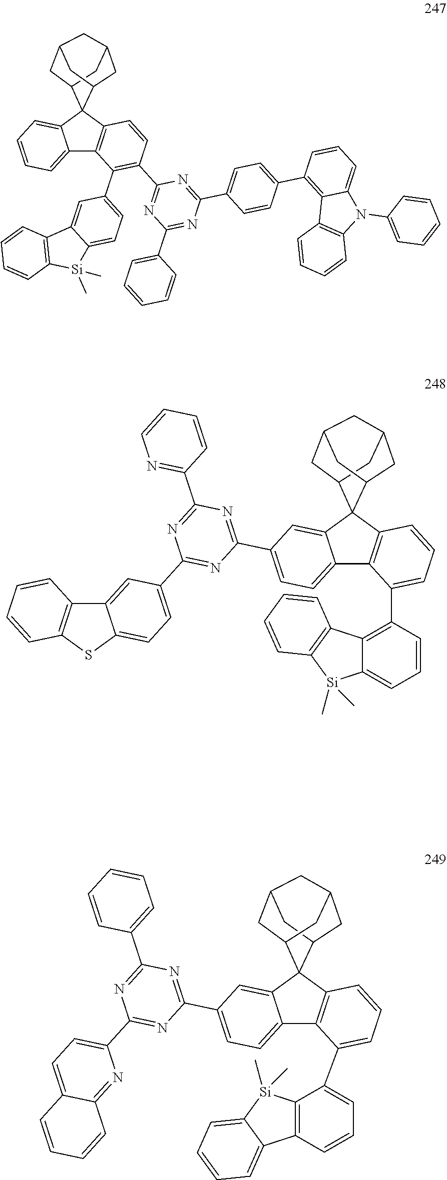 [SMILES: C[Si]1(C)c2ccccc2-c2cc(-c3c(-c4nc(-c5ccccc5)nc(-c5ccc(-c6cccc7c6c6ccccc6n7-c6ccccc6)cc5)n4)ccc4c3-c3ccccc3C43C4CC5CC(C4)CC3C5)ccc21 ; C[Si]1(C)c2ccccc2-c2c(-c3cccc4c3-c3ccc(-c5nc(-c6ccc7sc8ccccc8c7c6)nc(-c6ccccn6)n5)cc3C43C4CC5CC(C4)CC3C5)cccc21 ; C[Si]1(C)c2ccccc2-c2cccc(-c3cccc4c3-c3ccc(-c5nc(-c6ccccc6)nc(-c6ccc7ccccc7n6)n5)cc3C43C4CC5CC(C4)CC3C5)c21]

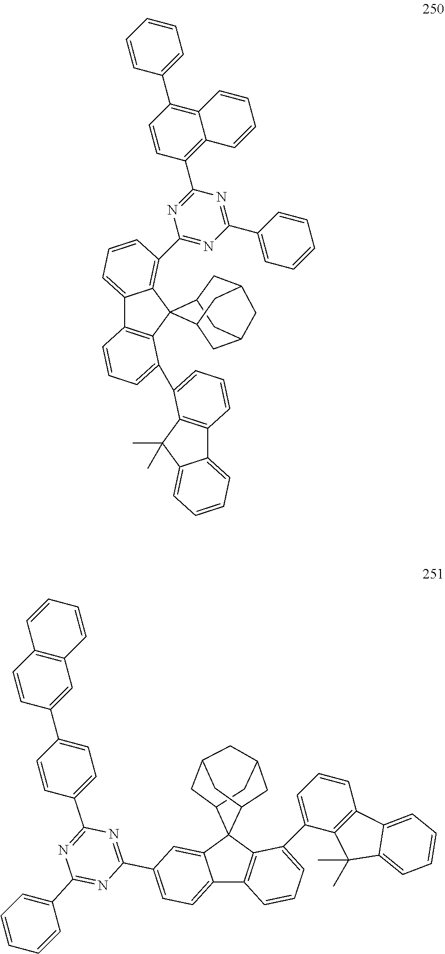 [SMILES: CC1(C)c2ccccc2-c2cccc(-c3cccc4c3C3(c5c(-c6nc(-c7ccccc7)nc(-c7ccc(-c8ccccc8)c8ccccc78)n6)cccc5-4)C4CC5CC(C4)CC3C5)c21 ; CC1(C)c2ccccc2-c2cccc(-c3cccc4c3C3(c5cc(-c6nc(-c7ccccc7)nc(-c7ccc(-c8ccc9ccccc9c8)cc7)n6)ccc5-4)C4CC5CC(C4)CC3C5)c21]

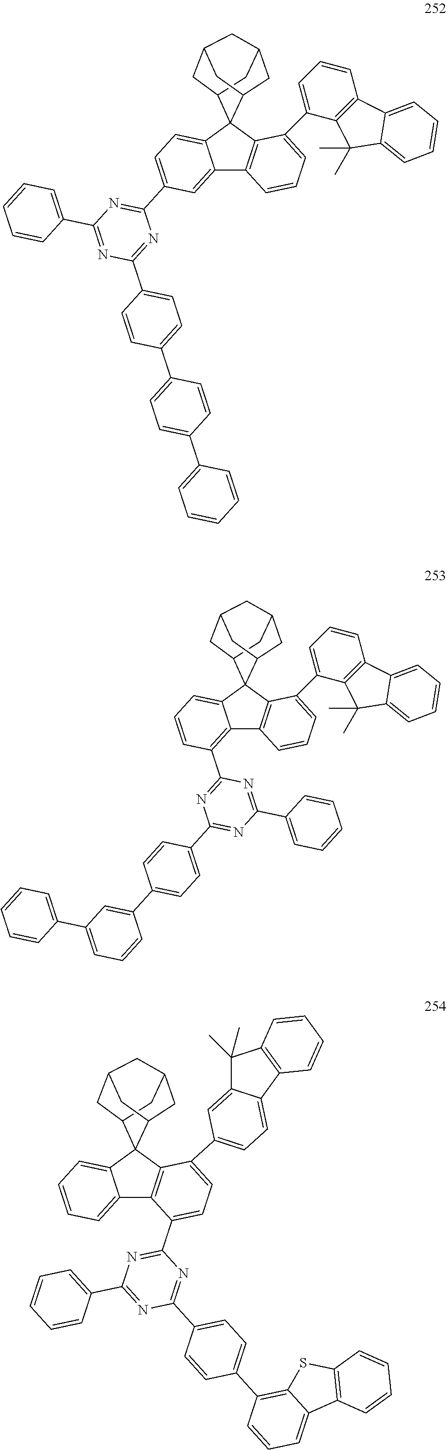 [SMILES: CC1(C)c2ccccc2-c2cccc(-c3cccc4c3C3(c5ccc(-c6nc(-c7ccccc7)nc(-c7ccc(-c8ccc(-c9ccccc9)cc8)cc7)n6)cc5-4)C4CC5CC(C4)CC3C5)c21 ; CC1(C)c2ccccc2-c2cccc(-c3cccc4c3C3(c5cccc(-c6nc(-c7ccccc7)nc(-c7ccc(-c8cccc(-c9ccccc9)c8)cc7)n6)c5-4)C4CC5CC(C4)CC3C5)c21 ; CC1(C)c2ccccc2-c2ccc(-c3ccc(-c4nc(-c5ccccc5)nc(-c5ccc(-c6cccc7c6sc6ccccc67)cc5)n4)c4c3C3(c5ccccc5-4)C4CC5CC(C4)CC3C5)cc21]

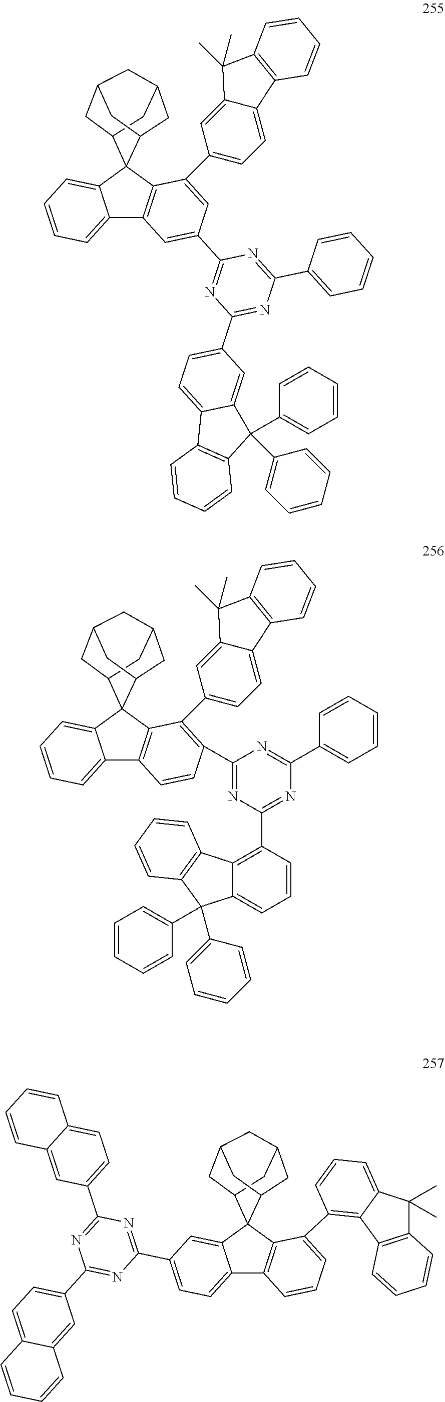 [SMILES: CC1(C)c2ccccc2-c2ccc(-c3cc(-c4nc(-c5ccccc5)nc(-c5ccc6c(c5)C(c5ccccc5)(c5ccccc5)c5ccccc5-6)n4)cc4c3C3(c5ccccc5-4)C4CC5CC(C4)CC3C5)cc21 ; CC1(C)c2ccccc2-c2ccc(-c3c(-c4nc(-c5ccccc5)nc(-c5cccc6c5-c5ccccc5C6(c5ccccc5)c5ccccc5)n4)ccc4c3C3(c5ccccc5-4)C4CC5CC(C4)CC3C5)cc21 ; CC1(C)c2ccccc2-c2c(-c3cccc4c3C3(c5cc(-c6nc(-c7ccc8ccccc8c7)nc(-c7ccc8ccccc8c7)n6)ccc5-4)C4CC5CC(C4)CC3C5)cccc21]

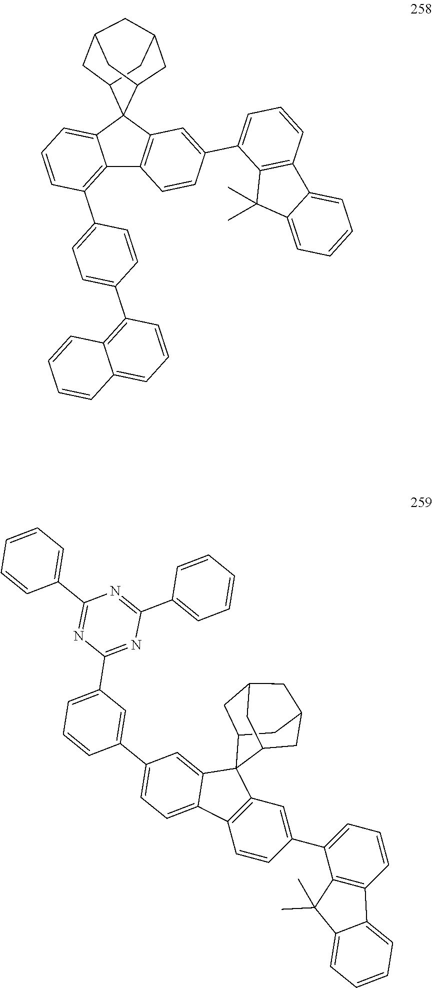

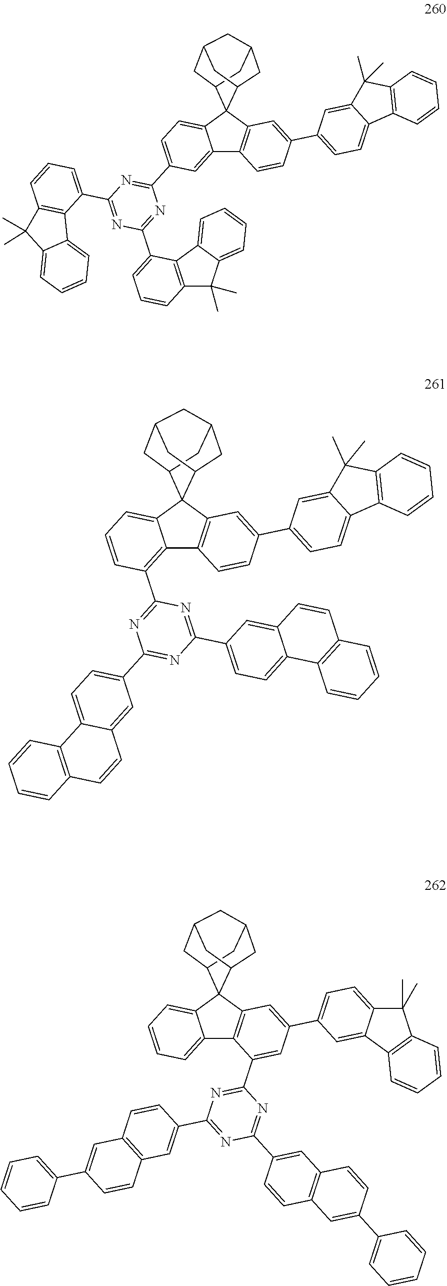 [SMILES: CC1(C)c2ccccc2-c2ccc(-c3ccc4c(c3)C3(c5ccc(-c6nc(-c7cccc8c7-c7ccccc7C8(C)C)nc(-c7cccc8c7-c7ccccc7C8(C)C)n6)cc5-4)C4CC5CC(C4)CC3C5)cc21 ; CC1(C)c2ccccc2-c2ccc(-c3ccc4c(c3)C3(c5cccc(-c6nc(-c7ccc8c(ccc9ccccc98)c7)nc(-c7ccc8c(ccc9ccccc98)c7)n6)c5-4)C4CC5CC(C4)CC3C5)cc21 ; CC1(C)c2ccccc2-c2cc(-c3cc(-c4nc(-c5ccc6cc(-c7ccccc7)ccc6c5)nc(-c5ccc6cc(-c7ccccc7)ccc6c5)n4)c4c(c3)C3(c5ccccc5-4)C4CC5CC(C4)CC3C5)ccc21]

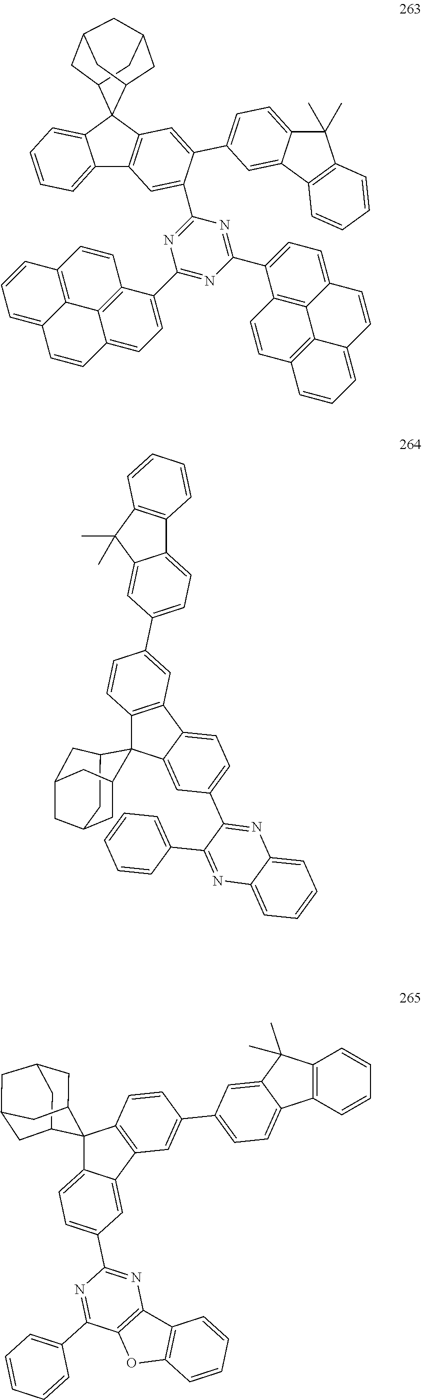 [SMILES: CC1(C)c2ccccc2-c2cc(-c3cc4c(cc3-c3nc(-c5ccc6ccc7cccc8ccc5c6c78)nc(-c5ccc6ccc7cccc8ccc5c6c78)n3)-c3ccccc3C43C4CC5CC(C4)CC3C5)ccc21 ; CC1(C)c2ccccc2-c2ccc(-c3ccc4c(c3)-c3ccc(-c5nc6ccccc6nc5-c5ccccc5)cc3C43C4CC5CC(C4)CC3C5)cc21 ; CC1(C)c2ccccc2-c2ccc(-c3ccc4c(c3)-c3cc(-c5nc(-c6ccccc6)c6oc7ccccc7c6n5)ccc3C43C4CC5CC(C4)CC3C5)cc21]

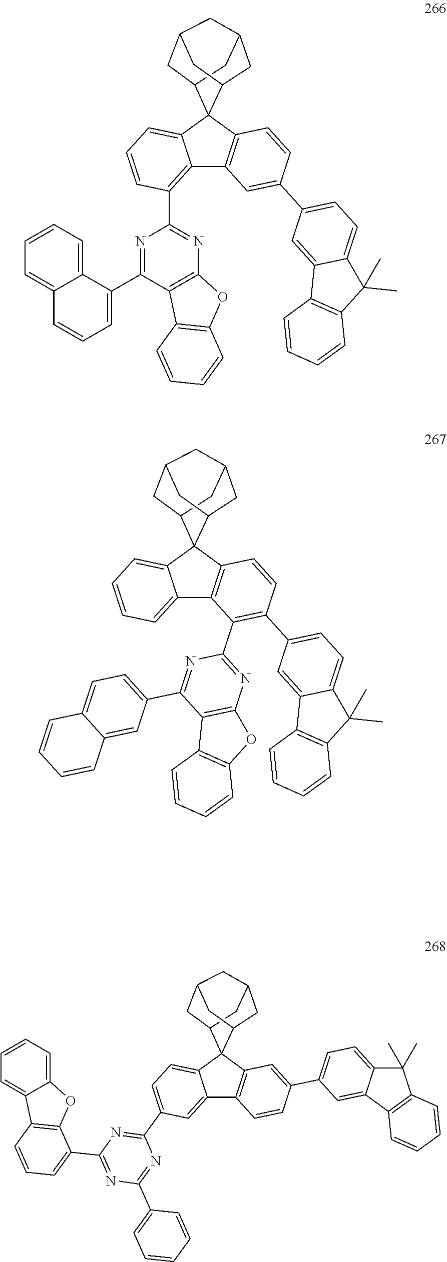 [SMILES: CC1(C)c2ccccc2-c2cc(-c3ccc4c(c3)-c3c(-c5nc(-c6cccc7ccccc67)c6c(n5)oc5ccccc56)cccc3C43C4CC5CC(C4)CC3C5)ccc21 ; CC1(C)c2ccccc2-c2cc(-c3ccc4c(c3-c3nc(-c5ccc6ccccc6c5)c5c(n3)oc3ccccc35)-c3ccccc3C43C4CC5CC(C4)CC3C5)ccc21 ; CC1(C)c2ccccc2-c2cc(-c3ccc4c(c3)C3(c5ccc(-c6nc(-c7ccccc7)nc(-c7cccc8c7oc7ccccc78)n6)cc5-4)C4CC5CC(C4)CC3C5)ccc21]

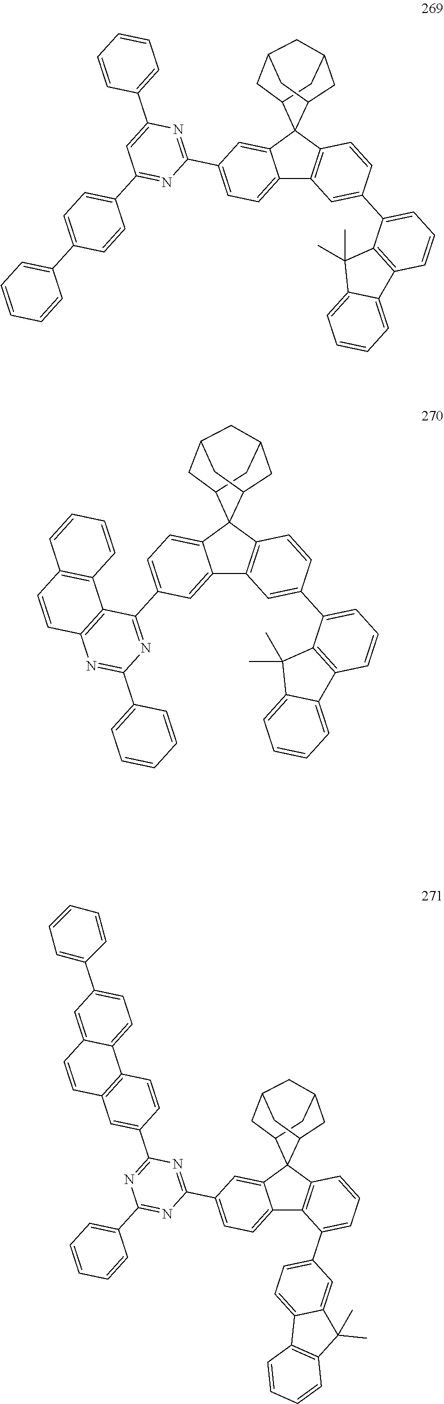 [SMILES: CC1(C)c2ccccc2-c2cccc(-c3ccc4c(c3)-c3ccc(-c5nc(-c6ccccc6)cc(-c6ccc(-c7ccccc7)cc6)n5)cc3C43C4CC5CC(C4)CC3C5)c21 ; CC1(C)c2ccccc2-c2cccc(-c3ccc4c(c3)-c3cc(-c5nc(-c6ccccc6)nc6ccc7ccccc7c56)ccc3C43C4CC5CC(C4)CC3C5)c21 ; CC1(C)c2ccccc2-c2ccc(-c3cccc4c3-c3ccc(-c5nc(-c6ccccc6)nc(-c6ccc7c(ccc8cc(-c9ccccc9)ccc87)c6)n5)cc3C43C4CC5CC(C4)CC3C5)cc21]

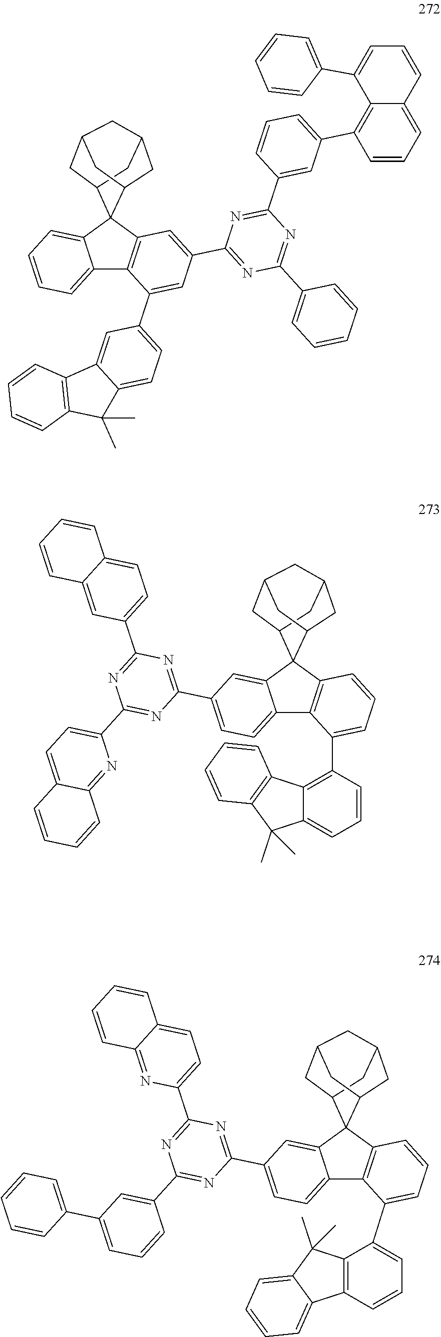 [SMILES: CC1(C)c2ccccc2-c2cc(-c3cc(-c4nc(-c5ccccc5)nc(-c5cccc(-c6cccc7cccc(-c8ccccc8)c67)c5)n4)cc4c3-c3ccccc3C43C4CC5CC(C4)CC3C5)ccc21 ; CC1(C)c2ccccc2-c2c(-c3cccc4c3-c3ccc(-c5nc(-c6ccc7ccccc7c6)nc(-c6ccc7ccccc7n6)n5)cc3C43C4CC5CC(C4)CC3C5)cccc21 ; CC1(C)c2ccccc2-c2cccc(-c3cccc4c3-c3ccc(-c5nc(-c6cccc(-c7ccccc7)c6)nc(-c6ccc7ccccc7n6)n5)cc3C43C4CC5CC(C4)CC3C5)c21]

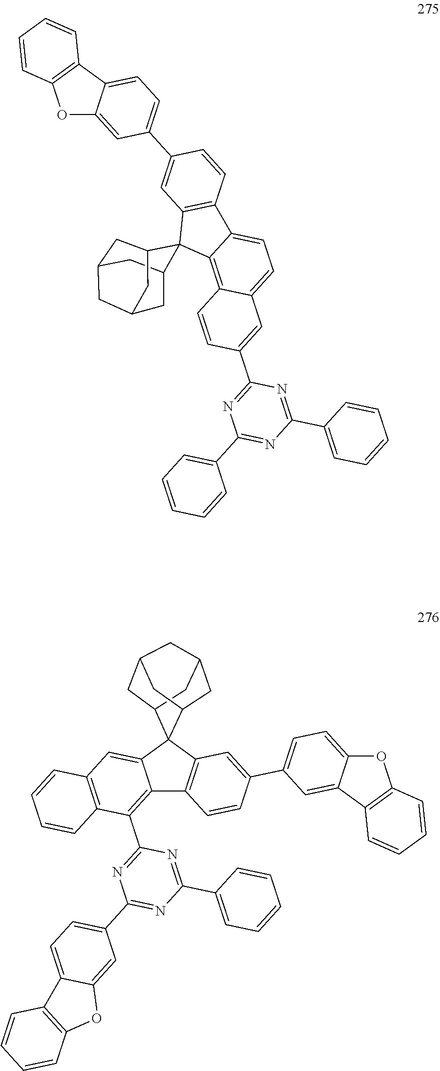 [SMILES: c1ccc(-c2nc(-c3ccccc3)nc(-c3ccc4c5c(ccc4c3)-c3ccc(-c4ccc6c(c4)oc4ccccc46)cc3C53C4CC5CC(C4)CC3C5)n2)cc1 ; c1ccc(-c2nc(-c3ccc4c(c3)oc3ccccc34)nc(-c3c4c(cc5ccccc35)C3(c5cc(-c6ccc7oc8ccccc8c7c6)ccc5-4)C4CC5CC(C4)CC3C5)n2)cc1]

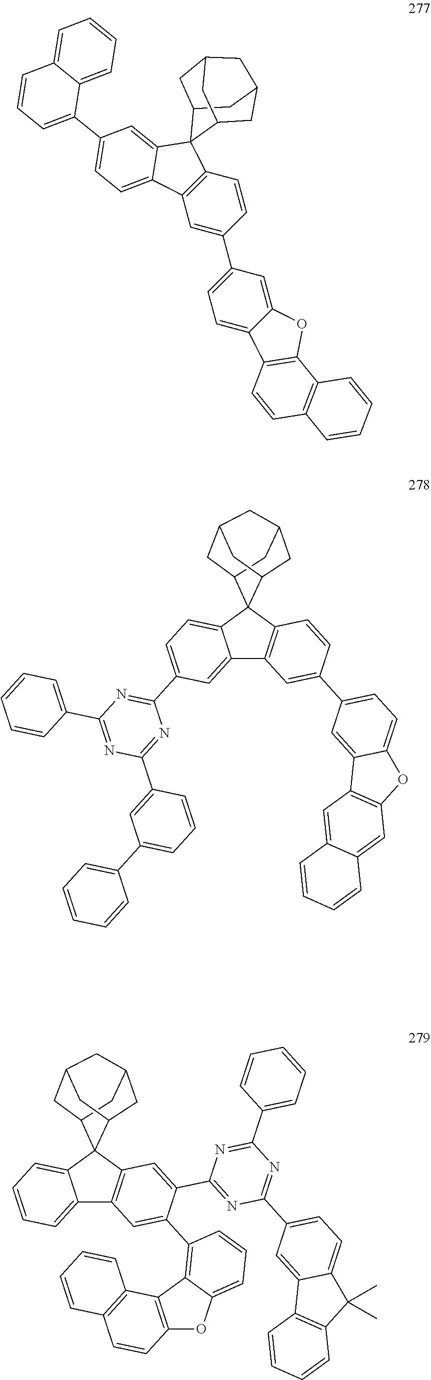 [SMILES: c1ccc2c(-c3ccc4c(c3)C3(c5ccc(-c6ccc7c(c6)oc6c8ccccc8ccc76)cc5-4)C4CC5CC(C4)CC3C5)cccc2c1 ; c1ccc(-c2cccc(-c3nc(-c4ccccc4)nc(-c4ccc5c(c4)-c4cc(-c6ccc7oc8cc9ccccc9cc8c7c6)ccc4C54C5CC6CC(C5)CC4C6)n3)c2)cc1 ; CC1(C)c2ccccc2-c2cc(-c3nc(-c4ccccc4)nc(-c4cc5c(cc4-c4cccc6oc7ccc8ccccc8c7c46)-c4ccccc4C54C5CC6CC(C5)CC4C6)n3)ccc21]

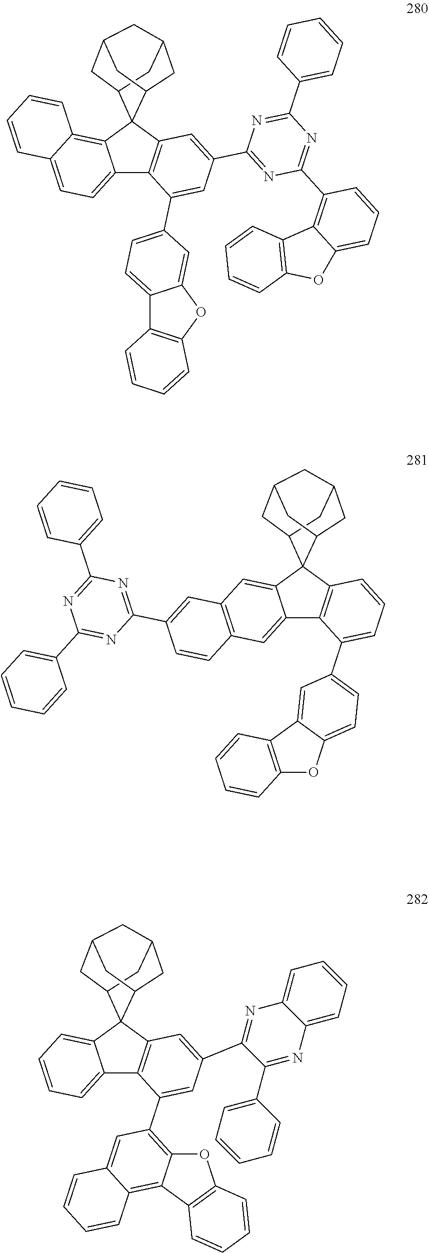 [SMILES: c1ccc(-c2nc(-c3cc(-c4ccc5c(c4)oc4ccccc45)c4c(c3)C3(c5c-4ccc4ccccc54)C4CC5CC(C4)CC3C5)nc(-c3cccc4oc5ccccc5c34)n2)cc1 ; c1ccc(-c2nc(-c3ccccc3)nc(-c3ccc4cc5c(cc4c3)C3(c4cccc(-c6ccc7oc8ccccc8c7c6)c4-5)C4CC5CC(C4)CC3C5)n2)cc1 ; c1ccc(-c2nc3ccccc3nc2-c2cc(-c3cc4ccccc4c4c3oc3ccccc34)c3c(c2)C2(c4ccccc4-3)C3CC4CC(C3)CC2C4)cc1]

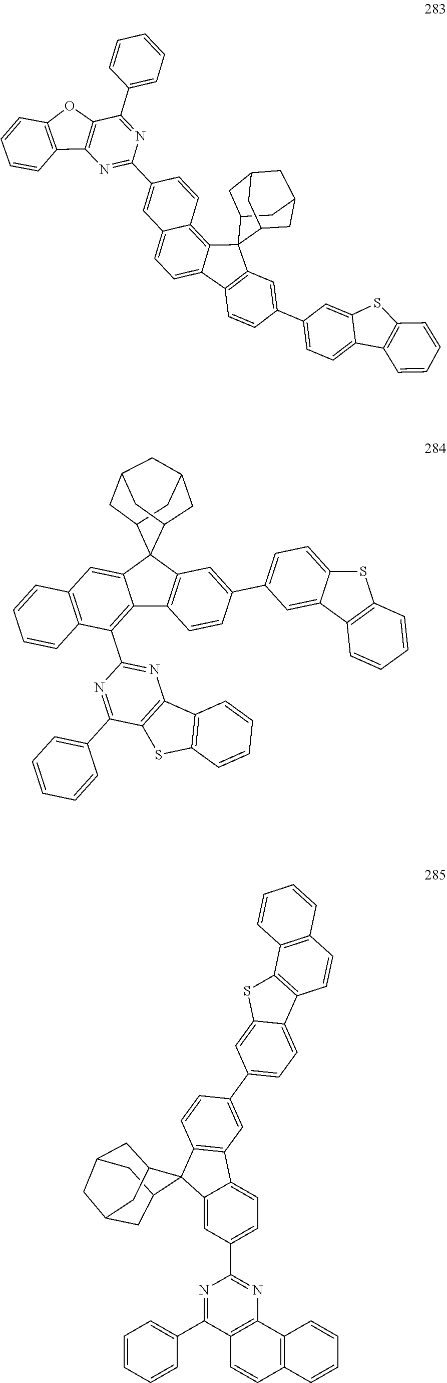 [SMILES: c1ccc(-c2nc(-c3ccc4c5c(ccc4c3)-c3ccc(-c4ccc6c(c4)sc4ccccc46)cc3C53C4CC5CC(C4)CC3C5)nc3c2oc2ccccc23)cc1 ; c1ccc(-c2nc(-c3c4c(cc5ccccc35)C3(c5cc(-c6ccc7sc8ccccc8c7c6)ccc5-4)C4CC5CC(C4)CC3C5)nc3c2sc2ccccc23)cc1 ; c1ccc(-c2nc(-c3ccc4c(c3)C3(c5ccc(-c6ccc7c(c6)sc6c8ccccc8ccc76)cc5-4)C4CC5CC(C4)CC3C5)nc3c2ccc2ccccc23)cc1]

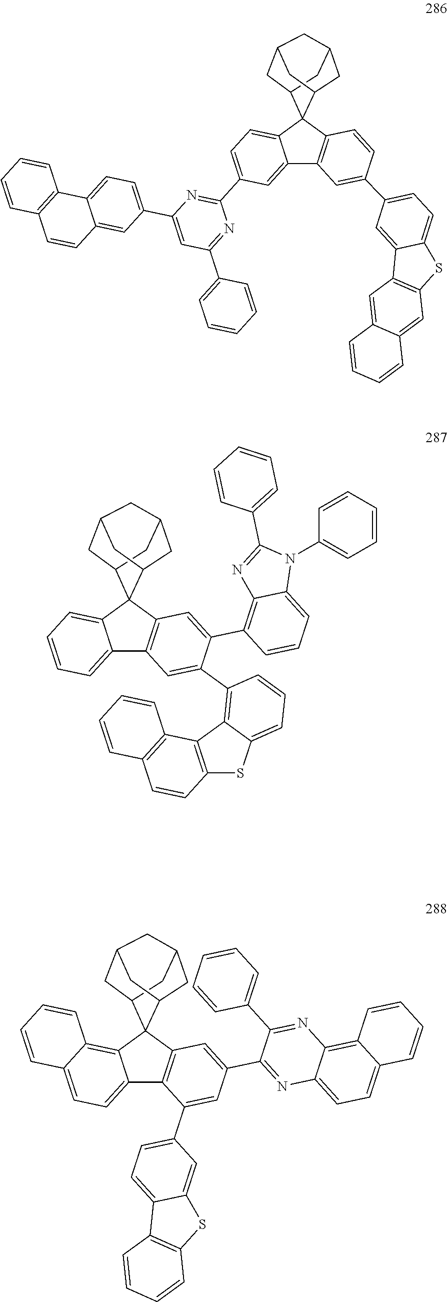 [SMILES: c1ccc(-c2cc(-c3ccc4c(ccc5ccccc54)c3)nc(-c3ccc4c(c3)-c3cc(-c5ccc6sc7cc8ccccc8cc7c6c5)ccc3C43C4CC5CC(C4)CC3C5)n2)cc1 ; c1ccc(-c2nc3c(-c4cc5c(cc4-c4cccc6sc7ccc8ccccc8c7c46)-c4ccccc4C54C5CC6CC(C5)CC4C6)cccc3n2-c2ccccc2)cc1 ; c1ccc(-c2nc3c(ccc4ccccc43)nc2-c2cc(-c3ccc4c(c3)sc3ccccc34)c3c(c2)C2(c4c-3ccc3ccccc43)C3CC4CC(C3)CC2C4)cc1]

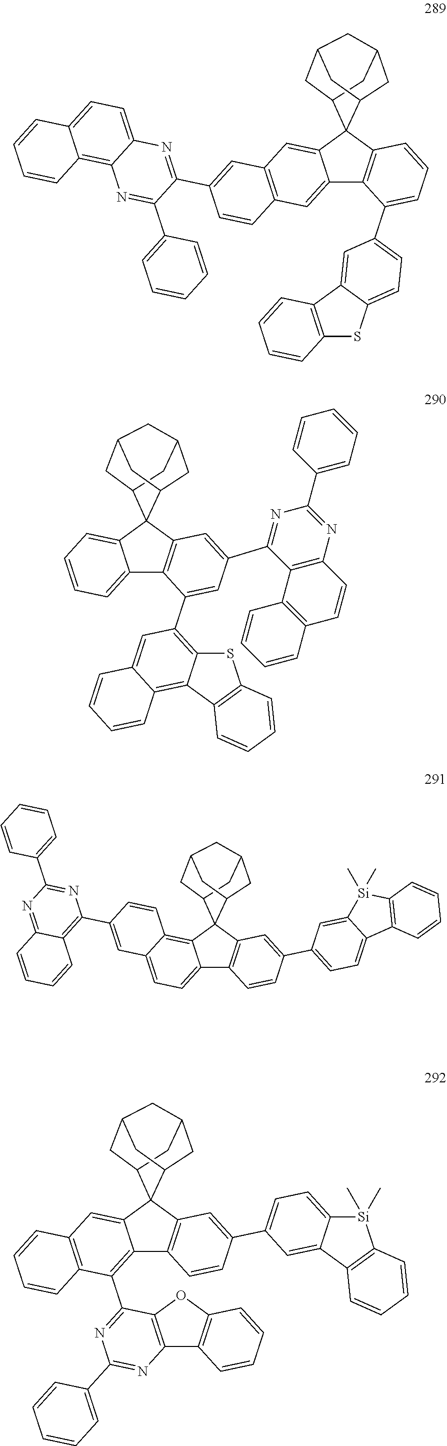 [SMILES: c1ccc(-c2nc3c(ccc4ccccc43)nc2-c2ccc3cc4c(cc3c2)C2(c3cccc(-c5ccc6sc7ccccc7c6c5)c3-4)C3CC4CC(C3)CC2C4)cc1 ; c1ccc(-c2nc(-c3cc(-c4cc5ccccc5c5c4sc4ccccc45)c4c(c3)C3(c5ccccc5-4)C4CC5CC(C4)CC3C5)c3c(ccc4ccccc43)n2)cc1 ; C[Si]1(C)c2ccccc2-c2ccc(-c3ccc4c(c3)C3(c5c-4ccc4cc(-c6nc(-c7ccccc7)nc7ccccc67)ccc54)C4CC5CC(C4)CC3C5)cc21 ; C[Si]1(C)c2ccccc2-c2cc(-c3ccc4c(c3)C3(c5cc6ccccc6c(-c6nc(-c7ccccc7)nc7c6oc6ccccc67)c5-4)C4CC5CC(C4)CC3C5)ccc21]

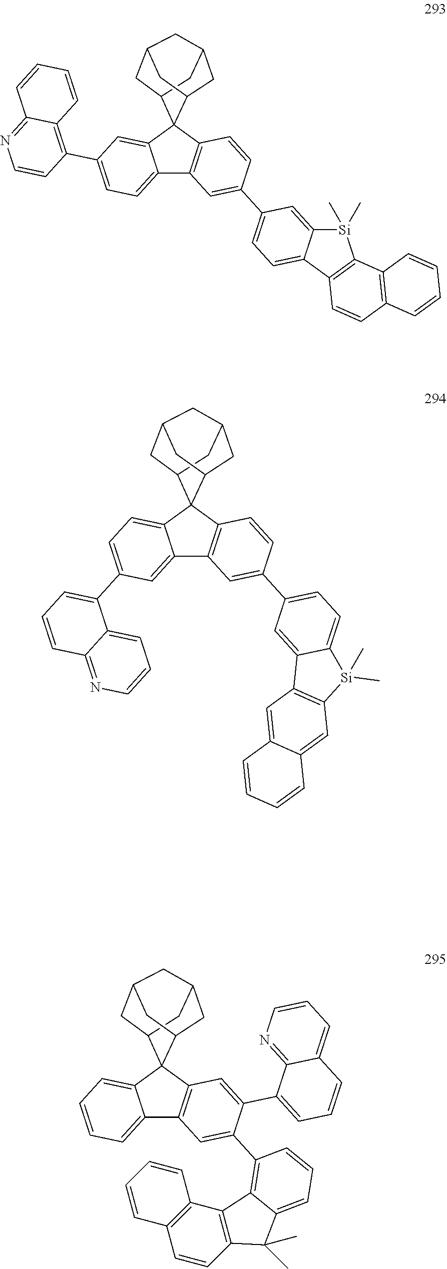 [SMILES: C[Si]1(C)c2cc(-c3ccc4c(c3)-c3ccc(-c5ccnc6ccccc56)cc3C43C4CC5CC(C4)CC3C5)ccc2-c2ccc3ccccc3c21 ; C[Si]1(C)c2ccc(-c3ccc4c(c3)-c3cc(-c5cccc6ncccc56)ccc3C43C4CC5CC(C4)CC3C5)cc2-c2cc3ccccc3cc21 ; CC1(C)c2cccc(-c3cc4c(cc3-c3cccc5cccnc35)C3(c5ccccc5-4)C4CC5CC(C4)CC3C5)c2-c2c1ccc1ccccc21]

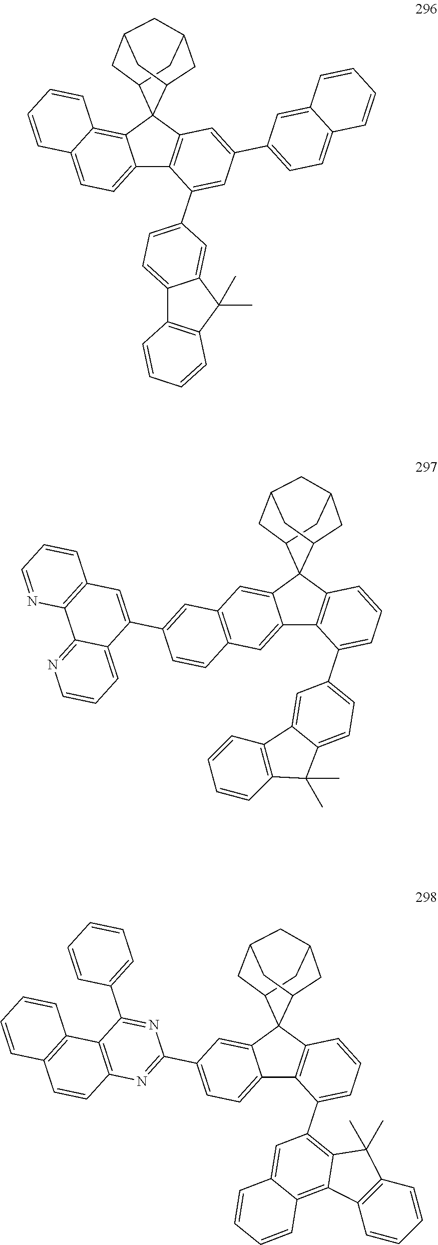

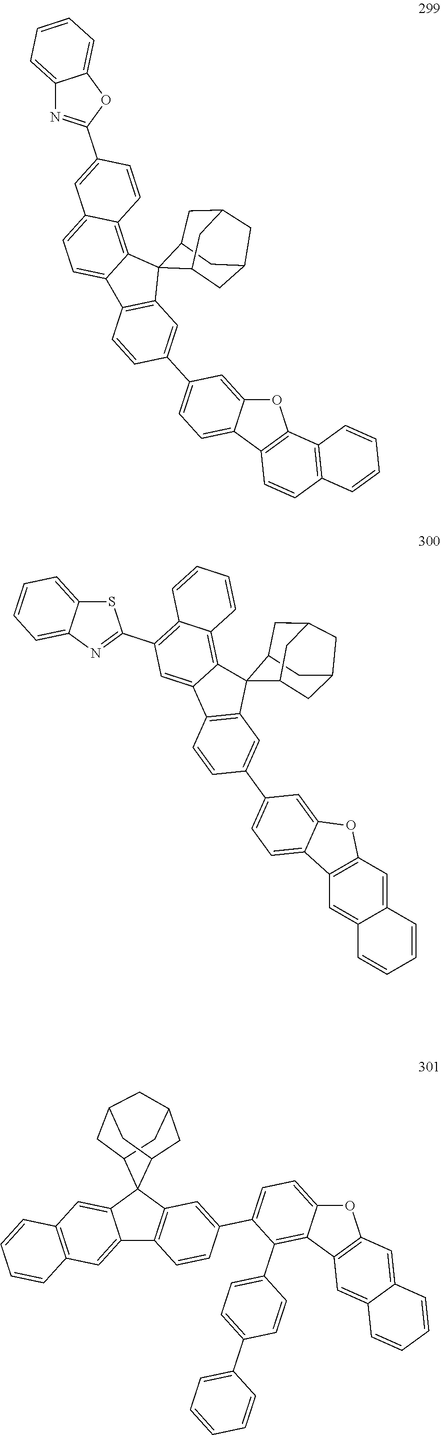 [SMILES: c1ccc2c(c1)ccc1c3ccc(-c4ccc5c(c4)C4(c6c-5ccc5cc(-c7nc8ccccc8o7)ccc65)C5CC6CC(C5)CC4C6)cc3oc21 ; c1ccc2cc3c(cc2c1)oc1cc(-c2ccc4c(c2)C2(c5c-4cc(-c4nc6ccccc6s4)c4ccccc54)C4CC5CC(C4)CC2C5)ccc13 ; c1ccc(-c2ccc(-c3c(-c4ccc5c(c4)C4(c6cc7ccccc7cc6-5)C5CC6CC(C5)CC4C6)ccc4oc5cc6ccccc6cc5c34)cc2)cc1]

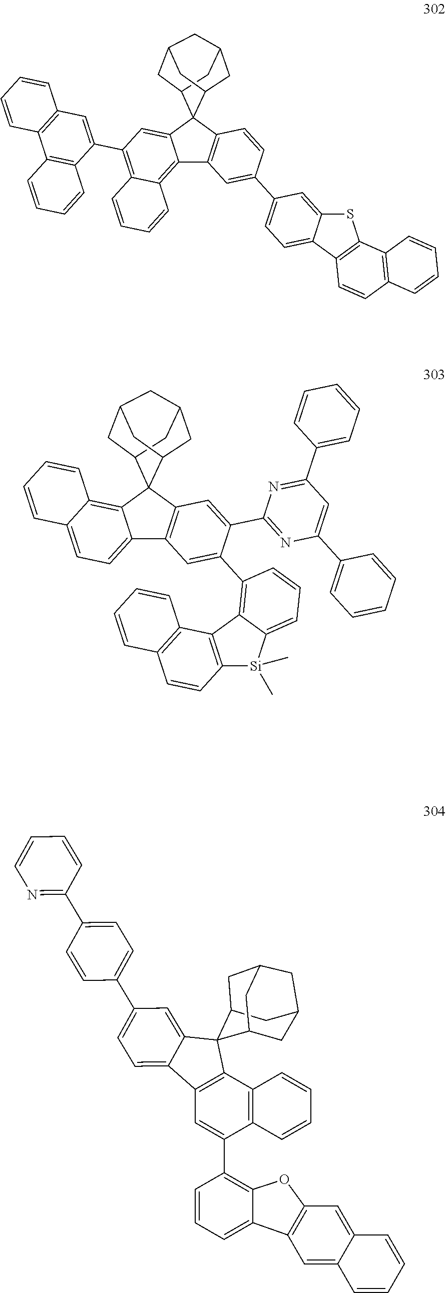 [SMILES: c1ccc2c(c1)cc(-c1cc3c(c4ccccc14)-c1cc(-c4ccc5c(c4)sc4c6ccccc6ccc54)ccc1C31C3CC4CC(C3)CC1C4)c1ccccc12 ; C[Si]1(C)c2cccc(-c3cc4c(cc3-c3nc(-c5ccccc5)cc(-c5ccccc5)n3)C3(c5c-4ccc4ccccc54)C4CC5CC(C4)CC3C5)c2-c2c1ccc1ccccc21 ; c1ccc(-c2ccc(-c3ccc4c(c3)C3(c5c-4cc(-c4cccc6c4oc4cc7ccccc7cc46)c4ccccc54)C4CC5CC(C4)CC3C5)cc2)nc1]

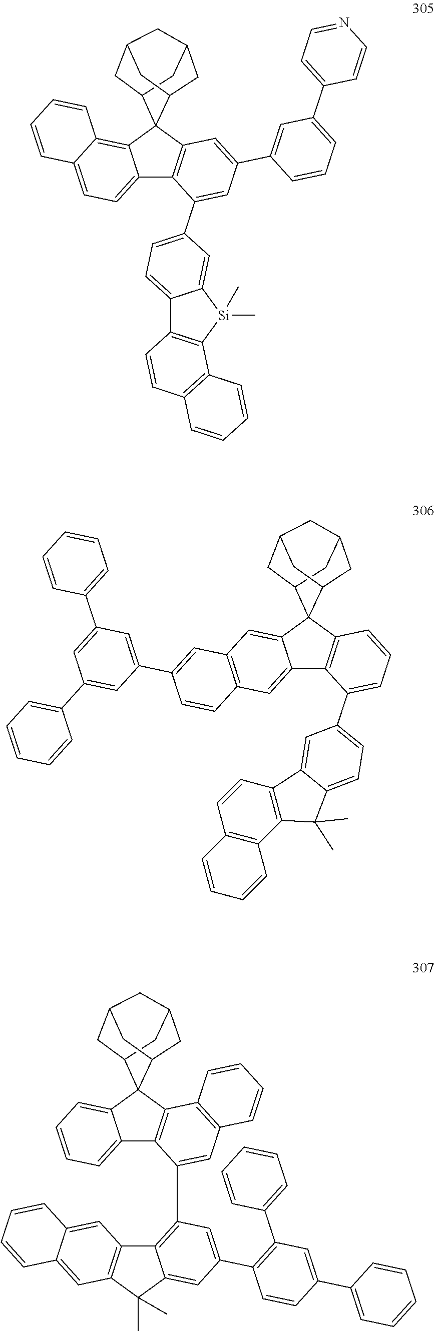

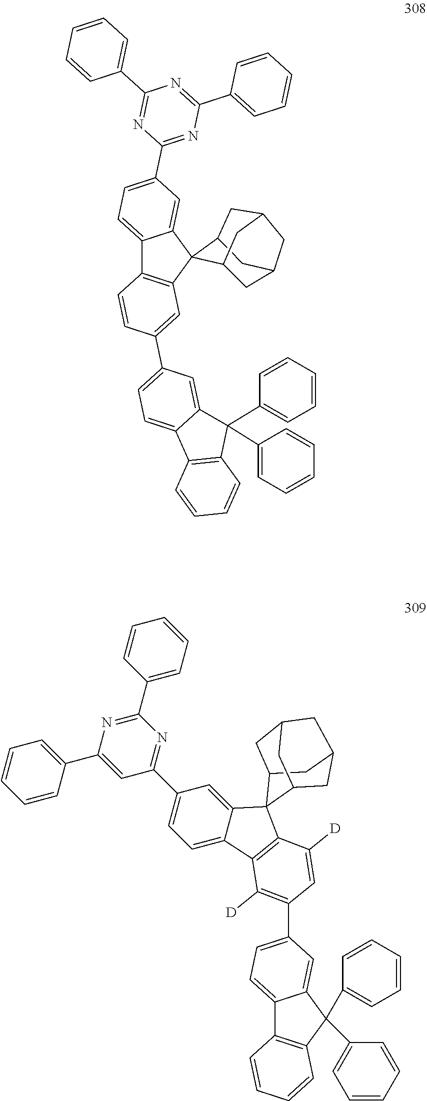 [SMILES: c1ccc(-c2nc(-c3ccccc3)nc(-c3ccc4c(c3)C3(c5cc(-c6ccc7c(c6)C(c6ccccc6)(c6ccccc6)c6ccccc6-7)ccc5-4)C4CC5CC(C4)CC3C5)n2)cc1 ; [2H]c1cc(-c2ccc3c(c2)C(c2ccccc2)(c2ccccc2)c2ccccc2-3)c([2H])c2c1C1(c3cc(-c4cc(-c5ccccc5)nc(-c5ccccc5)n4)ccc3-2)C2CC3CC(C2)CC1C3]

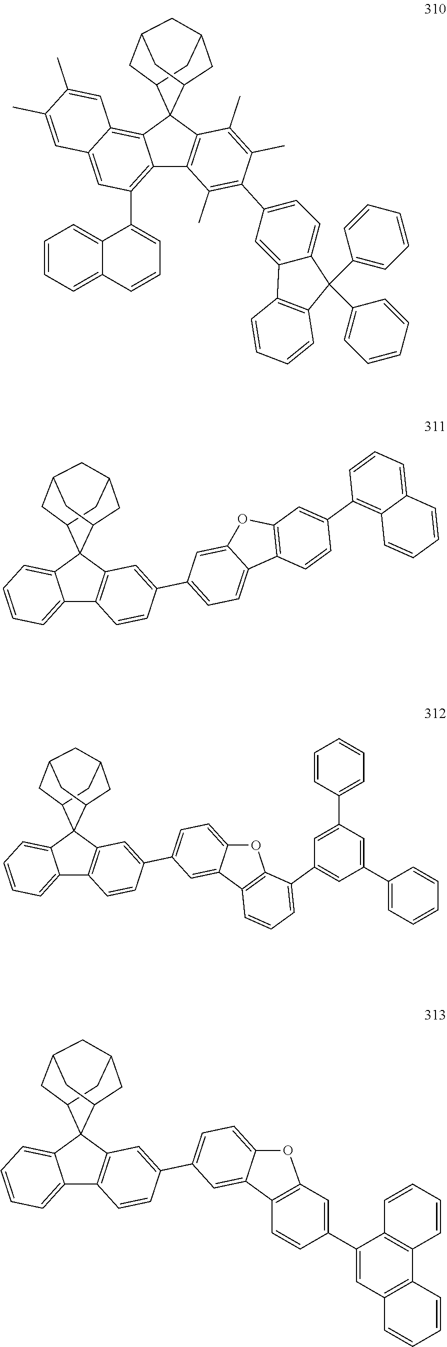

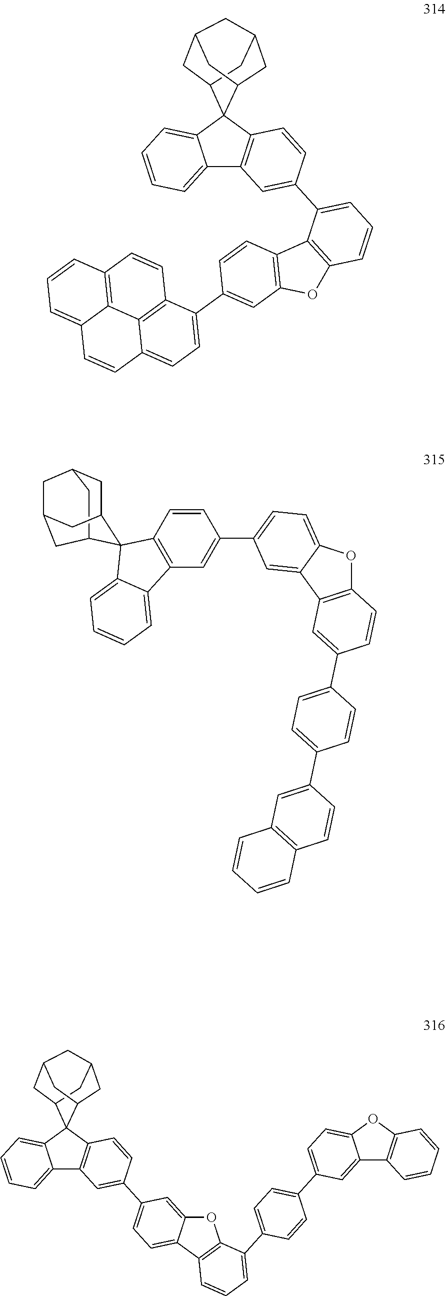 [SMILES: c1ccc2c(c1)-c1cc(-c3cccc4oc5cc(-c6ccc7ccc8cccc9ccc6c7c89)ccc5c34)ccc1C21C2CC3CC(C2)CC1C3 ; c1ccc2c(c1)-c1cc(-c3ccc4oc5ccc(-c6ccc(-c7ccc8ccccc8c7)cc6)cc5c4c3)ccc1C21C2CC3CC(C2)CC1C3 ; c1ccc2c(c1)-c1cc(-c3ccc4c(c3)oc3c(-c5ccc(-c6ccc7oc8ccccc8c7c6)cc5)cccc34)ccc1C21C2CC3CC(C2)CC1C3]

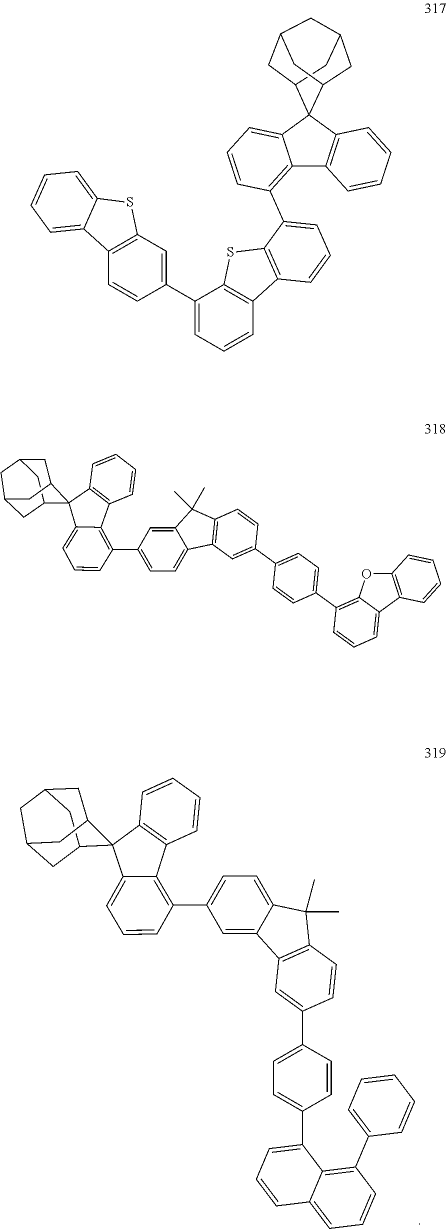

The present disclosure further provides an organic electroluminescence device. The organic electroluminescence device includes an anode and a cathode which are disposed oppositely, and an organic luminescent layer disposed between the anode and the cathode. The organic luminescent layer contains the above compound, so as to improve a voltage characteristic, an efficiency characteristic and a service life characteristic of the organic electroluminescence device.

The compound of the present disclosure may be used as a one-component host material or one of two-component hybrid host material.

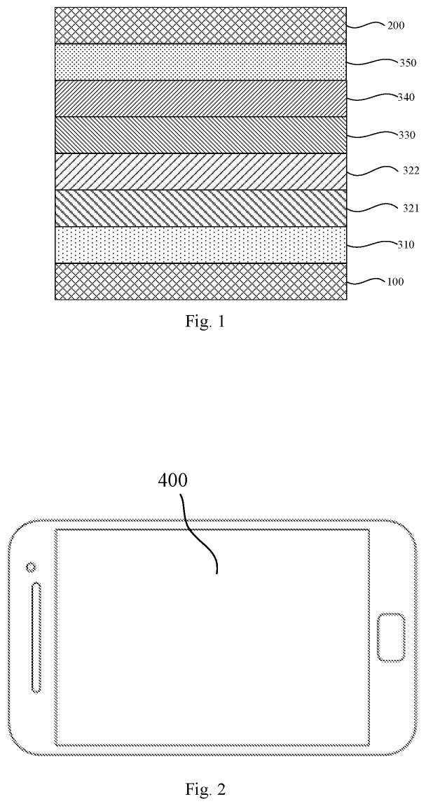

For example, as shown in FIG. 1, the organic electroluminescence device may include an anode 100, a hole transport layer 321, an organic luminescent layer 330, an electron transport layer 340 and a cathode 200 which are stacked sequentially. The compound provided by the present disclosure can be applied to the organic luminescent layer 330 of the organic electroluminescence device to increase a service life of the organic electroluminescence device, improve a luminescence efficiency of the organic electroluminescence device or reduce a driving voltage of the organic electroluminescence device.

Optionally, the anode 100 includes an anode material, which is optionally a material with a large work function that facilitates hole injection into the functional layer. Specific examples of the anode material include, but are not limited to: metals such as nickel, platinum, vanadium, chromium, copper, zinc and gold or their alloys; metal oxides such as zinc oxide, indium oxide, indium tin oxide (ITO) and indium zinc oxide (IZO); combined metal and oxides such as ZnO:Al or SnO₂:Sb; or conducting polymers such as poly(3-methylthiophene), poly[3,4-(ethylene-1,2-dioxy)thiophene] (PEDT), polypyrrole and polyaniline. Optionally, it includes a transparent electrode containing an indium tin oxide (ITO) as the anode.

Optionally, the hole transport layer 321 may include one or more hole transport materials, and the hole transport materials may be selected from a carbazole polymer, a carbazole-connected triarylamine compound or other types of compounds, which is not particularly limited in the present disclosure.

Optionally, the organic luminescent layer 330 may include a host material and a dopant material. Holes injected into the organic luminescent layer 330 and electrons injected into the organic luminescent layer 330 may be recombined in the organic luminescent layer 330 to form excitons, the excitons transfer energy to the host material, and the host material transfers the energy to the dopant material, so that the dopant material can emit light.

In one embodiment of the present disclosure, the host material may be composed of the compound of the present disclosure, especially be composed of the compound including electron-deficient aromatic heterocycle in a group R₁. Such compound can simultaneously transport the electrons and the holes, and can balance the transport efficiency of the holes and the electrons, so that the electrons and the holes can be efficiently recombined in the organic luminescent layer, thus improving the luminescence efficiency of the organic electroluminescence device.

In another embodiment of the present disclosure, the host material may be a composite material, for example, may include the compound of the present disclosure and the host material of an electron-type organic luminescent layer. The compound of the present disclosure can effectively transport the holes, to make the hole transport efficiency and the electron transport efficiency of the organic luminescent layer be balanced, so that the electrons and the holes can be efficiently recombined in the organic luminescent layer, thus improving the luminescence efficiency of the organic electroluminescence device. For example, the host material may include the compound of the present disclosure and GH-n1.

The dopant material of the organic luminescent layer 330 may be a compound having a condensed aryl ring or its derivative, a compound having a heteroaryl ring or its derivative, an aromatic amine derivative or other materials, which is not particularly limited in the present disclosure. In one embodiment of the present disclosure, the dopant material of the organic luminescent layer 330 may be Ir(piq)₂(acac) and the like. In another embodiment of the present disclosure, the dopant material of the organic luminescent layer 330 may be Ir(ppy)₃ and the like.

Optionally, the electron transport layer 340 may be a single-layer structure or a multi-layer structure, and may include one or more electron transport materials. The electron transport materials may be selected from, but are not limited to, a benzimidazole derivative, an oxadiazole derivative, a quinoxaline derivative or other electron transport materials.

Optionally, the cathode 200 may include the cathode material, which is a material with a small work function that facilitates electron injection into the functional layer. Specific examples of the cathode material include, but are not limited to: metals such as magnesium, calcium, sodium, potassium, titanium, indium, yttrium, lithium, gadolinium, aluminum, silver, tin and lead or their alloys; or a multi-layer material such as LiF/Al, Liq/Al, LiO₂/Al, LiF/Ca, LiF/Al and BaF₂/Ca, but are not limited to these. Optionally, it includes a metal electrode containing silver as the cathode. In one embodiment of the present disclosure, the material of the cathode 200 may be a alloy of magnesium and silver.

Optionally, as shown in FIG. 1, a hole injection layer 310 may further be disposed between the anode 100 and the hole transport layer 321 to enhance a capability of injecting holes into the first hole transport layer 321. The hole injection layer 310 may be selected from a benzidine derivative, a starburst arylamine compound, a phthalocyanine derivative or other materials, which is not particularly limited in the present disclosure. For example, the hole injection layer 310 may be composed of F₄-TCNQ.

Optionally, as shown in FIG. 1, an electron blocking layer 322 may further be disposed between the hole transport layer 321 and the organic luminescent layer 330 to block the electrons from being transported to a side of the hole transport layer 321, improve a recombination rate of the electrons and the holes in the organic luminescent layer 330, and protect the hole transport layer 321 from the impact of the electrons. A material of the electron blocking layer 322 may be a carbazole polymer, a carbazole-connected triarylamine compound, or other feasible structures.

Optionally, as shown in FIG. 1, an electron injection layer 350 may further be disposed between the cathode 200 and the electron transport layer 340 to enhance a capability of injecting the electrons into the electron transport layer 340. The electron injection layer 350 may include inorganic materials such as alkali metal sulfide and alkali metal halide, or may include a complex of alkali metal and organic matter. For example, the electron injection layer 350 may include LiQ.

The present disclosure further provides an electronic apparatus 400, as shown in FIG. 2, the electronic apparatus 400 includes any organic electroluminescence device described in the above organic electroluminescence device embodiments. The electronic apparatus 400 may be a display apparatus, a lighting apparatus, an optical communication apparatus, or other types of electronic apparatuses, which may include, for example, but are not limited to a computer screen, a mobile phone screen, a television, electronic paper, an emergency lighting lamp, an optical module, and the like. Since the electronic apparatus 400 has any of the organic electroluminescence devices described in the above organic electroluminescence device embodiments, it has the same beneficial effects, which is not repeated here in the present disclosure.

### SYNTHESIS EXAMPLE

In the synthesis examples described below, unless otherwise stated, all temperatures are in degrees Celsius. Some reagents are purchased from commodity suppliers such as Aldrich Chemical Company, Arco Chemical Company and Alfa Chemical Company, and some intermediates that cannot be purchased directly were prepared by simple reactions from commercially available raw materials, and were used without further purification unless otherwise stated. The rest of conventional reagents are purchased from Shantou Xilong Chemical Factory, Guangdong Guanghua Chemical Reagent Factory, Guangzhou Chemical Reagent Factory, Tianjin Haoyuyu Chemical Co., Ltd., Tianjin Fuchen Chemical Reagent Factory, Wuhan Xinhuayuan Technology Development Co., Ltd., Qingdao Tenglong Chemical Reagent Co., Ltd., and Qingdao Ocean Chemical Factory. Anhydrous tetrahydrofuran, dioxane, toluene, ether and other anhydrous solvents are obtained by refluxing and drying with metallic sodium. A reaction in each synthesis example is generally carried out under a positive pressure of nitrogen or argon, or a drying tube is set on the anhydrous solvent (unless otherwise stated); in the reaction, a reaction flask is plugged with a suitable rubber stopper, and a substrate is injected into the reaction flask through a syringe. All glassware used is dried.

During purification, a chromatographic column is a silica gel column, and silica gel (100 to 200 meshes) is purchased from Qingdao Ocean Chemical Factory.

In each synthesis example, a measurement condition of low-resolution mass spectrometry (MS) data is: Agilent 6120 quadrupole HPLC-M (a column model: Zorbax SB-C18, 2.1×30 mm, 3.5 μm, 6 min, and a flow rate of 0.6 mL/min. Mobile phase: 5% to 95% (a ratio of acetonitrile containing 0.1% formic acid in water containing 0.1% formic acid), using electrospray ionization (ESI) at 210 nm/254 nm with UV detection.

H-nuclear magnetic resonance: Bruker 400 MHz nuclear magnetic instrument at a room temperature, with CDCl₃ as a solvent (in ppm), and TMS (0 ppm) as a reference standard. when multiplets are present, the following abbreviations will be used: s (singlet), d (doublet), t (triplet), and m (multiplet).

A target compound is detected by UV at 210 nm/254 nm using Agilent 1260pre-HPLC or Calesep pump 250pre-HPLC (column model: NOVASEP 50/80 mm DAC).

Synthesis of the compound of the present disclosure is carried out using the following methods:

### PREPARATION EXAMPLES 1 TO 13

1. Synthesis of Intermediate a-1

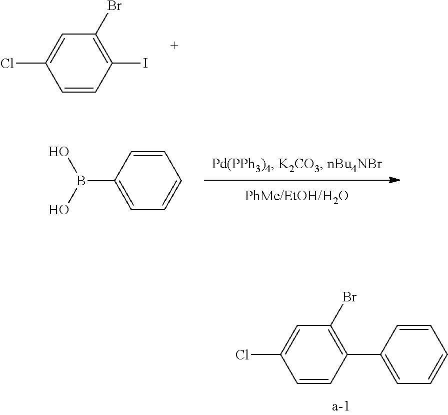 [SMILES: Clc1ccc(I)c(Br)c1 ; OB(O)c1ccccc1 ; Clc1ccc(-c2ccccc2)c(Br)c1]

2-bromo-4-chloro-1-iodobenzene (80.0 g, 252.1 mmol), phenylboronic acid (33.8 g, 277.3 mmol), tetrakis(triphenylphosphine)palladium (5.8 g, 5.0 mmol), potassium carbonate (76.5 g, 554.6 mmol), tetrabutylammonium bromide (16.2 g, 50.4 mmol) were added into a flask, a mixed solvent of toluene (640 mL), ethanol (320 mL) and water (160 mL) was added too. In nitrogen atmosphere, the reaction mixture was heated to to 80° C., and stirred for 8 hours. The reaction mixture was cooled to the room temperature, stirring was stopped. The reaction solution was washed with water, an separated organic phase was dried with anhydrous magnesium sulfate, and concentrated in vacuum to obtain a crude product. The crude product was purified by silica gel column chromatography and eluted with n-heptane to obtain the intermediate a-1 (40.5 g, yield 60%) as a light-gray solid product.

Intermediates a-2 to a-4 were synthesized by using a method similar to the synthesis of the intermediate a-1, and by using a compound shown as a reactant A in Table 1 in place of 2-bromo-4-chloro-1-iodobenzene and a compound shown as a reactant B in place of phenylboronic acid.

TABLE 1
Synthesis of intermediate a-2 to intermediate a-4

| Intermediate | | | | Yield |
|---|---|---|---|---|
| No. | Reactant A | Reactant B | Structure | (%) |
| Intermediate a-2 | 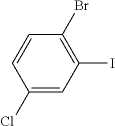 | 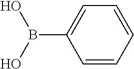 | 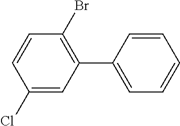 [SMILES: Clc1ccc(Br)c(-c2ccccc2)c1] | 65% |
| Intermediate a-3 | 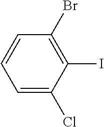 | 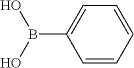 | 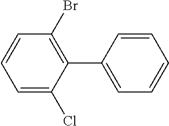 [SMILES: Clc1cccc(Br)c1-c1ccccc1] | 62% |
| Intermediate a-4 | 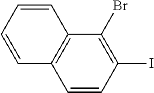 [SMILES: Brc1c(I)ccc2ccccc12] | 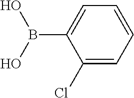 | 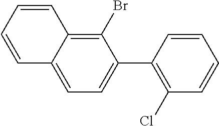 [SMILES: Clc1ccccc1-c1ccc2ccccc2c1Br] | 64% |

2. Synthesis of Intermediate b-1

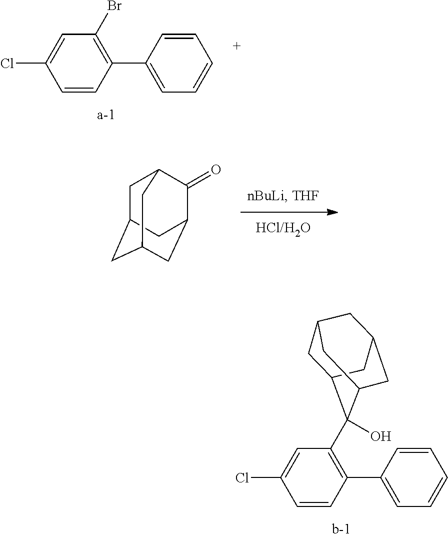 [SMILES: Clc1ccc(-c2ccccc2)c(Br)c1 ; O=C1C2CC3CC(C2)CC1C3 ; OC1(c2cc(Cl)ccc2-c2ccccc2)C2CC3CC(C2)CC1C3]

The intermediate a-1 (40.5 g, 151.37 mmol) and tetrahydrofuran (320 ml) were added into a flask in a nitrogen atmosphere, the mixture was cooled to −78° C., and under a stirring condition, a tetrahydrofuran (2.5 M) solution (72.6 mL, 181.6 mmol) of n-butyllithium was added dropwise with keeping −78° C. After the dropwise addition, the mixture was stirred for 1 hour while keeping the same temperature, and then a tetrahydrofuran (100 mL) solution in which adamantanone was dissolved (25.0 g, 166.5 mmol) was added dropwise. After the dropwise addition, the mixture was stirred for 1 hour while keeping the same temperature and then moved to room temperature, and stirred for another 24 hours. A water (120 mL) solution of hydrochloric acid (12 M) (22 mL, 272.4 mmol) was added into the reaction solution, and the resulted mixture was stirred for 0.5 hour, and then the mixture was allowed to stand and stratify. An separated organic phase was washed with water until to be neutral, and dried by adding anhydrous magnesium sulfate, and then concentrated in vacuum to obtain a crude product. The crude product was purified by silica gel column chromatography eluted with a mixture of ethyl acetate and n-heptane to obtain the intermediate b-1 (28.2 g, 50%) as a white solid product.

Intermediate b-2 to intermediate b-4 were synthesized by using a method similar to the synthesis of the intermediate b-1, and by using a reactant A shown in Table 2 in place of the intermediate a-1.

TABLE 2
Synthesis of intermediate b-2 to intermediate b-4

| Intermediate | | | |
|---|---|---|---|
| No. | Reactant A | Structure | Yield (%) |
| b-2 | 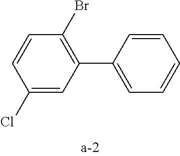 [SMILES: Clc1ccc(Br)c(-c2ccccc2)c1] | 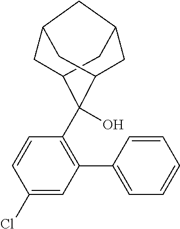 [SMILES: OC1(c2ccc(Cl)cc2-c2ccccc2)C2CC3CC(C2)CC1C3] | 55% |
| b-3 | 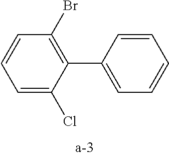 [SMILES: Clc1cccc(Br)c1-c1ccccc1] | 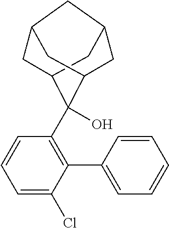 [SMILES: OC1(c2cccc(Cl)c2-c2ccccc2)C2CC3CC(C2)CC1C3] | 58% |
| b-4 | 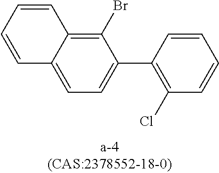 [SMILES: Clc1ccccc1-c1ccc2ccccc2c1Br] | 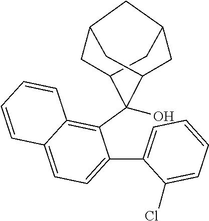 [SMILES: OC1(c2c(-c3ccccc3Cl)ccc3ccccc23)C2CC3CC(C2)CC1C3] | 56% |

3. Synthesis of Intermediate c-1

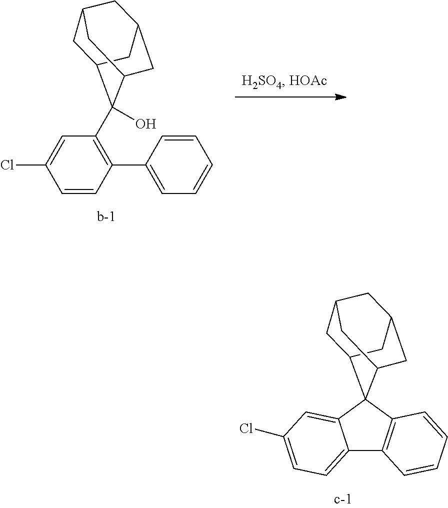 [SMILES: OC1(c2cc(Cl)ccc2-c2ccccc2)C2CC3CC(C2)CC1C3 ; Clc1ccc2c(c1)C1(c3ccccc3-2)C2CC3CC(C2)CC1C3]

An intermediate b-1 (28.2 g, 83.2 mmol) and glacial acetic acid (280 mL) were added into a flask, and an acetic acid (40 mL) solution of concentrated sulfuric acid (98%) (1.7 mL, 16.7 mmol) was slowly added dropwise with stirring at a room temperature in a nitrogen atmosphere. The mixture was heated to 80° C. after the dropwise addition, and stirred for 2 hours. Then the mixture was cooled to the room temperature and solid appeared. A separated solid was filtered, a filter cake was subjected to drip washing with water and ethanol, and the solid was collected and dried to obtain a crude product. The crude product was purified by silica gel column chromatography and eluted with a mixture of dichloromethane and n-heptane to obtain a white solid intermediate c-1 (20.0 g, yield 75%).

Intermediates c-2 to c-4 were synthesized by using a method similar to that described above and by replacing the intermediate b-1 with a reactant A in Table 3 below.

TABLE 3
Synthesis of intermediate c-2 to intermediate c-4

| Intermediate | | | Yield |
|---|---|---|---|
| No. | Reactant A | Structure | (%) |
| c-2 | 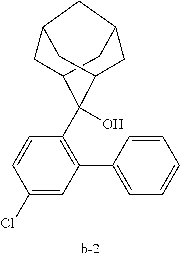 [SMILES: OC1(c2ccc(Cl)cc2-c2ccccc2)C2CC3CC(C2)CC1C3] | 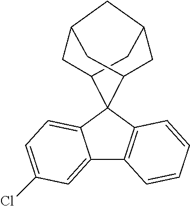 [SMILES: Clc1ccc2c(c1)-c1ccccc1C21C2CC3CC(C2)CC1C3] | 70% |
| c-3 | 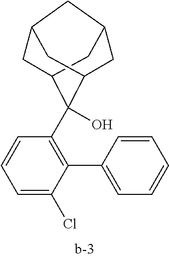 [SMILES: OC1(c2cccc(Cl)c2-c2ccccc2)C2CC3CC(C2)CC1C3] | 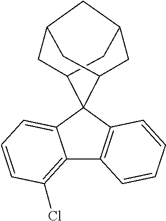 [SMILES: Clc1cccc2c1-c1ccccc1C21C2CC3CC(C2)CC1C3] | 72% |
| c-4 | 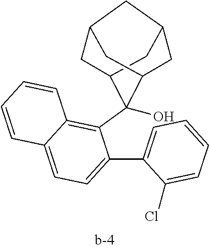 [SMILES: OC1(c2c(-c3ccccc3Cl)ccc3ccccc23)C2CC3CC(C2)CC1C3] | 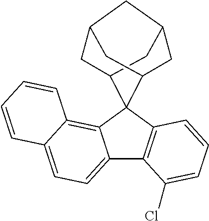 [SMILES: Clc1cccc2c1-c1ccc3ccccc3c1C21C2CC3CC(C2)CC1C3] | 70% |

4. Synthesis of Intermediate d-1

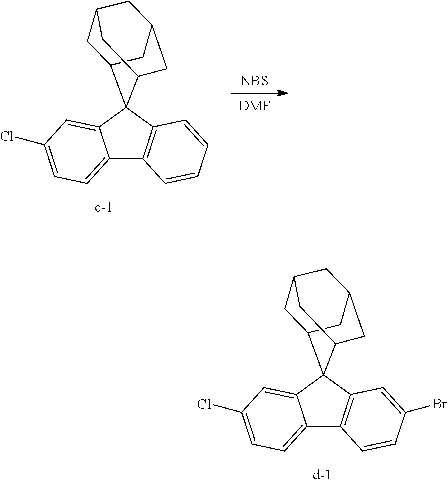

The intermediate c-1 (20.0 g, 62.3 mmol) and a solvent DMF (N,N-dimethylformamide) (160 mL) were added into a flask, and stirred at a room temperature for 10 min in a nitrogen atmosphere, then N-bromosuccinimide (NBS) (16.6 g, 93.5 mmol) was added, and the resulted mixture was heated to 80° C., and stirred for 4 h while keeping the same temperature. After the reaction was completed, the reaction mixture was cooled to the room temperature, a reaction solution was extracted with dichloromethane and water. The separated organic phase dried with anhydrous magnesium sulfate, and concentrated in vacuum to obtain a crude product. The crude product was purified by silica gel column chromatography and eluted with a mixture of dichloromethane and n-heptane system as a mobile phase to obtain the intermediate d-1 (18.9, yield 76%) as a white solid product.

Intermediates d-2 to d-4 were synthesized by using a method similar to that described above and by replacing the intermediate c-1 with a reactant A in Table 4 below.

TABLE 4
Synthesis of intermediate d-2 to intermediate d-4

| Intermediate | | | Yield |
|---|---|---|---|
| No. | Reactant A | Structure | (%) |
| d-2 | 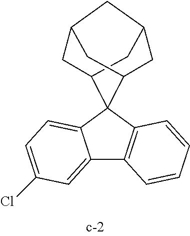 [SMILES: Clc1ccc2c(c1)-c1ccccc1C21C2CC3CC(C2)CC1C3] | 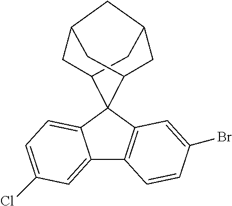 [SMILES: Clc1ccc2c(c1)-c1ccc(Br)cc1C21C2CC3CC(C2)CC1C3] | 78% |
| d-3 | 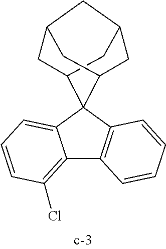 [SMILES: Clc1cccc2c1-c1ccccc1C21C2CC3CC(C2)CC1C3] | 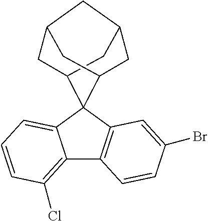 [SMILES: Clc1cccc2c1-c1ccc(Br)cc1C21C2CC3CC(C2)CC1C3] | 74% |
| d-4 | 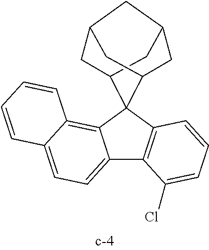 [SMILES: Clc1cccc2c1-c1ccc3ccccc3c1C21C2CC3CC(C2)CC1C3] | 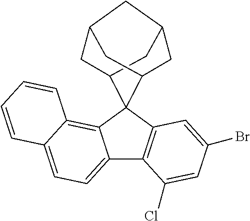 [SMILES: Clc1cc(Br)cc2c1-c1ccc3ccccc3c1C21C2CC3CC(C2)CC1C3] | 70% |

5. Synthesis of Intermediate e-1

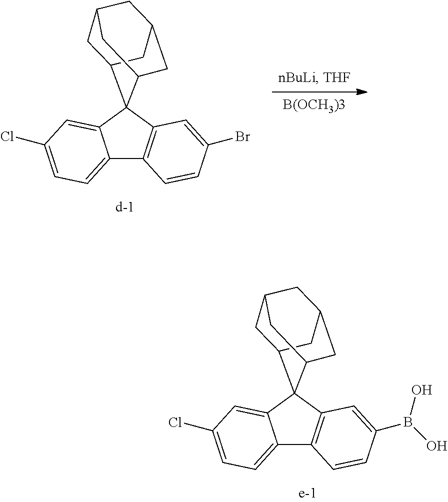

The intermediate d-1 (18.9 g, 47.3 mmol) and tetrahydrofuran (150 ml) were added into a flask in a nitrogen atmosphere, the reaction mixture was cooled to −78° C., and under a stirring condition, a tetrahydrofuran (2.5 M) solution (22.7 mL, 56.73 mmol) of n-butyllithium was added dropwise with keeping −78° C. After the dropwise addition, stirring was performed for 1 hour while keeping the same temperature, and a tetrahydrofuran (20 mL) solution in which trimethyl borate (5.4 g, 52 mmol) was dissolved was added dropwise. After the dropwise addition, the mixture was stirred for 1 hour while keeping the same temperature and then moved to a room temperature, and stirred for another 24 hours. A water (35.5 mL) solution of hydrochloric acid (12 M) (7.1 mL, 85.1 mmol) was added into the reaction solution, and the resulted mixture was stirred for 1 hour, and then the mixture was allowed to stand and stratify. An separated organic phase was washed with water until neutral, and dried with anhydrous magnesium sulfate, and concentrated in vacuum to obtain a crude product. The crude product was purified by silica gel column chromatography and eluted with a mixture of dichloromethane and n-heptane to obtain the intermediate e-1 (9.5 g, 55%) as a white solid product.

Intermediate e-2 to intermediate e-4 were synthesized by using a method similar to the synthesis of the intermediate e-1, and by using a reactant A shown in Table 5 in place of the intermediate d-1.

TABLE 5
Synthesis of intermediate e-2 to intermediate e-4

| Intermediate | | | Yield |
|---|---|---|---|
| No. | Reactant A | Structure | (%) |
| e-2 | 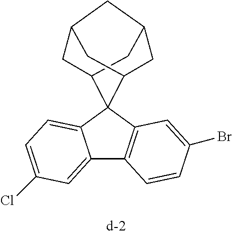 [SMILES: Clc1ccc2c(c1)-c1ccc(Br)cc1C21C2CC3CC(C2)CC1C3] | 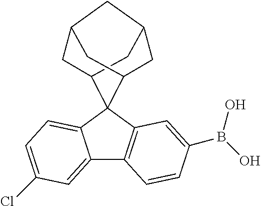 [SMILES: OB(O)c1ccc2c(c1)C1(c3ccc(Cl)cc3-2)C2CC3CC(C2)CC1C3] | 56% |
| e-3 | 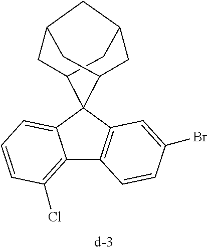 [SMILES: Clc1cccc2c1-c1ccc(Br)cc1C21C2CC3CC(C2)CC1C3] | 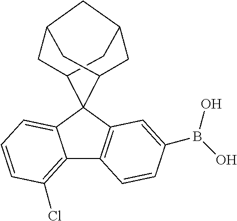 [SMILES: OB(O)c1ccc2c(c1)C1(c3cccc(Cl)c3-2)C2CC3CC(C2)CC1C3] | 58% |
| e-4 | 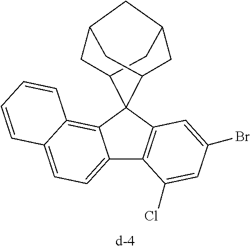 [SMILES: Clc1cc(Br)cc2c1-c1ccc3ccccc3c1C21C2CC3CC(C2)CC1C3] | 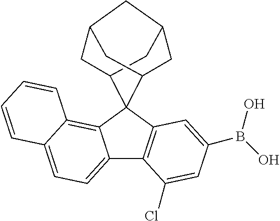 [SMILES: OB(O)c1cc(Cl)c2c(c1)C1(c3c-2ccc2ccccc32)C2CC3CC(C2)CC1C3] | 52% |

6. Synthesis of Intermediate f-1

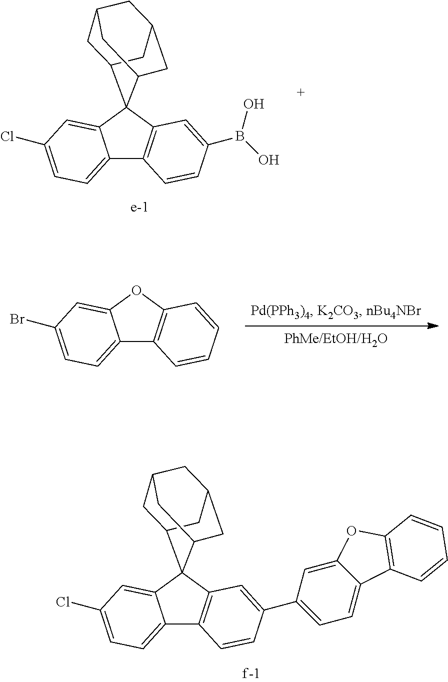

The intermediate e-1 (9.5 g, 26.1 mmol), 3-bromodibenzofuran (6.1 g, 24.8 mmol), tetrakis(triphenylphosphine)palladium (0.57 g, 0.5 mmol), potassium carbonate (7.5 g, 54.6 mmol), tetrabutylammonium bromide (1.6 g, 5.0 mmol) were added into a flask, a mixed solvent of toluene (76 mL), ethanol (38 mL) and water (19 mL) was added in a nitrogen atmosphere, the reaction mixture was heated to 80° C. and stirred for 8 hours while keeping the same temperature. The mixture was cooled to a room temperature, stirring was stopped, washed with water, an separated organic phase was dried with anhydrous magnesium sulfate, and concentrated in vacuum to obtain a crude product. The crude product was purified by silica gel column chromatography and eluted with n-heptane to obtain the intermediate f-1 (9.1 g, yield 75%) as a white solid product.

Intermediates f-2 to f-14 were synthesized by using a method similar to the synthesis of the intermediate f-1, and by using a compound shown as a reactant A in Table 6 in place of the intermediate e-1 and a compound shown as a reactant B in place of 3-bromodibenzofuran.

TABLE 6
Synthesis of intermediate f-2 to intermediate f-14

| Intermediate | | |
|---|---|---|
| No. | Reactant A | Reactant B |
| Intermediate f-2 | 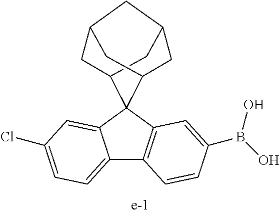 [SMILES: OB(O)c1ccc2c(c1)C1(c3cc(Cl)ccc3-2)C2CC3CC(C2)CC1C3] | 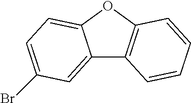 [SMILES: Brc1ccc2oc3ccccc3c2c1] |
| Intermediate f-3 | 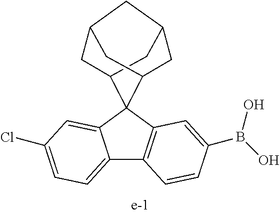 [SMILES: OB(O)c1ccc2c(c1)C1(c3cc(Cl)ccc3-2)C2CC3CC(C2)CC1C3] | 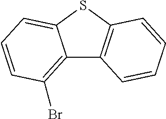 [SMILES: Brc1cccc2sc3ccccc3c12] |
| Intermediate f-4 | 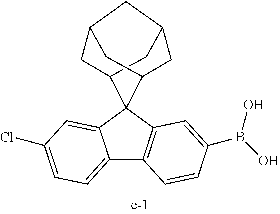 [SMILES: OB(O)c1ccc2c(c1)C1(c3cc(Cl)ccc3-2)C2CC3CC(C2)CC1C3] | 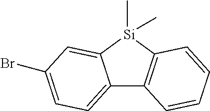 [SMILES: C[Si]1(C)c2ccccc2-c2ccc(Br)cc21] |
| Intermediate f-5 | 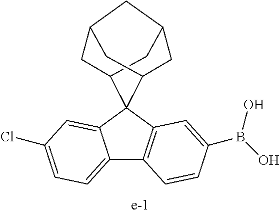 [SMILES: OB(O)c1ccc2c(c1)C1(c3cc(Cl)ccc3-2)C2CC3CC(C2)CC1C3] | 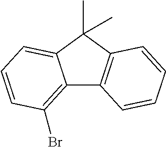 [SMILES: CC1(C)c2ccccc2-c2c(Br)cccc21] |
| Intermediate f-6 | 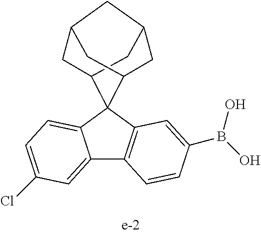 [SMILES: OB(O)c1ccc2c(c1)C1(c3ccc(Cl)cc3-2)C2CC3CC(C2)CC1C3] | 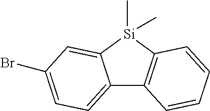 [SMILES: C[Si]1(C)c2ccccc2-c2ccc(Br)cc21] |
| Intermediate f-7 | 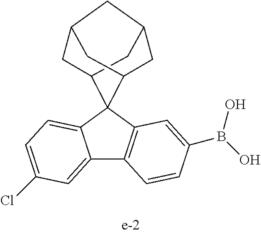 [SMILES: OB(O)c1ccc2c(c1)C1(c3ccc(Cl)cc3-2)C2CC3CC(C2)CC1C3] | 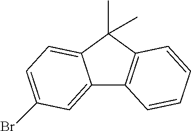 [SMILES: CC1(C)c2ccccc2-c2cc(Br)ccc21] |
| Intermediate f-8 | 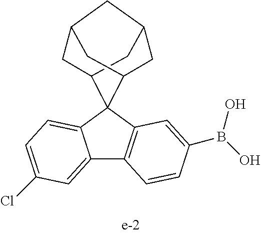 [SMILES: OB(O)c1ccc2c(c1)C1(c3ccc(Cl)cc3-2)C2CC3CC(C2)CC1C3] | 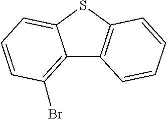 [SMILES: Brc1cccc2sc3ccccc3c12] |
| Intermediate f-9 | 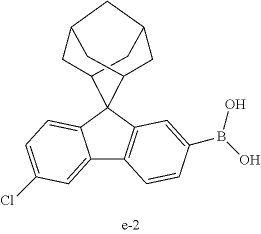 [SMILES: OB(O)c1ccc2c(c1)C1(c3ccc(Cl)cc3-2)C2CC3CC(C2)CC1C3] | 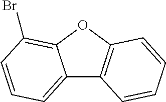 [SMILES: Brc1cccc2c1oc1ccccc12] |
| Intermediate f-10 | 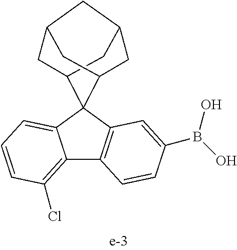 [SMILES: OB(O)c1ccc2c(c1)C1(c3cccc(Cl)c3-2)C2CC3CC(C2)CC1C3] | 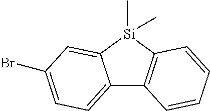 [SMILES: C[Si]1(C)c2ccccc2-c2ccc(Br)cc21] |
| Intermediate f-11 | 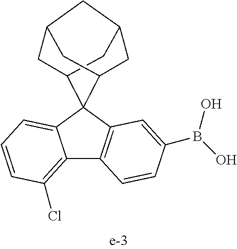 [SMILES: OB(O)c1ccc2c(c1)C1(c3cccc(Cl)c3-2)C2CC3CC(C2)CC1C3] | 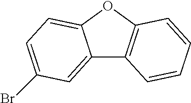 [SMILES: Brc1ccc2oc3ccccc3c2c1] |
| Intermediate f-12 | 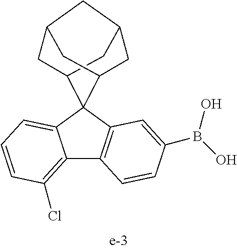 [SMILES: OB(O)c1ccc2c(c1)C1(c3cccc(Cl)c3-2)C2CC3CC(C2)CC1C3] | 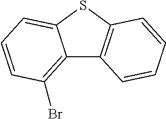 [SMILES: Brc1cccc2sc3ccccc3c12] |
| Intermediate f-13 | 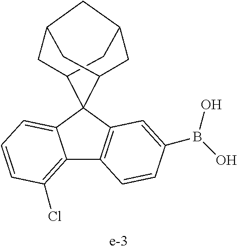 [SMILES: OB(O)c1ccc2c(c1)C1(c3cccc(Cl)c3-2)C2CC3CC(C2)CC1C3] | 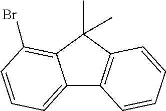 [SMILES: CC1(C)c2ccccc2-c2cccc(Br)c21] |
| Intermediate f-14 | 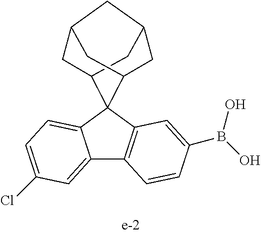 [SMILES: OB(O)c1ccc2c(c1)C1(c3ccc(Cl)cc3-2)C2CC3CC(C2)CC1C3] | 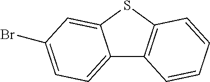 [SMILES: Brc1ccc2c(c1)sc1ccccc12] |
| Intermediate | | Yield |
| No. | Structure | (%) |
| Intermediate f-2 | 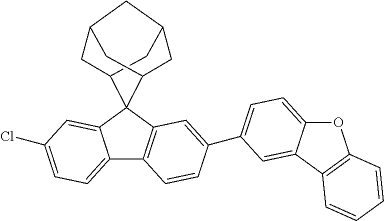 [SMILES: Clc1ccc2c(c1)C1(c3cc(-c4ccc5oc6ccccc6c5c4)ccc3-2)C2CC3CC(C2)CC1C3] | 63% |
| Intermediate f-3 | 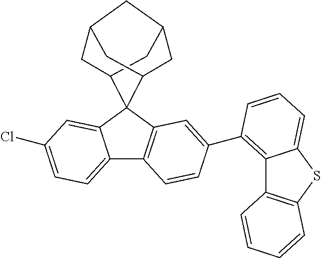 [SMILES: Clc1ccc2c(c1)C1(c3cc(-c4cccc5sc6ccccc6c45)ccc3-2)C2CC3CC(C2)CC1C3] | 67% |
| Intermediate f-4 | 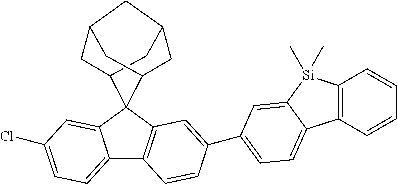 [SMILES: C[Si]1(C)c2ccccc2-c2ccc(-c3ccc4c(c3)C3(c5cc(Cl)ccc5-4)C4CC5CC(C4)CC3C5)cc21] | 66% |
| Intermediate f-5 | 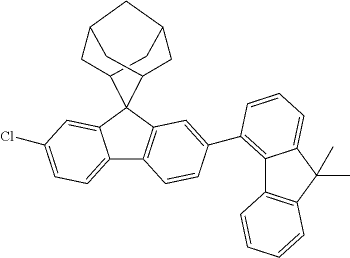 [SMILES: CC1(C)c2ccccc2-c2c(-c3ccc4c(c3)C3(c5cc(Cl)ccc5-4)C4CC5CC(C4)CC3C5)cccc21] | 68% |
| Intermediate f-6 | 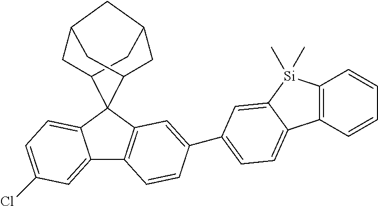 [SMILES: C[Si]1(C)c2ccccc2-c2ccc(-c3ccc4c(c3)C3(c5ccc(Cl)cc5-4)C4CC5CC(C4)CC3C5)cc21] | 70% |
| Intermediate f-7 | 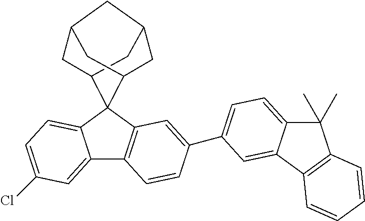 [SMILES: CC1(C)c2ccccc2-c2cc(-c3ccc4c(c3)C3(c5ccc(Cl)cc5-4)C4CC5CC(C4)CC3C5)ccc21] | 72% |
| Intermediate f-8 | 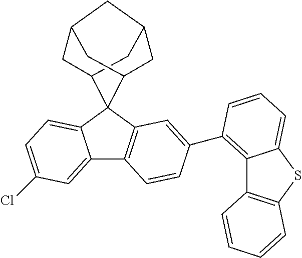 [SMILES: Clc1ccc2c(c1)-c1ccc(-c3cccc4sc5ccccc5c34)cc1C21C2CC3CC(C2)CC1C3] | 78% |
| Intermediate f-9 | 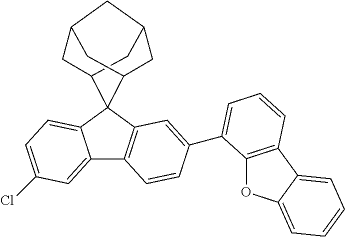 [SMILES: Clc1ccc2c(c1)-c1ccc(-c3cccc4c3oc3ccccc34)cc1C21C2CC3CC(C2)CC1C3] | 72% |
| Intermediate f-10 | 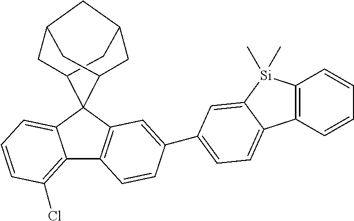 [SMILES: C[Si]1(C)c2ccccc2-c2ccc(-c3ccc4c(c3)C3(c5cccc(Cl)c5-4)C4CC5CC(C4)CC3C5)cc21] | 71% |
| Intermediate f-11 | 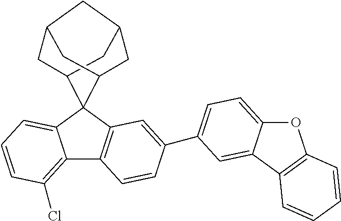 [SMILES: Clc1cccc2c1-c1ccc(-c3ccc4oc5ccccc5c4c3)cc1C21C2CC3CC(C2)CC1C3] | 69% |
| Intermediate f-12 | 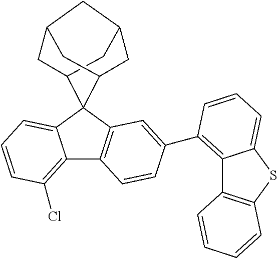 [SMILES: Clc1cccc2c1-c1ccc(-c3cccc4sc5ccccc5c34)cc1C21C2CC3CC(C2)CC1C3] | 70% |
| Intermediate f-13 | 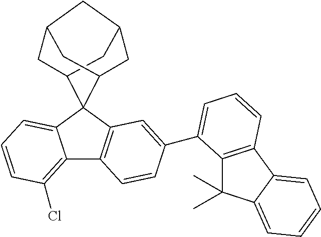 [SMILES: CC1(C)c2ccccc2-c2cccc(-c3ccc4c(c3)C3(c5cccc(Cl)c5-4)C4CC5CC(C4)CC3C5)c21] | 68% |
| Intermediate f-14 | 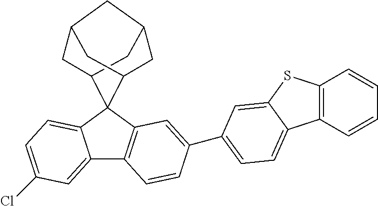 [SMILES: Clc1ccc2c(c1)-c1ccc(-c3ccc4c(c3)sc3ccccc34)cc1C21C2CC3CC(C2)CC1C3] | 65% |

Intermediates h-1 to h-9 were synthesized by using a method similar to the synthesis of the intermediate f-1, and by using a compound shown as a reactant C in Table 7 in place of the intermediate e-1 and a compound shown in a reactant D in place of 3-bromodibenzofuran.

TABLE 7
Synthesis of intermediate h-1 to intermediate h-9

| Inter- | | |
|---|---|---|
| mediate | | |
| No. | Reactant C | Reactant D |
| Inter- mediate h-1 | 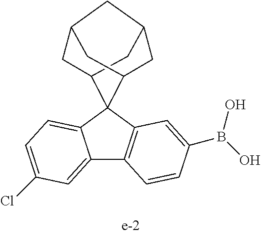 [SMILES: OB(O)c1ccc2c(c1)C1(c3ccc(Cl)cc3-2)C2CC3CC(C2)CC1C3] | 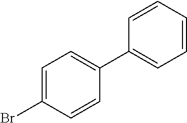 [SMILES: Brc1ccc(-c2ccccc2)cc1] |
| Inter- mediate h-2 | 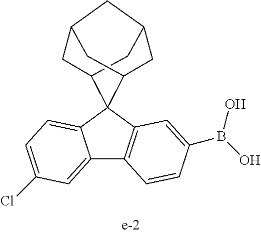 [SMILES: OB(O)c1ccc2c(c1)C1(c3ccc(Cl)cc3-2)C2CC3CC(C2)CC1C3] | 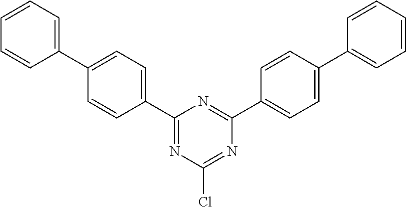 [SMILES: Clc1nc(-c2ccc(-c3ccccc3)cc2)nc(-c2ccc(-c3ccccc3)cc2)n1] |
| Inter- mediate h-3 | 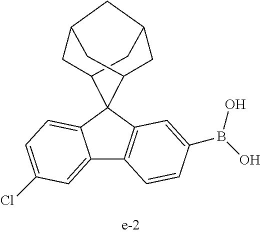 [SMILES: OB(O)c1ccc2c(c1)C1(c3ccc(Cl)cc3-2)C2CC3CC(C2)CC1C3] | 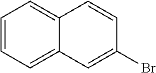 [SMILES: Brc1ccc2ccccc2c1] |
| Inter- mediate h-4 | 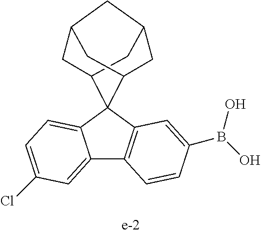 [SMILES: OB(O)c1ccc2c(c1)C1(c3ccc(Cl)cc3-2)C2CC3CC(C2)CC1C3] | 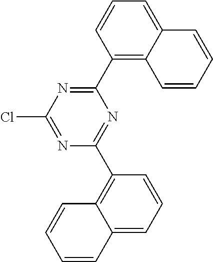 [SMILES: Clc1nc(-c2cccc3ccccc23)nc(-c2cccc3ccccc23)n1] |
| Inter- mediate h-5 | 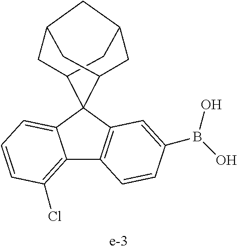 [SMILES: OB(O)c1ccc2c(c1)C1(c3cccc(Cl)c3-2)C2CC3CC(C2)CC1C3] | 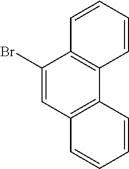 [SMILES: Brc1cc2ccccc2c2ccccc12] |
| Inter- mediate h-6 | 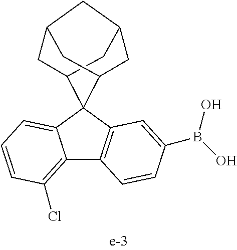 [SMILES: OB(O)c1ccc2c(c1)C1(c3cccc(Cl)c3-2)C2CC3CC(C2)CC1C3] | 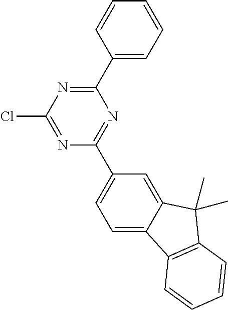 [SMILES: CC1(C)c2ccccc2-c2ccc(-c3nc(Cl)nc(-c4ccccc4)n3)cc21] |
| Inter- mediate h-7 | 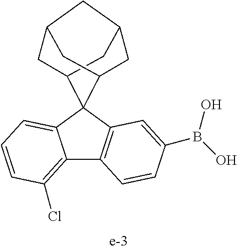 [SMILES: OB(O)c1ccc2c(c1)C1(c3cccc(Cl)c3-2)C2CC3CC(C2)CC1C3] | 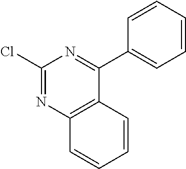 [SMILES: Clc1nc(-c2ccccc2)c2ccccc2n1] |
| Inter- mediate h-8 | 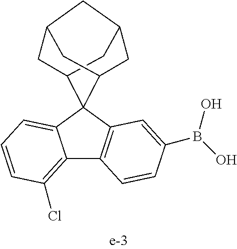 [SMILES: OB(O)c1ccc2c(c1)C1(c3cccc(Cl)c3-2)C2CC3CC(C2)CC1C3] | 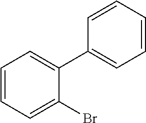 [SMILES: Brc1ccccc1-c1ccccc1] |
| Inter- mediate h-9 | 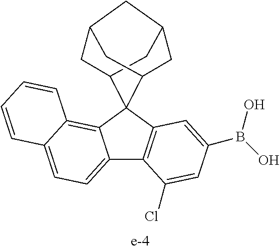 [SMILES: OB(O)c1cc(Cl)c2c(c1)C1(c3c-2ccc2ccccc32)C2CC3CC(C2)CC1C3] | 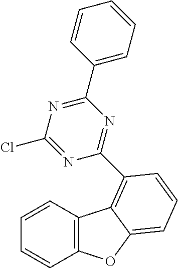 [SMILES: Clc1nc(-c2ccccc2)nc(-c2cccc3oc4ccccc4c23)n1] |
| Inter- | | |
| mediate | | Yield |
| No. | Structure | (%) |
| Inter- mediate h-1 | 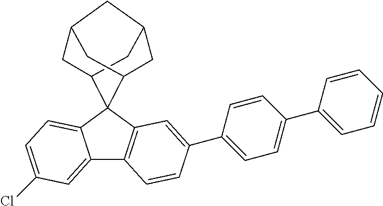 [SMILES: Clc1ccc2c(c1)-c1ccc(-c3ccc(-c4ccccc4)cc3)cc1C21C2CC3CC(C2)CC1C3] | 66% |
| Inter- mediate h-2 | 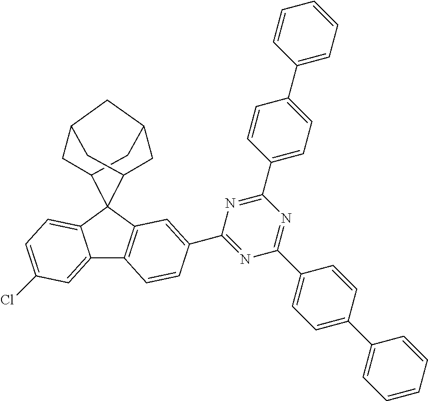 [SMILES: Clc1ccc2c(c1)-c1ccc(-c3nc(-c4ccc(-c5ccccc5)cc4)nc(-c4ccc(-c5ccccc5)cc4)n3)cc1C21C2CC3CC(C2)CC1C3] | 69% |
| Inter- mediate h-3 | 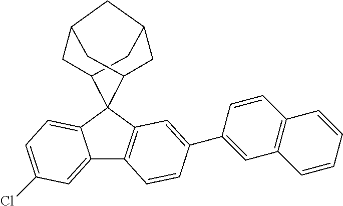 [SMILES: Clc1ccc2c(c1)-c1ccc(-c3ccc4ccccc4c3)cc1C21C2CC3CC(C2)CC1C3] | 76% |
| Inter- mediate h-4 | 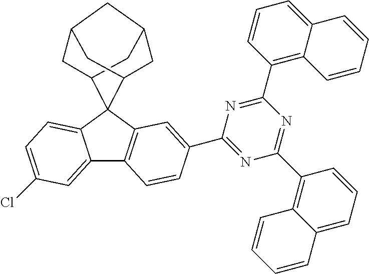 [SMILES: Clc1ccc2c(c1)-c1ccc(-c3nc(-c4cccc5ccccc45)nc(-c4cccc5ccccc45)n3)cc1C21C2CC3CC(C2)CC1C3] | 70% |
| Inter- mediate h-5 | 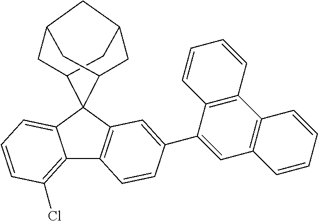 [SMILES: Clc1cccc2c1-c1ccc(-c3cc4ccccc4c4ccccc34)cc1C21C2CC3CC(C2)CC1C3] | 73% |
| Inter- mediate h-6 | 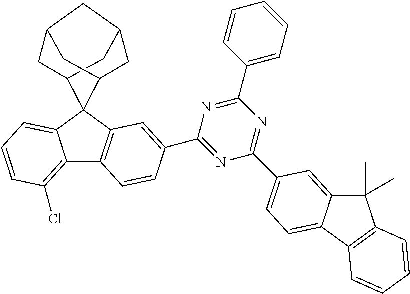 [SMILES: CC1(C)c2ccccc2-c2ccc(-c3nc(-c4ccccc4)nc(-c4ccc5c(c4)C4(c6cccc(Cl)c6-5)C5CC6CC(C5)CC4C6)n3)cc21] | 75% |
| Inter- mediate h-7 | 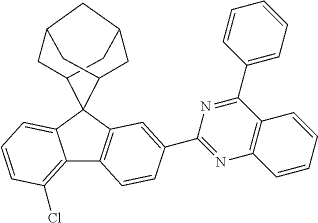 [SMILES: Clc1cccc2c1-c1ccc(-c3nc(-c4ccccc4)c4ccccc4n3)cc1C21C2CC3CC(C2)CC1C3] | 77% |
| Inter- mediate h-8 | 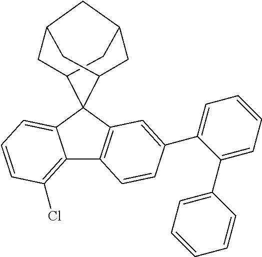 [SMILES: Clc1cccc2c1-c1ccc(-c3ccccc3-c3ccccc3)cc1C21C2CC3CC(C2)CC1C3] | 73% |
| Inter- mediate h-9 | 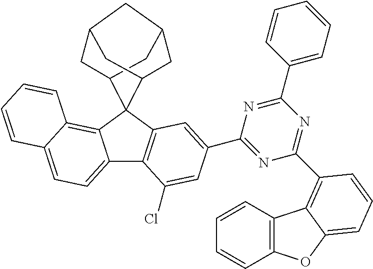 [SMILES: Clc1cc(-c2nc(-c3ccccc3)nc(-c3cccc4oc5ccccc5c34)n2)cc2c1-c1ccc3ccccc3c1C21C2CC3CC(C2)CC1C3] | 72% |

8. Synthesis of Intermediate g-1

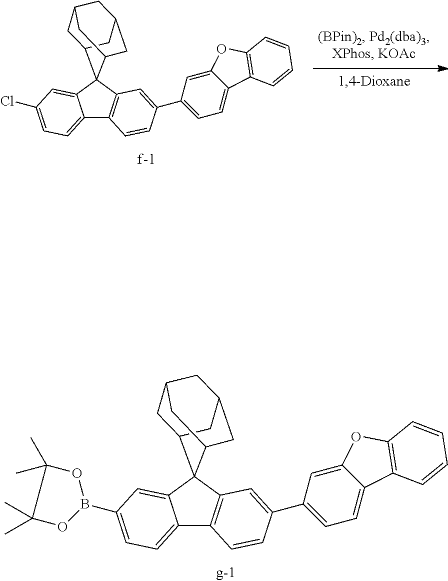 [SMILES: Clc1ccc2c(c1)C1(c3cc(-c4ccc5c(c4)oc4ccccc45)ccc3-2)C2CC3CC(C2)CC1C3 ; "(BPin)2, Pd2(dba)3,XPhos, KOAc" ; "1,4-Dioxane" ; CC1(C)OB(c2ccc3c(c2)C2(c4cc(-c5ccc6c(c5)oc5ccccc56)ccc4-3)C3CC4CC(C3)CC2C4)OC1(C)C]

The intermediate f-1 (9.1 g, 18.9 mmol), bis(pinacolato)diboron (5.7 g, 22.4 mmol), tris(dibenzylideneacetone)dipalladium (0.17 g, 0.19 mmol), 2-(dicyclohexylphosphino)-2′,4′,6′-tri(isopropyl)biphenyl (0.17 g, 0.37 mmol), potassium acetate (4.4 g, 41.1 mmol) and 1,4-dioxane (72 mL) were added into a flask, and stirred under reflux at 100° C. for 18 hours in a nitrogen atmosphere. The mixture was cooled to room temperature, dichloromethane and water were added into the mixture. An separated organic phase was washed with water and then dried with anhydrous magnesium sulfate, and concentrated in vacuum to obtain a crude product. The crude product was purified by silica gel column chromatography and eluted with a mixture of dichloromethane and n-heptane to obtain a white solid intermediate g-1 (7.0 g, 65%).

Intermediates g-2 to g-14 were synthesized by using a method similar to that described above and by replacing the intermediate f-1 with a reactant A in Table 8 below.

TABLE 8
Synthesis of intermediate g-2 to intermediate g-14

| In- | | | |
|---|---|---|---|
| ter- | | | |
| me- | | | |
| di- | | | |
| ate | | | Yield |
| No. | Reactant A | Structure | (%) |
| In- ter- me- di- ate g-2 | 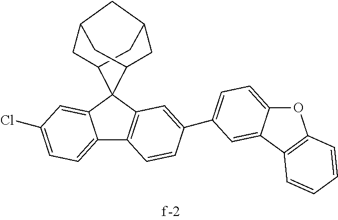 [SMILES: Clc1ccc2c(c1)C1(c3cc(-c4ccc5oc6ccccc6c5c4)ccc3-2)C2CC3CC(C2)CC1C3] | 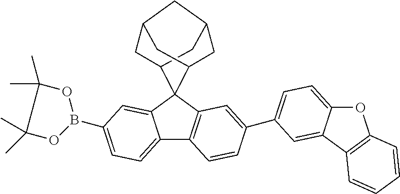 [SMILES: CC1(C)OB(c2ccc3c(c2)C2(c4cc(-c5ccc6oc7ccccc7c6c5)ccc4-3)C3CC4CC(C3)CC2C4)OC1(C)C] | 64% |
| In- ter- me- di- ate g-3 | 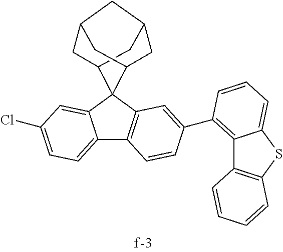 [SMILES: Clc1ccc2c(c1)C1(c3cc(-c4cccc5sc6ccccc6c45)ccc3-2)C2CC3CC(C2)CC1C3] | 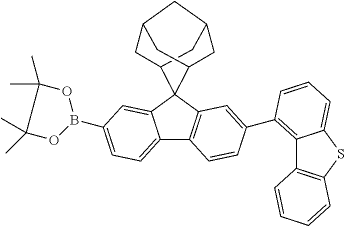 [SMILES: CC1(C)OB(c2ccc3c(c2)C2(c4cc(-c5cccc6sc7ccccc7c56)ccc4-3)C3CC4CC(C3)CC2C4)OC1(C)C] | 66% |
| In- ter- me- di- ate g-4 | 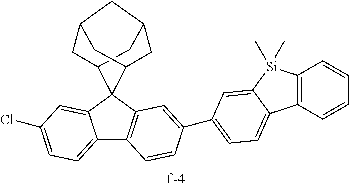 [SMILES: C[Si]1(C)c2ccccc2-c2ccc(-c3ccc4c(c3)C3(c5cc(Cl)ccc5-4)C4CC5CC(C4)CC3C5)cc21] | 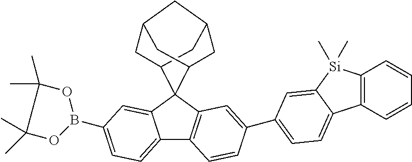 [SMILES: CC1(C)OB(c2ccc3c(c2)C2(c4cc(-c5ccc6c(c5)[Si](C)(C)c5ccccc5-6)ccc4-3)C3CC4CC(C3)CC2C4)OC1(C)C] | 65% |
| In- ter- me- di- ate g-5 | 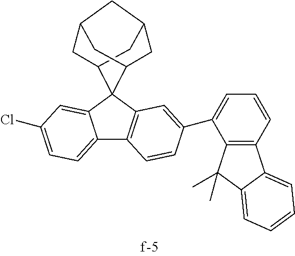 [SMILES: CC1(C)c2ccccc2-c2cccc(-c3ccc4c(c3)C3(c5cc(Cl)ccc5-4)C4CC5CC(C4)CC3C5)c21] | 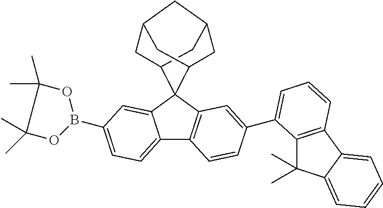 [SMILES: CC1(C)c2ccccc2-c2cccc(-c3ccc4c(c3)C3(c5cc(B6OC(C)(C)C(C)(C)O6)ccc5-4)C4CC5CC(C4)CC3C5)c21] | 63% |
| In- ter- me- di- ate g-6 | 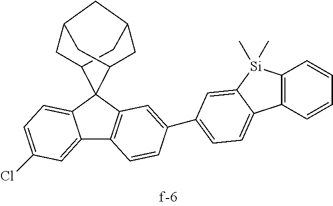 [SMILES: C[Si]1(C)c2ccccc2-c2ccc(-c3ccc4c(c3)C3(c5ccc(Cl)cc5-4)C4CC5CC(C4)CC3C5)cc21] | 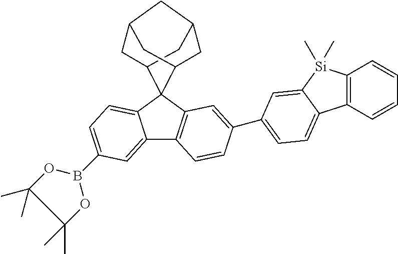 [SMILES: CC1(C)OB(c2ccc3c(c2)-c2ccc(-c4ccc5c(c4)[Si](C)(C)c4ccccc4-5)cc2C32C3CC4CC(C3)CC2C4)OC1(C)C] | 65% |
| In- ter- me- di- ate g-7 | 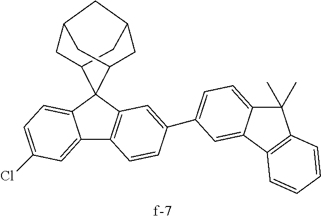 [SMILES: CC1(C)c2ccccc2-c2cc(-c3ccc4c(c3)C3(c5ccc(Cl)cc5-4)C4CC5CC(C4)CC3C5)ccc21] | 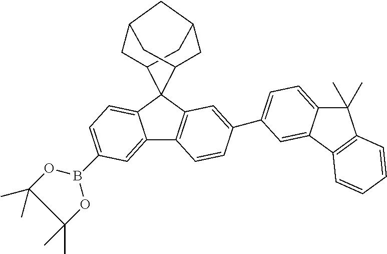 [SMILES: CC1(C)c2ccccc2-c2cc(-c3ccc4c(c3)C3(c5ccc(B6OC(C)(C)C(C)(C)O6)cc5-4)C4CC5CC(C4)CC3C5)ccc21] | 67% |
| In- ter- me- di- ate g-8 | 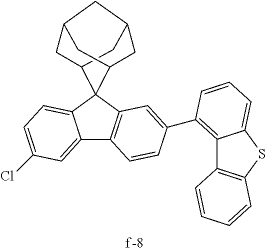 [SMILES: Clc1ccc2c(c1)-c1ccc(-c3cccc4sc5ccccc5c34)cc1C21C2CC3CC(C2)CC1C3] | 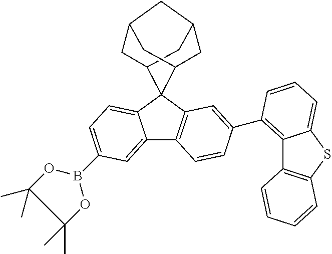 [SMILES: CC1(C)OB(c2ccc3c(c2)-c2ccc(-c4cccc5sc6ccccc6c45)cc2C32C3CC4CC(C3)CC2C4)OC1(C)C] | 73% |
| In- ter- me- di- ate g-9 | 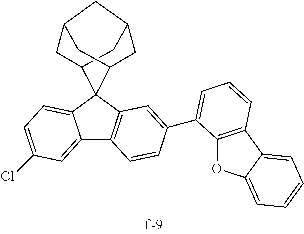 [SMILES: Clc1ccc2c(c1)-c1ccc(-c3cccc4c3oc3ccccc34)cc1C21C2CC3CC(C2)CC1C3] | 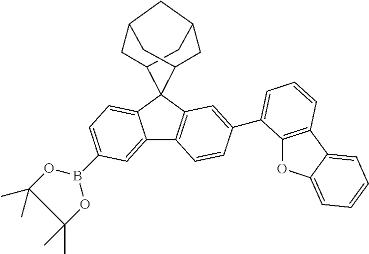 [SMILES: CC1(C)OB(c2ccc3c(c2)-c2ccc(-c4cccc5c4oc4ccccc45)cc2C32C3CC4CC(C3)CC2C4)OC1(C)C] | 64% |
| In- ter- me- di- ate g- 10 | 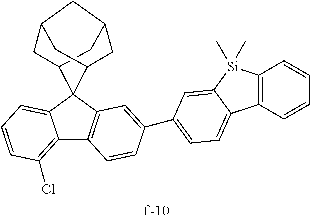 [SMILES: C[Si]1(C)c2ccccc2-c2ccc(-c3ccc4c(c3)C3(c5cccc(Cl)c5-4)C4CC5CC(C4)CC3C5)cc21] | 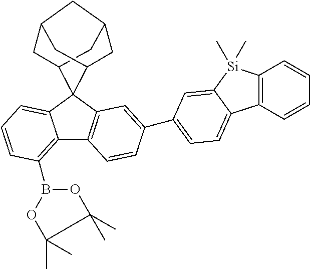 [SMILES: CC1(C)OB(c2cccc3c2-c2ccc(-c4ccc5c(c4)[Si](C)(C)c4ccccc4-5)cc2C32C3CC4CC(C3)CC2C4)OC1(C)C] | 58% |
| In- ter- me- di- ate g- 11 | 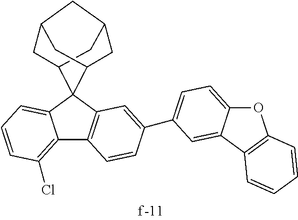 [SMILES: Clc1cccc2c1-c1ccc(-c3ccc4oc5ccccc5c4c3)cc1C21C2CC3CC(C2)CC1C3] | 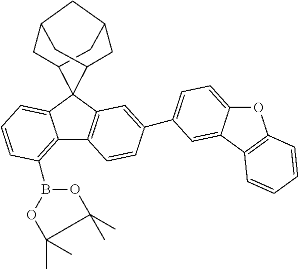 [SMILES: CC1(C)OB(c2cccc3c2-c2ccc(-c4ccc5oc6ccccc6c5c4)cc2C32C3CC4CC(C3)CC2C4)OC1(C)C] | 64% |
| In- ter- me- di- ate g- 12 | 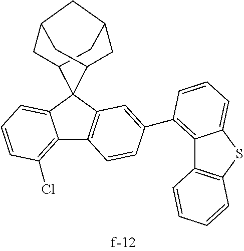 [SMILES: Clc1cccc2c1-c1ccc(-c3cccc4sc5ccccc5c34)cc1C21C2CC3CC(C2)CC1C3] | 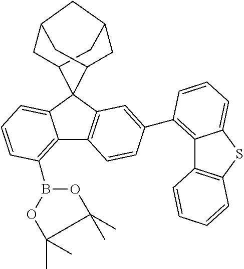 [SMILES: CC1(C)OB(c2cccc3c2-c2ccc(-c4cccc5sc6ccccc6c45)cc2C32C3CC4CC(C3)CC2C4)OC1(C)C] | 65% |
| In- ter- me- di- ate g- 13 | 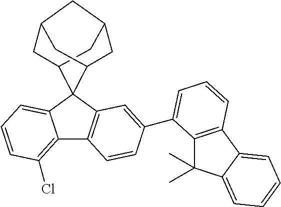 [SMILES: CC1(C)c2ccccc2-c2cccc(-c3ccc4c(c3)C3(c5cccc(Cl)c5-4)C4CC5CC(C4)CC3C5)c21] | 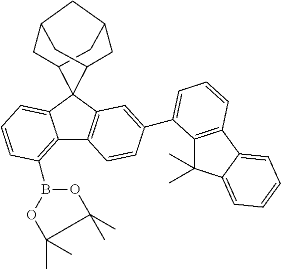 [SMILES: CC1(C)c2ccccc2-c2cccc(-c3ccc4c(c3)C3(c5cccc(B6OC(C)(C)C(C)(C)O6)c5-4)C4CC5CC(C4)CC3C5)c21] | 63% |
| In- ter- me- di- ate g- 14 | 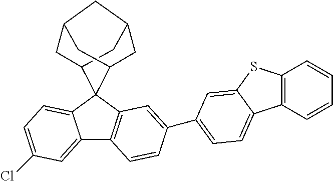 [SMILES: Clc1ccc2c(c1)-c1ccc(-c3ccc4c(c3)sc3ccccc34)cc1C21C2CC3CC(C2)CC1C3] | 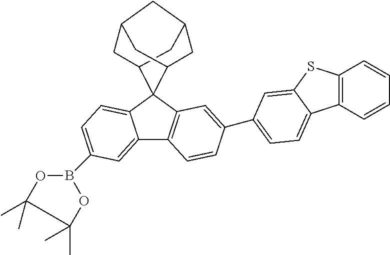 [SMILES: CC1(C)OB(c2ccc3c(c2)-c2ccc(-c4ccc5c(c4)sc4ccccc45)cc2C32C3CC4CC(C3)CC2C4)OC1(C)C] | 65% |

9. Synthesis of Compound 16

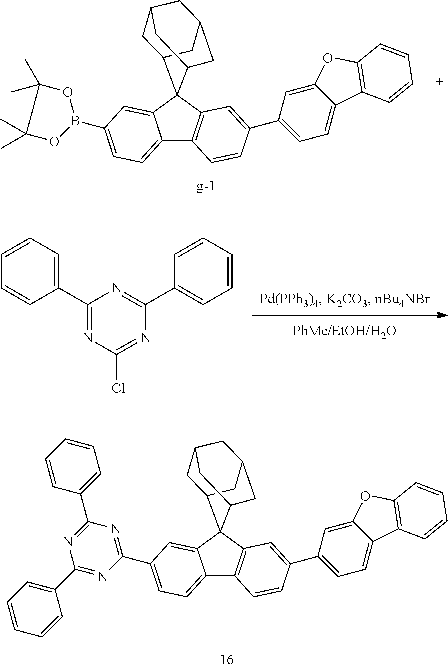 [SMILES: CC1(C)OB(c2ccc3c(c2)C2(c4cc(-c5ccc6c(c5)oc5ccccc56)ccc4-3)C3CC4CC(C3)CC2C4)OC1(C)C ; Clc1nc(-c2ccccc2)nc(-c2ccccc2)n1 ; c1ccc(-c2nc(-c3ccccc3)nc(-c3ccc4c(c3)C3(c5cc(-c6ccc7c(c6)oc6ccccc67)ccc5-4)C4CC5CC(C4)CC3C5)n2)cc1]

The intermediate g-1 (7.0 g, 12.1 mmol), 2-chloro-4,6-diphenyl-1,3,5-triazine (3.1 g, 11.5 mmol), tetrakis(triphenylphosphine)palladium (0.27 g, 0.23 mmol), potassium carbonate (3.5 g, 25.3 mmol), tetrabutylammonium bromide (0.7 g, 2.3 mmol) were added into a flask, a mixed solvent of toluene (56 mL), ethanol (28 mL) and water (14 mL) was added in a nitrogen atmosphere. The mixture was heated to 80° C., and stirred for 12 hours while keeping the same temperature and then cooled to the room temperature, stirring was stopped. The reaction solution was washed with water, an separated organic phase was dried with anhydrous magnesium sulfate, and concentrated in vacuum to obtain a crude product. The crude product was purified by silica gel column chromatography and eluted with a mixture of dichloromethane and n-heptane to obtain the compound 16 (4 g, yield 50%) as a solid product.

Preparation examples 2 to 14 in Table 10 were synthesized by using a method similar to the synthesis of preparation example 1, and by using a reactant A shown in Table 9 in place of the intermediate g-1 and a reactant B shown in Table 9 in place of 2-chloro-4,6-diphenyl-1,3,5-triazine.

TABLE 9
Preparation examples 2 to 14

| Prep- | | | |
|---|---|---|---|
| ara- | | | |
| tion | | | |
| exam- | Com- | | |
| ple | pound | | |
| No. | No. | Reactant A | Reactant B |
| 2 | 23 | 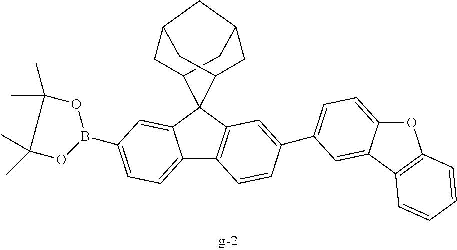 [SMILES: CC1(C)OB(c2ccc3c(c2)C2(c4cc(-c5ccc6oc7ccccc7c6c5)ccc4-3)C3CC4CC(C3)CC2C4)OC1(C)C] | 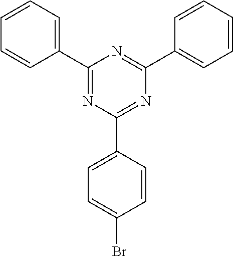 [SMILES: Brc1ccc(-c2nc(-c3ccccc3)nc(-c3ccccc3)n2)cc1] |
| 3 | 141 | 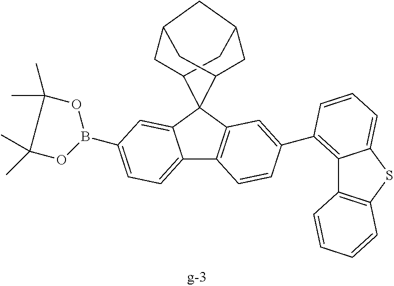 [SMILES: CC1(C)OB(c2ccc3c(c2)C2(c4cc(-c5cccc6sc7ccccc7c56)ccc4-3)C3CC4CC(C3)CC2C4)OC1(C)C] | 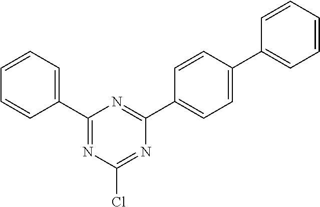 [SMILES: Clc1nc(-c2ccccc2)nc(-c2ccc(-c3ccccc3)cc2)n1] |
| 4 | 238 | 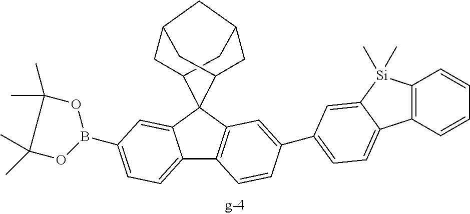 [SMILES: CC1(C)OB(c2ccc3c(c2)C2(c4cc(-c5ccc6c(c5)[Si](C)(C)c5ccccc5-6)ccc4-3)C3CC4CC(C3)CC2C4)OC1(C)C] | 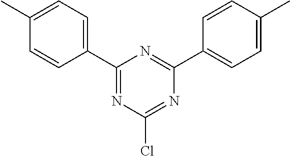 |
| 5 | 259 | 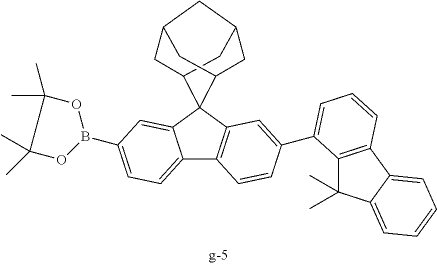 [SMILES: CC1(C)c2ccccc2-c2cccc(-c3ccc4c(c3)C3(c5cc(B6OC(C)(C)C(C)(C)O6)ccc5-4)C4CC5CC(C4)CC3C5)c21] | 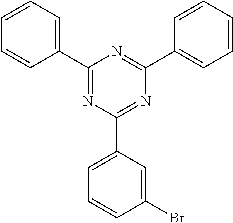 [SMILES: Brc1cccc(-c2nc(-c3ccccc3)nc(-c3ccccc3)n2)c1] |
| 6 | 234 | 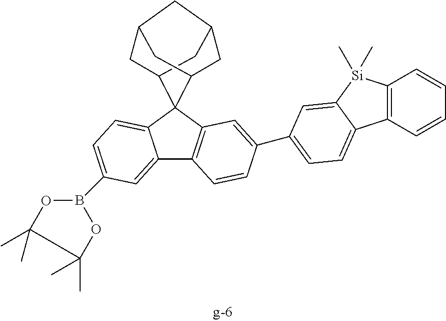 [SMILES: CC1(C)OB(c2ccc3c(c2)-c2ccc(-c4ccc5c(c4)[Si](C)(C)c4ccccc4-5)cc2C32C3CC4CC(C3)CC2C4)OC1(C)C] | 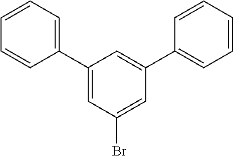 [SMILES: Brc1cc(-c2ccccc2)cc(-c2ccccc2)c1] |
| 7 | 268 | 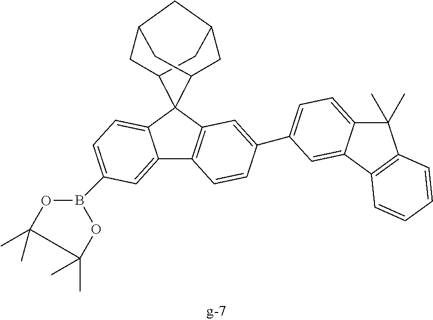 [SMILES: CC1(C)c2ccccc2-c2cc(-c3ccc4c(c3)C3(c5ccc(B6OC(C)(C)C(C)(C)O6)cc5-4)C4CC5CC(C4)CC3C5)ccc21] | 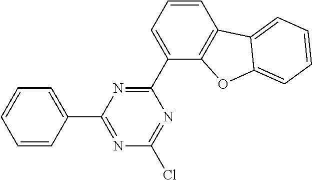 [SMILES: Clc1nc(-c2ccccc2)nc(-c2cccc3c2oc2ccccc23)n1] |
| 8 | 142 | 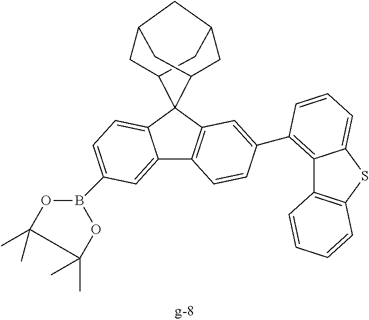 [SMILES: CC1(C)OB(c2ccc3c(c2)-c2ccc(-c4cccc5sc6ccccc6c45)cc2C32C3CC4CC(C3)CC2C4)OC1(C)C] | 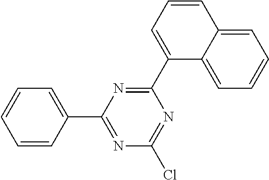 [SMILES: Clc1nc(-c2ccccc2)nc(-c2cccc3ccccc23)n1] |
| 9 | 10 | 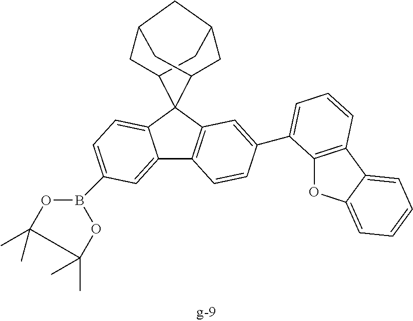 [SMILES: CC1(C)OB(c2ccc3c(c2)-c2ccc(-c4cccc5c4oc4ccccc45)cc2C32C3CC4CC(C3)CC2C4)OC1(C)C] | 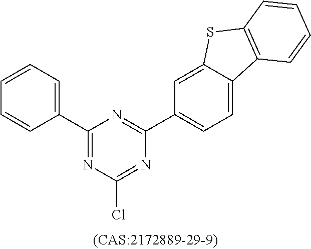 [SMILES: Clc1nc(-c2ccccc2)nc(-c2ccc3c(c2)sc2ccccc23)n1] |
| 10 | 239 | 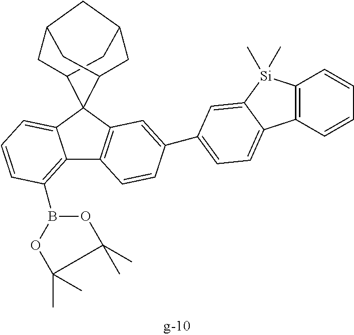 [SMILES: CC1(C)OB(c2cccc3c2-c2ccc(-c4ccc5c(c4)[Si](C)(C)c4ccccc4-5)cc2C32C3CC4CC(C3)CC2C4)OC1(C)C] | 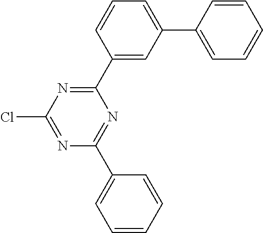 [SMILES: Clc1nc(-c2ccccc2)nc(-c2cccc(-c3ccccc3)c2)n1] |
| 11 | 25 | 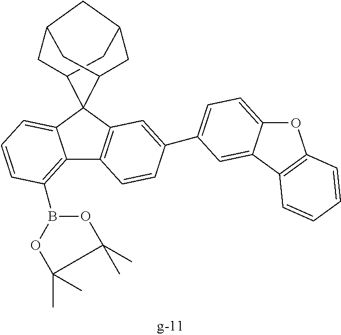 [SMILES: CC1(C)OB(c2cccc3c2-c2ccc(-c4ccc5oc6ccccc6c5c4)cc2C32C3CC4CC(C3)CC2C4)OC1(C)C] | 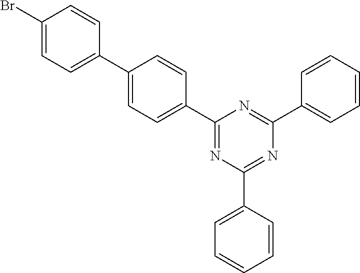 [SMILES: Brc1ccc(-c2ccc(-c3nc(-c4ccccc4)nc(-c4ccccc4)n3)cc2)cc1] |
| 12 | 143 | 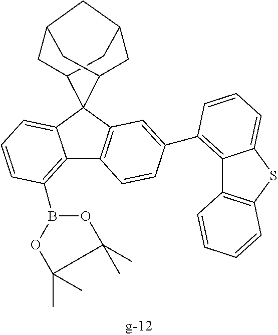 [SMILES: CC1(C)OB(c2cccc3c2-c2ccc(-c4cccc5sc6ccccc6c45)cc2C32C3CC4CC(C3)CC2C4)OC1(C)C] | 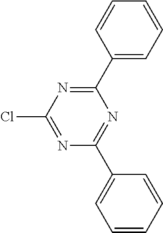 [SMILES: Clc1nc(-c2ccccc2)nc(-c2ccccc2)n1] |
| 13 | 258 | 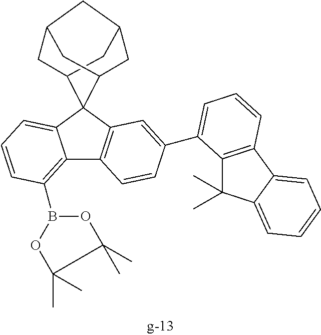 [SMILES: CC1(C)c2ccccc2-c2cccc(-c3ccc4c(c3)C3(c5cccc(B6OC(C)(C)C(C)(C)O6)c5-4)C4CC5CC(C4)CC3C5)c21] | 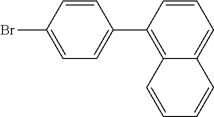 [SMILES: Brc1ccc(-c2cccc3ccccc23)cc1] |
| 14 | 128 | 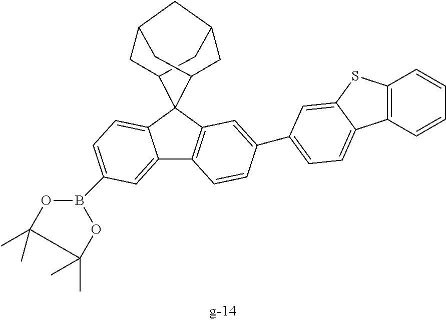 [SMILES: CC1(C)OB(c2ccc3c(c2)-c2ccc(-c4ccc5c(c4)sc4ccccc45)cc2C32C3CC4CC(C3)CC2C4)OC1(C)C] | 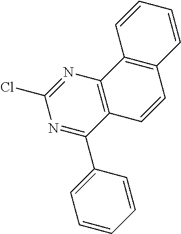 [SMILES: Clc1nc(-c2ccccc2)c2ccc3ccccc3c2n1] |
| Prep- | | | |
| ara- | | | |
| tion | | | |
| exam- | Com- | | |
| ple | pound | | |
| No. | No. | Structure | Yield |
| 2 | 23 | 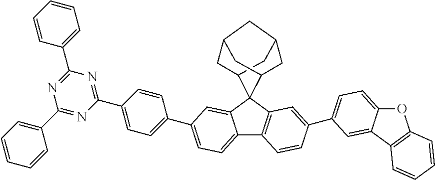 [SMILES: c1ccc(-c2nc(-c3ccccc3)nc(-c3ccc(-c4ccc5c(c4)C4(c6cc(-c7ccc8oc9ccccc9c8c7)ccc6-5)C5CC6CC(C5)CC4C6)cc3)n2)cc1] | 54% |
| 3 | 141 | 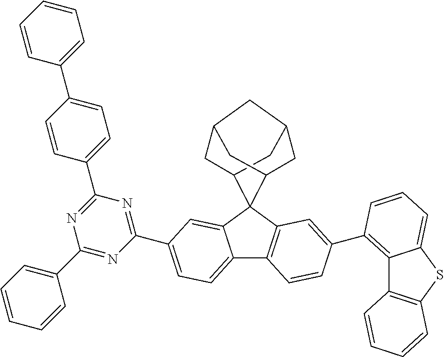 [SMILES: c1ccc(-c2ccc(-c3nc(-c4ccccc4)nc(-c4ccc5c(c4)C4(c6cc(-c7cccc8sc9ccccc9c78)ccc6-5)C5CC6CC(C5)CC4C6)n3)cc2)cc1] | 56% |
| 4 | 238 | 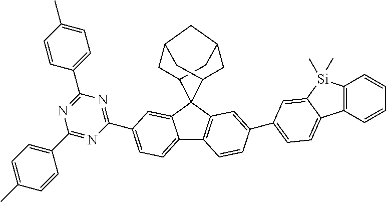 [SMILES: Cc1ccc(-c2nc(-c3ccc(C)cc3)nc(-c3ccc4c(c3)C3(c5cc(-c6ccc7c(c6)[Si](C)(C)c6ccccc6-7)ccc5-4)C4CC5CC(C4)CC3C5)n2)cc1] | 55% |
| 5 | 259 | 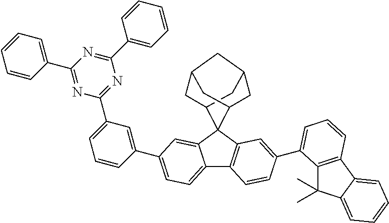 [SMILES: CC1(C)c2ccccc2-c2cccc(-c3ccc4c(c3)C3(c5cc(-c6cccc(-c7nc(-c8ccccc8)nc(-c8ccccc8)n7)c6)ccc5-4)C4CC5CC(C4)CC3C5)c21] | 53% |
| 6 | 234 | 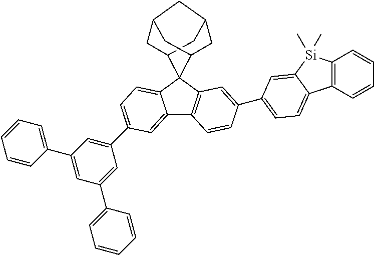 [SMILES: C[Si]1(C)c2ccccc2-c2ccc(-c3ccc4c(c3)C3(c5ccc(-c6cc(-c7ccccc7)cc(-c7ccccc7)c6)cc5-4)C4CC5CC(C4)CC3C5)cc21] | 55% |
| 7 | 268 | 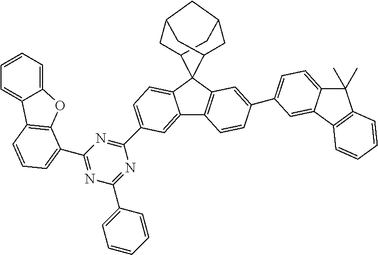 [SMILES: CC1(C)c2ccccc2-c2cc(-c3ccc4c(c3)C3(c5ccc(-c6nc(-c7ccccc7)nc(-c7cccc8c7oc7ccccc78)n6)cc5-4)C4CC5CC(C4)CC3C5)ccc21] | 57% |
| 8 | 142 | 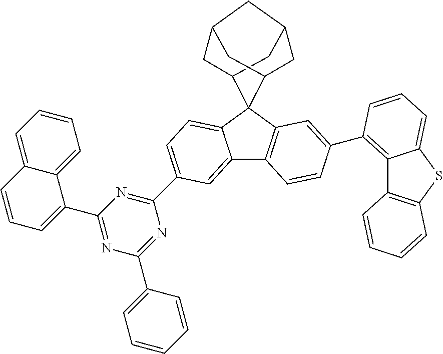 [SMILES: c1ccc(-c2nc(-c3ccc4c(c3)-c3ccc(-c5cccc6sc7ccccc7c56)cc3C43C4CC5CC(C4)CC3C5)nc(-c3cccc4ccccc34)n2)cc1] | 53% |
| 9 | 10 | 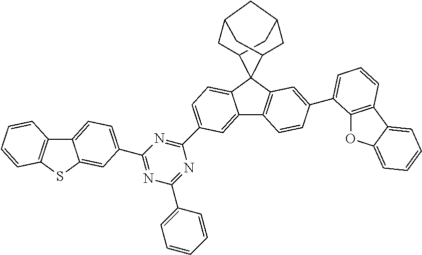 [SMILES: c1ccc(-c2nc(-c3ccc4c(c3)-c3ccc(-c5cccc6c5oc5ccccc56)cc3C43C4CC5CC(C4)CC3C5)nc(-c3ccc4c(c3)sc3ccccc34)n2)cc1] | 54% |
| 10 | 239 | 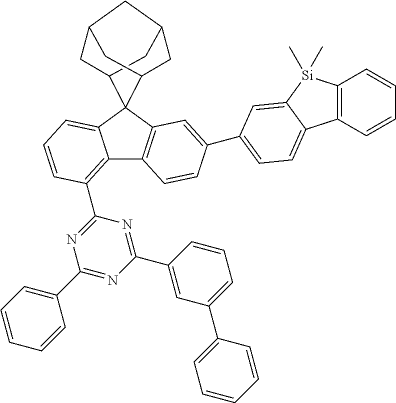 [SMILES: C[Si]1(C)c2ccccc2-c2ccc(-c3ccc4c(c3)C3(c5cccc(-c6nc(-c7ccccc7)nc(-c7cccc(-c8ccccc8)c7)n6)c5-4)C4CC5CC(C4)CC3C5)cc21] | 48% |
| 11 | 25 | 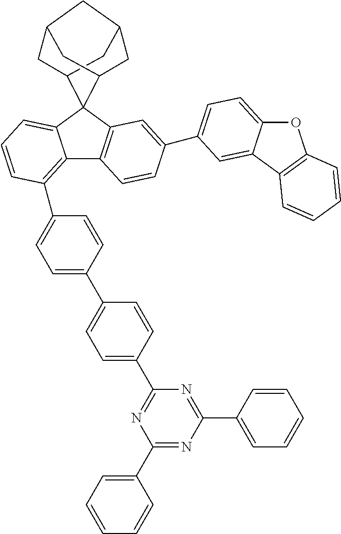 [SMILES: c1ccc(-c2nc(-c3ccccc3)nc(-c3ccc(-c4ccc(-c5cccc6c5-c5ccc(-c7ccc8oc9ccccc9c8c7)cc5C65C6CC7CC(C6)CC5C7)cc4)cc3)n2)cc1] | 54% |
| 12 | 143 | 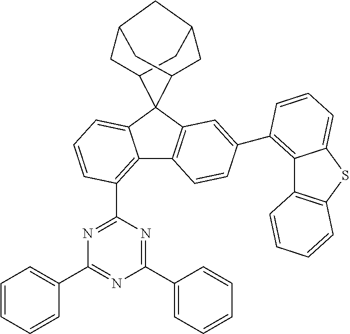 [SMILES: c1ccc(-c2nc(-c3ccccc3)nc(-c3cccc4c3-c3ccc(-c5cccc6sc7ccccc7c56)cc3C43C4CC5CC(C4)CC3C5)n2)cc1] | 55% |
| 13 | 258 | 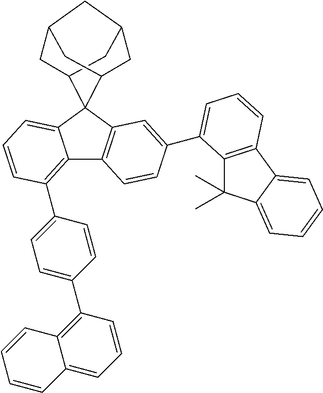 [SMILES: CC1(C)c2ccccc2-c2cccc(-c3ccc4c(c3)C3(c5cccc(-c6ccc(-c7cccc8ccccc78)cc6)c5-4)C4CC5CC(C4)CC3C5)c21] | 53% |
| 14 | 128 | 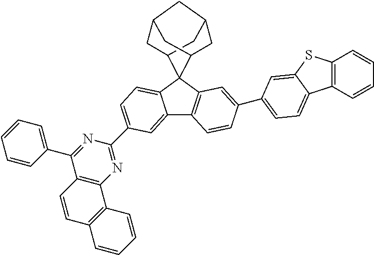 [SMILES: c1ccc(-c2nc(-c3ccc4c(c3)-c3ccc(-c5ccc6c(c5)sc5ccccc56)cc3C43C4CC5CC(C4)CC3C5)nc3c2ccc2ccccc23)cc1] | 57% |

2. Synthesis of Preparation Examples 15 to 23

Taking preparation example 15 as an example:

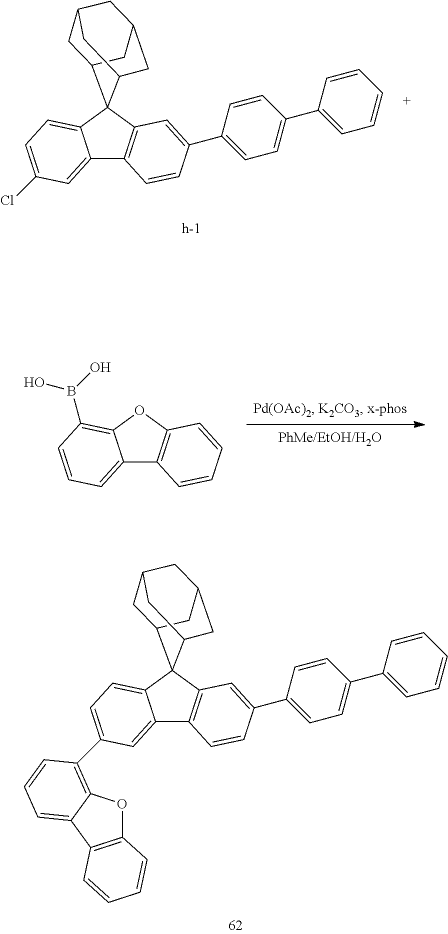 [SMILES: Clc1ccc2c(c1)-c1ccc(-c3ccc(-c4ccccc4)cc3)cc1C21C2CC3CC(C2)CC1C3 ; OB(O)c1cccc2c1oc1ccccc12 ; "Pd(OAc)2, K2CO3, x-phos" ; "PhMe/EtOH/H2O" ; c1ccc(-c2ccc(-c3ccc4c(c3)C3(c5ccc(-c6cccc7c6oc6ccccc67)cc5-4)C4CC5CC(C4)CC3C5)cc2)cc1]

The intermediate h-1 (9 g, 19.0 mmol), 4-dibenzofuran (3.67 g, 17.3 mmol), Pd(OAc)₂ (0.04 g, 0.17 mmol), K₂CO₃ (5.25 g, 38.05 mmol), xphos (0.16 g, 0.34 mmol) were added into toluene (72 mL), absolute ethanol (36 mL), and deionized water (18 mL) in a three-necked flask, and were heated to reflux at 78° C. and stirred for 12 h. After the reaction was completed, the mixture was extracted with dichloromethane and water, and an separated organic phase was dried with anhydrous MgSO₄, filtrated, and the filtrate was concentrated in vacuum to obtain a crude product. The crude product was purified by silica gel column to obtain a compound 62 (5.5 g, yield 53%).

Preparation examples 16 to 23 shown in Table 10 were synthesized by using a method similar to the synthesis of preparation example 15, and by using a reactant A shown in Table 10 in place of the intermediate h-1 and a reactant B shown in Table 10 in place of 4-dibenzofuran.

TABLE 10
Preparation examples 16 to 23

| Prepar- | | | |
|---|---|---|---|
| ation | Com- | | |
| example | pound | | |
| No. | No. | Reactant A | Reactant B |
| 16 | 51 | 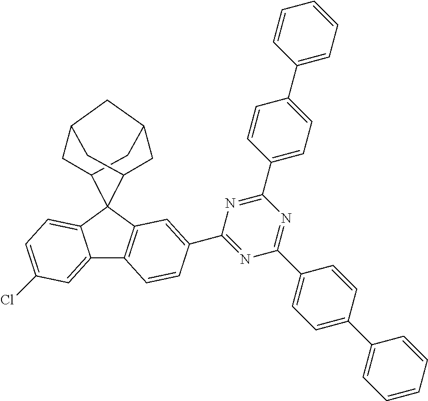 [SMILES: Clc1ccc2c(c1)-c1ccc(-c3nc(-c4ccc(-c5ccccc5)cc4)nc(-c4ccc(-c5ccccc5)cc4)n3)cc1C21C2CC3CC(C2)CC1C3] | 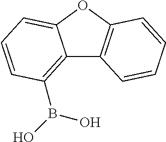 [SMILES: OB(O)c1cccc2oc3ccccc3c12] |
| 17 | 147 | 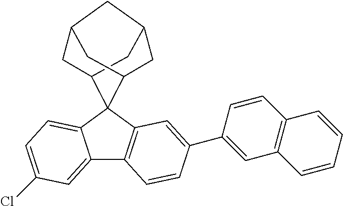 [SMILES: Clc1ccc2c(c1)-c1ccc(-c3ccc4ccccc4c3)cc1C21C2CC3CC(C2)CC1C3] | 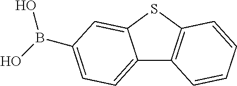 [SMILES: OB(O)c1ccc2c(c1)sc1ccccc12] |
| 18 | 154 | 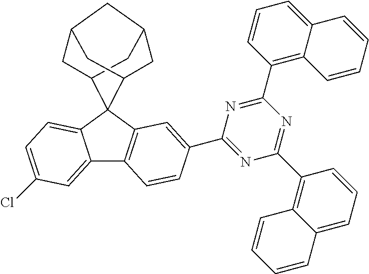 [SMILES: Clc1ccc2c(c1)-c1ccc(-c3nc(-c4cccc5ccccc45)nc(-c4cccc5ccccc45)n3)cc1C21C2CC3CC(C2)CC1C3] | 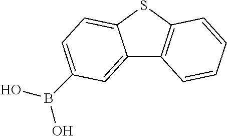 [SMILES: OB(O)c1ccc2sc3ccccc3c2c1] |
| 19 | 107 | 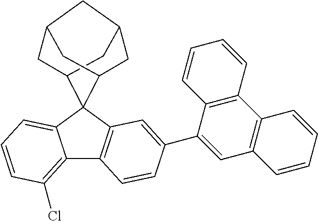 [SMILES: Clc1cccc2c1-c1ccc(-c3cc4ccccc4c4ccccc34)cc1C21C2CC3CC(C2)CC1C3] | 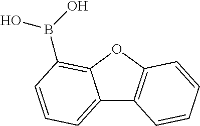 [SMILES: OB(O)c1cccc2c1oc1ccccc12] |
| 20 | 93 | 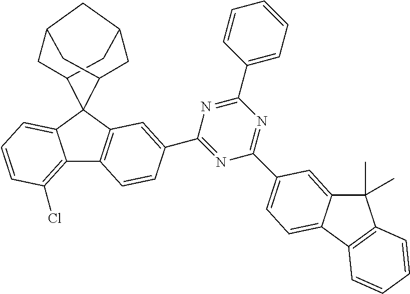 [SMILES: CC1(C)c2ccccc2-c2ccc(-c3nc(-c4ccccc4)nc(-c4ccc5c(c4)C4(c6cccc(Cl)c6-5)C5CC6CC(C5)CC4C6)n3)cc21] | 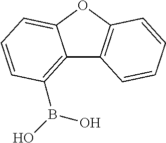 [SMILES: OB(O)c1cccc2oc3ccccc3c12] |
| 21 | 185 | 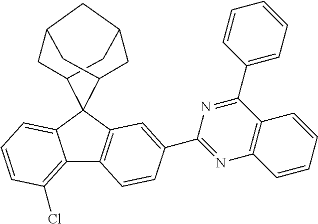 [SMILES: Clc1cccc2c1-c1ccc(-c3nc(-c4ccccc4)c4ccccc4n3)cc1C21C2CC3CC(C2)CC1C3] | 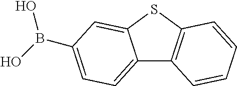 [SMILES: OB(O)c1ccc2c(c1)sc1ccccc12] |
| 22 | 192 | 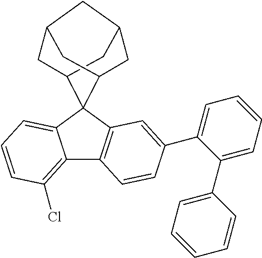 [SMILES: Clc1cccc2c1-c1ccc(-c3ccccc3-c3ccccc3)cc1C21C2CC3CC(C2)CC1C3] | 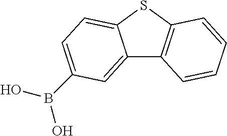 [SMILES: OB(O)c1ccc2sc3ccccc3c2c1] |
| 23 | 280 | 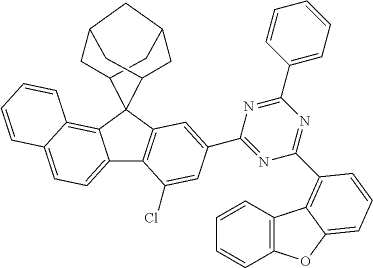 [SMILES: Clc1cc(-c2nc(-c3ccccc3)nc(-c3cccc4oc5ccccc5c34)n2)cc2c1-c1ccc3ccccc3c1C21C2CC3CC(C2)CC1C3] | 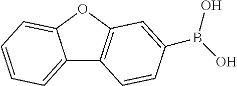 [SMILES: OB(O)c1ccc2c(c1)oc1ccccc12] |
| Prepar- | | | |
| ation | Com- | | |
| example | pound | | |
| No. | No. | Structure | Yield |
| 16 | 51 | 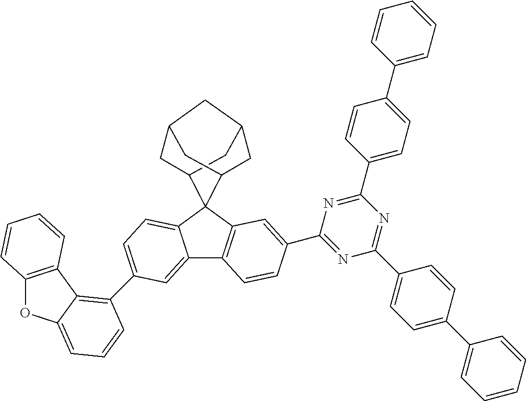 [SMILES: c1ccc(-c2ccc(-c3nc(-c4ccc(-c5ccccc5)cc4)nc(-c4ccc5c(c4)C4(c6ccc(-c7cccc8oc9ccccc9c78)cc6-5)C5CC6CC(C5)CC4C6)n3)cc2)cc1] | 54% |
| 17 | 147 | 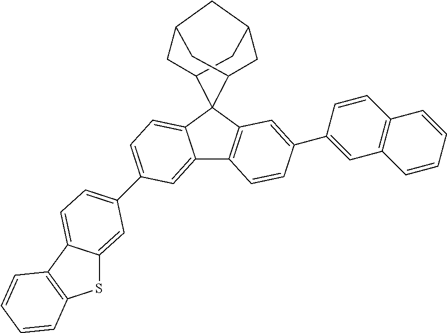 [SMILES: c1ccc2cc(-c3ccc4c(c3)C3(c5ccc(-c6ccc7c(c6)sc6ccccc67)cc5-4)C4CC5CC(C4)CC3C5)ccc2c1] | 56% |
| 18 | 154 | 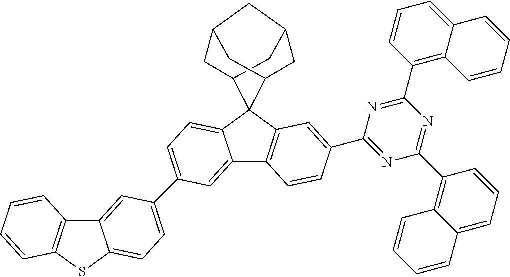 [SMILES: c1ccc2c(-c3nc(-c4ccc5c(c4)C4(c6ccc(-c7ccc8sc9ccccc9c8c7)cc6-5)C5CC6CC(C5)CC4C6)nc(-c4cccc5ccccc45)n3)cccc2c1] | 55% |
| 19 | 107 | 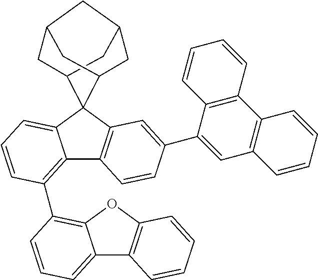 [SMILES: c1cc(-c2cccc3c2oc2ccccc23)c2c(c1)C1(c3cc(-c4cc5ccccc5c5ccccc45)ccc3-2)C2CC3CC(C2)CC1C3] | 53% |
| 20 | 93 | 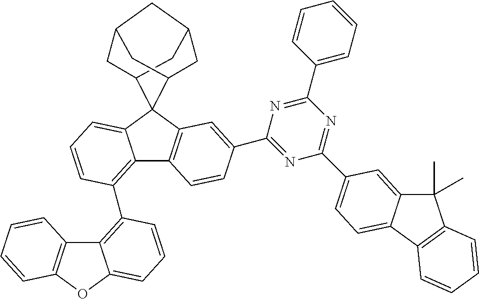 [SMILES: CC1(C)c2ccccc2-c2ccc(-c3nc(-c4ccccc4)nc(-c4ccc5c(c4)C4(c6cccc(-c7cccc8oc9ccccc9c78)c6-5)C5CC6CC(C5)CC4C6)n3)cc21] | 55% |
| 21 | 185 | 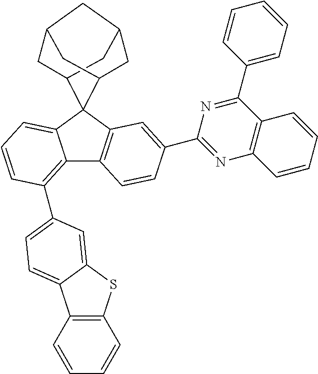 [SMILES: c1ccc(-c2nc(-c3ccc4c(c3)C3(c5cccc(-c6ccc7c(c6)sc6ccccc67)c5-4)C4CC5CC(C4)CC3C5)nc3ccccc23)cc1] | 57% |
| 22 | 192 | 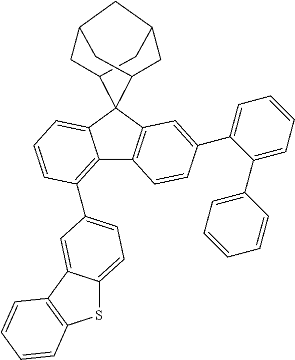 [SMILES: c1ccc(-c2ccccc2-c2ccc3c(c2)C2(c4cccc(-c5ccc6sc7ccccc7c6c5)c4-3)C3CC4CC(C3)CC2C4)cc1] | 54% |
| 23 | 280 | 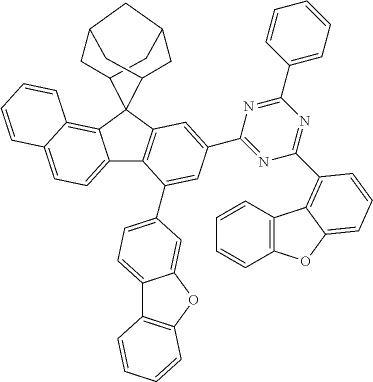 [SMILES: c1ccc(-c2nc(-c3cc(-c4ccc5c(c4)oc4ccccc45)c4c(c3)C3(c5c-4ccc4ccccc54)C4CC5CC(C4)CC3C5)nc(-c3cccc4oc5ccccc5c34)n2)cc1] | 52% |

3. Synthesis of Preparation Examples 24 to 35

1. Synthesis of Intermediate I-1

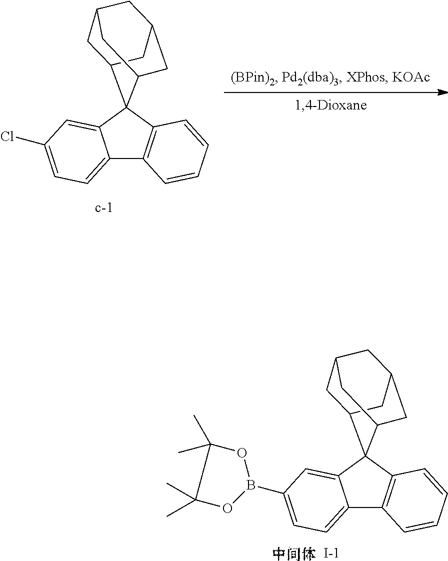 [SMILES: Clc1ccc2c(c1)C1(c3ccccc3-2)C2CC3CC(C2)CC1C3 ; "(BPin)2, Pd2(dba)3, XPhos, KOAc" ; "1,4-Dioxane" ; CC1(C)OB(c2ccc3c(c2)C2(c4ccccc4-3)C3CC4CC(C3)CC2C4)OC1(C)C ; [I-]]

The intermediate c-1 (20 g, 62.3 mmol), bis(pinacolato)diboron (19.0 g, 74.8 mmol), tris(dibenzylideneacetone)dipalladium (0.57 g, 0.62 mmol), 2-(dicyclohexylphosphino)-2′,4′,6′-tri(isopropyl)biphenyl (0.59 g, 1.25 mmol), potassium acetate (13.4 g, 137.1 mmol) and 1,4-dioxane (160 mL) were added into a flask, and stirred under reflux at 100° C. for 18 hours in a nitrogen atmosphere. The mixture was cooled to the room temperature, dichloromethane and water were added into the reaction solution for extraction, the separated organic phase was washed with water and then dried with anhydrous magnesium sulfate, and concentrated in vacuum to obtain a crude product. The crude product was purified by silica gel column chromatography and eluted with a mixture of dichloromethan and n-heptane system to obtain a white solid intermediate I-1 (15.4 g, yield 60%).

Intermediates I-2 to I-3 were synthesized by using a method similar to that described above and by replacing the intermediate c-1 with a reactant A in Table 11 below.

TABLE 11
Synthesis of intermediate I-2 to intermediate I-3

| Intermediate | | | Yield |
|---|---|---|---|
| No. | Reactant A | Structure | (%) |
| Intermediate I-2 | 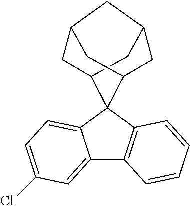 [SMILES: Clc1ccc2c(c1)-c1ccccc1C21C2CC3CC(C2)CC1C3] | 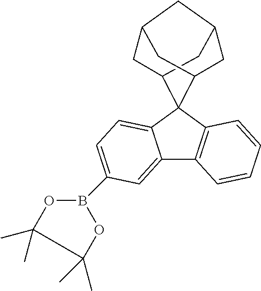 [SMILES: CC1(C)OB(c2ccc3c(c2)-c2ccccc2C32C3CC4CC(C3)CC2C4)OC1(C)C] | 64% |
| Intermediate I-3 | 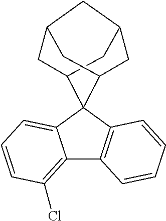 [SMILES: Clc1cccc2c1-c1ccccc1C21C2CC3CC(C2)CC1C3] | 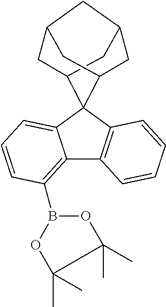 [SMILES: CC1(C)OB(c2cccc3c2-c2ccccc2C32C3CC4CC(C3)CC2C4)OC1(C)C] | 60% |

2. Synthesis of Intermediate J-1

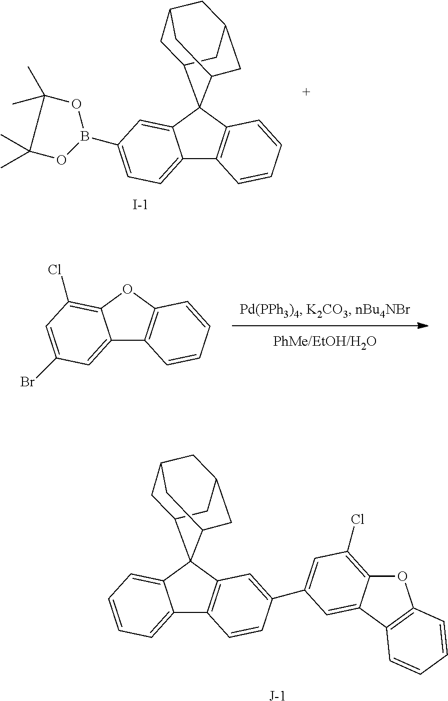 [SMILES: CC1(C)OB(c2ccc3c(c2)C2(c4ccccc4-3)C3CC4CC(C3)CC2C4)OC1(C)C ; [I-] ; Clc1cc(Br)cc2c1oc1ccccc12 ; Clc1cc(-c2ccc3c(c2)C2(c4ccccc4-3)C3CC4CC(C3)CC2C4)cc2c1oc1ccccc12 ; "J-1"]

The intermediate I-1 (15 g, 36.4 mmol), 2-bromo-4-chlorodibenzo[B,D]furan (8.5 g, 30.3 mmol), tetrakis(triphenylphosphine)palladium (0.7 g, 0.61 mmol), potassium carbonate (9.2 g, 66.7 mmol), tetrabutylammonium bromide (1.9 g, 6.1 mmol) were added into a flask, a mixed solvent of toluene (120 mL), ethanol (60 mL) and water (30 mL) was added in a nitrogen atmosphere, the mixture was heated to 80° C., and stirred for 12 hours while keeping the same temperature. The mixture was cooled to the room temperature, stirring was stopped, after a reaction solution was washed with water. The separated organic phase was dried with anhydrous magnesium sulfate, and concentrated in vacuum to obtain a crude product. The crude product was purified by silica gel column chromatography and eluted with a mixture of dichloromethane and n-heptane to obtain the intermediate J-1 (8.1 g, yield 55%) as a solid product.

Intermediates J-2 to J-11 shown in Table 12 were synthesized by using a method similar to the synthesis of the intermediate J-1, and by using a reactant A shown in Table 12 in place of the intermediate I-1 and a reactant B shown in Table 12 in place of 2-bromo-4-chlorodibenzo[B,D]furan.

TABLE 12
intermediates J-2 to J-11

| Intermediate | | |
|---|---|---|
| No. | Reactant A | Reactant B |
| J-2 | 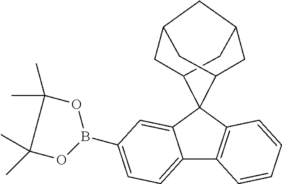 [SMILES: CC1(C)OB(c2ccc3c(c2)C2(c4ccccc4-3)C3CC4CC(C3)CC2C4)OC1(C)C] | 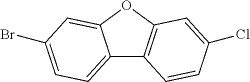 [SMILES: Clc1ccc2c(c1)oc1cc(Br)ccc12] |
| J-3 | 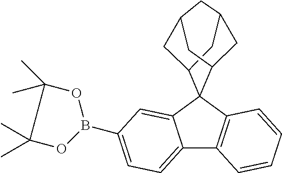 [SMILES: CC1(C)OB(c2ccc3c(c2)C2(c4ccccc4-3)C3CC4CC(C3)CC2C4)OC1(C)C] | 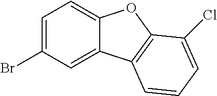 [SMILES: Clc1cccc2c1oc1ccc(Br)cc12] |
| J-4 | 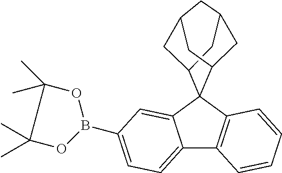 [SMILES: CC1(C)OB(c2ccc3c(c2)C2(c4ccccc4-3)C3CC4CC(C3)CC2C4)OC1(C)C] | 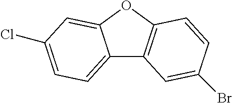 [SMILES: Clc1ccc2c(c1)oc1ccc(Br)cc12] |
| J-5 | 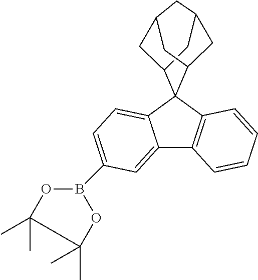 [SMILES: CC1(C)OB(c2ccc3c(c2)-c2ccccc2C32C3CC4CC(C3)CC2C4)OC1(C)C] | 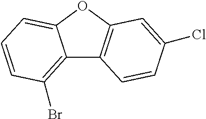 [SMILES: Clc1ccc2c(c1)oc1cccc(Br)c12] |
| J-6 | 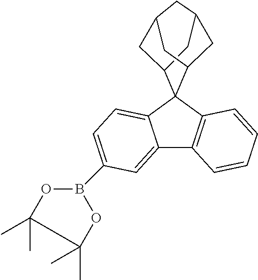 [SMILES: CC1(C)OB(c2ccc3c(c2)-c2ccccc2C32C3CC4CC(C3)CC2C4)OC1(C)C] | 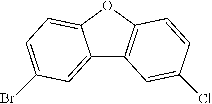 [SMILES: Clc1ccc2oc3ccc(Br)cc3c2c1] |
| J-7 | 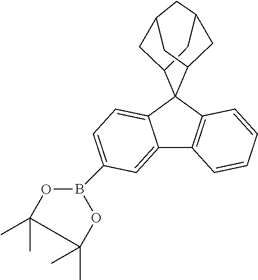 [SMILES: CC1(C)OB(c2ccc3c(c2)-c2ccccc2C32C3CC4CC(C3)CC2C4)OC1(C)C] | 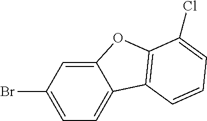 [SMILES: Clc1cccc2c1oc1cc(Br)ccc12] |
| J-8 | 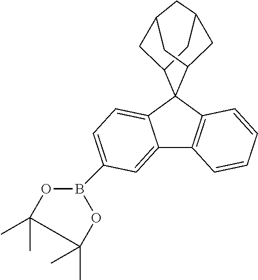 [SMILES: CC1(C)OB(c2ccc3c(c2)-c2ccccc2C32C3CC4CC(C3)CC2C4)OC1(C)C] | 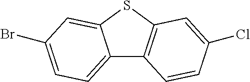 [SMILES: Clc1ccc2c(c1)sc1cc(Br)ccc12] |
| J-9 | 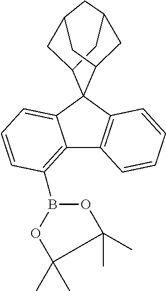 [SMILES: CC1(C)OB(c2cccc3c2-c2ccccc2C32C3CC4CC(C3)CC2C4)OC1(C)C] | 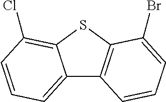 [SMILES: Clc1cccc2c1sc1c(Br)cccc12] |
| J-10 | 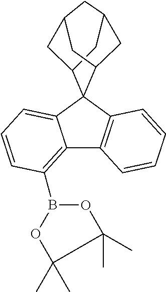 [SMILES: CC1(C)OB(c2cccc3c2-c2ccccc2C32C3CC4CC(C3)CC2C4)OC1(C)C] | 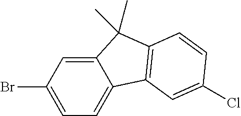 [SMILES: CC1(C)c2ccc(Cl)cc2-c2ccc(Br)cc21] |
| J-11 | 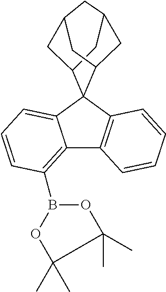 [SMILES: CC1(C)OB(c2cccc3c2-c2ccccc2C32C3CC4CC(C3)CC2C4)OC1(C)C] | 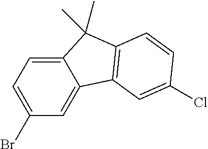 [SMILES: CC1(C)c2ccc(Cl)cc2-c2cc(Br)ccc21] |
| Intermediate | | |
| No. | Structure | Yield |
| J-2 | 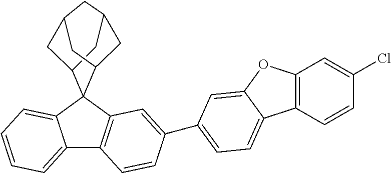 [SMILES: Clc1ccc2c(c1)oc1cc(-c3ccc4c(c3)C3(c5ccccc5-4)C4CC5CC(C4)CC3C5)ccc12] | 53% |
| J-3 | 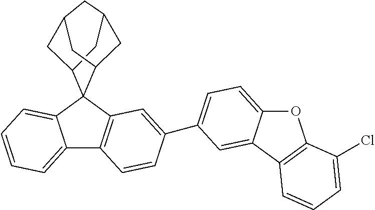 [SMILES: Clc1cccc2c1oc1ccc(-c3ccc4c(c3)C3(c5ccccc5-4)C4CC5CC(C4)CC3C5)cc12] | 55% |
| J-4 | 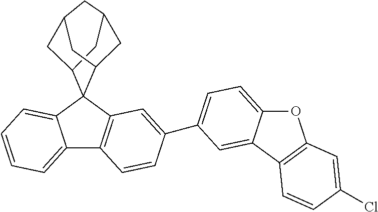 [SMILES: Clc1ccc2c(c1)oc1ccc(-c3ccc4c(c3)C3(c5ccccc5-4)C4CC5CC(C4)CC3C5)cc12] | 54% |
| J-5 | 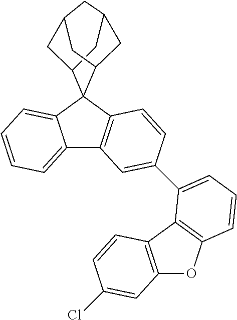 [SMILES: Clc1ccc2c(c1)oc1cccc(-c3ccc4c(c3)-c3ccccc3C43C4CC5CC(C4)CC3C5)c12] | 52% |
| J-6 | 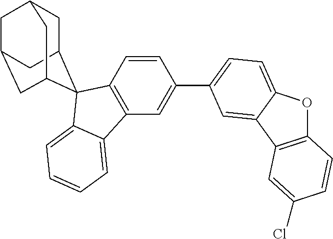 [SMILES: Clc1ccc2oc3ccc(-c4ccc5c(c4)-c4ccccc4C54C5CC6CC(C5)CC4C6)cc3c2c1] | 54% |
| J-7 | 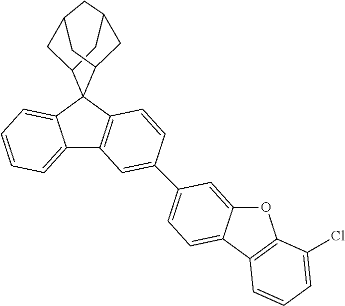 [SMILES: Clc1cccc2c1oc1cc(-c3ccc4c(c3)-c3ccccc3C43C4CC5CC(C4)CC3C5)ccc12] | 56% |
| J-8 | 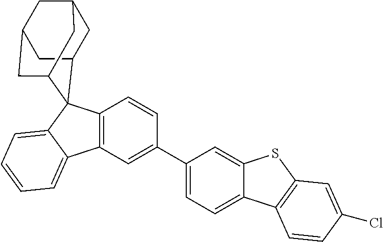 [SMILES: Clc1ccc2c(c1)sc1cc(-c3ccc4c(c3)-c3ccccc3C43C4CC5CC(C4)CC3C5)ccc12] | 52% |
| J-9 | 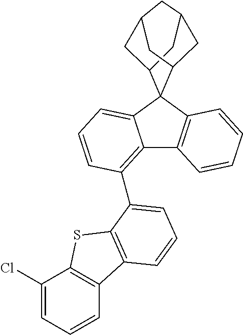 [SMILES: Clc1cccc2c1sc1c(-c3cccc4c3-c3ccccc3C43C4CC5CC(C4)CC3C5)cccc12] | 53% |
| J-10 | 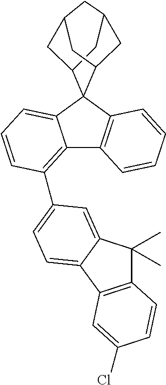 [SMILES: CC1(C)c2ccc(Cl)cc2-c2ccc(-c3cccc4c3-c3ccccc3C43C4CC5CC(C4)CC3C5)cc21] | 47% |
| J-11 | 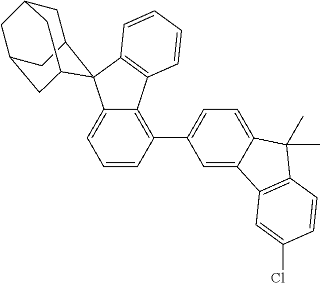 [SMILES: CC1(C)c2ccc(Cl)cc2-c2cc(-c3cccc4c3-c3ccccc3C43C4CC5CC(C4)CC3C5)ccc21] | 53% |

3. Synthesis of Intermediate k-1

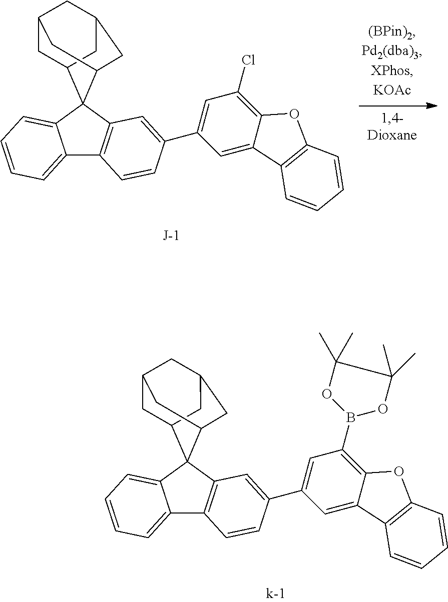 [SMILES: Clc1cc(-c2ccc3c(c2)C2(c4ccccc4-3)C3CC4CC(C3)CC2C4)cc2c1oc1ccccc12 ; CC1(C)OB(c2cc(-c3ccc4c(c3)C3(c5ccccc5-4)C4CC5CC(C4)CC3C5)cc3c2oc2ccccc23)OC1(C)C]

The intermediate J-1 (8.1 g, 16.6 mmol), bis(pinacolato)diboron (5.1 g, 19.9 mmol), tris(dibenzylideneacetone)dipalladium (0.15 g, 0.17 mmol), 2-(dicyclohexylphosphino)-2′,4′,6′-tri(isopropyl)biphenyl (0.15 g, 0.33 mmol), potassium acetate (3.6 g, 36.6 mmol) and 1,4-dioxane (64 mL) were added into a flask, and stirred under reflux at 100° C. for 18 hours in a nitrogen atmosphere. The mixture was cooled to room temperature, then dichloromethane and water were added into the solution, the solution was allowed to stand and stratify, an separated organic phase was washed with water and then dried with anhydrous magnesium sulfate, and concentrated in vacuum to obtain a crude product. The crude product was purified by silica gel column chromatography and eluted with a mixture of dichloromethane and n-heptane system to obtain a white solid intermediate k-1 (5.2 g, 54%).

Intermediates k-2 to k-11 were synthesized by using a method similar to that described above and by replacing the intermediate J-1 with a reactant A in Table 13 below.

TABLE 13
intermediates k-2 to k-11

| Intermediate | | | |
|---|---|---|---|
| No. | Reactant A | Structure | Yield |
| Intermediate k-2 | 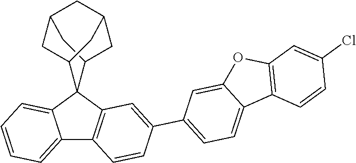 [SMILES: Clc1ccc2c(c1)oc1cc(-c3ccc4c(c3)C3(c5ccccc5-4)C4CC5CC(C4)CC3C5)ccc12] | 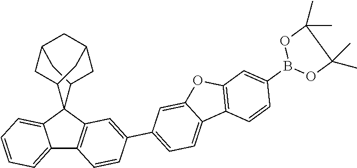 [SMILES: CC1(C)OB(c2ccc3c(c2)oc2cc(-c4ccc5c(c4)C4(c6ccccc6-5)C5CC6CC(C5)CC4C6)ccc23)OC1(C)C] | 55% |
| Intermediate k-3 | 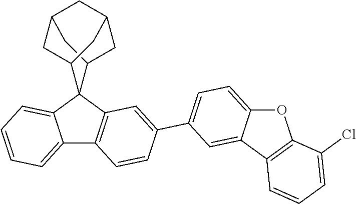 [SMILES: Clc1cccc2c1oc1ccc(-c3ccc4c(c3)C3(c5ccccc5-4)C4CC5CC(C4)CC3C5)cc12] | 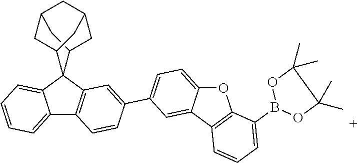 [SMILES: CC1(C)OB(c2cccc3c2oc2ccc(-c4ccc5c(c4)C4(c6ccccc6-5)C5CC6CC(C5)CC4C6)cc23)OC1(C)C] | 57% |
| Intermediate k-4 | 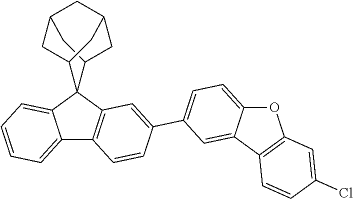 [SMILES: Clc1ccc2c(c1)oc1ccc(-c3ccc4c(c3)C3(c5ccccc5-4)C4CC5CC(C4)CC3C5)cc12] | 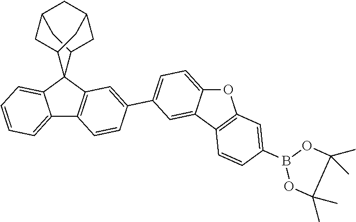 [SMILES: CC1(C)OB(c2ccc3c(c2)oc2ccc(-c4ccc5c(c4)C4(c6ccccc6-5)C5CC6CC(C5)CC4C6)cc23)OC1(C)C] | 56% |
| Intermediate k-5 | 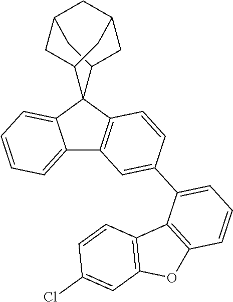 [SMILES: Clc1ccc2c(c1)oc1cccc(-c3ccc4c(c3)-c3ccccc3C43C4CC5CC(C4)CC3C5)c12] | 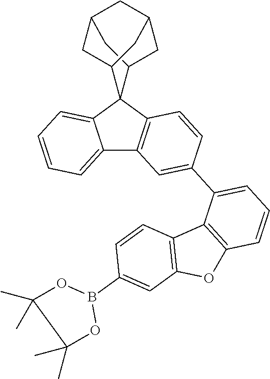 [SMILES: CC1(C)OB(c2ccc3c(c2)oc2cccc(-c4ccc5c(c4)-c4ccccc4C54C5CC6CC(C5)CC4C6)c23)OC1(C)C] | 54% |
| Intermediate k-6 | 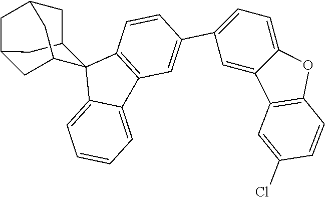 [SMILES: Clc1ccc2oc3ccc(-c4ccc5c(c4)-c4ccccc4C54C5CC6CC(C5)CC4C6)cc3c2c1] | 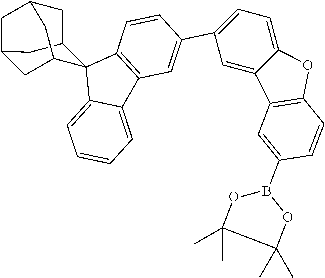 [SMILES: CC1(C)OB(c2ccc3oc4ccc(-c5ccc6c(c5)-c5ccccc5C65C6CC7CC(C6)CC5C7)cc4c3c2)OC1(C)C] | 56% |
| Intermediate k-7 | 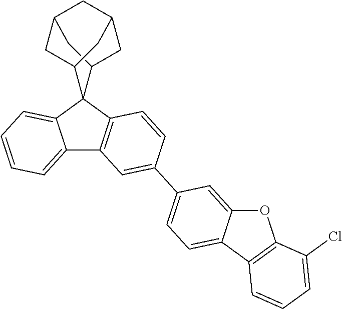 [SMILES: Clc1cccc2c1oc1cc(-c3ccc4c(c3)-c3ccccc3C43C4CC5CC(C4)CC3C5)ccc12] | 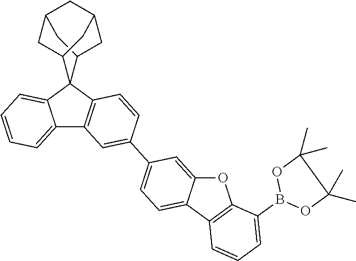 [SMILES: CC1(C)OB(c2cccc3c2oc2cc(-c4ccc5c(c4)-c4ccccc4C54C5CC6CC(C5)CC4C6)ccc23)OC1(C)C] | 58% |
| Intermediate k-8 | 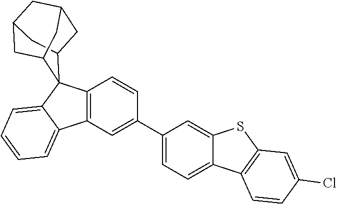 [SMILES: Clc1ccc2c(c1)sc1cc(-c3ccc4c(c3)-c3ccccc3C43C4CC5CC(C4)CC3C5)ccc12] | 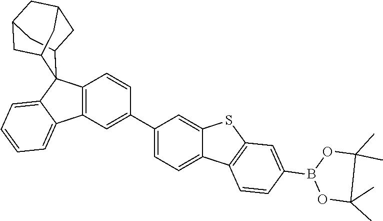 [SMILES: CC1(C)OB(c2ccc3c(c2)sc2cc(-c4ccc5c(c4)-c4ccccc4C54C5CC6CC(C5)CC4C6)ccc23)OC1(C)C] | 54% |
| Intermediate k-9 | 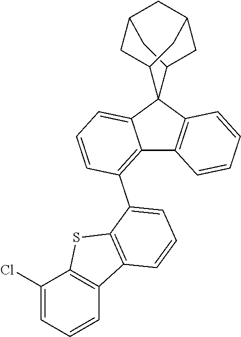 [SMILES: Clc1cccc2c1sc1c(-c3cccc4c3-c3ccccc3C43C4CC5CC(C4)CC3C5)cccc12] | 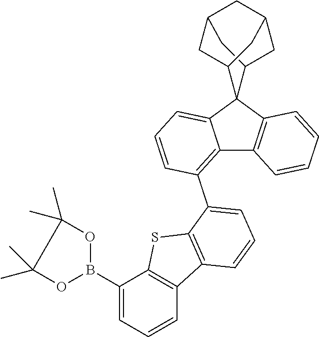 [SMILES: CC1(C)OB(c2cccc3c2sc2c(-c4cccc5c4-c4ccccc4C54C5CC6CC(C5)CC4C6)cccc23)OC1(C)C] | 55% |
| Intermediate k-10 | 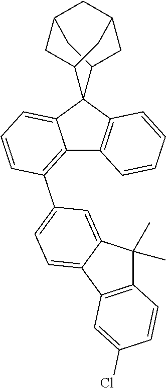 [SMILES: CC1(C)c2ccc(Cl)cc2-c2ccc(-c3cccc4c3-c3ccccc3C43C4CC5CC(C4)CC3C5)cc21] | 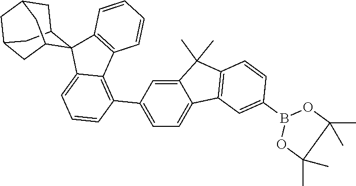 [SMILES: CC1(C)c2ccc(B3OC(C)(C)C(C)(C)O3)cc2-c2ccc(-c3cccc4c3-c3ccccc3C43C4CC5CC(C4)CC3C5)cc21] | 49% |
| Intermediate k-11 | 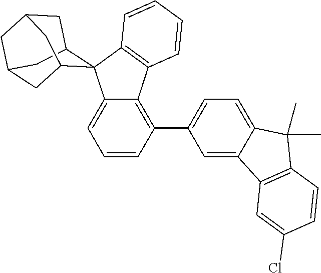 [SMILES: CC1(C)c2ccc(Cl)cc2-c2cc(-c3cccc4c3-c3ccccc3C43C4CC5CC(C4)CC3C5)ccc21] | 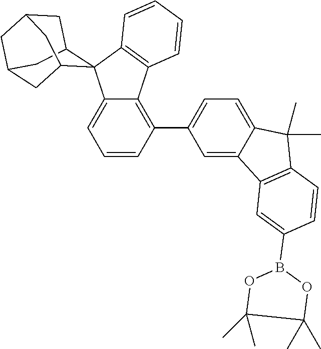 [SMILES: CC1(C)c2ccc(B3OC(C)(C)C(C)(C)O3)cc2-c2cc(-c3cccc4c3-c3ccccc3C43C4CC5CC(C4)CC3C5)ccc21] | 55% |

4. Synthesis of Preparation Example 24 (Compound 29)

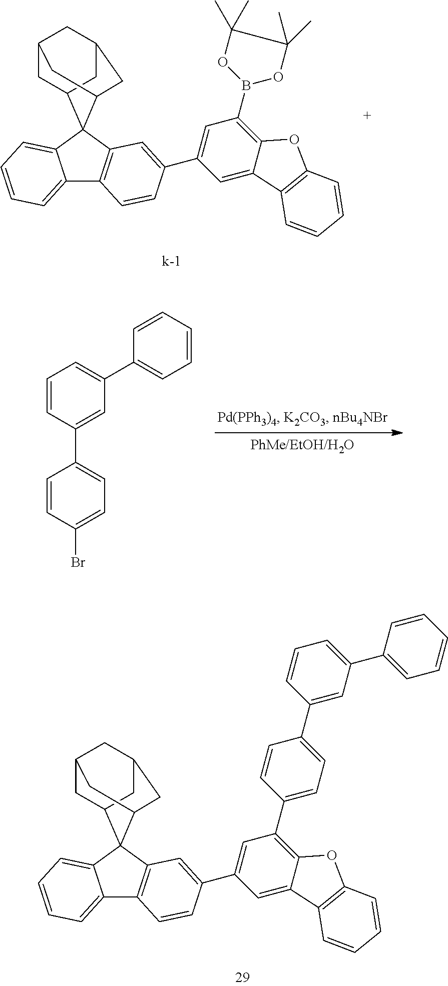 [SMILES: CC1(C)OB(c2cc(-c3ccc4c(c3)C3(c5ccccc5-4)C4CC5CC(C4)CC3C5)cc3c2oc2ccccc23)OC1(C)C ; Brc1ccc(-c2cccc(-c3ccccc3)c2)cc1 ; c1ccc(-c2cccc(-c3ccc(-c4cc(-c5ccc6c(c5)C5(c7ccccc7-6)C6CC7CC(C6)CC5C7)cc5c4oc4ccccc45)cc3)c2)cc1]

The intermediate k-1 (5.2 g, 9.0 mmol), 4-bromo-1,1′,3′,1″-terphenyl (2.3 g, 7.5 mmol), tetrakis(triphenylphosphine)palladium (0.17 g, 0.15 mmol), potassium carbonate (2.3 g, 16.5 mmol), tetrabutylammonium bromide (0.5 g, 1.5 mmol) were added into a flask, a mixed solvent of toluene (40 mL), ethanol (20 mL) and water (10 mL) was added in a nitrogen atmosphere. The mixture was heated to 80° C., and stirred for 12 hours while keeping the same temperature. The mixture was cooled to the room temperature, stirring was stopped, after a reaction solution was washed with water, an organic phase was separated. The separated organic phase was dried with anhydrous magnesium sulfate, and concentrated in vacuum to obtain a crude product. The crude product was purified by silica gel column chromatography and eluted with a mixture of dichloromethane and n-heptane to obtain the compound 29 (3.0 g, yield 58%) as a solid product.

Preparation examples 25 to 34 shown in Table 14 were synthesized by using a method similar to the synthesis of preparation example 24, and by using a reactant A shown in Table 14 in place of the intermediate k-1 and a reactant B shown in Table 14 in place of 4-bromo-1,1′,3′,1″-terphenyl.

TABLE 14
Preparation examples 25 to 34

| Prep- | | | | | |
|---|---|---|---|---|---|
| aration | | | | | |
| ex- | Com- | | | | |
| ample | pound | | | | |
| No. | No. | Reactant A | Reactant B | Structure | Yield |
| 25 | 160 | 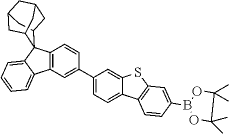 [SMILES: CC1(C)OB(c2ccc3c(c2)sc2cc(-c4ccc5c(c4)-c4ccccc4C54C5CC6CC(C5)CC4C6)ccc23)OC1(C)C] | 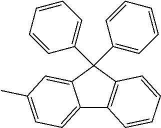 [SMILES: Cc1ccc2c(c1)C(c1ccccc1)(c1ccccc1)c1ccccc1-2] | 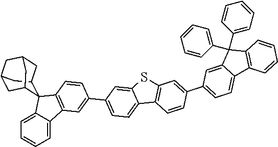 [SMILES: c1ccc(C2(c3ccccc3)c3ccccc3-c3ccc(-c4ccc5c(c4)sc4cc(-c6ccc7c(c6)-c6ccccc6C76C7CC8CC(C7)CC6C8)ccc45)cc32)cc1] | 52% |
| 26 | 311 | 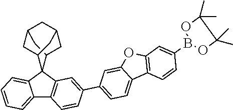 [SMILES: CC1(C)OB(c2ccc3c(c2)oc2cc(-c4ccc5c(c4)C4(c6ccccc6-5)C5CC6CC(C5)CC4C6)ccc23)OC1(C)C] | 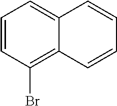 [SMILES: Brc1cccc2ccccc12] | 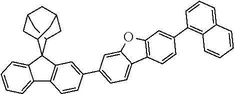 [SMILES: c1ccc2c(c1)-c1ccc(-c3ccc4c(c3)oc3cc(-c5cccc6ccccc56)ccc34)cc1C21C2CC3CC(C2)CC1C3] | 53% |
| 27 | 312 | 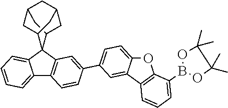 [SMILES: CC1(C)OB(c2cccc3c2oc2ccc(-c4ccc5c(c4)C4(c6ccccc6-5)C5CC6CC(C5)CC4C6)cc23)OC1(C)C] | 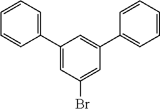 [SMILES: Brc1cc(-c2ccccc2)cc(-c2ccccc2)c1] | 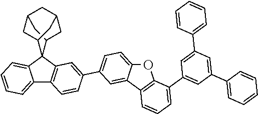 [SMILES: c1ccc(-c2cc(-c3ccccc3)cc(-c3cccc4c3oc3ccc(-c5ccc6c(c5)C5(c7ccccc7-6)C6CC7CC(C6)CC5C7)cc34)c2)cc1] | 56% |
| 28 | 313 | 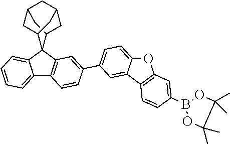 [SMILES: CC1(C)OB(c2ccc3c(c2)oc2ccc(-c4ccc5c(c4)C4(c6ccccc6-5)C5CC6CC(C5)CC4C6)cc23)OC1(C)C] | 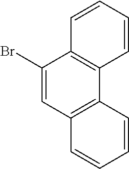 [SMILES: Brc1cc2ccccc2c2ccccc12] | 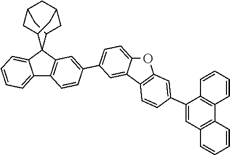 [SMILES: c1ccc2c(c1)-c1ccc(-c3ccc4oc5cc(-c6cc7ccccc7c7ccccc67)ccc5c4c3)cc1C21C2CC3CC(C2)CC1C3] | 51% |
| 29 | 314 | 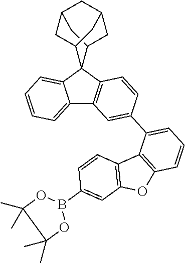 [SMILES: CC1(C)OB(c2ccc3c(c2)oc2cccc(-c4ccc5c(c4)-c4ccccc4C54C5CC6CC(C5)CC4C6)c23)OC1(C)C] | 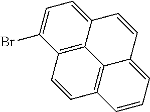 [SMILES: Brc1ccc2ccc3cccc4ccc1c2c34] | 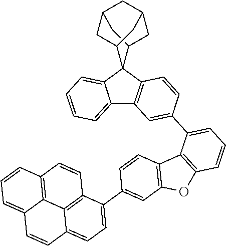 [SMILES: c1ccc2c(c1)-c1cc(-c3cccc4oc5cc(-c6ccc7ccc8cccc9ccc6c7c89)ccc5c34)ccc1C21C2CC3CC(C2)CC1C3] | 53% |
| 30 | 315 | 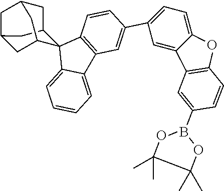 [SMILES: CC1(C)OB(c2ccc3oc4ccc(-c5ccc6c(c5)-c5ccccc5C65C6CC7CC(C6)CC5C7)cc4c3c2)OC1(C)C] | 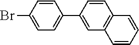 [SMILES: Brc1ccc(-c2ccc3ccccc3c2)cc1] | 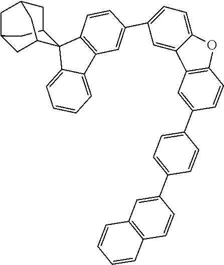 [SMILES: c1ccc2c(c1)-c1cc(-c3ccc4oc5ccc(-c6ccc(-c7ccc8ccccc8c7)cc6)cc5c4c3)ccc1C21C2CC3CC(C2)CC1C3] | 55% |
| 31 | 316 | 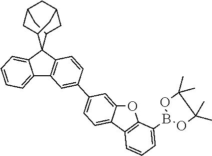 [SMILES: CC1(C)OB(c2cccc3c2oc2cc(-c4ccc5c(c4)-c4ccccc4C54C5CC6CC(C5)CC4C6)ccc23)OC1(C)C] | 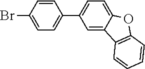 [SMILES: Brc1ccc(-c2ccc3oc4ccccc4c3c2)cc1] | 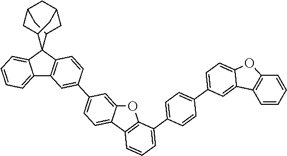 [SMILES: c1ccc2c(c1)-c1cc(-c3ccc4c(c3)oc3c(-c5ccc(-c6ccc7oc8ccccc8c7c6)cc5)cccc34)ccc1C21C2CC3CC(C2)CC1C3] | 56% |
| 32 | 317 | 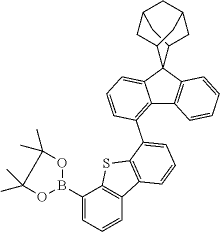 [SMILES: CC1(C)OB(c2cccc3c2sc2c(-c4cccc5c4-c4ccccc4C54C5CC6CC(C5)CC4C6)cccc23)OC1(C)C] | 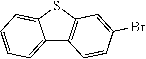 [SMILES: Brc1ccc2c(c1)sc1ccccc12] | 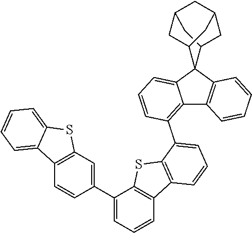 [SMILES: c1ccc2c(c1)-c1c(-c3cccc4c3sc3c(-c5ccc6c(c5)sc5ccccc56)cccc34)cccc1C21C2CC3CC(C2)CC1C3] | 53% |
| 33 | 318 | 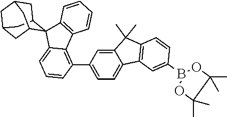 [SMILES: CC1(C)c2ccc(B3OC(C)(C)C(C)(C)O3)cc2-c2ccc(-c3cccc4c3-c3ccccc3C43C4CC5CC(C4)CC3C5)cc21] | 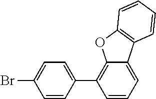 [SMILES: Brc1ccc(-c2cccc3c2oc2ccccc23)cc1] | 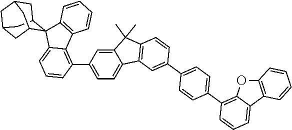 [SMILES: CC1(C)c2ccc(-c3ccc(-c4cccc5c4oc4ccccc45)cc3)cc2-c2ccc(-c3cccc4c3-c3ccccc3C43C4CC5CC(C4)CC3C5)cc21] | 54% |
| 34 | 319 | 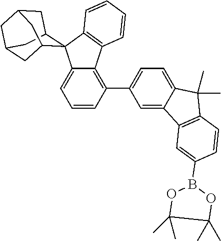 [SMILES: CC1(C)c2ccc(B3OC(C)(C)C(C)(C)O3)cc2-c2cc(-c3cccc4c3-c3ccccc3C43C4CC5CC(C4)CC3C5)ccc21] | 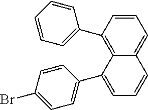 [SMILES: Brc1ccc(-c2cccc3cccc(-c4ccccc4)c23)cc1] | 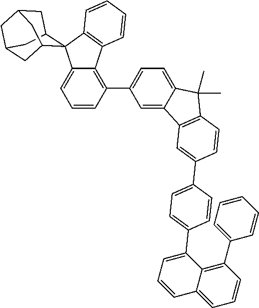 [SMILES: CC1(C)c2ccc(-c3ccc(-c4cccc5cccc(-c6ccccc6)c45)cc3)cc2-c2cc(-c3cccc4c3-c3ccccc3C43C4CC5CC(C4)CC3C5)ccc21] | 52% |

The above compounds were analyzed by mass spectrometry, and data are shown in Table 15 below.

TABLE 15
Mass spectrometry characterization data

| | LC-MS(ESI, pos.ion) | | LC-MS(ESI, pos.ion) |
|---|---|---|---|
| Compound# | m/z: [M + H]⁺ | Compound# | m/z: [M + H]⁺ |
| Compound 16 | m/z = 684.3[M + H]⁺ | Compound 154 | m/z = 800.3[M + H]⁺ |
| Compound 23 | m/z = 760.3[M + H]⁺ | Compound 107 | m/z = 629.3[M + H]⁺ |
| Compound 141 | m/z = 776.3[M + H]⁺ | Compound 93 | m/z = 800.4[M + H]⁺ |
| Compound 238 | m/z = 754.3[M + H]⁺ | Compound 185 | m/z = 673.3[M + H]⁺ |
| Compound 259 | m/z = 786.4[M + H]⁺ | Compound 192 | m/z = 621.3[M + H]⁺ |
| Compound 234 | m/z = 723.3[M + H]⁺ | Compound 29 | m/z = 681.3[M + H]⁺ |
| Compound 268 | m/z = 800.4[M + H]⁺ | Compound 143 | m/z = 700.3[M + H]⁺ |
| Compound 142 | m/z = 749.3[M + H]⁺ | Compound 258 | m/z = 681.3[M + H]⁺ |
| Compound 10 | m/z = 789.3[M + H]⁺ | Compound 62 | m/z = 605.3[M + H]⁺ |
| Compound 239 | m/z = 802.3[M + H]⁺ | Compound 51 | m/z = 836.4[M + H]⁺ |
| Compound 25 | m/z = 836.4[M + H]⁺ | Compound 147 | m/z = 595.2[M + H]⁺ |
| Compound 160 | m/z = 785.3[M + H]⁺ | Compound 128 | m/z = 723.3[M + H]⁺ |
| Compound 280 | m/z = 824.3[M + H]⁺ | Compound 311 | m/z = 579.3[M + H]⁺ |
| Compound 312 | m/z = 681.3[M + H]⁺ | Compound 313 | m/z = 629.3[M + H]⁺ |
| Compound 314 | m/z = 653.3[M + H]⁺ | Compound 315 | m/z = 655.3[M + H]⁺ |
| Compound 316 | m/z = 695.3[M + H]⁺ | Compound 317 | m/z = 651.2[M + H]⁺ |
| Compound 318 | m/z = 721.3[M + H]⁺ | Compound 319 | m/z = 757.4[M + H]⁺ |

Nuclear magnetic data of some compounds of the above examples:

TABLE 16

| Compound # | Compound structure | ¹HNMR (CD₂Cl₂, 400 MHz), δ(ppm): |
|---|---|---|
| Compound 16 | 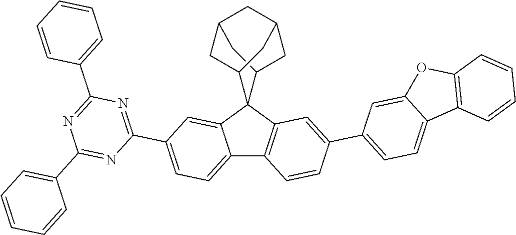 [SMILES: c1ccc(-c2nc(-c3ccccc3)nc(-c3ccc4c(c3)C3(c5cc(-c6ccc7c(c6)oc6ccccc67)ccc5-4)C4CC5CC(C4)CC3C5)n2)cc1] | ¹HNMR: 8.79 (d, 4H), 8.20 (s, 1H), 8.06 (d, 1H), 8.00 (d, 1H), 7.96-7.94 (m, 2H), 7.75 (d, 1H), 7.67-7.56 (m, 9H), 7.51 (d, 1H), 7.40-7.31 (m, 3H), 2.83 (d, 2H), 2.74 (d, 2H), 2.18 (s, 1H), 2.12 (s, 1H), 1.94 (s, 2H), 1.73 (t, 4H), 1.41 (s, 2H). |
| Compound 62 | 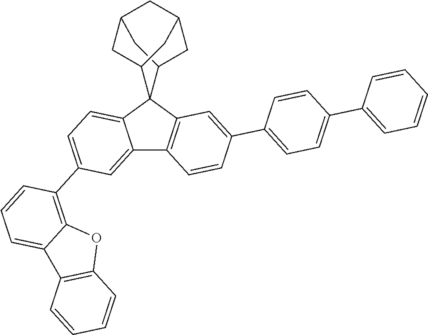 [SMILES: c1ccc(-c2ccc(-c3ccc4c(c3)C3(c5ccc(-c6cccc7c6oc6ccccc67)cc5-4)C4CC5CC(C4)CC3C5)cc2)cc1] | ¹HNMR (CD₂Cl₂, 400 MHz), δ(ppm): 8.16 (s, 1H), 8.00 (d, 1H), 7.95 (d, 1H), 7.81 (s, 1H), 7.70 (d, 1H), 7.67-7.64(m, 3H), 7.61-7.51 (m, 9H), 7.43-7.31 (m, 4H), 7.26 (d, 1H), 2.85 (d, 2H), 2.77 (d, 2H), 2.22 (s, 1H), 2.12 (s, 1H), 1.98 (s, 2H), 1.74 (t, 4H), 1.42 (s, 2H). |
| Compound 29 | 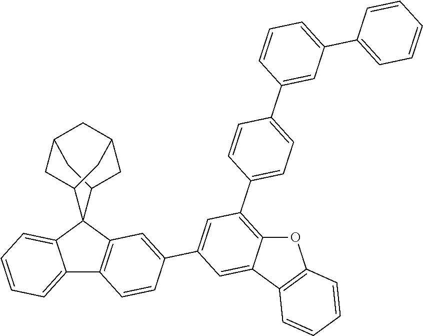 [SMILES: c1ccc(-c2cccc(-c3ccc(-c4cc(-c5ccc6c(c5)C5(c7ccccc7-6)C6CC7CC(C6)CC5C7)cc5c4oc4ccccc45)cc3)c2)cc1] | ¹HNMR (CD₂Cl₂, 400 MHz), δ(ppm): 8.33 (s, 1H), 8.05 (s, 2H), 7.95 (d, 1H), 7.85 (d, 1H), 7.69 (t, 3H), 7.62-7.59(m, 3H), 7.55-7.47 (m, 7H), 7.45-7.31 (m, 7H), 7.21 (t, 1H), 2.81 (d, 2H), 2.76 (d, 2H), 2.20 (s, 1H), 2.10 (s, 1H), 1.96 (s, 2H), 1.75 (t, 4H), 1.39 (s, 2H). |
| Compound 23 | 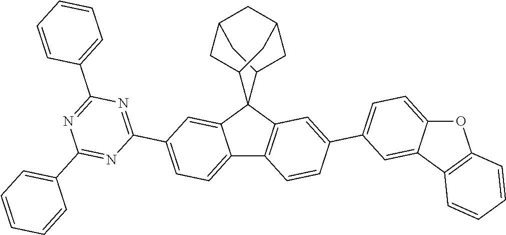 [SMILES: c1ccc(-c2nc(-c3ccccc3)nc(-c3ccc4c(c3)C3(c5cc(-c6ccc7oc8ccccc8c7c6)ccc5-4)C4CC5CC(C4)CC3C5)n2)cc1] | ¹HNMR (CD₂Cl₂, 400 MHz), δ(ppm): 8.79 (d, 4H), 8.17 (d, 2H), 7.96 (d, 1H), 7.87 (s, 1H), 7.82 (d, 2H), 7.77 (s, 1H), 7.68-7.62(m, 7H), 7.60-7.50 (m, 5H), 7.42-7.31 (m, 4H), 2.81 (d, 2H), 2.75 (d, 2H), 2.24 (s, 1H), 2.15 (s, 1H), 1.96 (s, 2H), 1.72 (t, 4H), 1.40 (s, 2H). |
| Compound 311 | 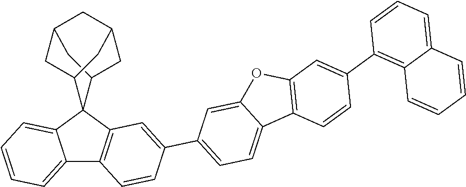 [SMILES: c1ccc2c(c1)-c1ccc(-c3ccc4c(c3)oc3cc(-c5cccc6ccccc56)ccc34)cc1C21C2CC3CC(C2)CC1C3] | ¹HNMR (CD₂Cl₂, 400 MHz), δ(ppm): 8.17 (d,1H), 8.06 (d, 1H), 7.98 (d, 2H), 7.91 (d, 1H), 7.84 (t, 2H), 7.78-7.68(m, 4H), 7.59-7.55 (m, 2H), 7.47 (t, 1H), 7.42-7.33(m, 5H), 7.21 (t, 1H), 2.83 (d, 2H), 2.72 (d, 2H), 2.20 (s, 1H), 2.14 (s, 1H), 1.95 (s, 2H), 1.69 (t, 4H), 1.39 (s, 2H). |

Preparation and Performance Evaluation of Organic Electroluminescence Device

### EXAMPLE 1

### Green Organic Electroluminescence Device

The green organic electroluminescence device is manufactured by using the following methods:

An anode is prepared through the following processes: an ITO substrate with an ITO thickness of 1500 Å is cut into a size of 40 mm (length)×40 mm (width)×0.7 mm (thickness) to be prepared into an experimental substrate with pattern of a cathode, an anode and an insulating layer by adopting a photoetching process, and surface treatment is performed by utilizing ultraviolet ozone and O₂:N₂ plasma to increase a work function of the anode. A surface of the ITO substrate may be cleaned with an organic solvent to remove impurities and oil stains on the surface of the ITO substrate. It should be noted that the ITO substrate may further be cut into other sizes according to actual needs, and the size of the ITO substrate in the present disclosure is not particularly limited.

F4-TCNQ is vacuum-evaporated on the experiment substrate (the anode) to form a hole injection layer (HIL) with a thickness of 100 Å, and HT-01 is evaporated on the hole injection layer to form a first hole transport layer with a thickness of 850 Å.

HT-02 is vacuum-evaporated on the first hole transport layer to form a second hole transport layer with a thickness of 330 Å.

On the second hole transport layer, the compound 62, GH-n1 and Ir(ppy)₃ are co-evaporated at a ratio of 52%:43%:5% (an evaporation rate) to form a green luminescent layer (EML) with a thickness of 500 Å.

ET-01 and LiQ are mixed at a weight ratio of 1:1 and a mixture is evaporated to form an electron transport layer (ETL) with a thickness of 300 Å, LiQ is evaporated on the electron transport layer to form an electron injection layer (EIL) with a thickness of 10 Å, and then magnesium (Mg) and silver (Ag) are mixed at an evaporation rate of 1:9 and a mixture is vacuum-evaporated on the electron injection layer to form a cathode with a thickness of 110 Å.

In addition, CP-01 with a thickness of 650 Å is evaporated on the above cathode to form an organic capping layer (CPL), thus completing preparation of the whole organic light-emitting device.

### EXAMPLE 2 TO EXAMPLE 26

Except for replacing mixture components in example 1 with mixture components shown in Table 17 below at the time of forming the luminescent layer, the organic electroluminescence device is manufactured by adopting the same method as in example 1.

### COMPARATIVE EXAMPLE 1 TO COMPARATIVE EXAMPLE 5

Except for replacing mixture components in example 1 with mixture components shown in Table 17 below at the time of forming the luminescent layer, the organic electroluminescence device is prepared by adopting the same method as in example 1.

Material structures used in examples 1 to 26 and comparative examples 1 to 5 are shown as follows.

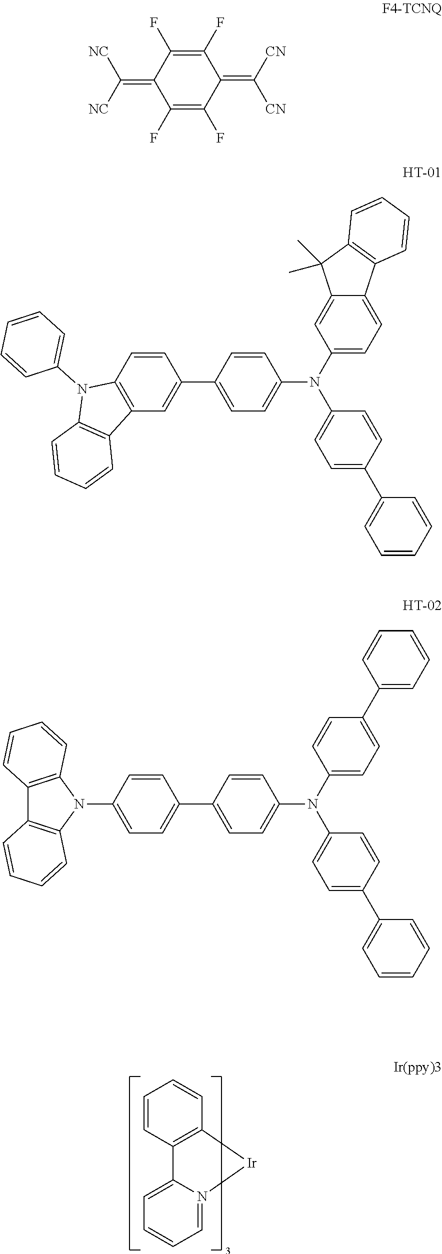

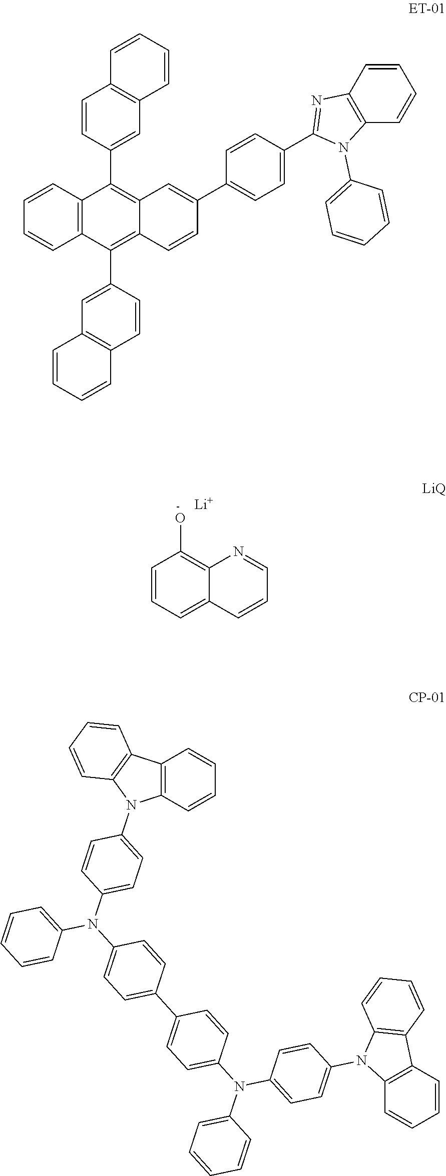 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; [Li+] ; Oc1cccc2cccnc12 ; c1ccc(N(c2ccc(-c3ccc(N(c4ccccc4)c4ccc(-n5c6ccccc6c6ccccc65)cc4)cc3)cc2)c2ccc(-n3c4ccccc4c4ccccc43)cc2)cc1]

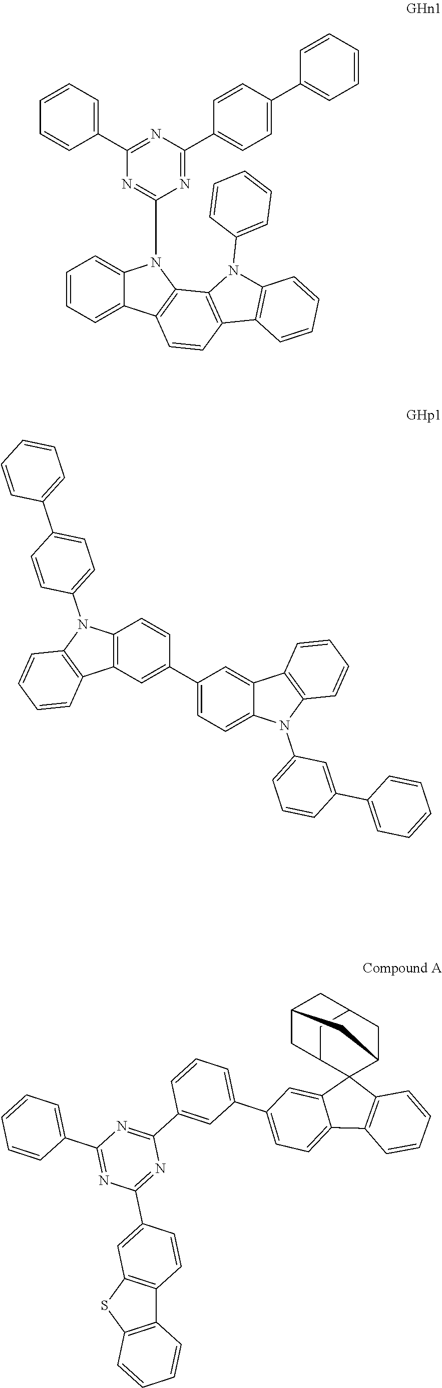 [SMILES: c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2nc(-c3cccc(-c4ccc5c(c4)C4(c6ccccc6-5)C5CC6C[C@H](C5)C[C@H]4C6)c3)nc(-c3ccc4c(c3)sc3ccccc34)n2)cc1]

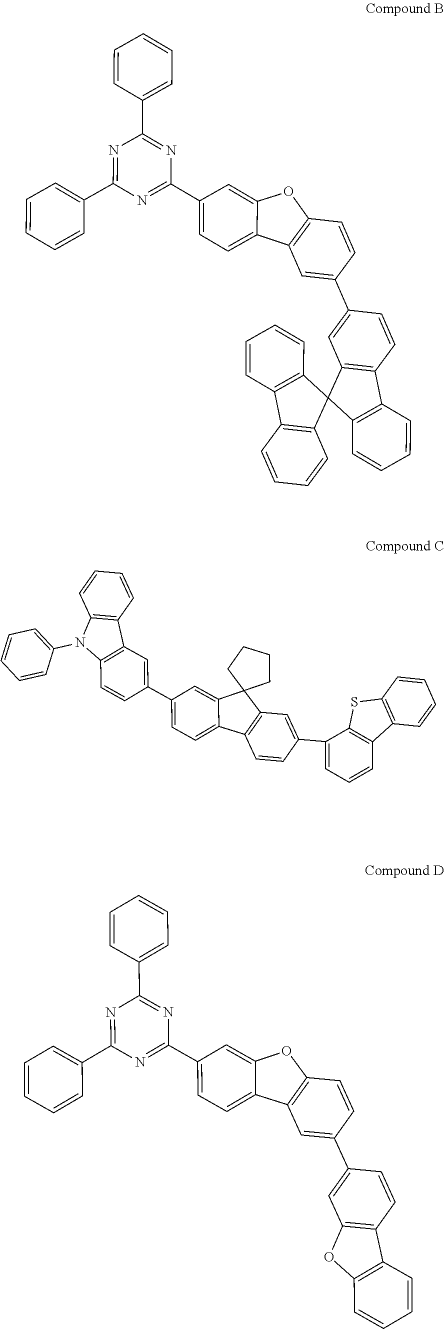 [SMILES: c1ccc(-c2nc(-c3ccccc3)nc(-c3ccc4c(c3)oc3ccc(-c5ccc6c(c5)C5(c7ccccc7-c7ccccc75)c5ccccc5-6)cc34)n2)cc1 ; c1ccc(-n2c3ccccc3c3cc(-c4ccc5c(c4)C4(CCCC4)c4cc(-c6cccc7c6sc6ccccc67)ccc4-5)ccc32)cc1 ; c1ccc(-c2nc(-c3ccccc3)nc(-c3ccc4c(c3)oc3ccc(-c5ccc6c(c5)oc5ccccc56)cc34)n2)cc1]

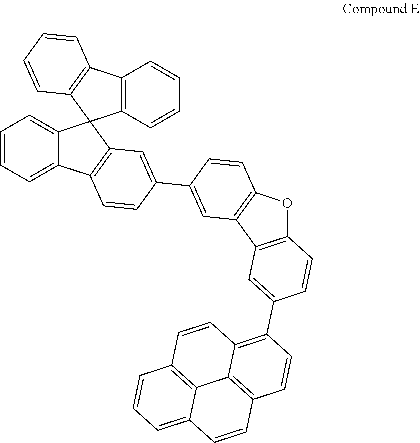 [SMILES: c1ccc2c(c1)-c1ccccc1C21c2ccccc2-c2ccc(-c3ccc4oc5ccc(-c6ccc7ccc8cccc9ccc6c7c89)cc5c4c3)cc21]

The organic electroluminescence devices prepared in examples 1 to 26 and comparative examples 1 to 5 are tested for performance under a condition of 20 mA/cm², and test results are shown in Table 17 below.

TABLE 17
performance test results of organic electroluminescence device

| | | | | | | | External | |
|---|---|---|---|---|---|---|---|---|
| | luminescent | | | | | | quantum | T95 |
| | layer: | Driving | Current | Power | Chromaticity | Chromaticity | efficiency | service |
| | 3 materials = | voltage | efficiency | efficiency | coordinate | coordinate | EQE | life |
| Example No. | 50%:45%:5% | (V) | (Cd/A) | (lm/W) | CIEx | CIEy | (%) | (h) |
| Example 1 | Compound | 3.72 | 80.5 | 67.9 | 0.22 | 0.73 | 19.3 | 219 |
| | 62:GHn1:Ir(ppy)₃ | | | | | | | |
| Example 2 | Compound | 3.75 | 81.3 | 68.1 | 0.22 | 0.73 | 19.5 | 239 |
| | 107:GHn1:Ir(ppy)₃ | | | | | | | |
| Example 3 | Compound | 3.73 | 83.8 | 70.5 | 0.22 | 0.73 | 20.1 | 244 |
| | 147:GHn1:Ir(ppy)₃ | | | | | | | |
| Example 4 | Compound | 3.74 | 83.2 | 69.9 | 0.22 | 0.73 | 20.0 | 251 |
| | 234:GHn1:Ir(ppy)₃ | | | | | | | |
| Example 5 | GHp1:compound | 3.78 | 84.2 | 69.9 | 0.22 | 0.73 | 20.2 | 246 |
| | 10:Ir(ppy)₃ | | | | | | | |
| Example 6 | GHp1:compound | 3.72 | 87.3 | 73.7 | 0.22 | 0.73 | 21.0 | 252 |
| | 16:Ir(ppy)₃ | | | | | | | |
| Example 7 | GHp1:compound | 3.71 | 86.8 | 73.5 | 0.22 | 0.73 | 20.8 | 253 |
| | 51:Ir(ppy)₃ | | | | | | | |
| Example 8 | GHp1:compound | 3.64 | 89.8 | 77.5 | 0.22 | 0.73 | 21.6 | 260 |
| | 141:Ir(ppy)₃ | | | | | | | |
| Example 9 | GHp1:compound | 3.63 | 88.3 | 76.4 | 0.22 | 0.73 | 21.2 | 257 |
| | 142:Ir(ppy)₃ | | | | | | | |
| Example 10 | GHp1:compound | 3.68 | 89.1 | 76.0 | 0.22 | 0.73 | 21.4 | 261 |
| | 154:Ir(ppy)₃ | | | | | | | |
| Example 11 | GHp1:compound | 3.72 | 84.5 | 71.3 | 0.22 | 0.73 | 20.3 | 254 |
| | 238:Ir(ppy)₃ | | | | | | | |
| Example 12 | GHp1:compound | 3.67 | 87.5 | 74.9 | 0.22 | 0.73 | 21.0 | 251 |
| | 268:Ir(ppy)₃ | | | | | | | |
| Example 13 | Compound | 3.65 | 80.8 | 69.5 | 0.22 | 0.73 | 19.3 | 242 |
| | 192:GHn1:Ir(ppy)₃ | | | | | | | |
| Example 14 | Compound | 3.67 | 82.2 | 70.3 | 0.23 | 0.73 | 20.0 | 252 |
| | 258:GHn1:Ir(ppy)₃ | | | | | | | |
| Example 15 | GHp1:compound | 3.68 | 83.2 | 80.0 | 0.22 | 0.73 | 19.9 | 244 |
| | 280:Ir(ppy)₃ | | | | | | | |
| Example 16 | Compound 160 | 3.72 | 81.5 | 68.3 | 0.22 | 0.73 | 19.5 | 251 |
| | GHn1:Ir(ppy)₃ | | | | | | | |
| Example 17 | Compound 311 | 3.73 | 84.8 | 70.4 | 0.22 | 0.73 | 20.5 | 246 |
| | GHn1:Ir(ppy)₃ | | | | | | | |
| Example 18 | Compound 312 | 3.65 | 83.3 | 69.9 | 0.22 | 0.73 | 19.6 | 257 |
| | GHn1:Ir(ppy)₃ | | | | | | | |
| Example 19 | Compound 313 | 3.66 | 83.1 | 70.5 | 0.22 | 0.73 | 19.6 | 260 |
| | GHn1:Ir(ppy)₃ | | | | | | | |
| Example 20 | Compound 314 | 3.67 | 82.9 | 69.7 | 0.22 | 0.73 | 19.5 | 257 |
| | GHn1:Ir(ppy)₃ | | | | | | | |
| Example 21 | Compound 315 | 3.68 | 83.8 | 72.5 | 0.22 | 0.73 | 19.4 | 223 |
| | GHn1:Ir(ppy)₃ | | | | | | | |
| Example 22 | Compound 316 | 3.65 | 83.1 | 72.3 | 0.22 | 0.73 | 19.5 | 250 |
| | GHn1:Ir(ppy)₃ | | | | | | | |
| Example 23 | Compound 317 | 3.64 | 82.1 | 71.6 | 0.22 | 0.73 | 19.4 | 262 |
| | GHn1:Ir(ppy)₃ | | | | | | | |
| Example 24 | Compound 318 | 3.68 | 83.1 | 73.0 | 0.22 | 0.73 | 19.6 | 255 |
| | GHn1:Ir(ppy)₃ | | | | | | | |
| Example 25 | Compound | 3.70 | 85.1 | 75.3 | 0.22 | 0.73 | 20.1 | 233 |
| | 319:GHn1:Ir(ppy)₃ | | | | | | | |
| Example 26 | Compound | 3.71 | 82.5 | 72.4 | 0.22 | 0.73 | 19.6 | 248 |
| | 29:GHn1:Ir(ppy)₃ | | | | | | | |
| Comparative | GHp1:compound | 3.76 | 59.2 | 57.8 | 0.22 | 0.73 | 16.6 | 182 |
| example 1 | A:Ir(ppy)₃ | | | | | | | |
| Comparative | GHp1:compound | 3.81 | 69.2 | 60.3 | 0.22 | 0.73 | 17.6 | 160 |
| example 2 | B:Ir(ppy)₃ | | | | | | | |
| Comparative | Compound | 3.72 | 64.2 | 59.3 | 0.22 | 0.74 | 16.8 | 165 |
| example 3 | C:GHn1:Ir(ppy)₃ | | | | | | | |
| Comparative | GHp1:compound | 3.85 | 60 | 55.7 | 0.23 | 0.74 | 16.1 | 152 |
| example 4 | D:Ir(ppy)₃ | | | | | | | |
| Comparative | Compound | 4.1 | 58 | 55.1 | 0.22 | 0.73 | 15.9 | 160 |
| example 5 | E:GHn1:Ir(ppy)₃ | | | | | | | |

It can be known according to the data shown in Table 17 that, compared with the organic electroluminescence devices prepared in comparative examples 1 to 5, the organic electroluminescence devices prepared in examples 1 to 26 have basically similar driving voltages, the luminescence efficiency is at least improved by 16.3%, and the device service life is increased by at least 20.3%. Hence, when the compound of the present disclosure is used as the organic luminescent layer material of the organic electroluminescence device, especially used as a host material of the organic luminescent layer of the organic electroluminescence device, the efficiency performance and the service life of the organic electroluminescence device may be effectively improved.

For example, compared with the compound of comparative example 1, a dibenzo five-membered ring is directly connected to an adamantane spirofluorene ring in a structure of the compound of the present disclosure, an energy transmission ability is improved, exciton tolerance is improved, thus the compound of the present disclosure is more suitable as an electron-type host material of the luminescent layer, while the compound A is more suitable for the electron transport layer.

### EXAMPLE 27

### Red Organic Electroluminescence Device

An anode is prepared through the following processes: an ITO substrate with an ITO thickness of 1500 Å is cut into a size of 40 mm (length)×40 mm (width)×0.7 mm (thickness) to be prepared into an experimental substrate with pattern of a cathode, an anode and an insulating layer by adopting a photoetching process, and surface treatment is performed by utilizing ultraviolet ozone and O₂:N₂ plasma to increase a work function of the anode (the experiment substrate). A surface of the ITO substrate may be cleaned with an organic solvent to remove impurities and oil stains on the surface of the ITO substrate. It should be noted that the ITO substrate may further be cut into other sizes according to actual needs, and the size of the ITO substrate in the present disclosure is not particularly limited.

F4-TCNQ is vacuum-evaporated on the experiment substrate (the anode) to form a hole injection layer (HIL) with a thickness of 100 Å, and HT-03 is evaporated on the hole injection layer to form a first hole transport layer with a thickness of 835 Å.

HT-04 is vacuum-evaporated on the first hole transport layer to form a second hole transport layer with a thickness of 800 Å.

On the second hole transport layer, the compound 23 and Ir(piq)₂(acac) are co-evaporated at a ratio of 85%:15% (an evaporation rate) to form a red luminescent layer (EML) with a thickness of 350 Å.

ET-01 and LiQ are mixed at a weight ratio of 1:1 and a mixture is evaporated to form an electron transport layer (ETL) with a thickness of 300 Å, LiQ is evaporated on the electron transport layer to form an electron injection layer (EIL) with a thickness of 13 Å, and then magnesium (Mg) and silver (Ag) are mixed at an evaporation rate of 1:9 and a mixture is vacuum-evaporated on the electron injection layer to form a cathode with a thickness of 105 Å.

In addition, CP-01 with a thickness of 650 Å is evaporated on the above cathode to form an organic capping layer (CPL), thus completing preparation of the organic light-emitting device.

### EXAMPLE 28 TO EXAMPLE 34

Except for replacing mixture components in example 27 with mixture components shown in Table 18 below at the time of forming the luminescent layer, the organic electroluminescence device is manufactured by adopting the same method as in example 27.

### COMPARATIVE EXAMPLE 6 TO COMPARATIVE EXAMPLE 8

Except for replacing a luminescent layer host in example 27 with mixture components shown in Table 18 below at the time of forming the luminescent layer, the organic electroluminescence device is prepared by adopting the same method as in example 27.

Material structures used in examples 27 to 34 and comparative examples 6 to 8 are shown as follows.

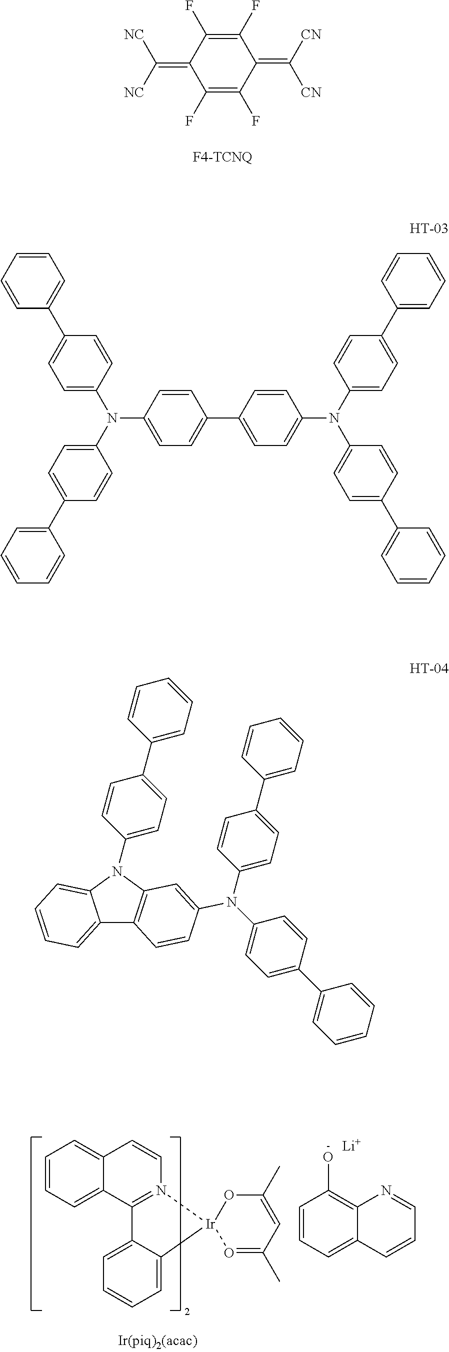

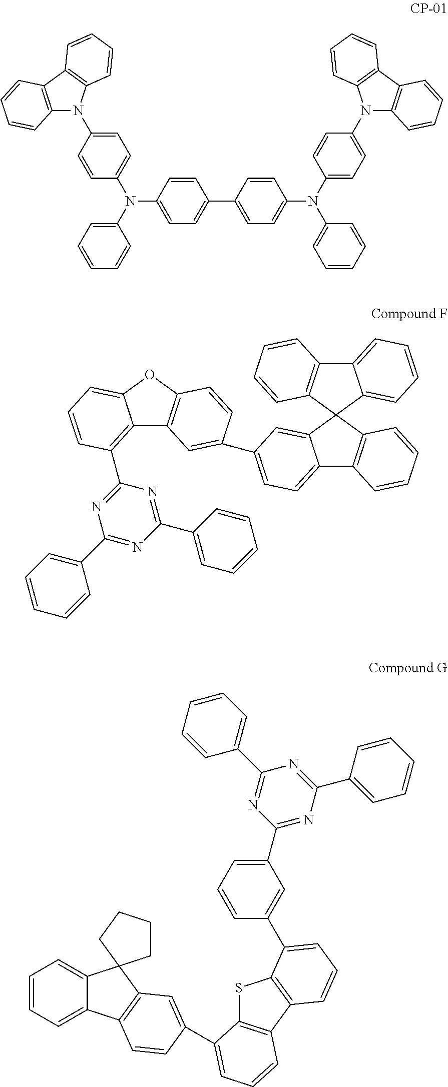 [SMILES: c1ccc(N(c2ccc(-c3ccc(N(c4ccccc4)c4ccc(-n5c6ccccc6c6ccccc65)cc4)cc3)cc2)c2ccc(-n3c4ccccc4c4ccccc43)cc2)cc1 ; c1ccc(-c2nc(-c3ccccc3)nc(-c3cccc4oc5ccc(-c6ccc7c(c6)C6(c8ccccc8-c8ccccc86)c6ccccc6-7)cc5c34)n2)cc1 ; c1ccc(-c2nc(-c3ccccc3)nc(-c3cccc(-c4cccc5c4sc4c(-c6ccc7c(c6)C6(CCCC6)c6ccccc6-7)cccc45)c3)n2)cc1]

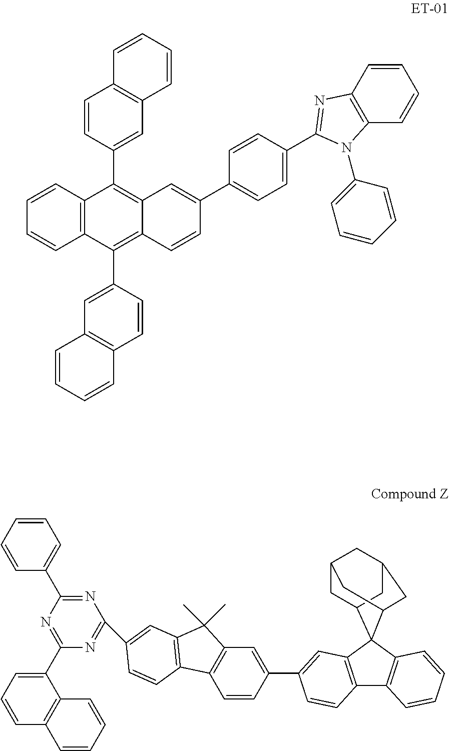 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; CC1(C)c2cc(-c3ccc4c(c3)C3(c5ccccc5-4)C4CC5CC(C4)CC3C5)ccc2-c2ccc(-c3nc(-c4ccccc4)nc(-c4cccc5ccccc45)n3)cc21]

The organic electroluminescence devices prepared in examples 27 to 34 and comparative examples 6 to 8 are tested for performance under a condition of 20 mA/cm², and test results are shown in Table 18 below.

TABLE 18
performance test results of organic electroluminescence device

| | | | | | | | External | |
|---|---|---|---|---|---|---|---|---|
| | luminescent | | | | | | quantum | T95 |
| | layer: Com- | Driving | Current | Power | Chromaticity | Chromaticity | efficiency | service |
| | pound:Ir(piq)₂acac = | voltage | efficiency | efficiency | coordinate | coordinate | EQE | life (h) |
| Example # | 95%:5% | (V) | (Cd/A) | (lm/W) | CIEx | CIEy | (%) | 20 mA/cm² |
| Example 27 | Compound 23 | 3.78 | 39.5 | 32.8 | 0.68 | 0.32 | 26.9 | 452 |
| Example 28 | Compound 25 | 3.72 | 39.7 | 33.5 | 0.68 | 0.32 | 27.1 | 449 |
| Example 29 | Compound 128 | 3.74 | 41.6 | 34.9 | 0.68 | 0.32 | 28.4 | 446 |
| Example 30 | Compound 239 | 3.73 | 38.7 | 32.6 | 0.68 | 0.32 | 26.4 | 479 |
| Example 31 | Compound 93 | 3.71 | 41.8 | 35.4 | 0.68 | 0.32 | 28.5 | 439 |
| Example 32 | Compound 259 | 3.70 | 40.9 | 34.7 | 0.68 | 0.32 | 27.9 | 419 |
| Example 33 | Compound 143 | 3.68 | 41.6 | 35.5 | 0.68 | 0.32 | 28.6 | 452 |
| Example 34 | Compound 185 | 3.72 | 40.3 | 34.9 | 0.68 | 0.32 | 27.9 | 448 |
| Comparative | Compound F | 4.03 | 31.6 | 21.5 | 0.68 | 0.32 | 18.8 | 310 |
| example 6 | | | | | | | | |
| Comparative | Compound G | 3.78 | 32.4 | 23.6 | 0.68 | 0.32 | 19.3 | 322 |
| example 7 | | | | | | | | |
| Comparative | Compound Z | 3.82 | 31.1 | 22.8 | 0.68 | 0.32 | 18.7 | 318 |
| example 8 | | | | | | | | |

It can be known according to the data shown in Table 18 that, compared with the organic electroluminescence devices prepared in comparative examples 6 to 8, the organic electroluminescence devices prepared in examples 27 to 34 have similar driving voltages, the device luminescence efficiency is at least improved by 19.4%, and the service life is increased by at least 30.1%. Hence, when the compound of the present disclosure is used as the organic luminescent layer material of the organic electroluminescence device, especially used as a host material of the organic luminescent layer of the organic electroluminescence device, the efficiency performance and the service life performance of the organic electroluminescence device can be improved.

For example, compared with the compound of comparative example 8, the dibenzo five-membered ring and the electron-deficient heteroaryl in the structure of the compound of the present disclosure are not directly connected, but are respectively connected with spirofluorene, so T₁ is apparently improved, excitation tolerance is improved, thus the compound of the present disclosure is more suitable as an electron-type host material of the luminescent layer, while the compound Z is more suitable for the electron transport layer.

In the compound of the present disclosure, adamantyl serving as a part of a compound core, is spiro-bonded with fluorenyl to obtain adamantyl spirofluorene, and the adamantyl spirofluorene and the dibenzo five-membered fused ring are connected through a single bond, so that the compound as a whole has strong rigidity, and the compound of the present disclosure is made to have a high first triplet energy level. The macromolecular structure has strong rigidity, and a freely rotating δ bond makes a certain twist angle be formed between two ring planes, so that the compound of the present disclosure has the high first triplet energy level and a suitable HOMO energy level distribution, and the dibenzo five-membered fused ring has an excellent energy transfer effect and thus can effectively promote the energy transfer between host and dopant materials of the luminescent layer, so it may serve as the host material of the luminescent layer in the organic electroluminescence material to improve the efficiency performance of the organic electroluminescence device.

Spiro combination of adamantyl and fluorenyl can greatly increase the electron cloud density of the large planar conjugated structures through a hyperconjugation effect, enhance the hole mobility of the compound, and help to promote transport balance of the holes and the electrons in the luminescent layer. Further, a recombination rate of the electrons and the holes in the organic luminescent layer can be increased, and transport of the electrons through the organic luminescent layer to the hole transport layer can be reduced or be avoided, so that the material of the hole transport layer can be effectively protected from the impact of the electrons, and the service life of the organic electroluminescence device can be increased. The adamantyl spiro-combined to the fluorenyl has a large steric volume and strong rigidity, so it can reduce an interaction force between the large planar conjugated structures, reduce intermolecular π-π stacking, and adjust an intermolecular stacking degree, so as to make the compound have a more stable amorphous state during film formation, improve a film-forming property of the compound, and further increase the service life of the organic electroluminescence device.

The optional embodiments of the present disclosure are described above in detail with reference to the accompanying drawings. However, the present disclosure is not limited to the specific details in the above embodiments. Within the scope of the technical concept of the present disclosure various simple modifications can be made to the technical solutions of the present disclosure, and these simple modifications belong to the protection scope of the present disclosure.

In addition, it should be noted that all the specific technical features described in the above specific embodiments can be combined in any suitable manner unless they are inconsistent. Various possible combination manners are not illustrated in the present disclosure in order to avoid unnecessary repetition.

In addition, the various embodiments of the present disclosure can also be combined arbitrarily, as long as they do not violate the idea of the present disclosure, and they should also be regarded as the content disclosed in the present disclosure.

1. A compound, wherein a structural formula of the compound is shown in a chemical formula 1: 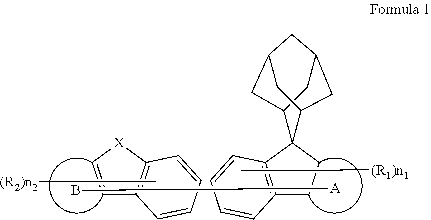
wherein, a ring A and a ring B are each independently selected from a benzene ring or a naphthalene ring;
X is selected from O, S, Si(R₃R₄) or C(R₃R₄); the R₃ and the R₄ are the same; and the R₃ and the R₄ are selected from methyl or phenyl; or the R₃ and the R₄ are connected to each other to form a cyclopentane ring, a cyclohexane ring or a fluorene ring together with the atoms to which they are jointly connected;
n₁ is 1, n₂ is 0, and R₁ is 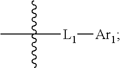
or, n₁ is 0, n₂ is 1, and R₂ is 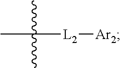
Ar₁ is selected from substituted or unsubstituted aryl with 6 to 40 carbon atoms, or substituted or unsubstituted heteroaryl with 3 to 40 carbon atoms;
L₁ is selected from one of a single bond, substituted or unsubstituted phenylene, substituted or unsubstituted biphenylene, substituted or unsubstituted terphenylene, substituted or unsubstituted naphthylene, substituted or unsubstituted 9,9-dimethylfluorenylene, substituted or unsubstituted phenanthrylene, substituted or unsubstituted anthrylene, spiro[cyclopentane-1,9′-fluorenylene], spiro[cyclohexane-1,9′-fluorenylene], substituted or unsubstituted pyridylene, substituted or unsubstituted pyrimidylene, substituted or unsubstituted 9,9-dimethyl-9H-9-silafluorenylene, substituted or unsubstituted dibenzofurylene, substituted or unsubstituted dibenzothienylene, substituted or unsubstituted quinolylene, substituted or unsubstituted isoquinolylene, substituted or unsubstituted carbazolylene, substituted or unsubstituted phenanthrolinylene, and substituted or unsubstituted spirobifluorenylene, or is a subunit group formed by connecting two or three of the above subunits through a single bond; and substituents in the L₁ are the same as or different from each other, and are each independently selected from the group consisting of deuterium, fluorine, chlorine, cyano, methyl, ethyl, isopropyl, n-propyl, tert-butyl, methoxy, ethoxy, trifluoromethyl, trimethylsilyl, phenyl and naphthyl;
substituents in Ar₁ are the same as or different from each other, and are each independently selected from the group consisting of: deuterium, cyano, a halogen group, nitro, a group U, alkoxy with 1 to 12 carbon atoms, haloalkyl with 1 to 12 carbon atoms, alkyl with 1 to 12 carbon atoms, cycloalkyl with 3 to 12 carbon atoms, heterocyclyl with 3 to 12 carbon atoms, alkylsilyl with 3 to 12 carbon atoms, arylsilyl with 6 to 18 carbon atoms, alkylthio with 1 to 12 carbon atoms, aryloxy with 6 to 18 carbon atoms, aralkyl with 7 to 18 carbon atoms, and aryloxy with 6 to 18 carbon atoms, the group U is selected from substituted or unsubstituted aryl with 6 to 30 carbon atoms, or substituted or unsubstituted heteroaryl with 3 to 30 carbon atoms, and substituents in the aryl and the heteroaryl are selected from the group consisting of deuterium, cyano, a halogen group, alkyl with 1 to 12 carbon atoms, alkoxy with 1 to 12 carbon atoms, cycloalkyl with 3 to 12 carbon atoms, haloalkyl with 1 to 12 carbon atoms, alkylsilyl with 3 to 12 carbon atoms, aryl with 6 to 20 carbon atoms, and heteroaryl with 3 to 18 carbon atoms;
or, in Ar₁, when there are two substituents on a same atom, optionally, the two substituents are connected to each other to form a 5- to 18-membered aliphatic ring or a 5- to 18-membered aromatic ring together with the atoms to which they are jointly connected;
L₂ is selected from one of a single bond, substituted or unsubstituted phenylene, substituted or unsubstituted biphenylene, substituted or unsubstituted terphenylene, substituted or unsubstituted naphthylene, substituted or unsubstituted 9,9-dimethylfluorenylene, substituted or unsubstituted phenanthrylene, spiro[cyclopentane-1,9′-fluorenylene], spiro[cyclohexane-1,9′-fluorenylene], substituted or unsubstituted 9,9-dimethyl-9H-9-silafluorenylene, substituted or unsubstituted dibenzofurylene, substituted or unsubstituted dibenzothienylene, and substituted or unsubstituted spirobifluorenylene, or is a subunit group formed by connecting two or three of the above subunits through a single bond; and substituents in the L₂ are the same as or different from each other, and are each independently selected from the group consisting of deuterium, fluorine, chlorine, cyano, methyl, ethyl, isopropyl, n-propyl, tert-butyl, methoxy, ethoxy, trifluoromethyl, trimethylsilyl, phenyl and naphthyl;
Ar₂ is selected from substituted or unsubstituted aryl with 6 to 30 carbon atoms or 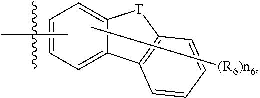
and the Ar₂ is not anthryl;
wherein, T is selected from O, S, C(R₇R₈) or Si(R₇R₈); the R₇ and the R₈ are the same, and the R₇ and the R₈ are selected from methyl or phenyl; or the R₇ and the R₈ are connected to each other to form a cyclopentane ring, a cyclohexane ring or a fluorene ring together with the atoms to which they are jointly connected;
R₅ and R₆ are the same as or different from each other, and are each independently selected from hydrogen, deuterium, a halogen group, cyano, alkyl with 1 to 12 carbon atoms, haloalkyl with 1 to 12 carbon atoms, alkoxy with 1 to 12 carbon atoms, cycloalkyl with 3 to 12 carbon atoms, heterocyclyl with 3 to 12 carbon atoms, alkylsilyl with 3 to 12 carbon atoms, arylsilyl with 6 to 18 carbon atoms, alkylthio with 1 to 12 carbon atoms, aryl with 6 to 18 carbon atoms, dibenzothienyl or dibenzofuranyl; and
n₆ represents the number of R₆, n₆ is selected from 0, 1, 2, 3, 4, 5, 6, 7 or 8; and when n₆ is greater than 1, any two R₆ are the same or different.

2-4. (canceled)

5. The compound according to claim 1, wherein the structural formula of the compound is shown in any one of formulae (f-1) to (f-16):

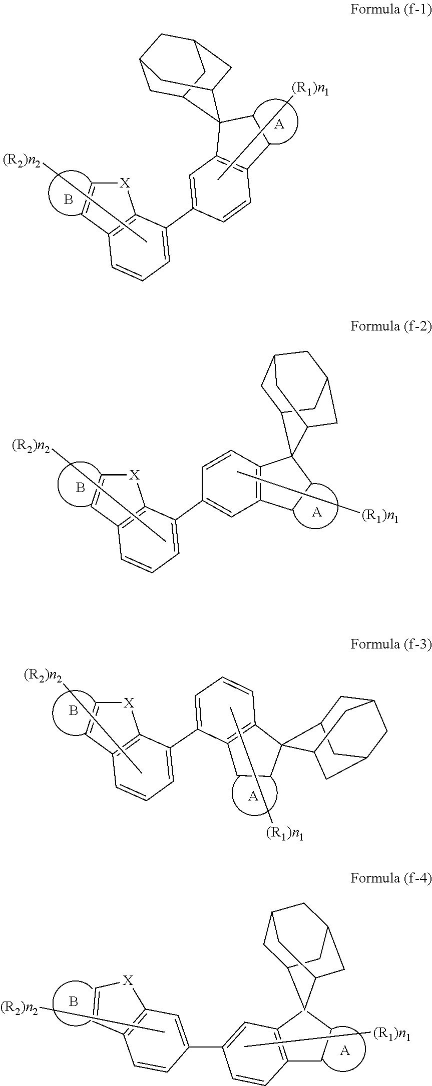

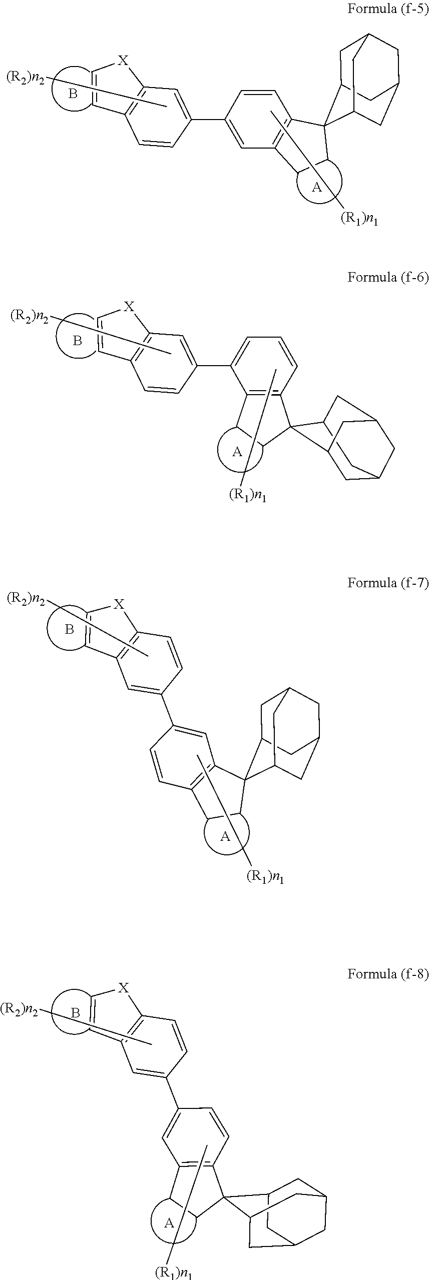

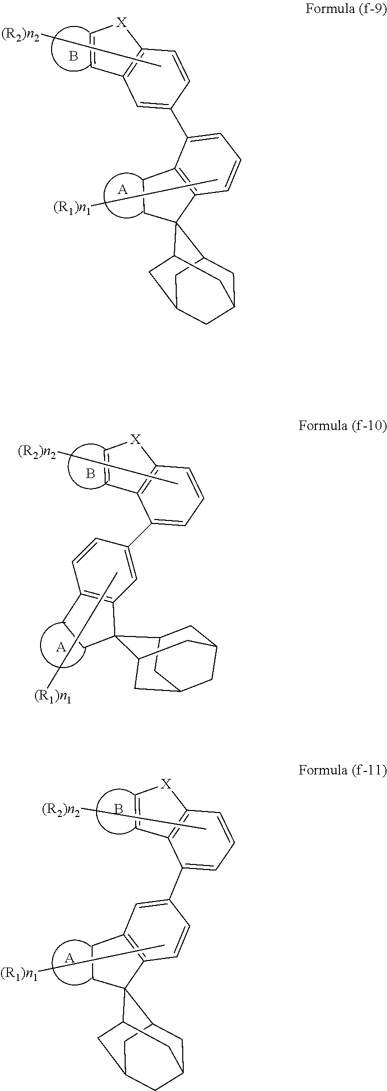

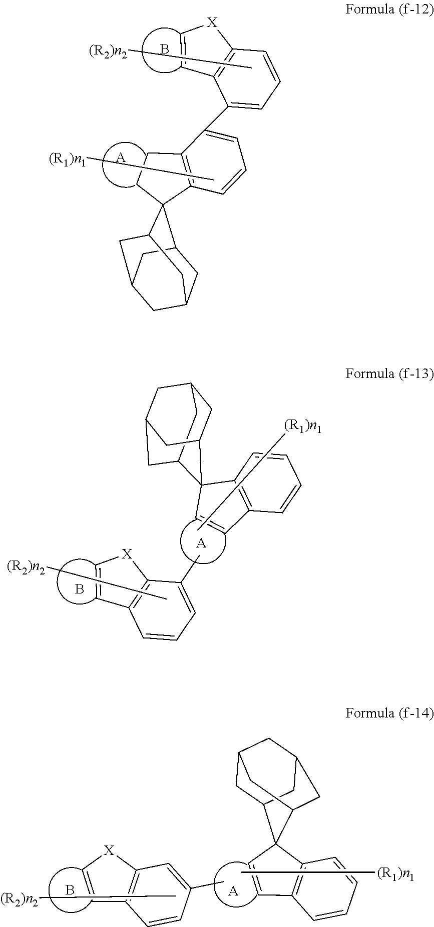

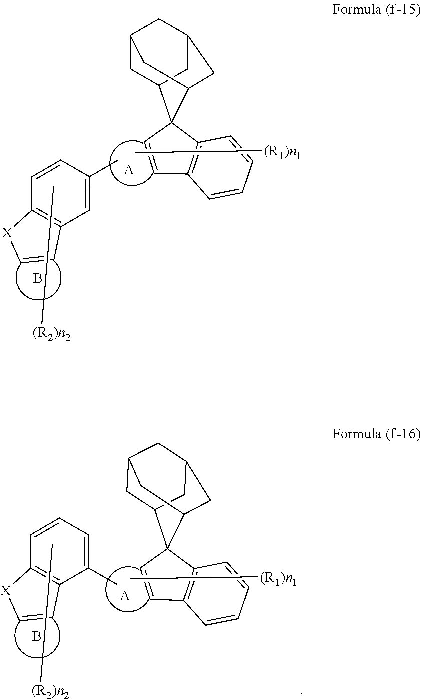

6-7. (canceled)

8. The compound according to claim 1, wherein the L₁ is selected from a single bond, or substituted or unsubstituted W₁, wherein the unsubstituted W₁ is selected from the group consisting of the following groups: 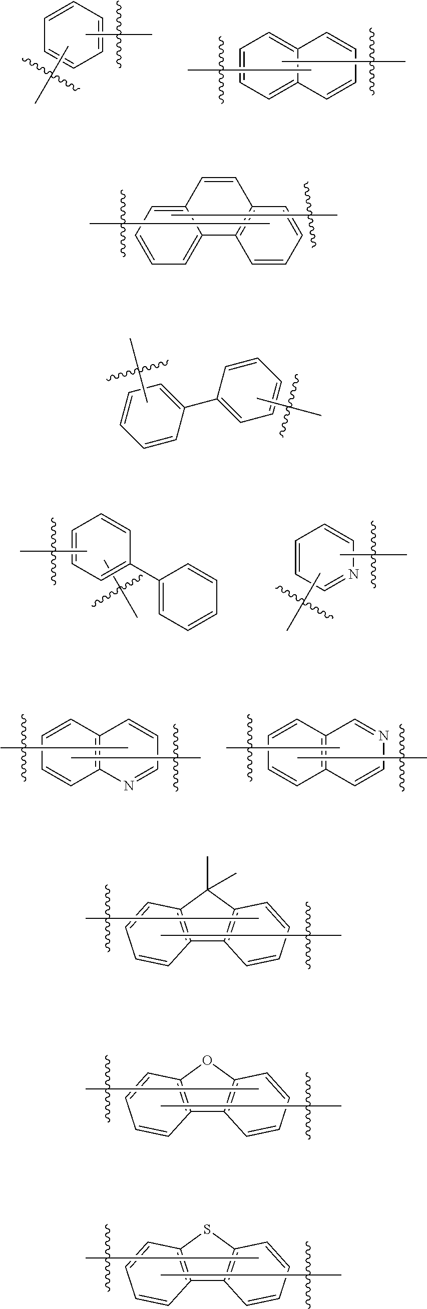 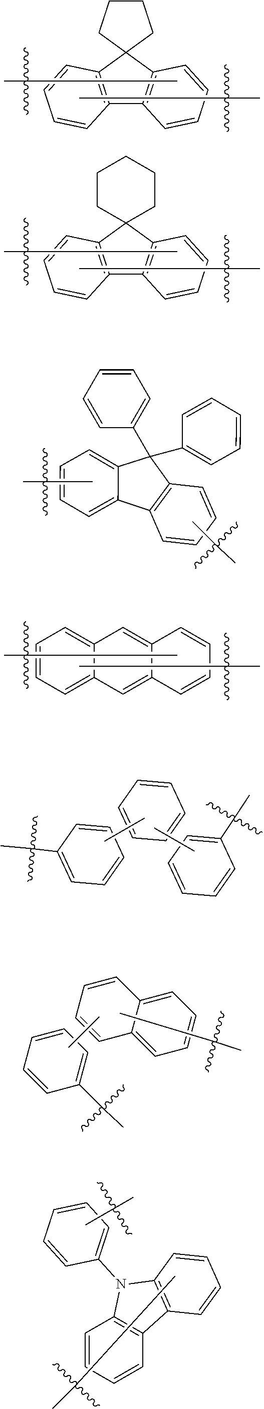 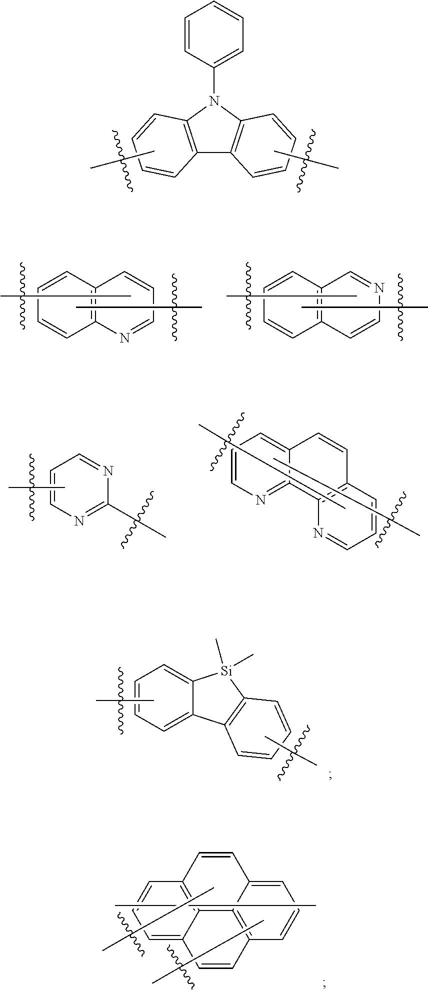
wherein, the substituted W₁ is a group formed by substituting the unsubstituted W₁ with one or more substituents selected from deuterium, fluorine, chlorine, bromine, cyano, and when there are a plurality of substituents on the substituted W₁, any two substituents are the same or different.

9. The compound according to claim 1, wherein the Ar₁ is selected from a substituted or unsubstituted group W₃, wherein the unsubstituted group W₃ is selected from the group consisting of the following groups: 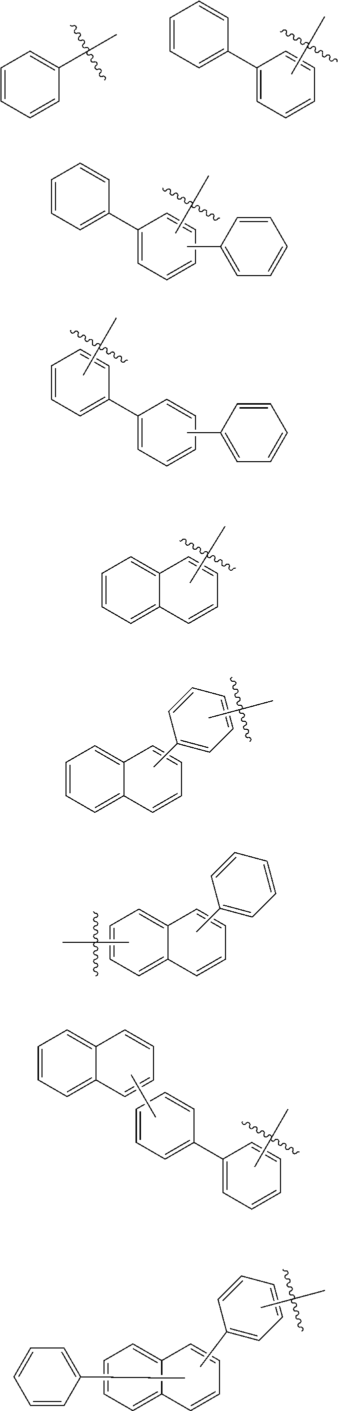 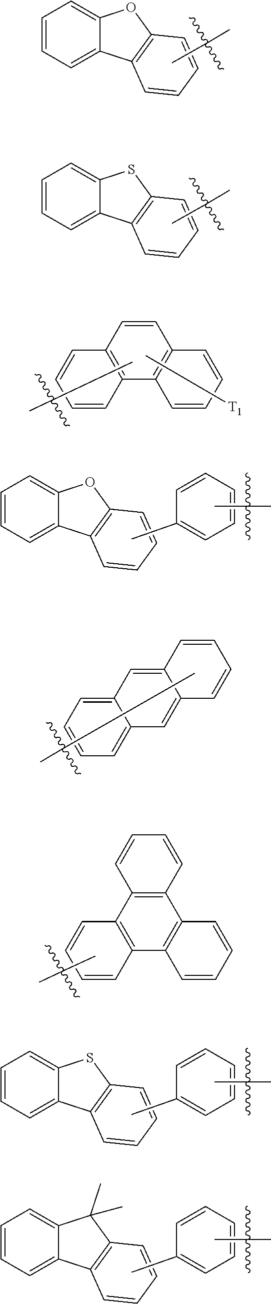 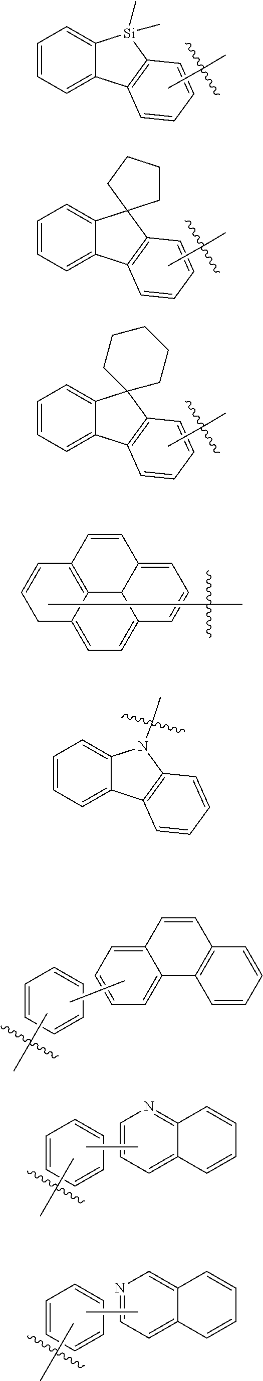 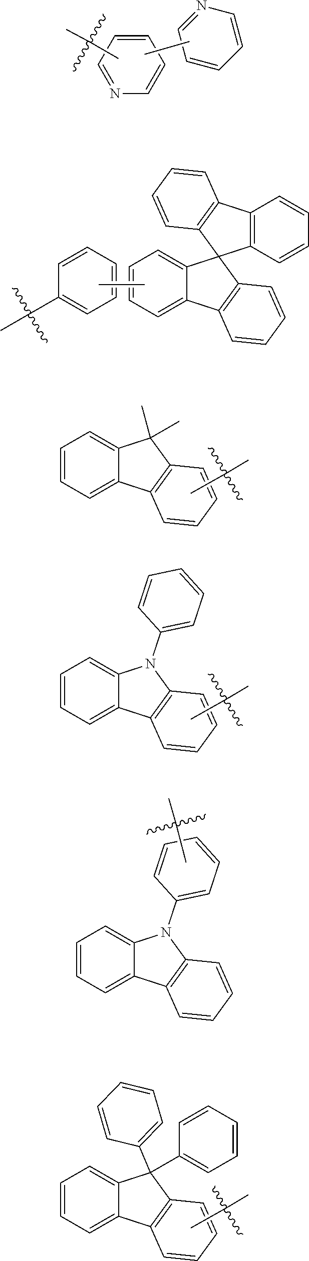 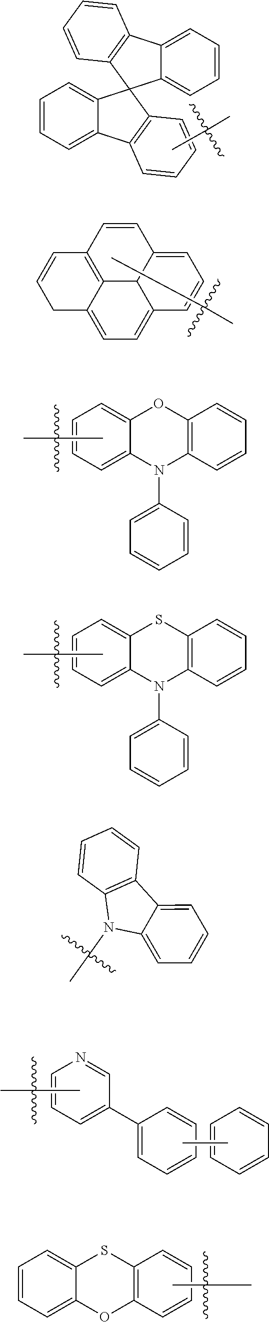 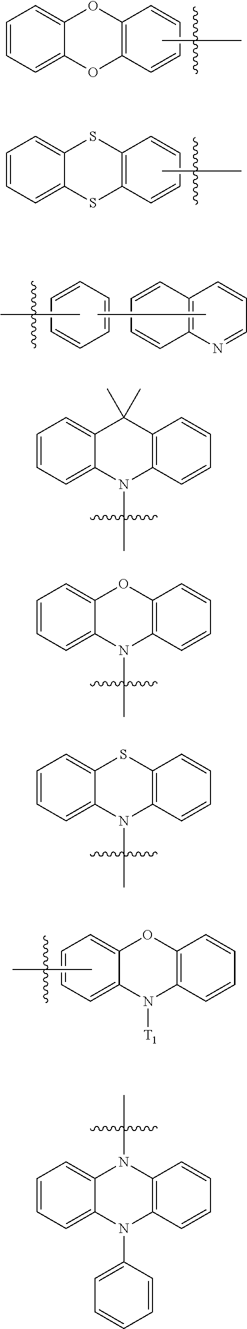 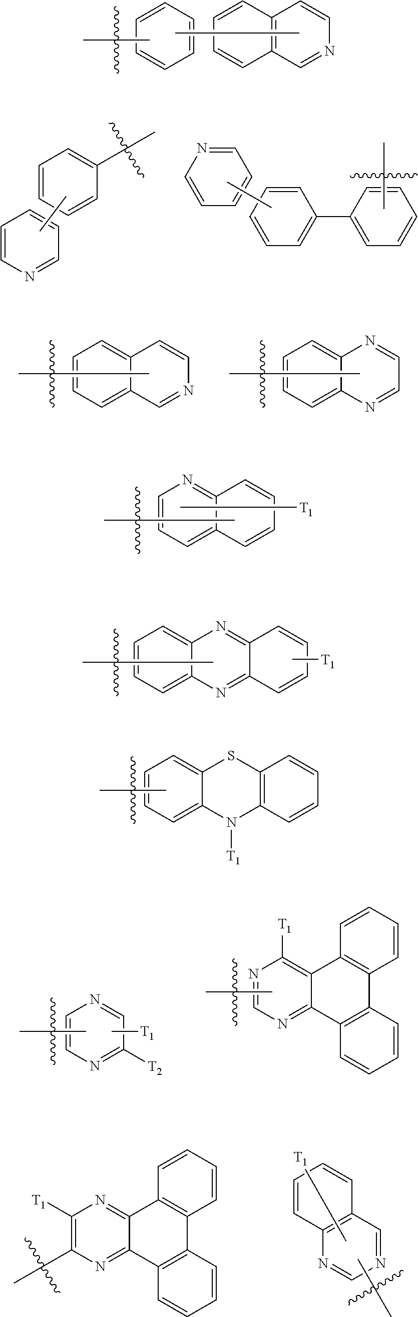 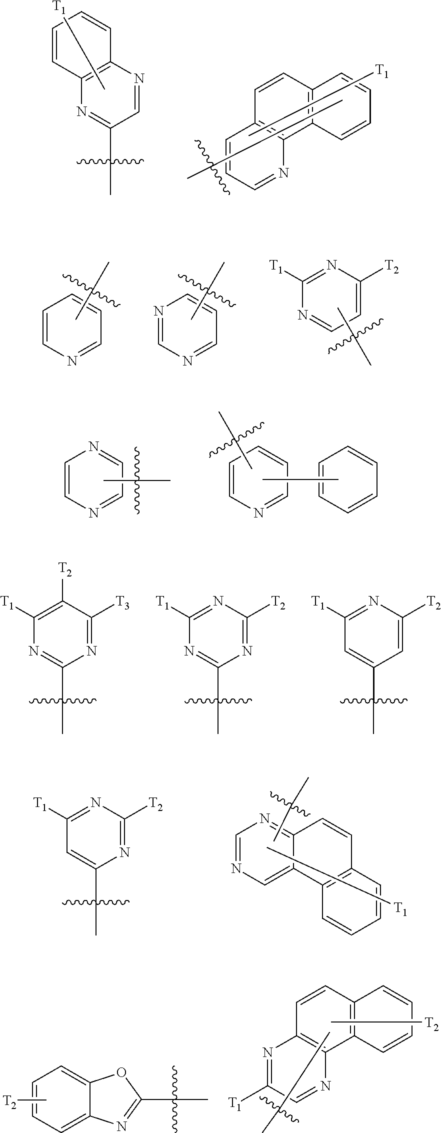 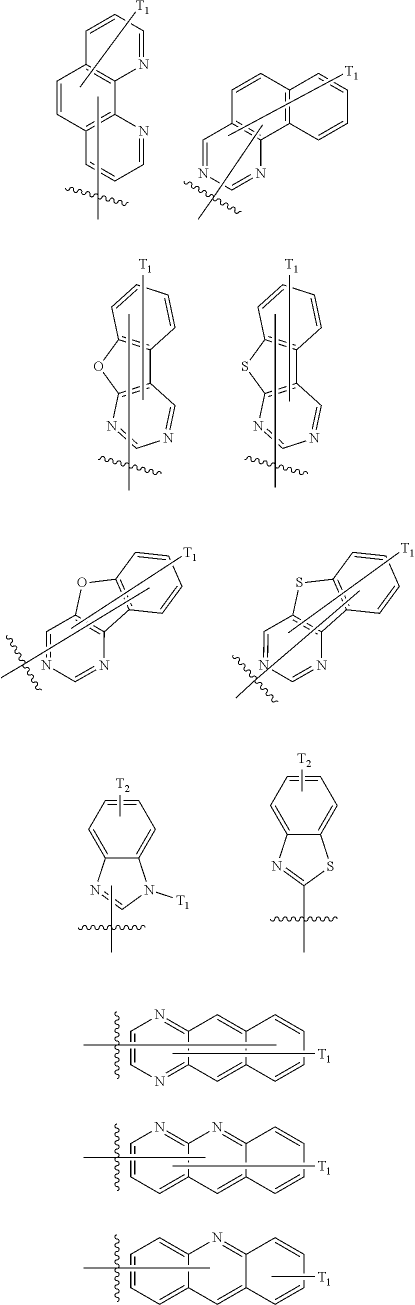 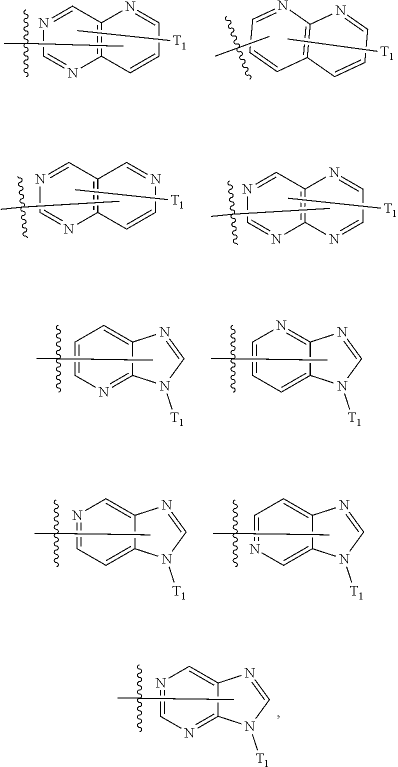
wherein, each T₁, each T₂ and each T₃ are the same as or different from each other, and are each independently selected from hydrogen, aryl with 6 to 20 carbon atoms, or heteroaryl with 3 to 20 carbon atoms; any two T₁ are the same or different, any two T₂ are the same or different, and any two T₃ are the same or different;
wherein, the substituted group W₃ is a group formed by substituting the unsubstituted group W₃ with one or more substituents selected from deuterium, fluorine, chlorine, cyano, alkyl with 1 to 4 carbon atoms, alkoxy with 1 to 4 carbon atoms, cycloalkyl with 3 to 7 carbon atoms, aryl with 6 to 15 carbon atoms, heteroaryl with 3 to 18 carbon atoms, alkylthio with 1 to 4 carbon atoms, haloalkyl with 1 to 4 carbon atoms, or alkylsilyl with 3 to 9 carbon atoms, and when there are a plurality of substituents on the W₃, any two substituents are the same or different.

10. The compound according to claim 9, wherein each T₁, each T₂ and each T₃ are the same as or different from each other, and are each independently selected from: hydrogen, phenyl, naphthyl, anthryl, phenanthryl, diphenyl, terphenyl, fluorenyl, dibenzothienyl, dibenzofuranyl, N-phenylcarbazolyl, carbazol-9-yl-phenyl, pyridyl, pyrimidinyl, pyrazinyl, quinolyl, isoquinolyl, quinazolinyl, benzoxazine, triphenylene or phenanthrolinyl; and
the substituted group W₃ is a group formed by substituting the unsubstituted group W₃ with one or more substituents selected from: fluorine, deuterium, cyano, trifluoromethyl, trimethylsilyl, methyl, ethyl, isopropyl, tert-butyl, methoxy, ethoxy, isopropoxy, trifluoromethyl, cyclopentyl, cyclohexyl, phenyl, biphenyl, naphthyl, fluorenyl, 9,9-dimethylfluorenyl, pyridyl, pyrimidinyl, quinolyl, isoquinolyl, carbazolyl, N-phenylcarbazolyl, dibenzofuranyl, or dibenzothienyl; and when there are a plurality of substituents on the W₃, any two substituents are the same or different.

11. (canceled)

12. The compound according to claim 1, wherein the L₂ is selected from a single bond, or substituted or unsubstituted W₂, wherein the unsubstituted W₂ is selected from the group consisting of the following groups: 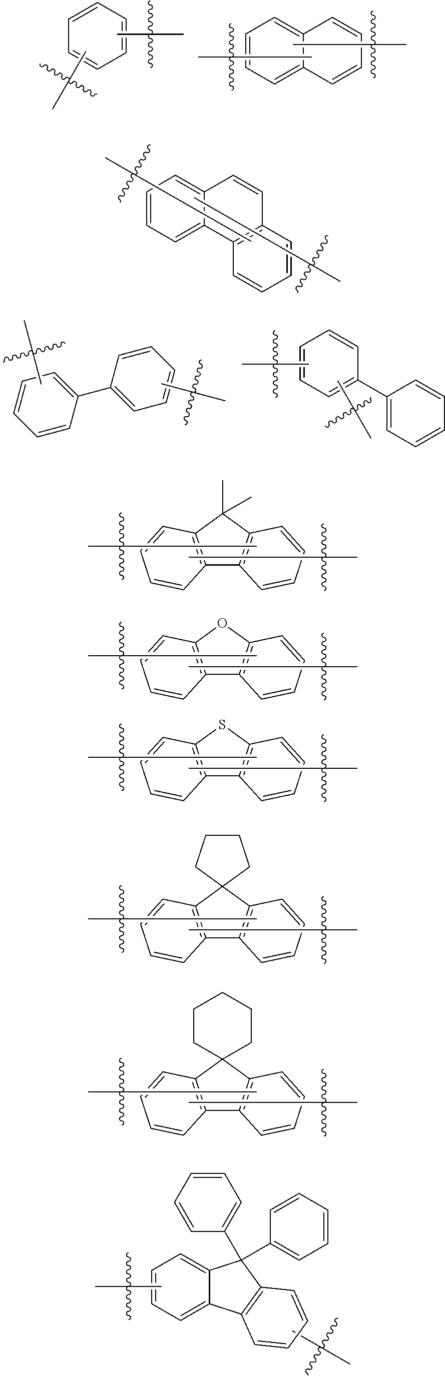 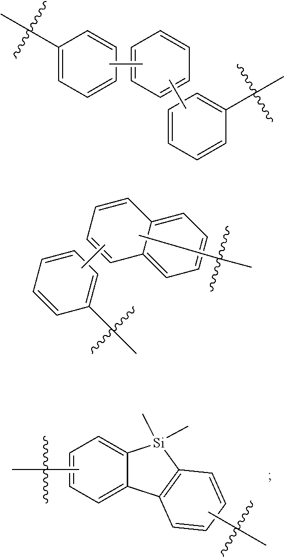
wherein, the substituted W₂ is a group formed by substituting the unsubstituted W₂ with one or more substituents selected from deuterium, fluorine, chlorine, bromine, cyano, and when there are a plurality of substituents on the substituted W₂, any two substituents are the same or different.

13. The compound according to claim 1, wherein the Ar₂ is selected from one of substituted or unsubstituted phenyl, substituted or unsubstituted biphenyl, substituted or unsubstituted terphenyl, substituted or unsubstituted naphthyl, substituted or unsubstituted fluorenyl, substituted or unsubstituted silafluorenyl, substituted or unsubstituted dibenzofuranyl, substituted or unsubstituted dibenzothienyl, substituted or unsubstituted phenanthryl, substituted or unsubstituted pyrenyl, substituted or unsubstituted perylenyl, substituted or unsubstituted triphenylene, substituted or unsubstituted spirobifluorenyl, spiro[cyclopentane-1,9′-fluorenyl], and spiro[cyclohexane-1,9′-fluorenyl], or is a group formed by connecting two or three of the above groups through a single bond; and substituents in the Ar₂ are the same as or different from each other, and are each independently selected from the group consisting of deuterium, fluorine, chlorine, cyano, methyl, ethyl, isopropyl, n-propyl, tert-butyl, methoxy, trifluoromethyl, trimethylsilyl, phenyl, fluorenyl, phenyl substituted by cyano, phenyl substituted by fluorine, naphthyl, dibenzothienyl, dibenzofuranyl, cyclohexyl and trimethylsilyl, and when there are a plurality of substituents in the Ar₂, the substituents are the same as or different from each other.

14. The compound according to claim 1, wherein the Ar₂ is selected from a substituted or unsubstituted group W₄, wherein the unsubstituted group W₄ is selected from the group consisting of the following groups: 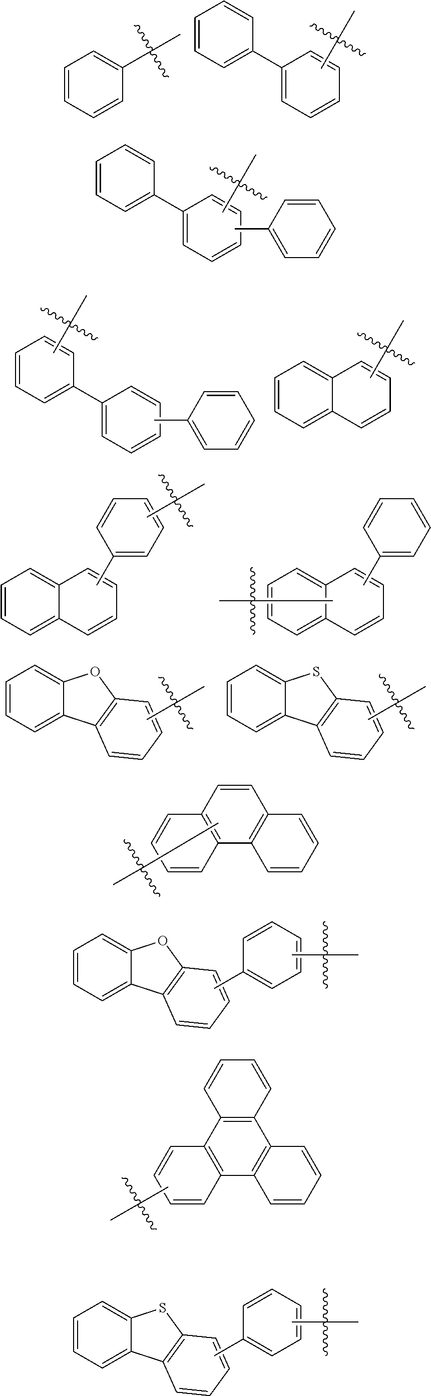 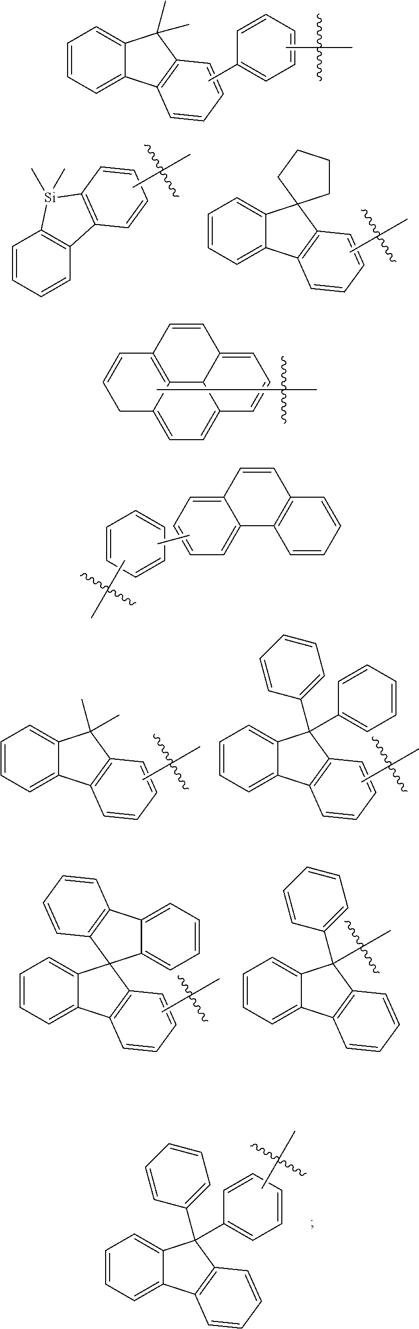
wherein, the substituted group W₄ is a group formed by substituting the unsubstituted group W₄ with one or more substituents selected from deuterium, fluorine, chlorine, cyano, alkyl with 1 to 4 carbon atoms, alkoxy with 1 to 4 carbon atoms, cycloalkyl with 3 to 7 carbon atoms, aryl with 6 to 14 carbon atoms, heteroaryl with 3 to 12 carbon atoms, alkylthio with 1 to 4 carbon atoms, haloalkyl with 1 to 4 carbon atoms, or alkylsilyl with 3 to 9 carbon atoms; and when there are a plurality of substituents on the W₄, any two substituents are the same or different.

15. The compound according to claim 1, wherein the compound is selected from the group consisting of the following compounds:

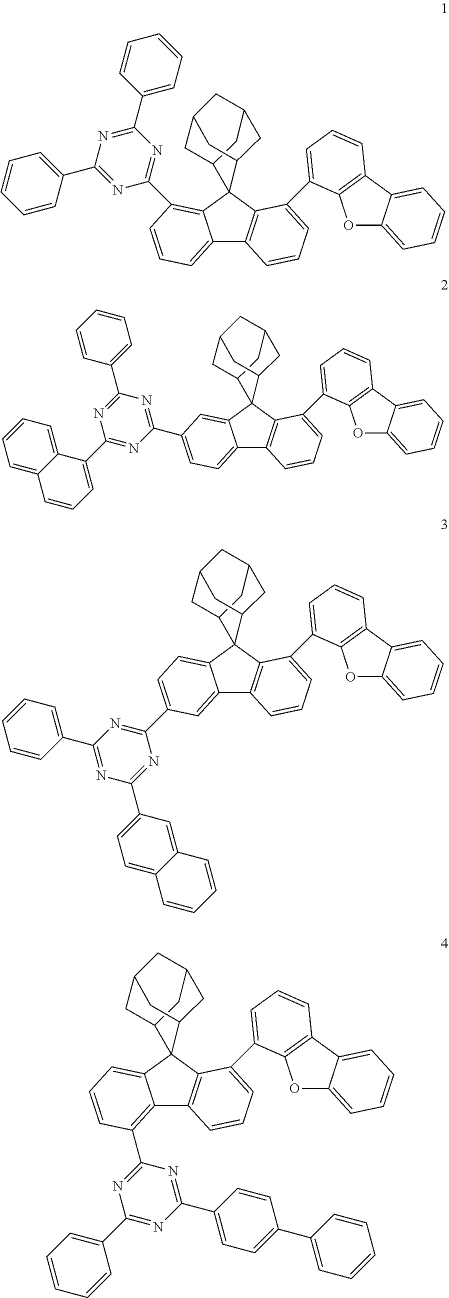 [SMILES: c1ccc(-c2nc(-c3ccccc3)nc(-c3cccc4c3C3(c5c-4cccc5-c4cccc5c4oc4ccccc45)C4CC5CC(C4)CC3C5)n2)cc1 ; c1ccc(-c2nc(-c3ccc4c(c3)C3(c5c-4cccc5-c4cccc5c4oc4ccccc45)C4CC5CC(C4)CC3C5)nc(-c3cccc4ccccc34)n2)cc1 ; c1ccc(-c2nc(-c3ccc4c(c3)-c3cccc(-c5cccc6c5oc5ccccc56)c3C43C4CC5CC(C4)CC3C5)nc(-c3ccc4ccccc4c3)n2)cc1 ; c1ccc(-c2ccc(-c3nc(-c4ccccc4)nc(-c4cccc5c4-c4cccc(-c6cccc7c6oc6ccccc67)c4C54C5CC6CC(C5)CC4C6)n3)cc2)cc1]

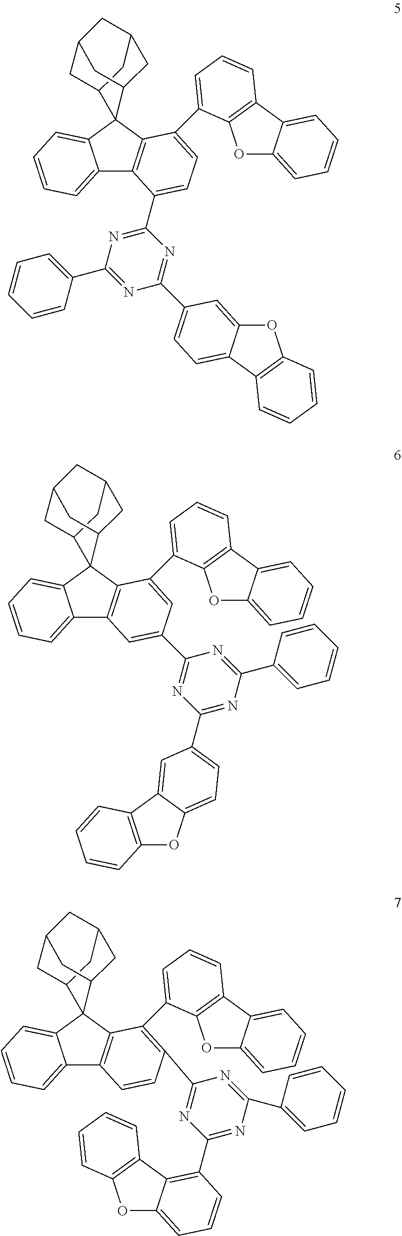 [SMILES: c1ccc(-c2nc(-c3ccc4c(c3)oc3ccccc34)nc(-c3ccc(-c4cccc5c4oc4ccccc45)c4c3-c3ccccc3C43C4CC5CC(C4)CC3C5)n2)cc1 ; c1ccc(-c2nc(-c3cc4c(c(-c5cccc6c5oc5ccccc56)c3)C3(c5ccccc5-4)C4CC5CC(C4)CC3C5)nc(-c3ccc4oc5ccccc5c4c3)n2)cc1 ; c1ccc(-c2nc(-c3ccc4c(c3-c3cccc5c3oc3ccccc35)C3(c5ccccc5-4)C4CC5CC(C4)CC3C5)nc(-c3cccc4oc5ccccc5c34)n2)cc1]

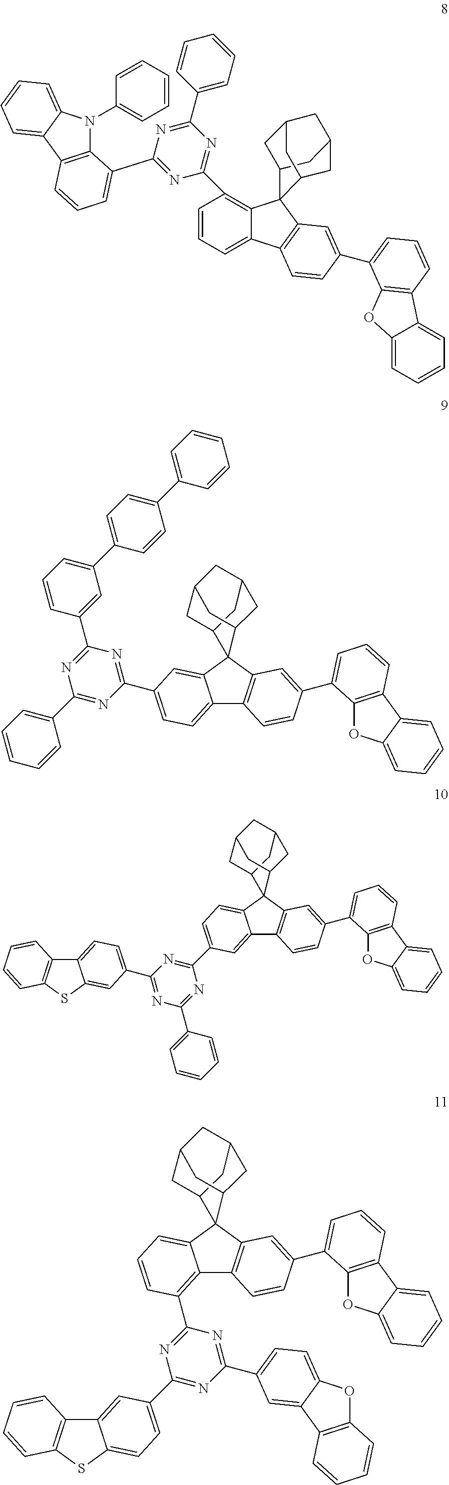 [SMILES: c1ccc(-c2nc(-c3cccc4c3C3(c5cc(-c6cccc7c6oc6ccccc67)ccc5-4)C4CC5CC(C4)CC3C5)nc(-c3cccc4c5ccccc5n(-c5ccccc5)c34)n2)cc1 ; c1ccc(-c2ccc(-c3cccc(-c4nc(-c5ccccc5)nc(-c5ccc6c(c5)C5(c7cc(-c8cccc9c8oc8ccccc89)ccc7-6)C6CC7CC(C6)CC5C7)n4)c3)cc2)cc1 ; c1ccc(-c2nc(-c3ccc4c(c3)-c3ccc(-c5cccc6c5oc5ccccc56)cc3C43C4CC5CC(C4)CC3C5)nc(-c3ccc4c(c3)sc3ccccc34)n2)cc1 ; c1cc(-c2nc(-c3ccc4oc5ccccc5c4c3)nc(-c3ccc4sc5ccccc5c4c3)n2)c2c(c1)C1(c3cc(-c4cccc5c4oc4ccccc45)ccc3-2)C2CC3CC(C2)CC1C3]

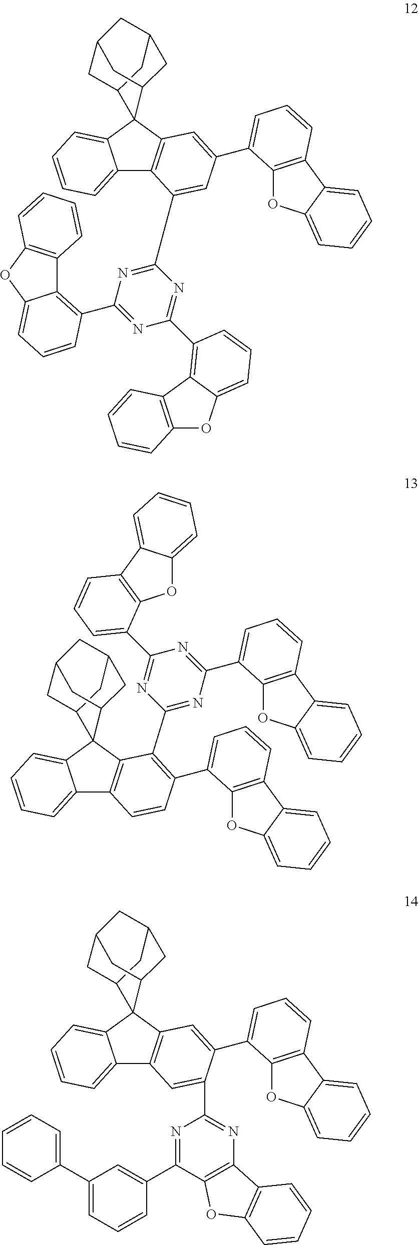

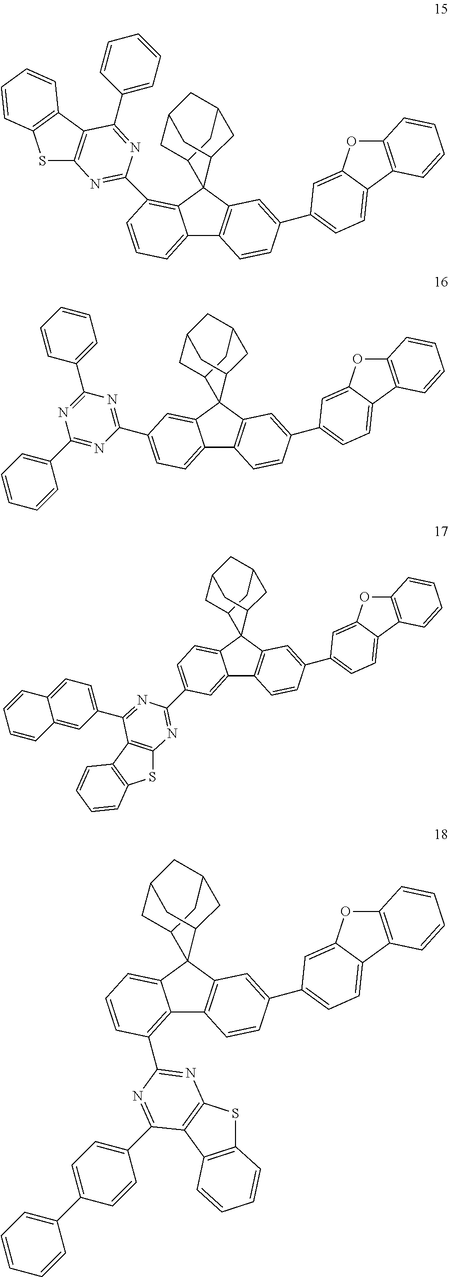 [SMILES: c1ccc(-c2nc(-c3cccc4c3C3(c5cc(-c6ccc7c(c6)oc6ccccc67)ccc5-4)C4CC5CC(C4)CC3C5)nc3sc4ccccc4c23)cc1 ; c1ccc(-c2nc(-c3ccccc3)nc(-c3ccc4c(c3)C3(c5cc(-c6ccc7c(c6)oc6ccccc67)ccc5-4)C4CC5CC(C4)CC3C5)n2)cc1 ; c1ccc2cc(-c3nc(-c4ccc5c(c4)-c4ccc(-c6ccc7c(c6)oc6ccccc67)cc4C54C5CC6CC(C5)CC4C6)nc4sc5ccccc5c34)ccc2c1 ; c1ccc(-c2ccc(-c3nc(-c4cccc5c4-c4ccc(-c6ccc7c(c6)oc6ccccc67)cc4C54C5CC6CC(C5)CC4C6)nc4sc5ccccc5c34)cc2)cc1]

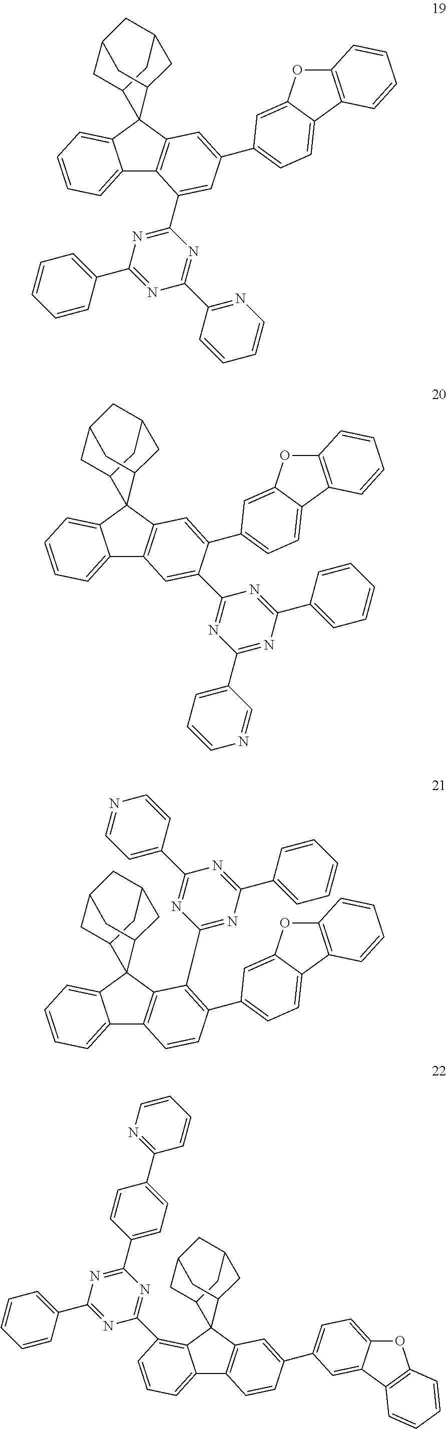 [SMILES: c1ccc(-c2nc(-c3ccccn3)nc(-c3cc(-c4ccc5c(c4)oc4ccccc45)cc4c3-c3ccccc3C43C4CC5CC(C4)CC3C5)n2)cc1 ; c1ccc(-c2nc(-c3cccnc3)nc(-c3cc4c(cc3-c3ccc5c(c3)oc3ccccc35)C3(c5ccccc5-4)C4CC5CC(C4)CC3C5)n2)cc1 ; c1ccc(-c2nc(-c3ccncc3)nc(-c3c(-c4ccc5c(c4)oc4ccccc45)ccc4c3C3(c5ccccc5-4)C4CC5CC(C4)CC3C5)n2)cc1 ; c1ccc(-c2nc(-c3ccc(-c4ccccn4)cc3)nc(-c3cccc4c3C3(c5cc(-c6ccc7oc8ccccc8c7c6)ccc5-4)C4CC5CC(C4)CC3C5)n2)cc1]

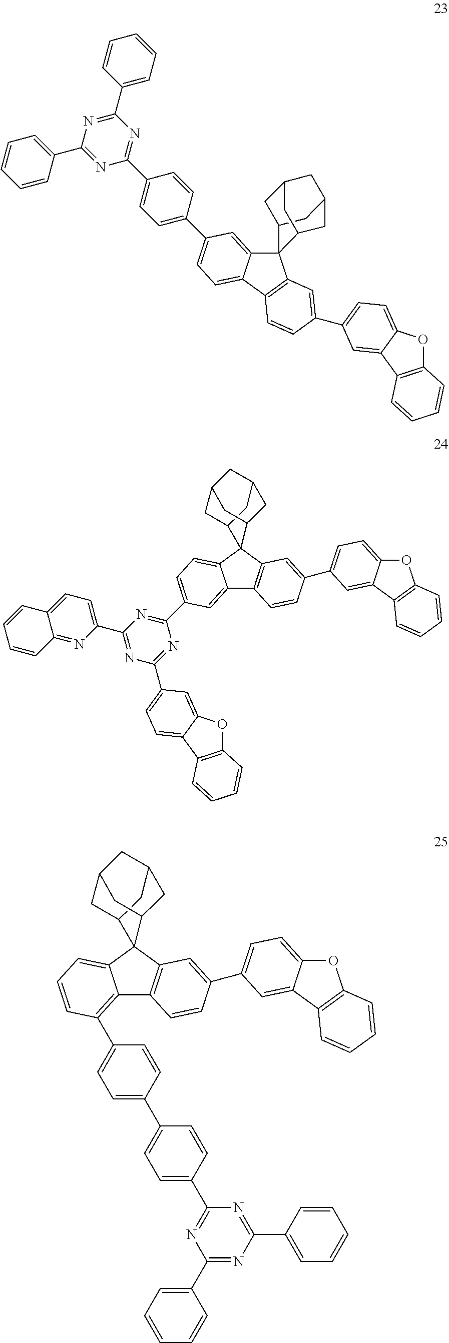 [SMILES: c1ccc(-c2nc(-c3ccccc3)nc(-c3ccc(-c4ccc5c(c4)C4(c6cc(-c7ccc8oc9ccccc9c8c7)ccc6-5)C5CC6CC(C5)CC4C6)cc3)n2)cc1 ; c1ccc2nc(-c3nc(-c4ccc5c(c4)-c4ccc(-c6ccc7oc8ccccc8c7c6)cc4C54C5CC6CC(C5)CC4C6)nc(-c4ccc5c(c4)oc4ccccc45)n3)ccc2c1 ; c1ccc(-c2nc(-c3ccccc3)nc(-c3ccc(-c4ccc(-c5cccc6c5-c5ccc(-c7ccc8oc9ccccc9c8c7)cc5C65C6CC7CC(C6)CC5C7)cc4)cc3)n2)cc1]

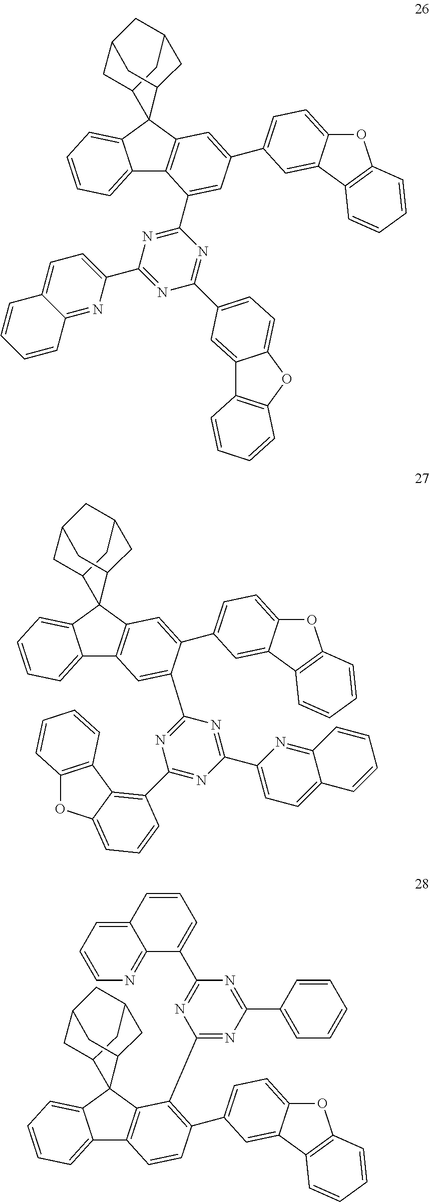

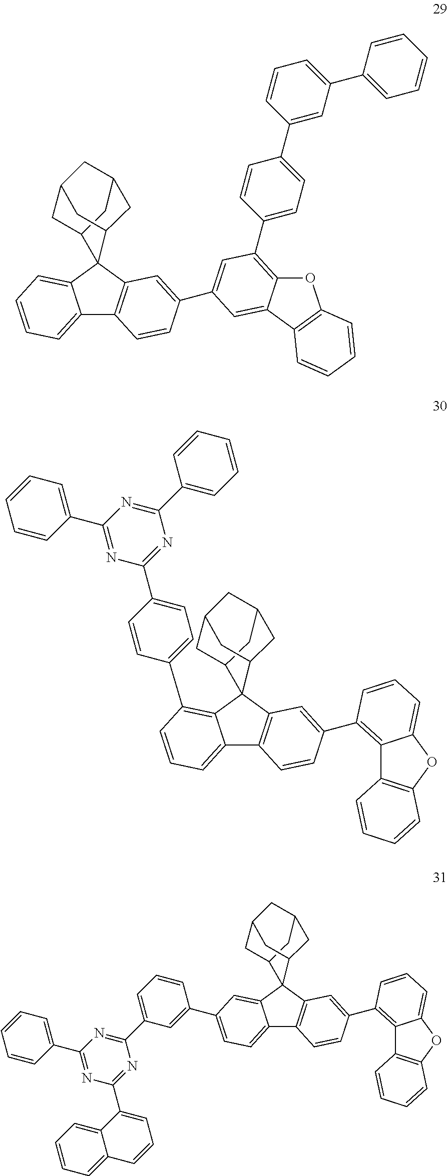 [SMILES: c1ccc(-c2cccc(-c3ccc(-c4cc(-c5ccc6c(c5)C5(c7ccccc7-6)C6CC7CC(C6)CC5C7)cc5c4oc4ccccc45)cc3)c2)cc1 ; c1ccc(-c2nc(-c3ccccc3)nc(-c3ccc(-c4cccc5c4C4(c6cc(-c7cccc8oc9ccccc9c78)ccc6-5)C5CC6CC(C5)CC4C6)cc3)n2)cc1 ; c1ccc(-c2nc(-c3cccc(-c4ccc5c(c4)C4(c6cc(-c7cccc8oc9ccccc9c78)ccc6-5)C5CC6CC(C5)CC4C6)c3)nc(-c3cccc4ccccc34)n2)cc1]

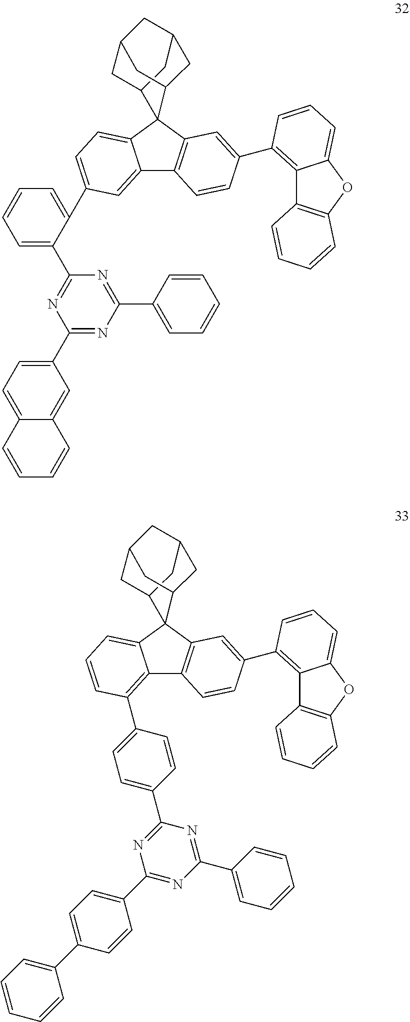 [SMILES: c1ccc(-c2nc(-c3ccc4ccccc4c3)nc(-c3ccccc3-c3ccc4c(c3)-c3ccc(-c5cccc6oc7ccccc7c56)cc3C43C4CC5CC(C4)CC3C5)n2)cc1 ; c1ccc(-c2ccc(-c3nc(-c4ccccc4)nc(-c4ccc(-c5cccc6c5-c5ccc(-c7cccc8oc9ccccc9c78)cc5C65C6CC7CC(C6)CC5C7)cc4)n3)cc2)cc1]

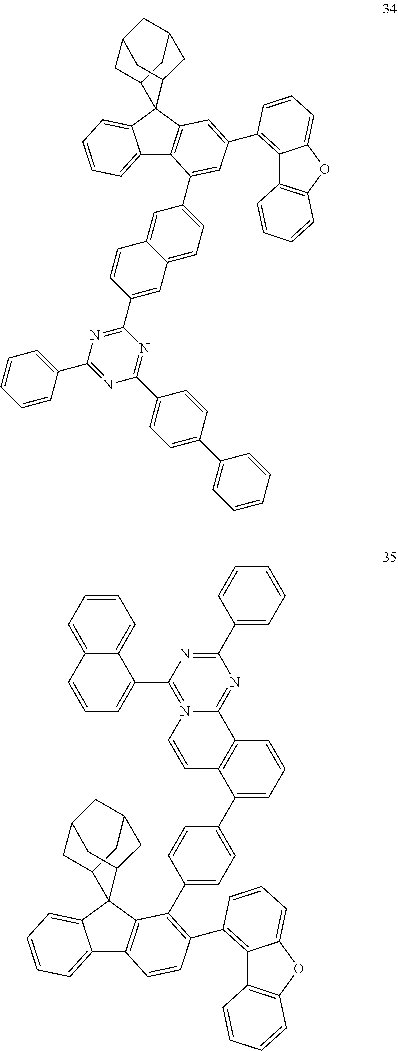

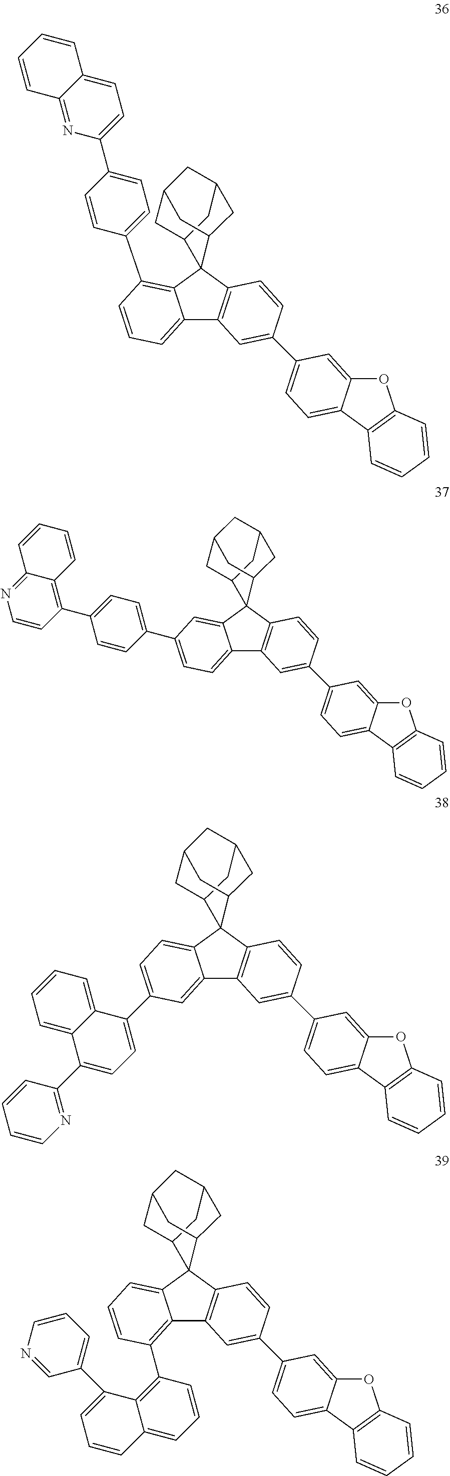 [SMILES: c1cc(-c2ccc(-c3ccc4ccccc4n3)cc2)c2c(c1)-c1cc(-c3ccc4c(c3)oc3ccccc34)ccc1C21C2CC3CC(C2)CC1C3 ; c1ccc2c(-c3ccc(-c4ccc5c(c4)C4(c6ccc(-c7ccc8c(c7)oc7ccccc78)cc6-5)C5CC6CC(C5)CC4C6)cc3)ccnc2c1 ; c1ccc(-c2ccc(-c3ccc4c(c3)-c3cc(-c5ccc6c(c5)oc5ccccc56)ccc3C43C4CC5CC(C4)CC3C5)c3ccccc23)nc1 ; c1cncc(-c2cccc3cccc(-c4cccc5c4-c4cc(-c6ccc7c(c6)oc6ccccc67)ccc4C54C5CC6CC(C5)CC4C6)c23)c1]

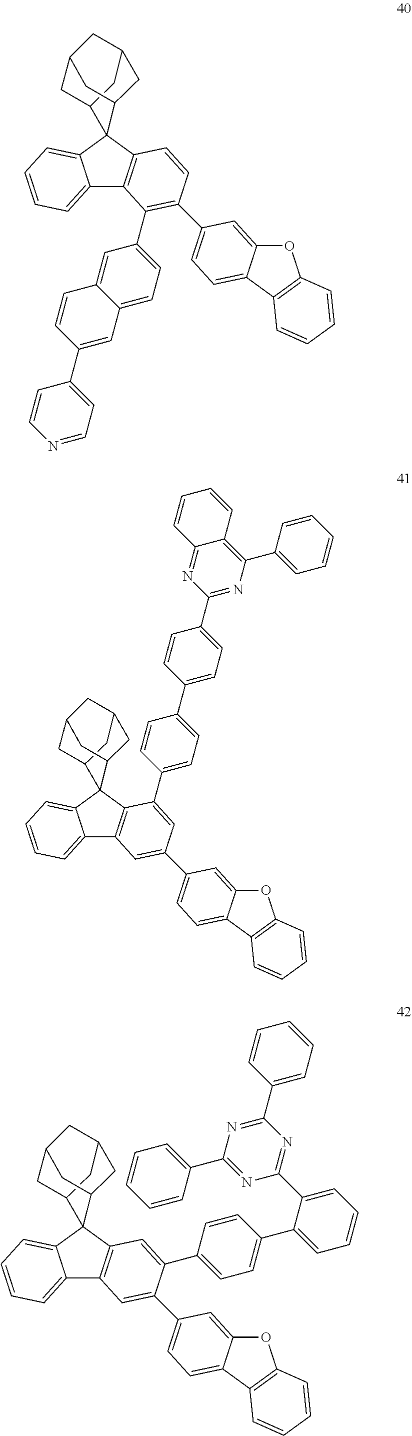

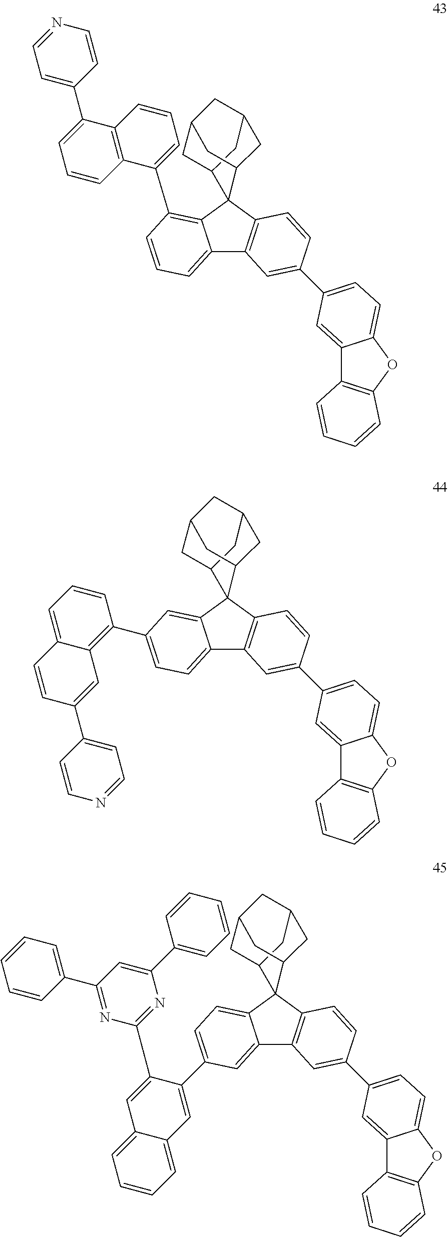 [SMILES: c1cc2c(c(-c3cccc4c(-c5ccncc5)cccc34)c1)C1(c3ccc(-c4ccc5oc6ccccc6c5c4)cc3-2)C2CC3CC(C2)CC1C3 ; c1cc(-c2ccc3c(c2)C2(c4ccc(-c5ccc6oc7ccccc7c6c5)cc4-3)C3CC4CC(C3)CC2C4)c2cc(-c3ccncc3)ccc2c1 ; c1ccc(-c2cc(-c3ccccc3)nc(-c3cc4ccccc4cc3-c3ccc4c(c3)-c3cc(-c5ccc6oc7ccccc7c6c5)ccc3C43C4CC5CC(C4)CC3C5)n2)cc1]

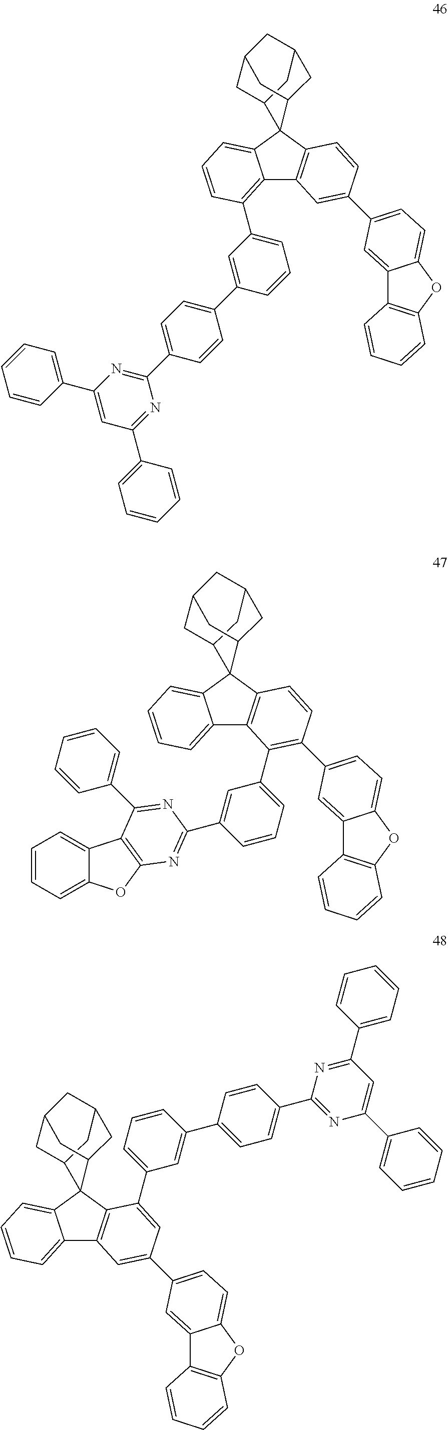 [SMILES: c1ccc(-c2cc(-c3ccccc3)nc(-c3ccc(-c4cccc(-c5cccc6c5-c5cc(-c7ccc8oc9ccccc9c8c7)ccc5C65C6CC7CC(C6)CC5C7)c4)cc3)n2)cc1 ; c1ccc(-c2nc(-c3cccc(-c4c(-c5ccc6oc7ccccc7c6c5)ccc5c4-c4ccccc4C54C5CC6CC(C5)CC4C6)c3)nc3oc4ccccc4c23)cc1 ; c1ccc(-c2cc(-c3ccccc3)nc(-c3ccc(-c4cccc(-c5cc(-c6ccc7oc8ccccc8c7c6)cc6c5C5(c7ccccc7-6)C6CC7CC(C6)CC5C7)c4)cc3)n2)cc1]

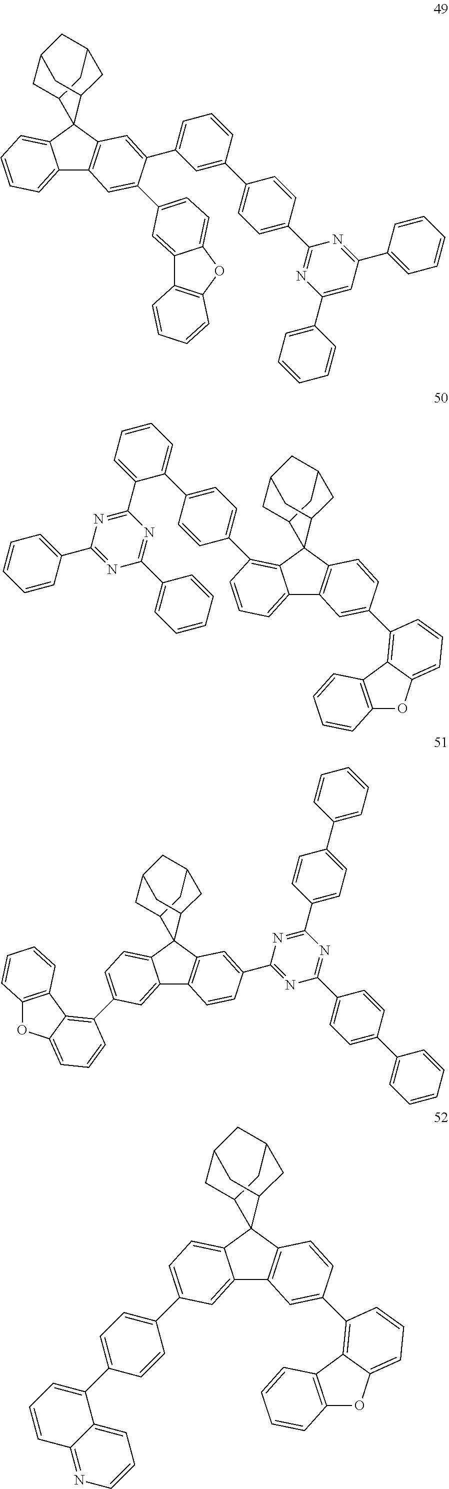

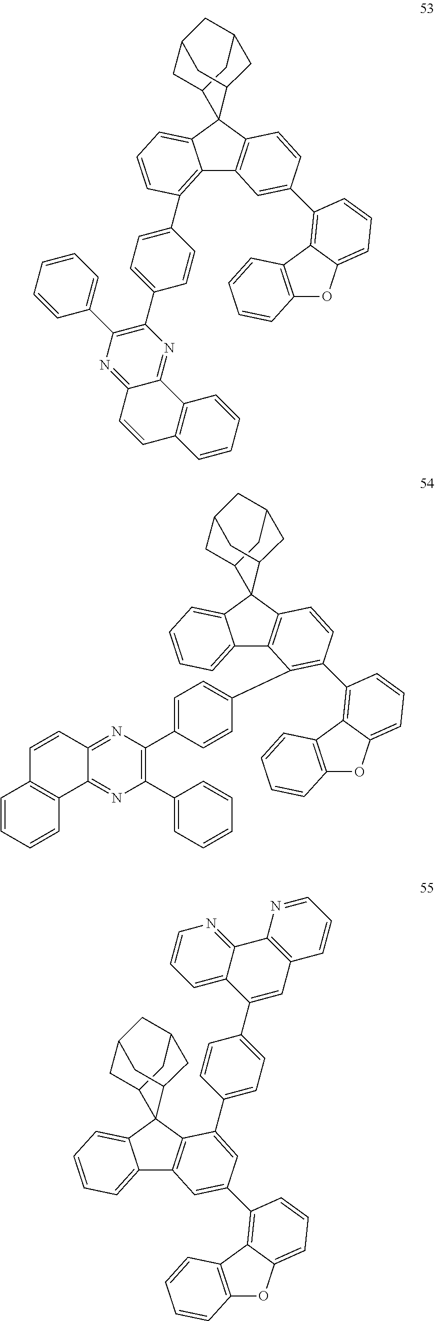

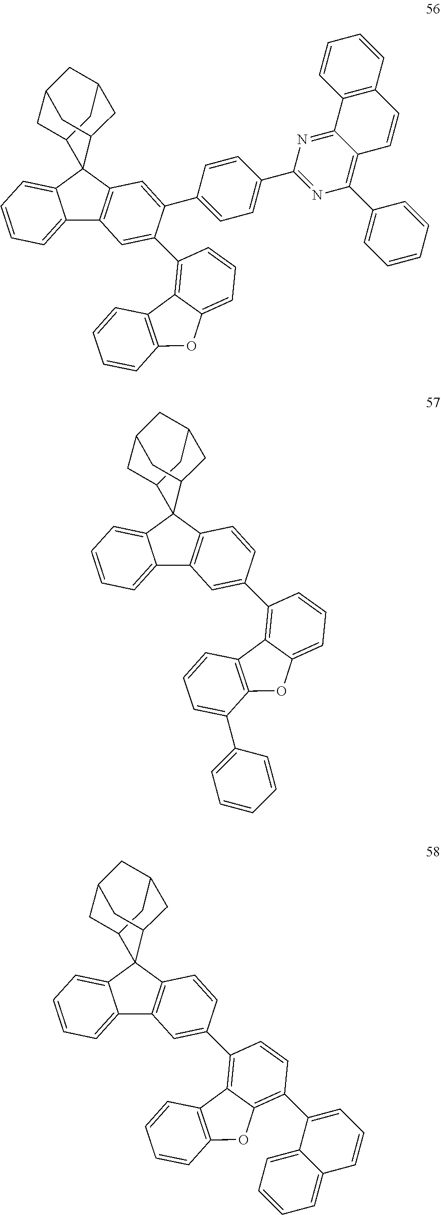 [SMILES: c1ccc(-c2nc(-c3ccc(-c4cc5c(cc4-c4cccc6oc7ccccc7c46)-c4ccccc4C54C5CC6CC(C5)CC4C6)cc3)nc3c2ccc2ccccc23)cc1 ; c1ccc(-c2cccc3c2oc2cccc(-c4ccc5c(c4)-c4ccccc4C54C5CC6CC(C5)CC4C6)c23)cc1 ; c1ccc2c(c1)-c1cc(-c3ccc(-c4cccc5ccccc45)c4oc5ccccc5c34)ccc1C21C2CC3CC(C2)CC1C3]

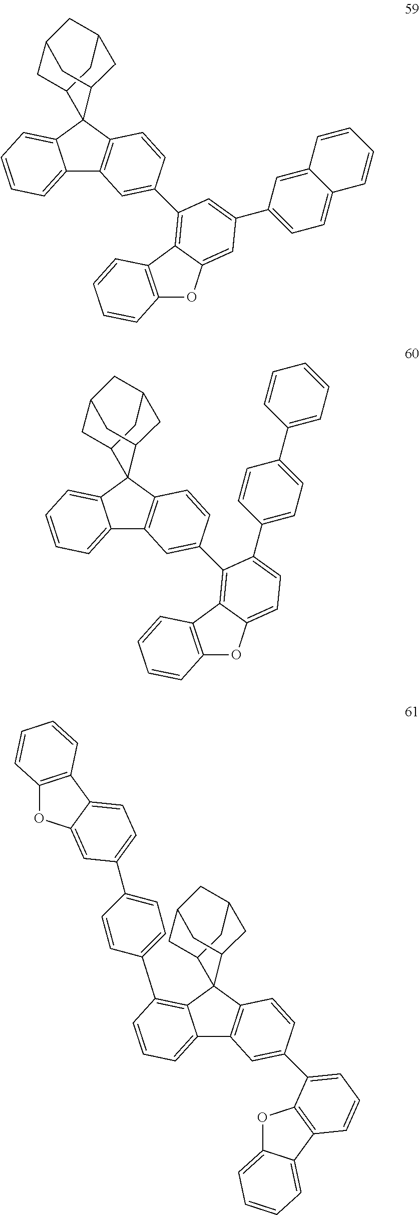 [SMILES: c1ccc2c(c1)-c1cc(-c3cc(-c4ccc5ccccc5c4)cc4oc5ccccc5c34)ccc1C21C2CC3CC(C2)CC1C3 ; c1ccc(-c2ccc(-c3ccc4oc5ccccc5c4c3-c3ccc4c(c3)-c3ccccc3C43C4CC5CC(C4)CC3C5)cc2)cc1 ; c1cc(-c2ccc(-c3ccc4c(c3)oc3ccccc34)cc2)c2c(c1)-c1cc(-c3cccc4c3oc3ccccc34)ccc1C21C2CC3CC(C2)CC1C3]

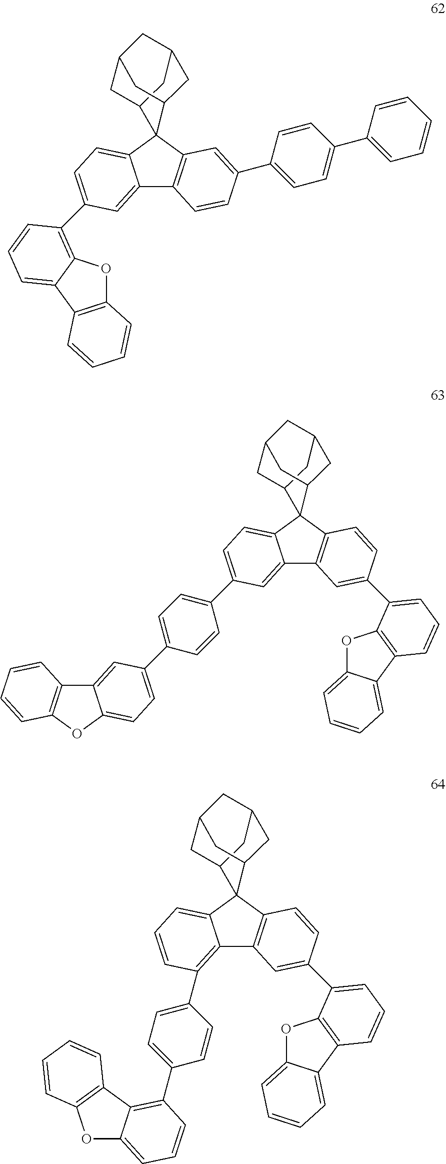 [SMILES: c1ccc(-c2ccc(-c3ccc4c(c3)C3(c5ccc(-c6cccc7c6oc6ccccc67)cc5-4)C4CC5CC(C4)CC3C5)cc2)cc1 ; c1ccc2c(c1)oc1ccc(-c3ccc(-c4ccc5c(c4)-c4cc(-c6cccc7c6oc6ccccc67)ccc4C54C5CC6CC(C5)CC4C6)cc3)cc12 ; c1cc(-c2ccc(-c3cccc4oc5ccccc5c34)cc2)c2c(c1)C1(c3ccc(-c4cccc5c4oc4ccccc45)cc3-2)C2CC3CC(C2)CC1C3]

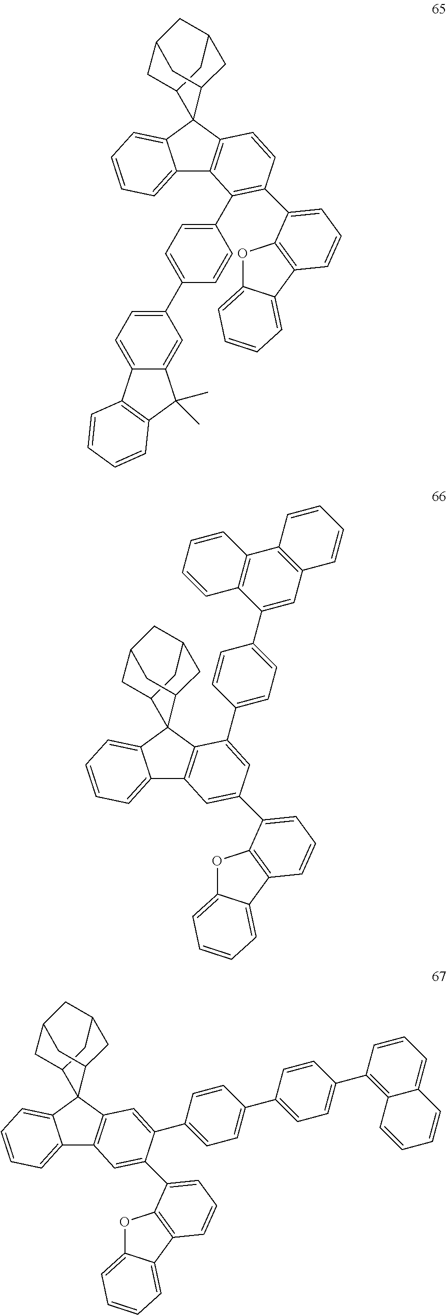 [SMILES: CC1(C)c2ccccc2-c2ccc(-c3ccc(-c4c(-c5cccc6c5oc5ccccc56)ccc5c4-c4ccccc4C54C5CC6CC(C5)CC4C6)cc3)cc21 ; c1ccc2c(c1)-c1cc(-c3cccc4c3oc3ccccc34)cc(-c3ccc(-c4cc5ccccc5c5ccccc45)cc3)c1C21C2CC3CC(C2)CC1C3 ; c1ccc2c(c1)-c1cc(-c3cccc4c3oc3ccccc34)c(-c3ccc(-c4ccc(-c5cccc6ccccc56)cc4)cc3)cc1C21C2CC3CC(C2)CC1C3]

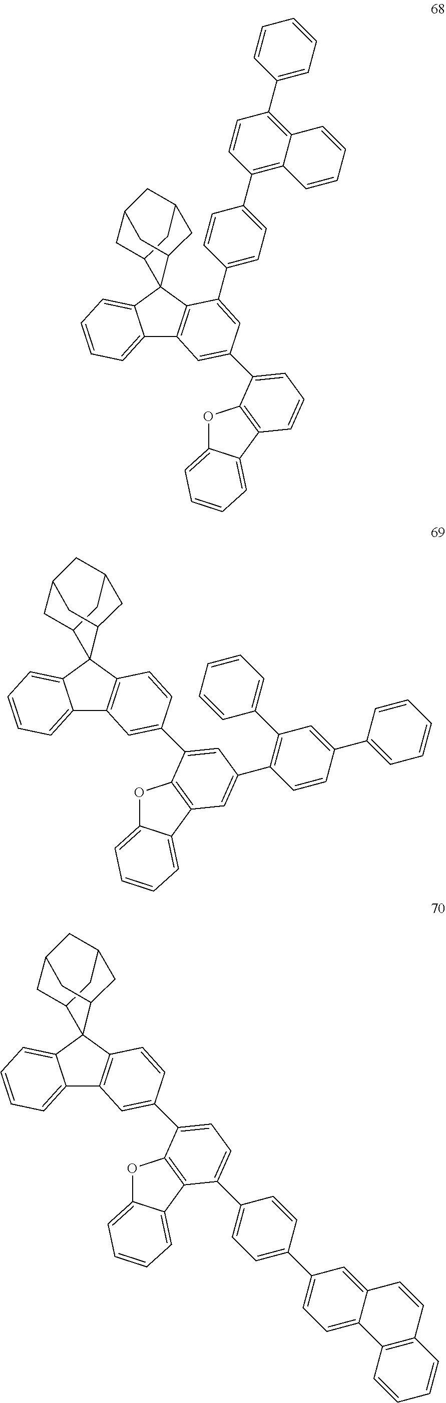 [SMILES: c1ccc(-c2ccc(-c3ccc(-c4cc(-c5cccc6c5oc5ccccc56)cc5c4C4(c6ccccc6-5)C5CC6CC(C5)CC4C6)cc3)c3ccccc23)cc1 ; c1ccc(-c2ccc(-c3cc(-c4ccc5c(c4)-c4ccccc4C54C5CC6CC(C5)CC4C6)c4oc5ccccc5c4c3)c(-c3ccccc3)c2)cc1 ; c1ccc2c(c1)-c1cc(-c3ccc(-c4ccc(-c5ccc6c(ccc7ccccc76)c5)cc4)c4c3oc3ccccc34)ccc1C21C2CC3CC(C2)CC1C3]

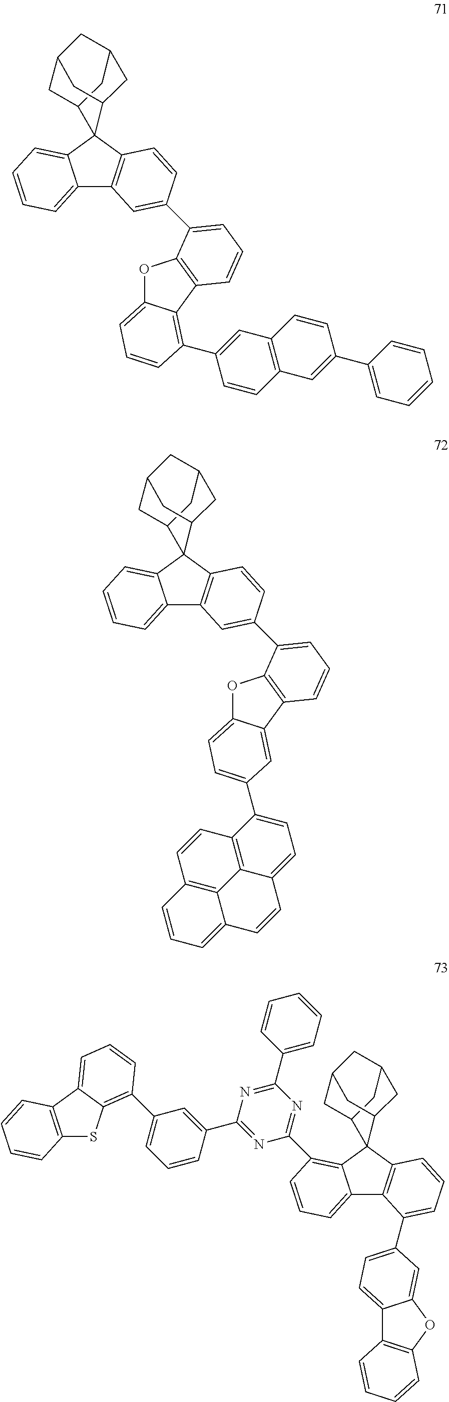 [SMILES: c1ccc(-c2ccc3cc(-c4cccc5oc6c(-c7ccc8c(c7)-c7ccccc7C87C8CC9CC(C8)CC7C9)cccc6c45)ccc3c2)cc1 ; c1ccc2c(c1)-c1cc(-c3cccc4c3oc3ccc(-c5ccc6ccc7cccc8ccc5c6c78)cc34)ccc1C21C2CC3CC(C2)CC1C3 ; c1ccc(-c2nc(-c3cccc(-c4cccc5c4sc4ccccc45)c3)nc(-c3cccc4c3C3(c5cccc(-c6ccc7c(c6)oc6ccccc67)c5-4)C4CC5CC(C4)CC3C5)n2)cc1]

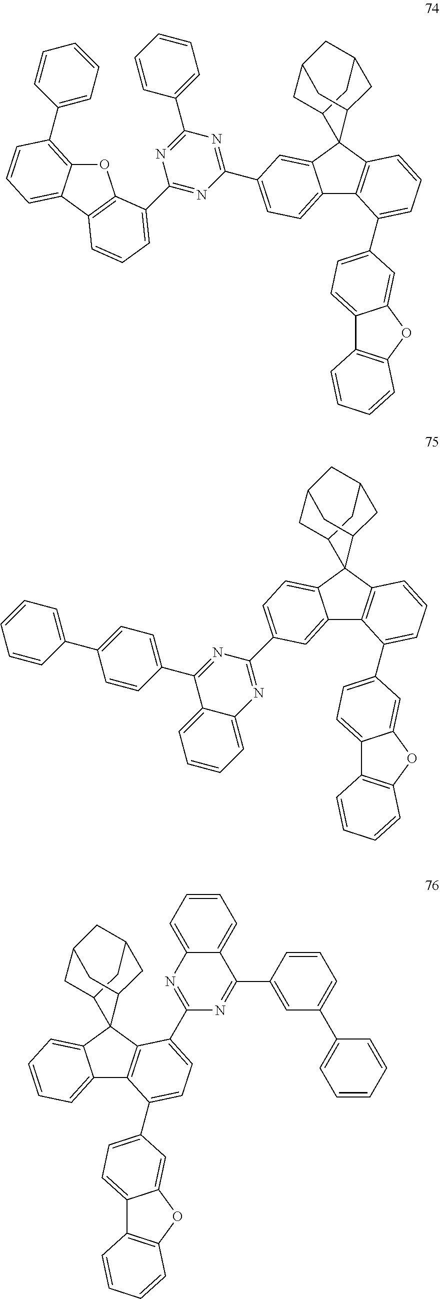 [SMILES: c1ccc(-c2nc(-c3ccc4c(c3)C3(c5cccc(-c6ccc7c(c6)oc6ccccc67)c5-4)C4CC5CC(C4)CC3C5)nc(-c3cccc4c3oc3c(-c5ccccc5)cccc34)n2)cc1 ; c1ccc(-c2ccc(-c3nc(-c4ccc5c(c4)-c4c(-c6ccc7c(c6)oc6ccccc67)cccc4C54C5CC6CC(C5)CC4C6)nc4ccccc34)cc2)cc1 ; c1ccc(-c2cccc(-c3nc(-c4ccc(-c5ccc6c(c5)oc5ccccc56)c5c4C4(c6ccccc6-5)C5CC6CC(C5)CC4C6)nc4ccccc34)c2)cc1]

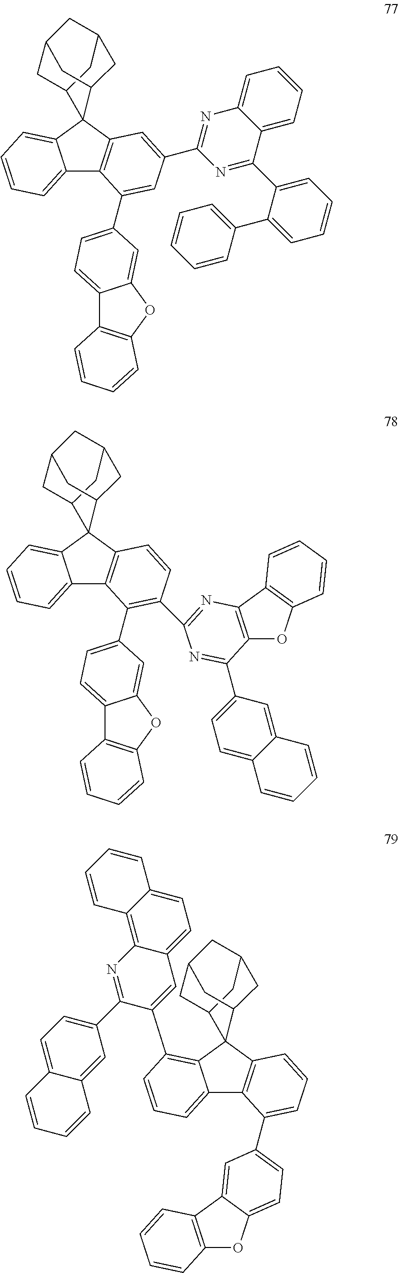

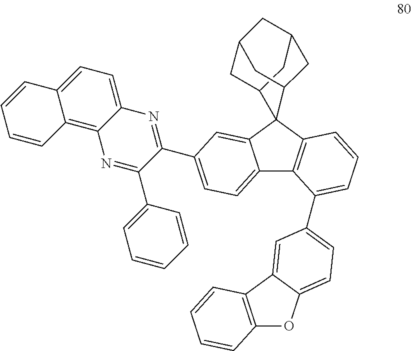 [SMILES: c1ccc(-c2nc3c(ccc4ccccc43)nc2-c2ccc3c(c2)C2(c4cccc(-c5ccc6oc7ccccc7c6c5)c4-3)C3CC4CC(C3)CC2C4)cc1]

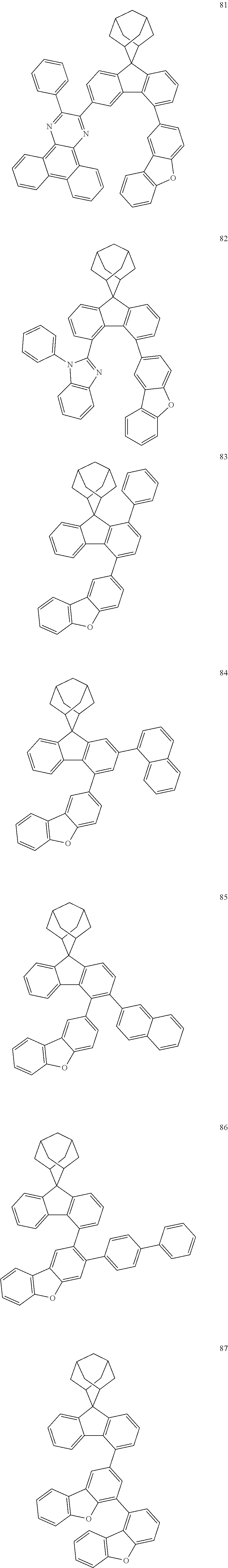

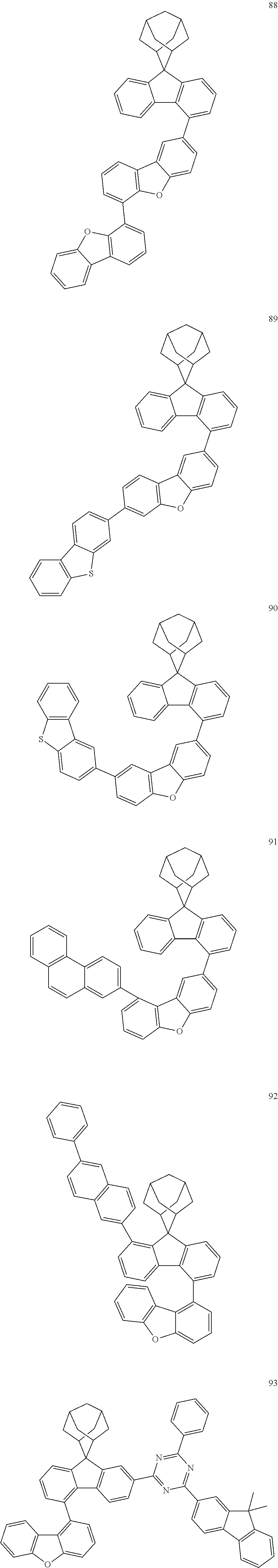

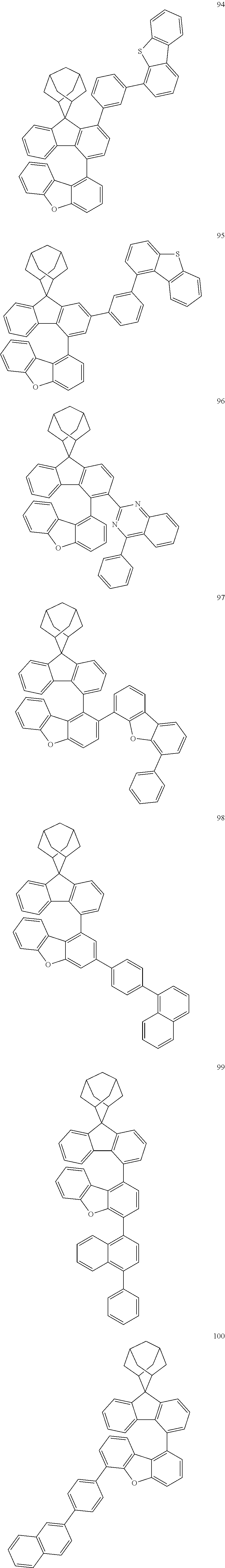

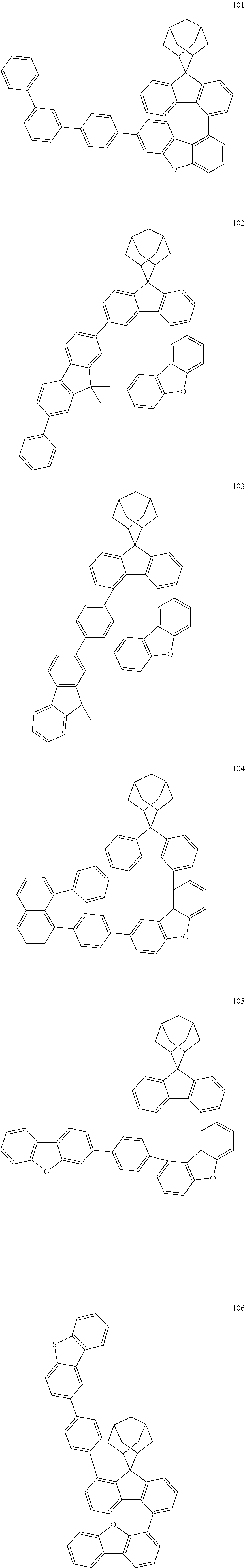

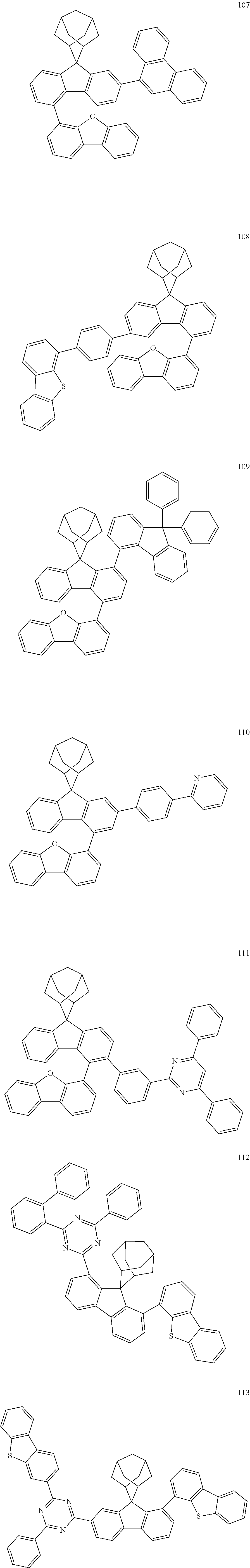

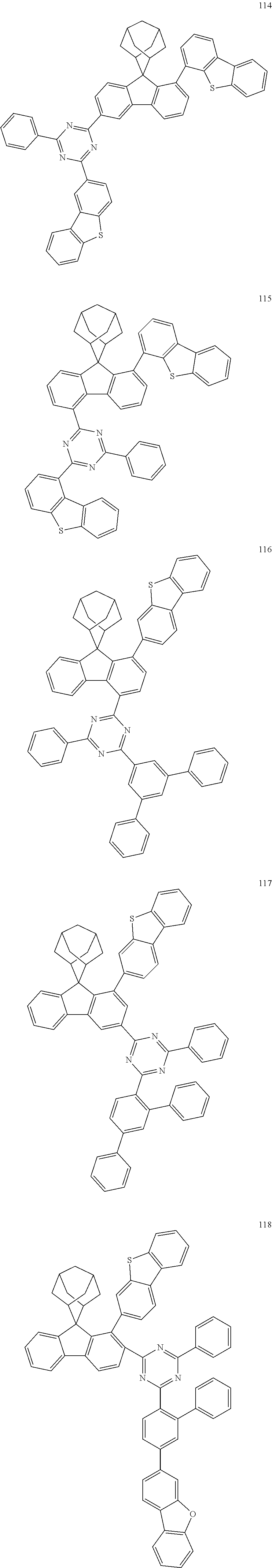

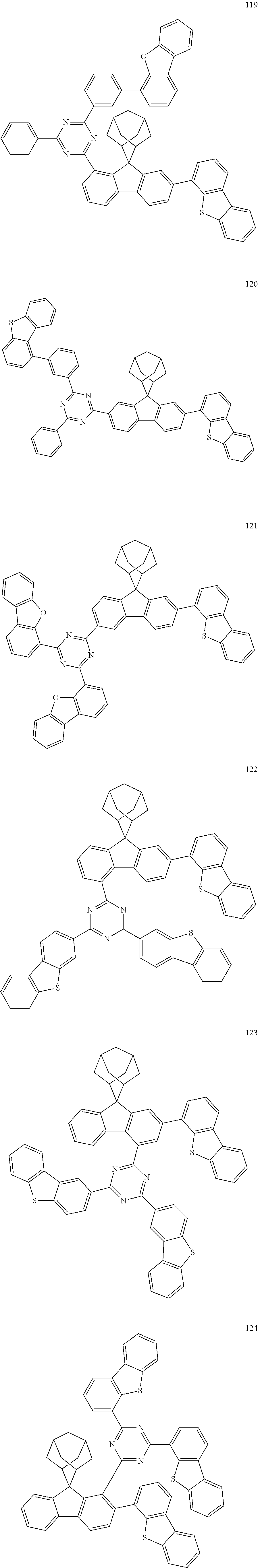

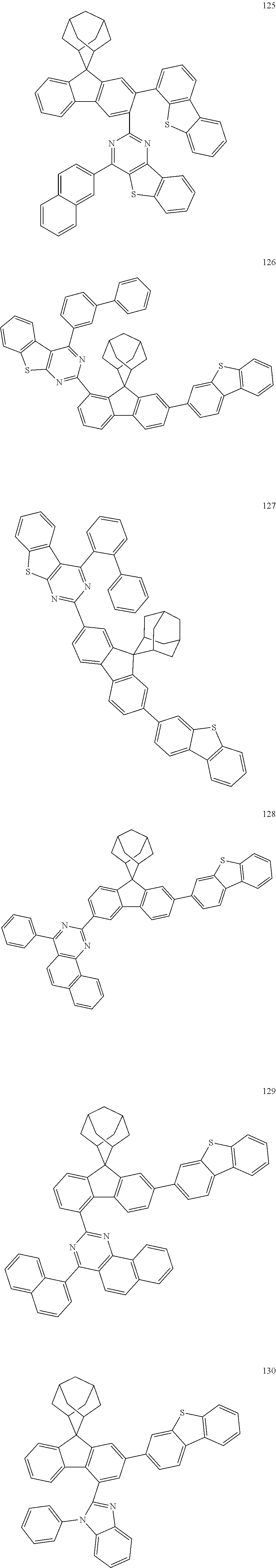

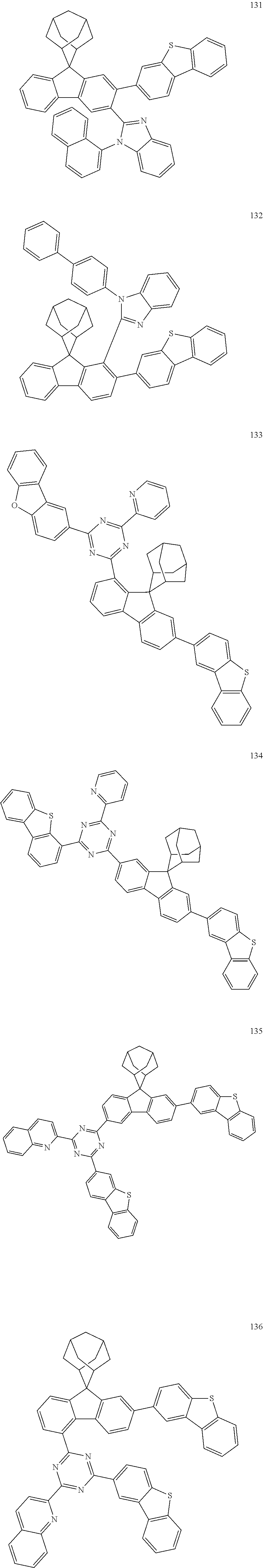

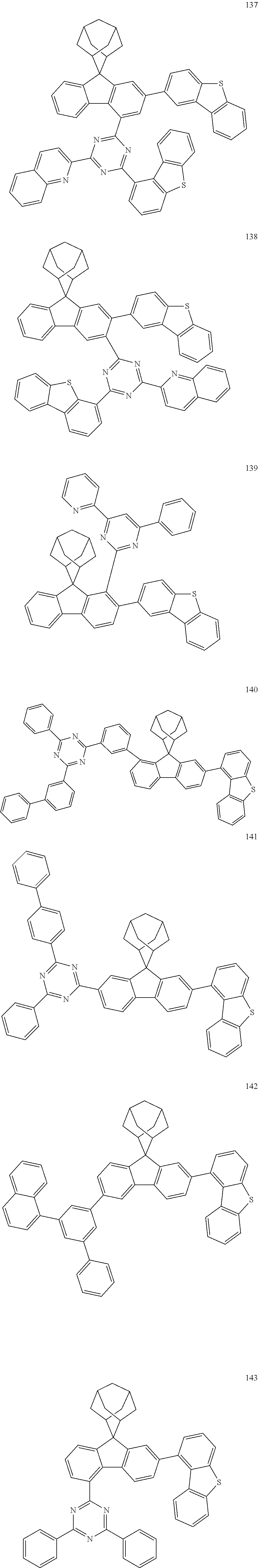

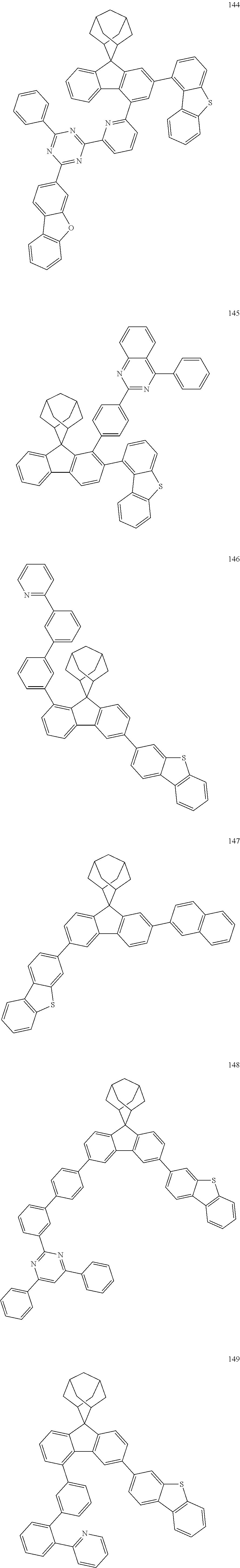

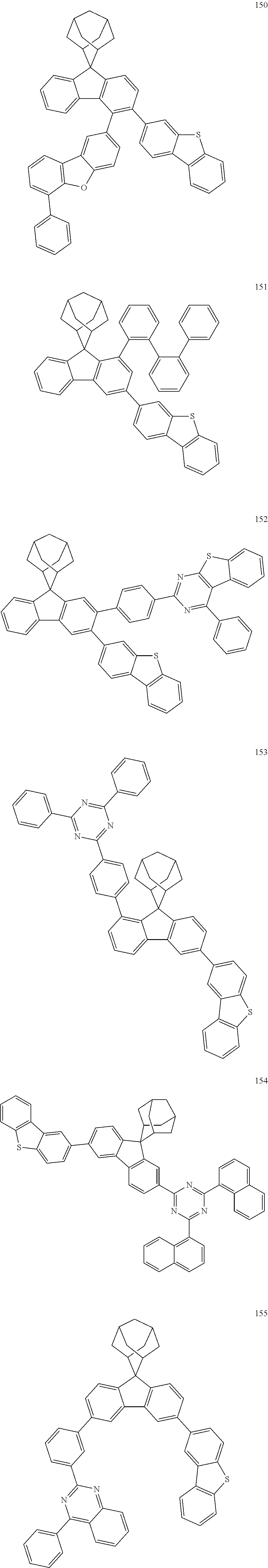

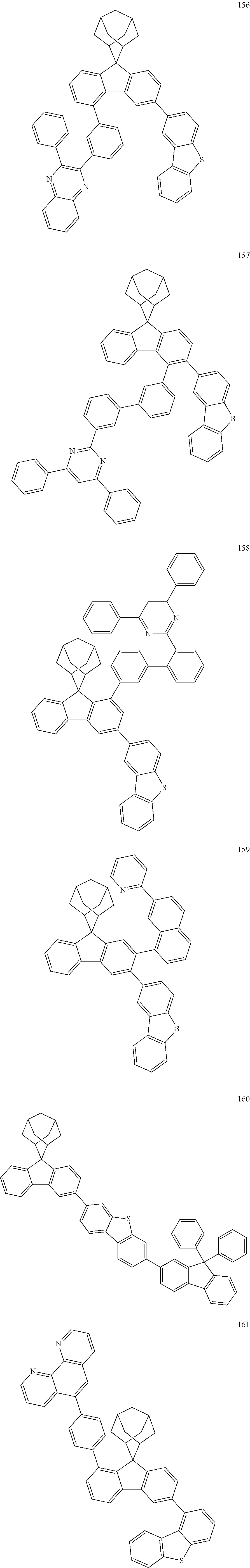

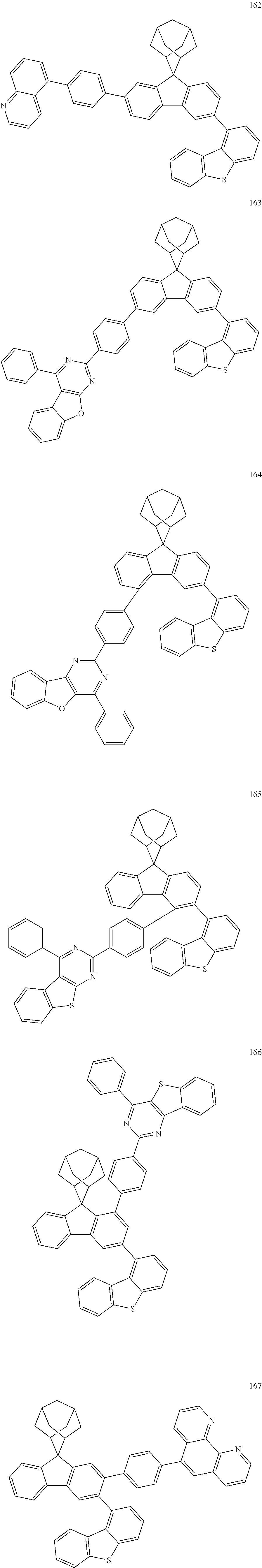

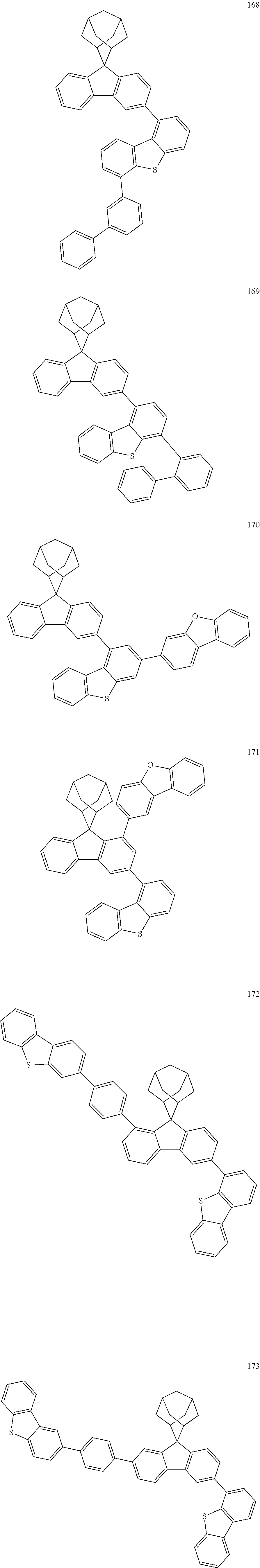

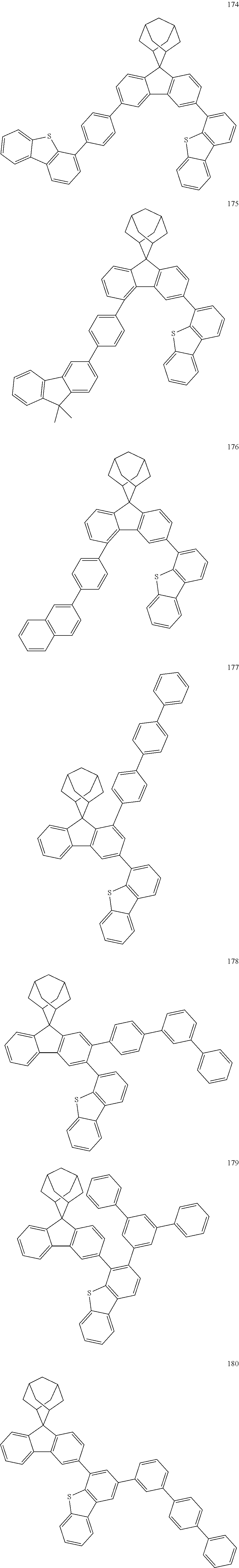

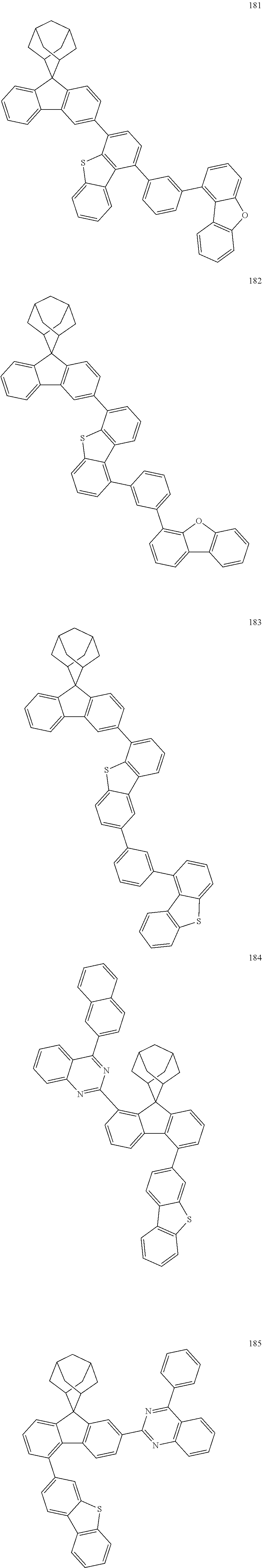 [SMILES: c1cc(-c2cccc3oc4ccccc4c23)cc(-c2ccc(-c3ccc4c(c3)-c3ccccc3C43C4CC5CC(C4)CC3C5)c3sc4ccccc4c23)c1 ; c1cc(-c2cccc3c2oc2ccccc23)cc(-c2cccc3sc4c(-c5ccc6c(c5)-c5ccccc5C65C6CC7CC(C6)CC5C7)cccc4c23)c1 ; c1cc(-c2ccc3sc4c(-c5ccc6c(c5)-c5ccccc5C65C6CC7CC(C6)CC5C7)cccc4c3c2)cc(-c2cccc3sc4ccccc4c23)c1 ; c1cc(-c2nc(-c3ccc4ccccc4c3)c3ccccc3n2)c2c(c1)-c1c(-c3ccc4c(c3)sc3ccccc34)cccc1C21C2CC3CC(C2)CC1C3 ; c1ccc(-c2nc(-c3ccc4c(c3)C3(c5cccc(-c6ccc7c(c6)sc6ccccc67)c5-4)C4CC5CC(C4)CC3C5)nc3ccccc23)cc1]

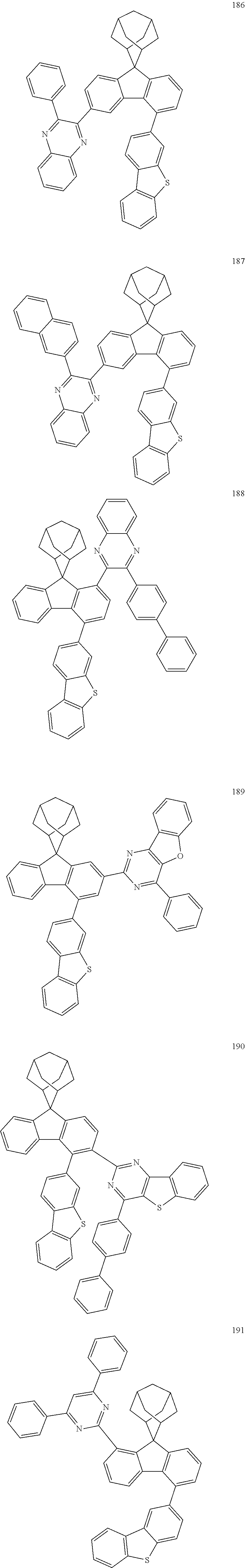

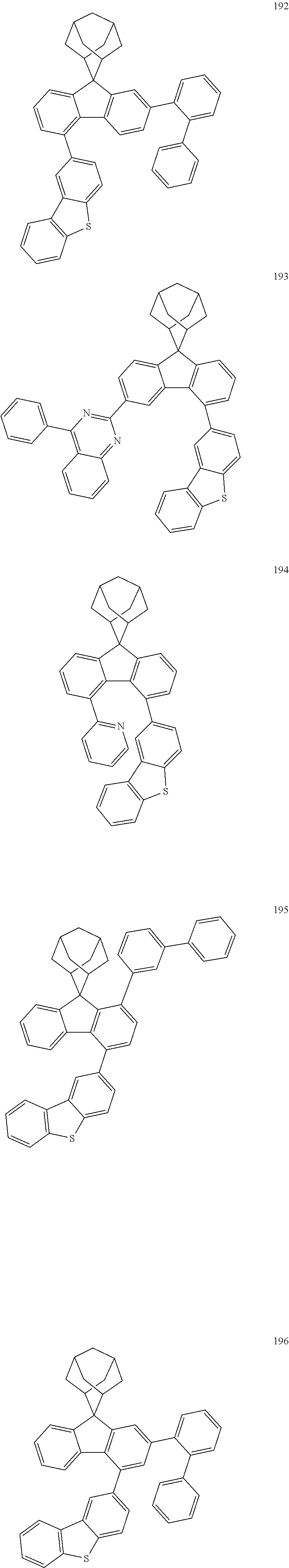 [SMILES: c1ccc(-c2ccccc2-c2ccc3c(c2)C2(c4cccc(-c5ccc6sc7ccccc7c6c5)c4-3)C3CC4CC(C3)CC2C4)cc1 ; c1ccc(-c2nc(-c3ccc4c(c3)-c3c(-c5ccc6sc7ccccc7c6c5)cccc3C43C4CC5CC(C4)CC3C5)nc3ccccc23)cc1 ; c1ccc(-c2cccc3c2-c2c(-c4ccc5sc6ccccc6c5c4)cccc2C32C3CC4CC(C3)CC2C4)nc1 ; c1ccc(-c2cccc(-c3ccc(-c4ccc5sc6ccccc6c5c4)c4c3C3(c5ccccc5-4)C4CC5CC(C4)CC3C5)c2)cc1 ; c1ccc(-c2ccccc2-c2cc(-c3ccc4sc5ccccc5c4c3)c3c(c2)C2(c4ccccc4-3)C3CC4CC(C3)CC2C4)cc1]

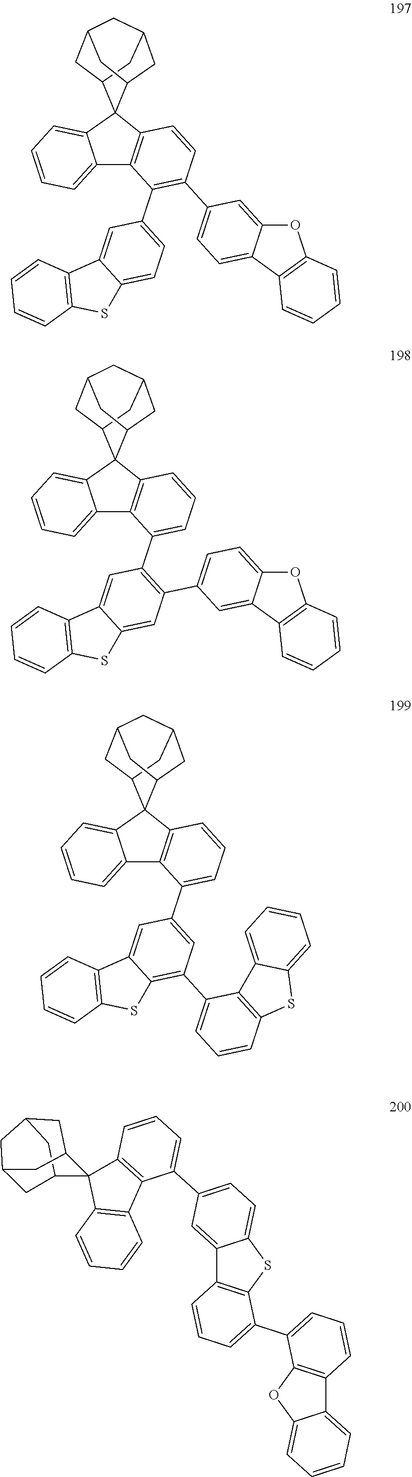 [SMILES: c1ccc2c(c1)-c1c(ccc(-c3ccc4c(c3)oc3ccccc34)c1-c1ccc3sc4ccccc4c3c1)C21C2CC3CC(C2)CC1C3 ; c1ccc2c(c1)-c1c(-c3cc4c(cc3-c3ccc5oc6ccccc6c5c3)sc3ccccc34)cccc1C21C2CC3CC(C2)CC1C3 ; c1ccc2c(c1)-c1c(-c3cc(-c4cccc5sc6ccccc6c45)c4sc5ccccc5c4c3)cccc1C21C2CC3CC(C2)CC1C3 ; c1ccc2c(c1)-c1c(-c3ccc4sc5c(-c6cccc7c6oc6ccccc67)cccc5c4c3)cccc1C21C2CC3CC(C2)CC1C3]

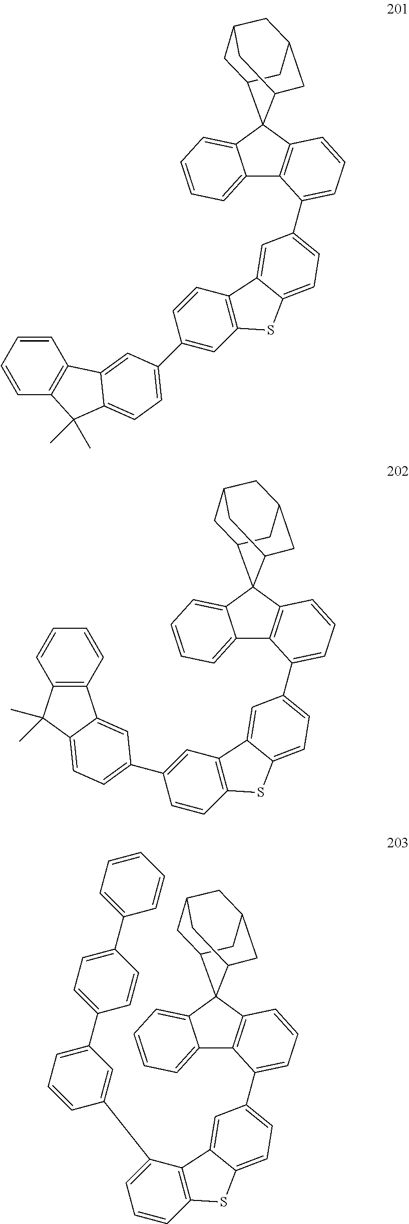 [SMILES: CC1(C)c2ccccc2-c2cc(-c3ccc4c(c3)sc3ccc(-c5cccc6c5-c5ccccc5C65C6CC7CC(C6)CC5C7)cc34)ccc21 ; CC1(C)c2ccccc2-c2cc(-c3ccc4sc5ccc(-c6cccc7c6-c6ccccc6C76C7CC8CC(C7)CC6C8)cc5c4c3)ccc21 ; c1ccc(-c2ccc(-c3cccc(-c4cccc5sc6ccc(-c7cccc8c7-c7ccccc7C87C8CC9CC(C8)CC7C9)cc6c45)c3)cc2)cc1]

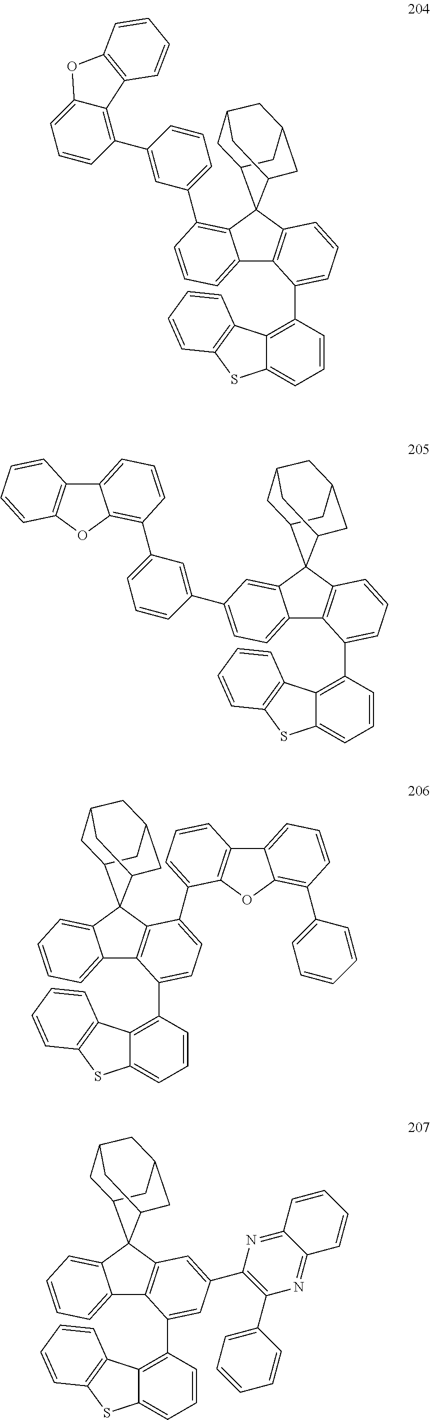 [SMILES: c1cc(-c2cccc3c2C2(c4cccc(-c5cccc6sc7ccccc7c56)c4-3)C3CC4CC(C3)CC2C4)cc(-c2cccc3oc4ccccc4c23)c1 ; c1cc(-c2ccc3c(c2)C2(c4cccc(-c5cccc6sc7ccccc7c56)c4-3)C3CC4CC(C3)CC2C4)cc(-c2cccc3c2oc2ccccc23)c1 ; c1ccc(-c2cccc3c2oc2c(-c4ccc(-c5cccc6sc7ccccc7c56)c5c4C4(c6ccccc6-5)C5CC6CC(C5)CC4C6)cccc23)cc1 ; c1ccc(-c2nc3ccccc3nc2-c2cc(-c3cccc4sc5ccccc5c34)c3c(c2)C2(c4ccccc4-3)C3CC4CC(C3)CC2C4)cc1]

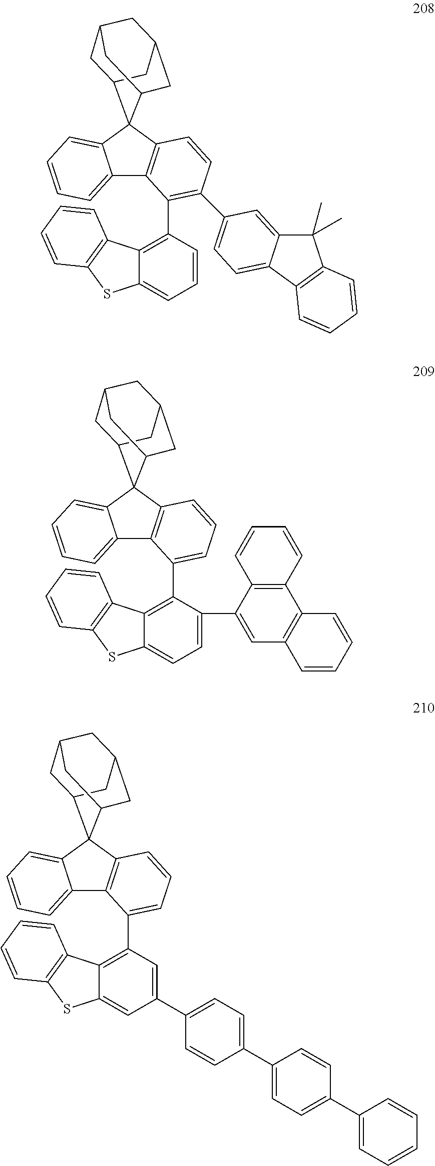 [SMILES: CC1(C)c2ccccc2-c2ccc(-c3ccc4c(c3-c3cccc5sc6ccccc6c35)-c3ccccc3C43C4CC5CC(C4)CC3C5)cc21 ; c1ccc2c(c1)-c1c(-c3c(-c4cc5ccccc5c5ccccc45)ccc4sc5ccccc5c34)cccc1C21C2CC3CC(C2)CC1C3 ; c1ccc(-c2ccc(-c3ccc(-c4cc(-c5cccc6c5-c5ccccc5C65C6CC7CC(C6)CC5C7)c5c(c4)sc4ccccc45)cc3)cc2)cc1]

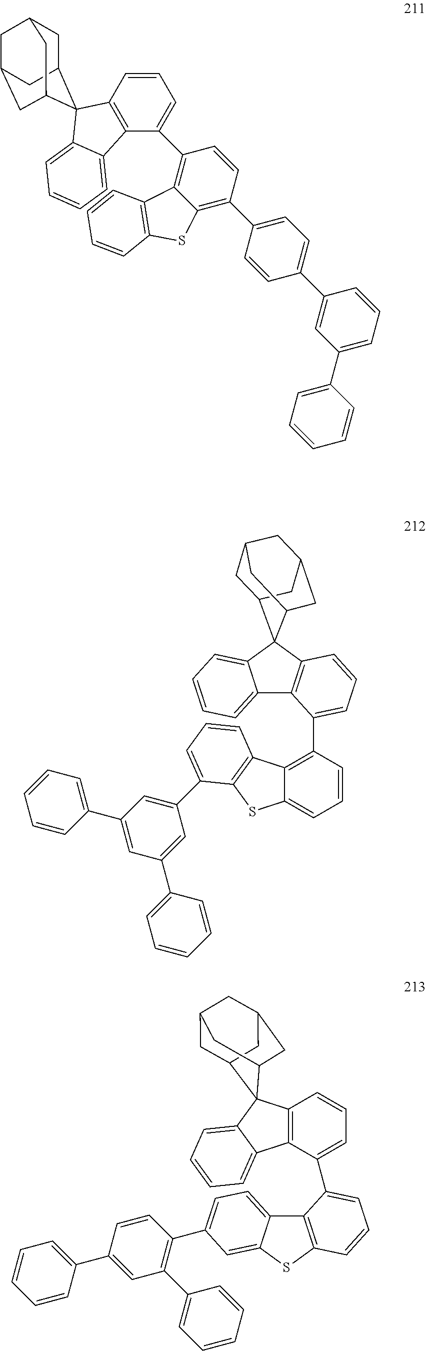 [SMILES: c1ccc(-c2cccc(-c3ccc(-c4ccc(-c5cccc6c5-c5ccccc5C65C6CC7CC(C6)CC5C7)c5c4sc4ccccc45)cc3)c2)cc1 ; c1ccc(-c2cc(-c3ccccc3)cc(-c3cccc4c3sc3cccc(-c5cccc6c5-c5ccccc5C65C6CC7CC(C6)CC5C7)c34)c2)cc1 ; c1ccc(-c2ccc(-c3ccc4c(c3)sc3cccc(-c5cccc6c5-c5ccccc5C65C6CC7CC(C6)CC5C7)c34)c(-c3ccccc3)c2)cc1]

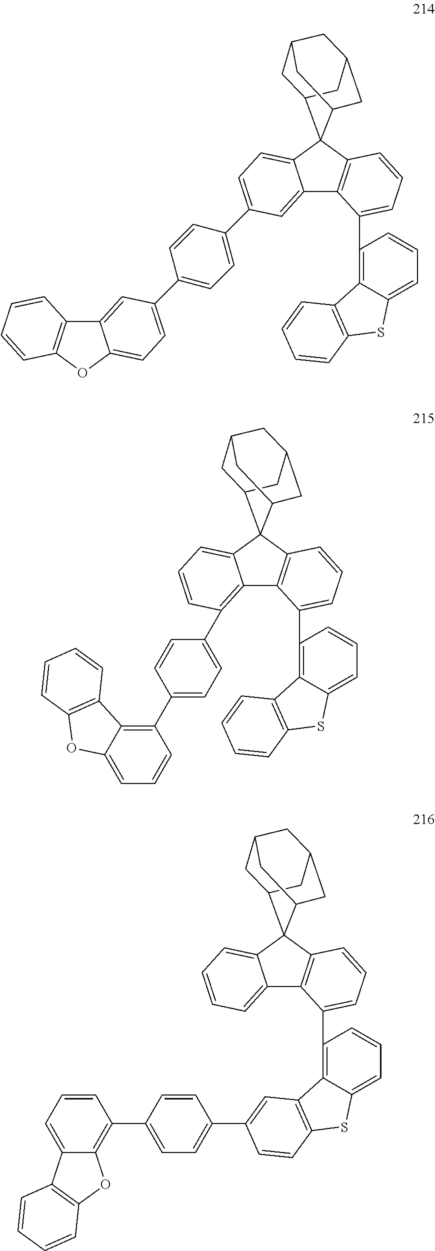 [SMILES: c1cc(-c2cccc3sc4ccccc4c23)c2c(c1)C1(c3ccc(-c4ccc(-c5ccc6oc7ccccc7c6c5)cc4)cc3-2)C2CC3CC(C2)CC1C3 ; c1cc(-c2ccc(-c3cccc4oc5ccccc5c34)cc2)c2c(c1)C1(c3cccc(-c4cccc5sc6ccccc6c45)c3-2)C2CC3CC(C2)CC1C3 ; c1ccc2c(c1)-c1c(-c3cccc4sc5ccc(-c6ccc(-c7cccc8c7oc7ccccc78)cc6)cc5c34)cccc1C21C2CC3CC(C2)CC1C3]

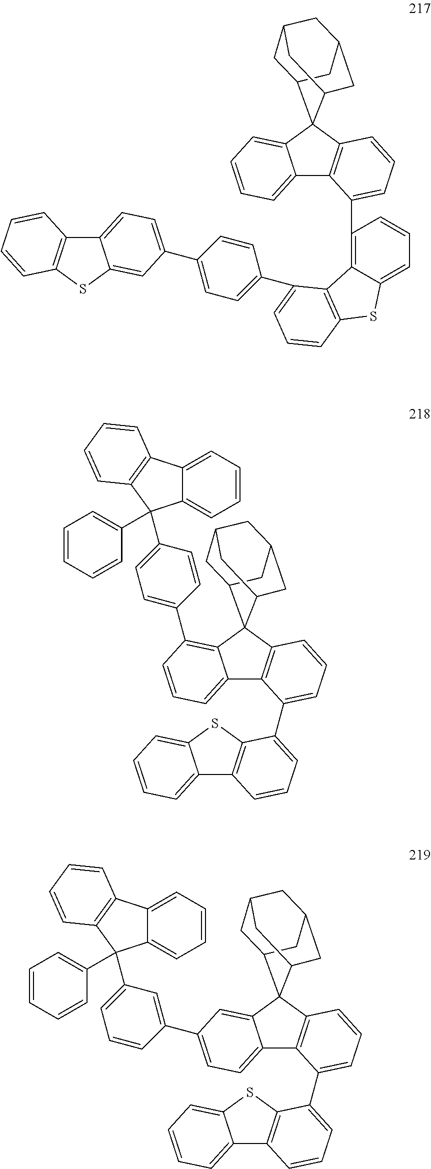 [SMILES: c1ccc2c(c1)-c1c(-c3cccc4sc5cccc(-c6ccc(-c7ccc8c(c7)sc7ccccc78)cc6)c5c34)cccc1C21C2CC3CC(C2)CC1C3 ; c1ccc(C2(c3ccc(-c4cccc5c4C4(c6cccc(-c7cccc8c7sc7ccccc78)c6-5)C5CC6CC(C5)CC4C6)cc3)c3ccccc3-c3ccccc32)cc1 ; c1ccc(C2(c3cccc(-c4ccc5c(c4)C4(c6cccc(-c7cccc8c7sc7ccccc78)c6-5)C5CC6CC(C5)CC4C6)c3)c3ccccc3-c3ccccc32)cc1]

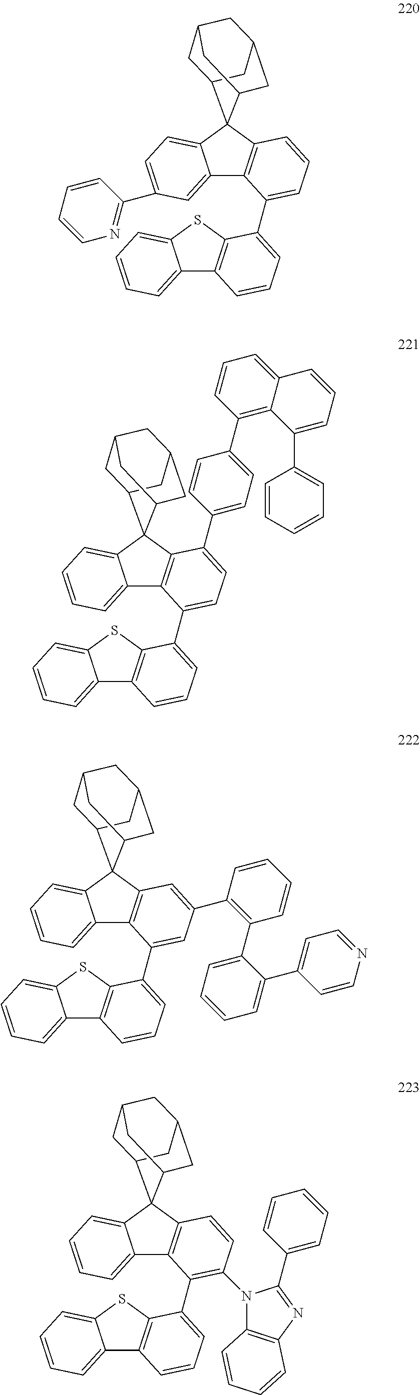 [SMILES: c1ccc(-c2ccc3c(c2)-c2c(-c4cccc5c4sc4ccccc45)cccc2C32C3CC4CC(C3)CC2C4)nc1 ; c1ccc(-c2cccc3cccc(-c4ccc(-c5ccc(-c6cccc7c6sc6ccccc67)c6c5C5(c7ccccc7-6)C6CC7CC(C6)CC5C7)cc4)c23)cc1 ; c1ccc(-c2ccccc2-c2cc(-c3cccc4c3sc3ccccc34)c3c(c2)C2(c4ccccc4-3)C3CC4CC(C3)CC2C4)c(-c2ccncc2)c1 ; c1ccc(-c2nc3ccccc3n2-c2ccc3c(c2-c2cccc4c2sc2ccccc24)-c2ccccc2C32C3CC4CC(C3)CC2C4)cc1]

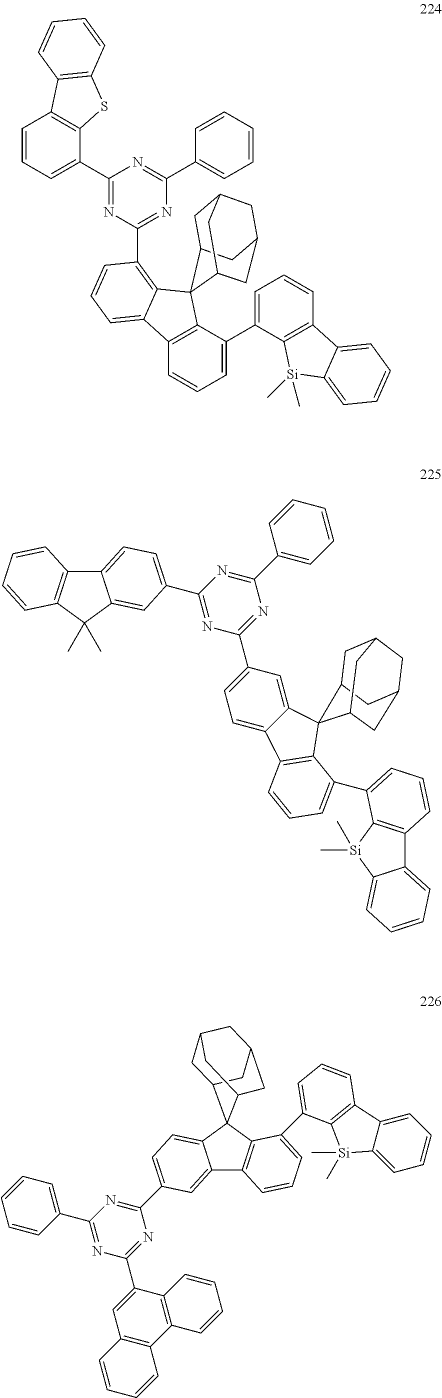 [SMILES: C[Si]1(C)c2ccccc2-c2cccc(-c3cccc4c3C3(c5c(-c6nc(-c7ccccc7)nc(-c7cccc8c7sc7ccccc78)n6)cccc5-4)C4CC5CC(C4)CC3C5)c21 ; CC1(C)c2ccccc2-c2ccc(-c3nc(-c4ccccc4)nc(-c4ccc5c(c4)C4(c6c-5cccc6-c5cccc6c5[Si](C)(C)c5ccccc5-6)C5CC6CC(C5)CC4C6)n3)cc21 ; C[Si]1(C)c2ccccc2-c2cccc(-c3cccc4c3C3(c5ccc(-c6nc(-c7ccccc7)nc(-c7cc8ccccc8c8ccccc78)n6)cc5-4)C4CC5CC(C4)CC3C5)c21]

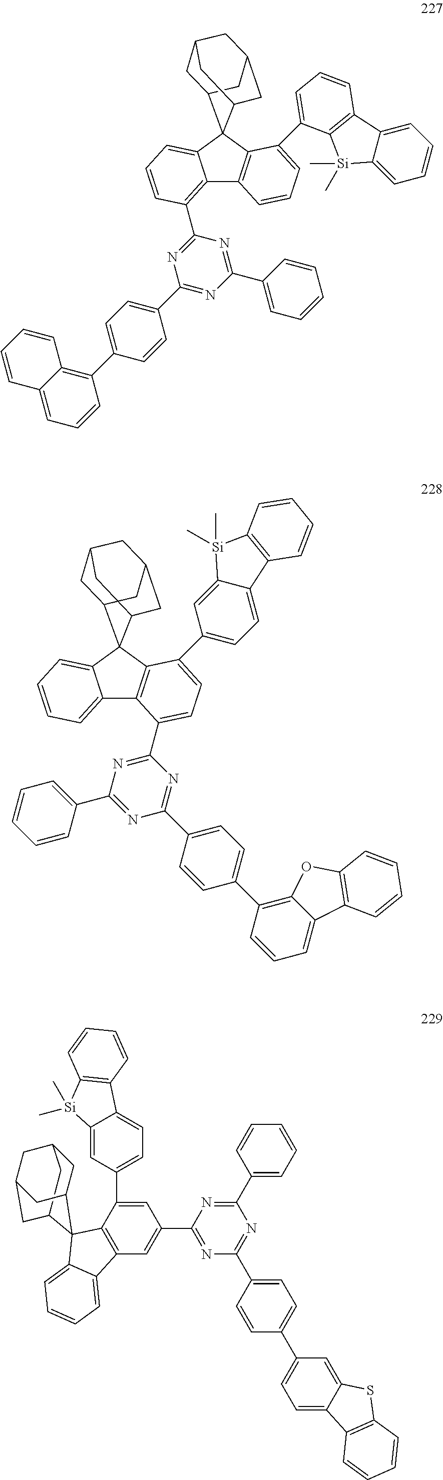 [SMILES: C[Si]1(C)c2ccccc2-c2cccc(-c3cccc4c3C3(c5cccc(-c6nc(-c7ccccc7)nc(-c7ccc(-c8cccc9ccccc89)cc7)n6)c5-4)C4CC5CC(C4)CC3C5)c21 ; C[Si]1(C)c2ccccc2-c2ccc(-c3ccc(-c4nc(-c5ccccc5)nc(-c5ccc(-c6cccc7c6oc6ccccc67)cc5)n4)c4c3C3(c5ccccc5-4)C4CC5CC(C4)CC3C5)cc21 ; C[Si]1(C)c2ccccc2-c2ccc(-c3cc(-c4nc(-c5ccccc5)nc(-c5ccc(-c6ccc7c(c6)sc6ccccc67)cc5)n4)cc4c3C3(c5ccccc5-4)C4CC5CC(C4)CC3C5)cc21]

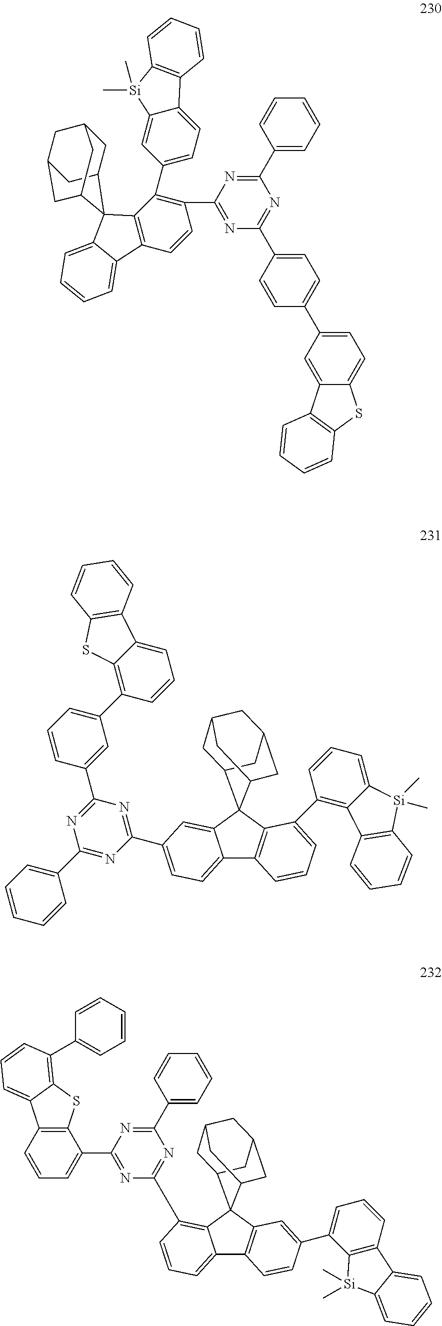 [SMILES: C[Si]1(C)c2ccccc2-c2ccc(-c3c(-c4nc(-c5ccccc5)nc(-c5ccc(-c6ccc7sc8ccccc8c7c6)cc5)n4)ccc4c3C3(c5ccccc5-4)C4CC5CC(C4)CC3C5)cc21 ; C[Si]1(C)c2ccccc2-c2c(-c3cccc4c3C3(c5cc(-c6nc(-c7ccccc7)nc(-c7cccc(-c8cccc9c8sc8ccccc89)c7)n6)ccc5-4)C4CC5CC(C4)CC3C5)cccc21 ; C[Si]1(C)c2ccccc2-c2cccc(-c3ccc4c(c3)C3(c5c(-c6nc(-c7ccccc7)nc(-c7cccc8c7sc7c(-c9ccccc9)cccc78)n6)cccc5-4)C4CC5CC(C4)CC3C5)c21]

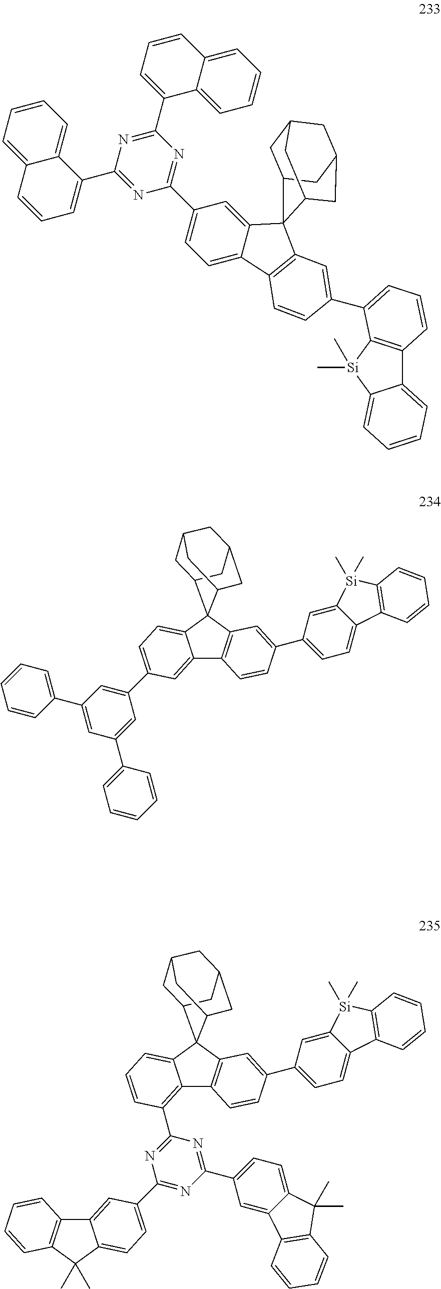 [SMILES: C[Si]1(C)c2ccccc2-c2cccc(-c3ccc4c(c3)C3(c5cc(-c6nc(-c7cccc8ccccc78)nc(-c7cccc8ccccc78)n6)ccc5-4)C4CC5CC(C4)CC3C5)c21 ; C[Si]1(C)c2ccccc2-c2ccc(-c3ccc4c(c3)C3(c5ccc(-c6cc(-c7ccccc7)cc(-c7ccccc7)c6)cc5-4)C4CC5CC(C4)CC3C5)cc21 ; CC1(C)c2ccccc2-c2cc(-c3nc(-c4ccc5c(c4)-c4ccccc4C5(C)C)nc(-c4cccc5c4-c4ccc(-c6ccc7c(c6)[Si](C)(C)c6ccccc6-7)cc4C54C5CC6CC(C5)CC4C6)n3)ccc21]

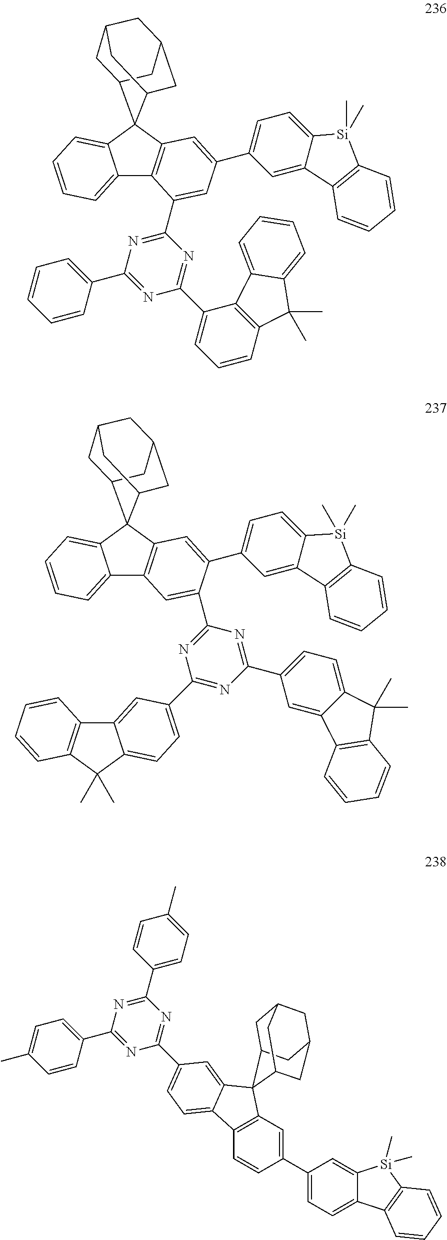

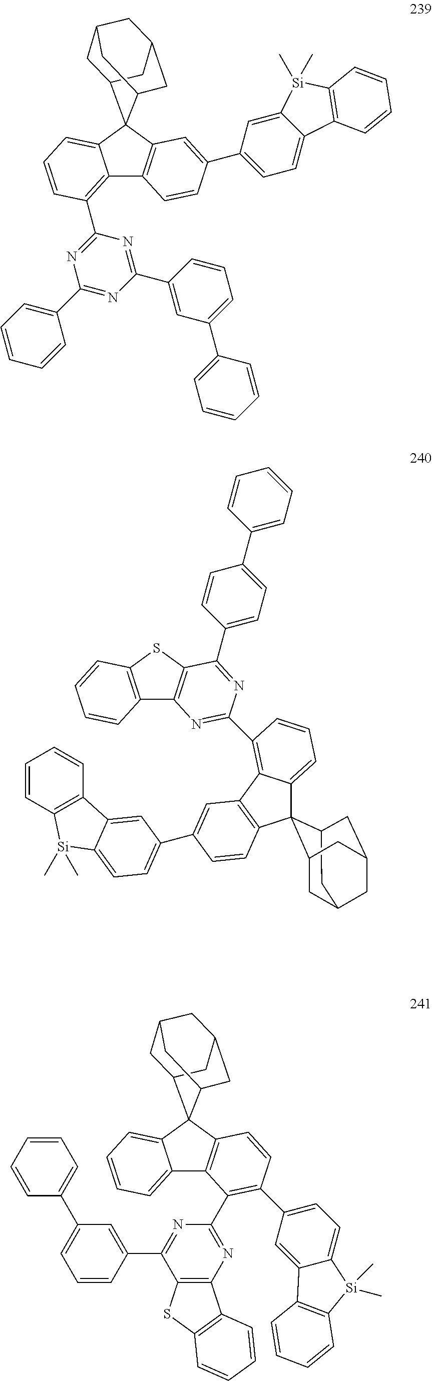 [SMILES: C[Si]1(C)c2ccccc2-c2ccc(-c3ccc4c(c3)C3(c5cccc(-c6nc(-c7ccccc7)nc(-c7cccc(-c8ccccc8)c7)n6)c5-4)C4CC5CC(C4)CC3C5)cc21 ; C[Si]1(C)c2ccccc2-c2cc(-c3ccc4c(c3)-c3c(-c5nc(-c6ccc(-c7ccccc7)cc6)c6sc7ccccc7c6n5)cccc3C43C4CC5CC(C4)CC3C5)ccc21 ; C[Si]1(C)c2ccccc2-c2cc(-c3ccc4c(c3-c3nc(-c5cccc(-c6ccccc6)c5)c5sc6ccccc6c5n3)-c3ccccc3C43C4CC5CC(C4)CC3C5)ccc21]

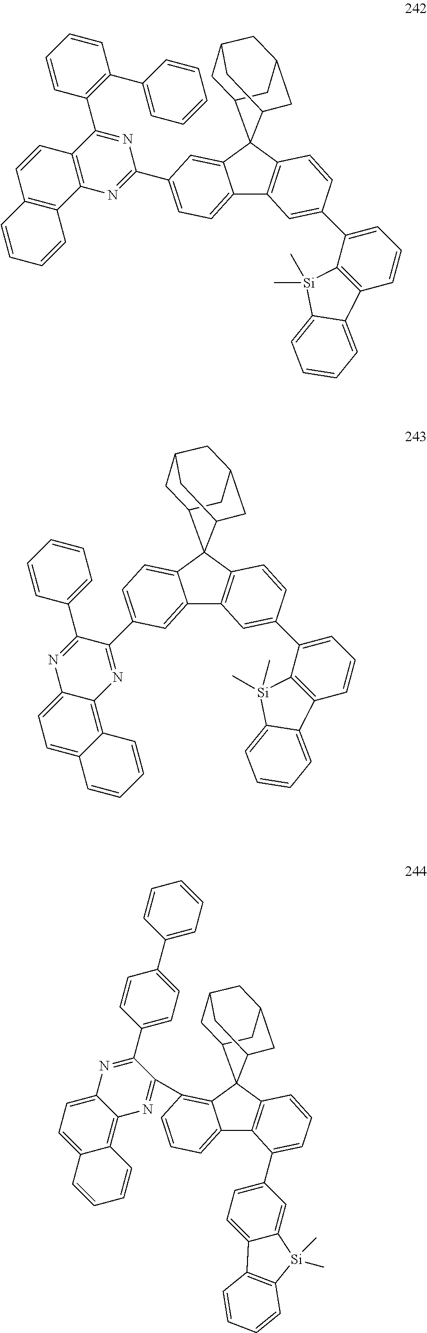 [SMILES: C[Si]1(C)c2ccccc2-c2cccc(-c3ccc4c(c3)-c3ccc(-c5nc(-c6ccccc6-c6ccccc6)c6ccc7ccccc7c6n5)cc3C43C4CC5CC(C4)CC3C5)c21 ; C[Si]1(C)c2ccccc2-c2cccc(-c3ccc4c(c3)-c3cc(-c5nc6c(ccc7ccccc76)nc5-c5ccccc5)ccc3C43C4CC5CC(C4)CC3C5)c21 ; C[Si]1(C)c2ccccc2-c2ccc(-c3cccc4c3-c3cccc(-c5nc6c(ccc7ccccc76)nc5-c5ccc(-c6ccccc6)cc5)c3C43C4CC5CC(C4)CC3C5)cc21]

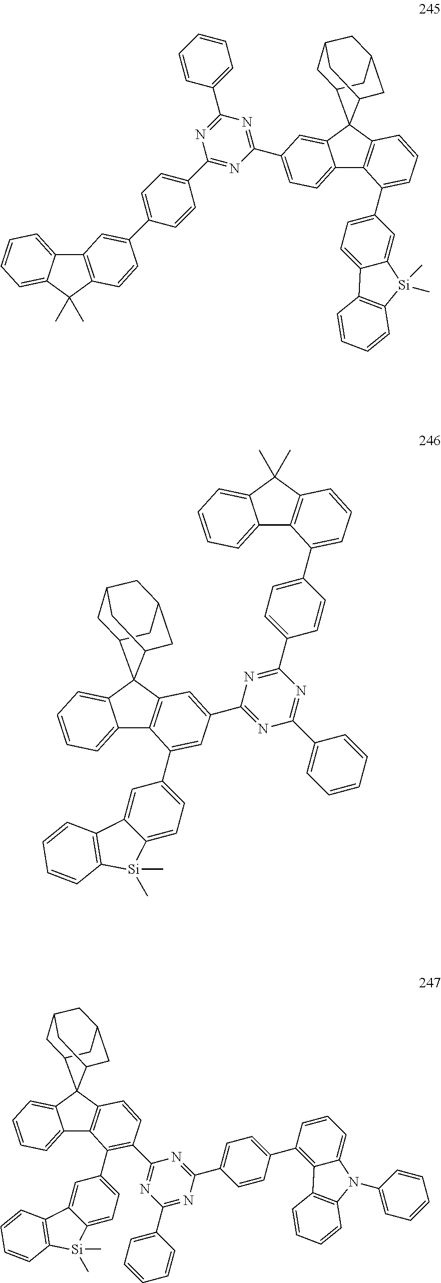 [SMILES: CC1(C)c2ccccc2-c2cc(-c3ccc(-c4nc(-c5ccccc5)nc(-c5ccc6c(c5)C5(c7cccc(-c8ccc9c(c8)[Si](C)(C)c8ccccc8-9)c7-6)C6CC7CC(C6)CC5C7)n4)cc3)ccc21 ; CC1(C)c2ccccc2-c2c(-c3ccc(-c4nc(-c5ccccc5)nc(-c5cc(-c6ccc7c(c6)-c6ccccc6[Si]7(C)C)c6c(c5)C5(c7ccccc7-6)C6CC7CC(C6)CC5C7)n4)cc3)cccc21 ; C[Si]1(C)c2ccccc2-c2cc(-c3c(-c4nc(-c5ccccc5)nc(-c5ccc(-c6cccc7c6c6ccccc6n7-c6ccccc6)cc5)n4)ccc4c3-c3ccccc3C43C4CC5CC(C4)CC3C5)ccc21]

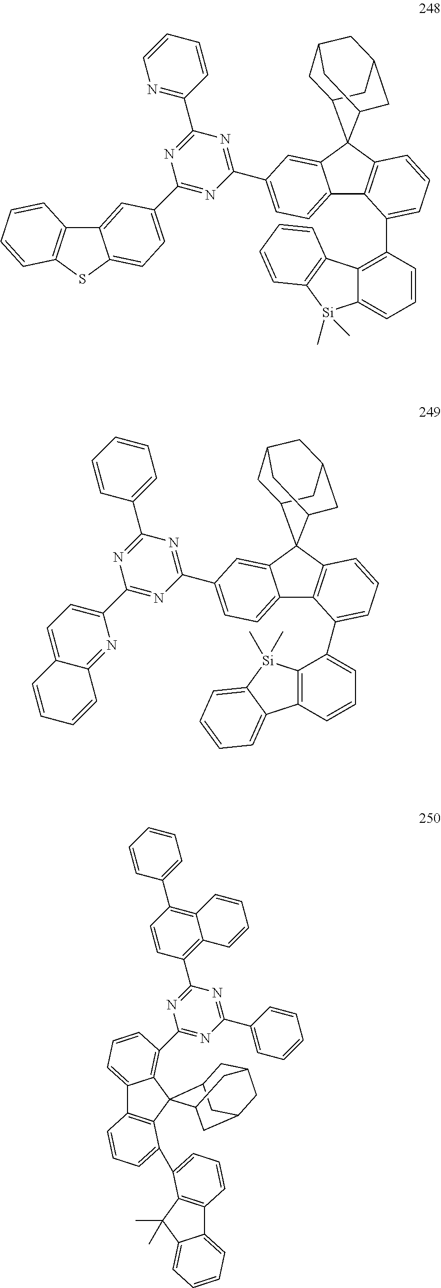 [SMILES: C[Si]1(C)c2ccccc2-c2c(-c3cccc4c3-c3ccc(-c5nc(-c6ccc7sc8ccccc8c7c6)nc(-c6ccccn6)n5)cc3C43C4CC5CC(C4)CC3C5)cccc21 ; C[Si]1(C)c2ccccc2-c2cccc(-c3cccc4c3-c3ccc(-c5nc(-c6ccccc6)nc(-c6ccc7ccccc7n6)n5)cc3C43C4CC5CC(C4)CC3C5)c21 ; CC1(C)c2ccccc2-c2cccc(-c3cccc4c3C3(c5c(-c6nc(-c7ccccc7)nc(-c7ccc(-c8ccccc8)c8ccccc78)n6)cccc5-4)C4CC5CC(C4)CC3C5)c21]

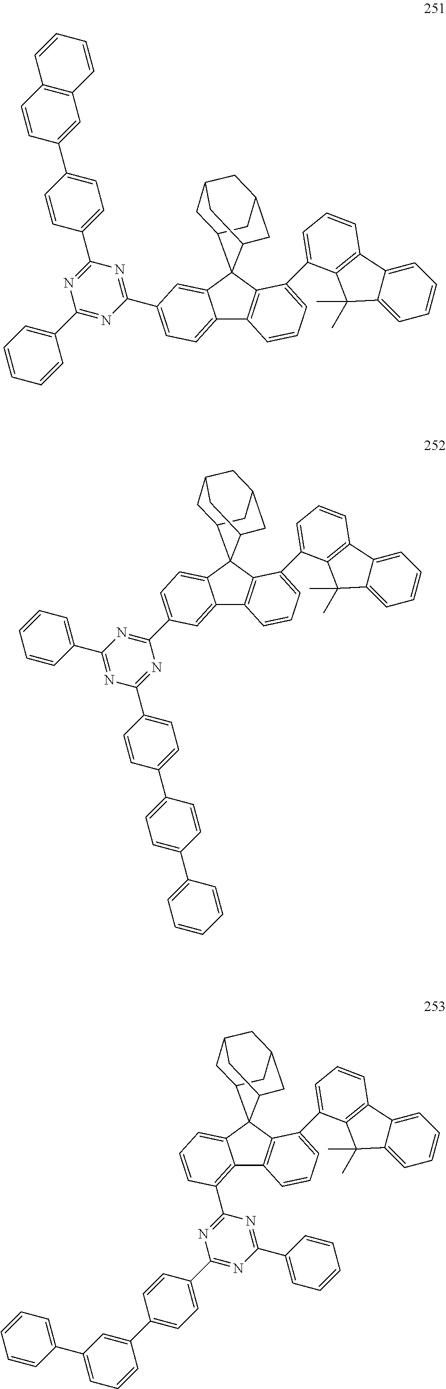 [SMILES: CC1(C)c2ccccc2-c2cccc(-c3cccc4c3C3(c5cc(-c6nc(-c7ccccc7)nc(-c7ccc(-c8ccc9ccccc9c8)cc7)n6)ccc5-4)C4CC5CC(C4)CC3C5)c21 ; CC1(C)c2ccccc2-c2cccc(-c3cccc4c3C3(c5ccc(-c6nc(-c7ccccc7)nc(-c7ccc(-c8ccc(-c9ccccc9)cc8)cc7)n6)cc5-4)C4CC5CC(C4)CC3C5)c21 ; CC1(C)c2ccccc2-c2cccc(-c3cccc4c3C3(c5cccc(-c6nc(-c7ccccc7)nc(-c7ccc(-c8cccc(-c9ccccc9)c8)cc7)n6)c5-4)C4CC5CC(C4)CC3C5)c21]

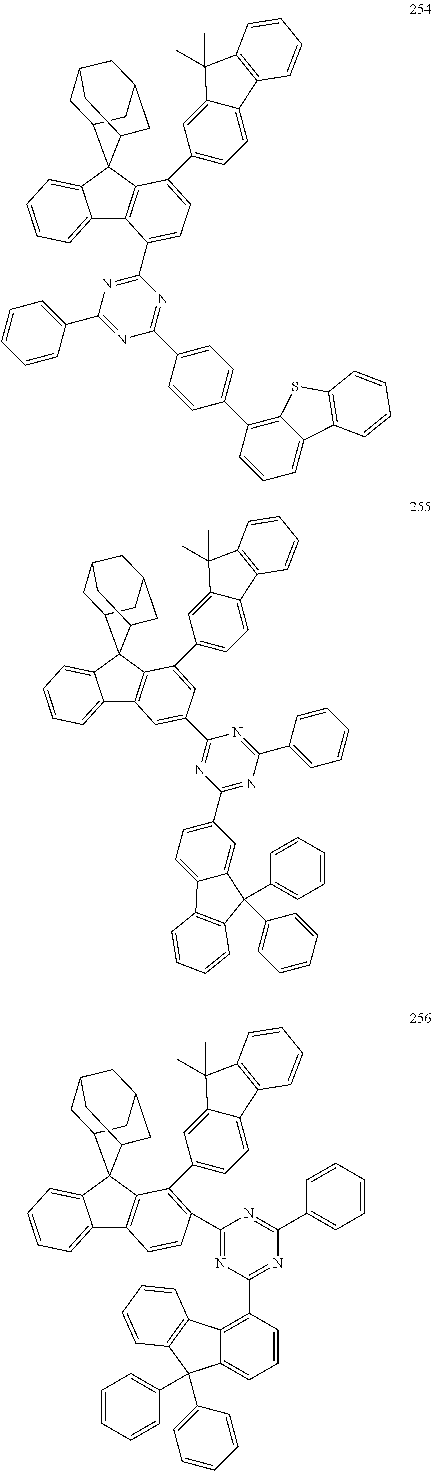

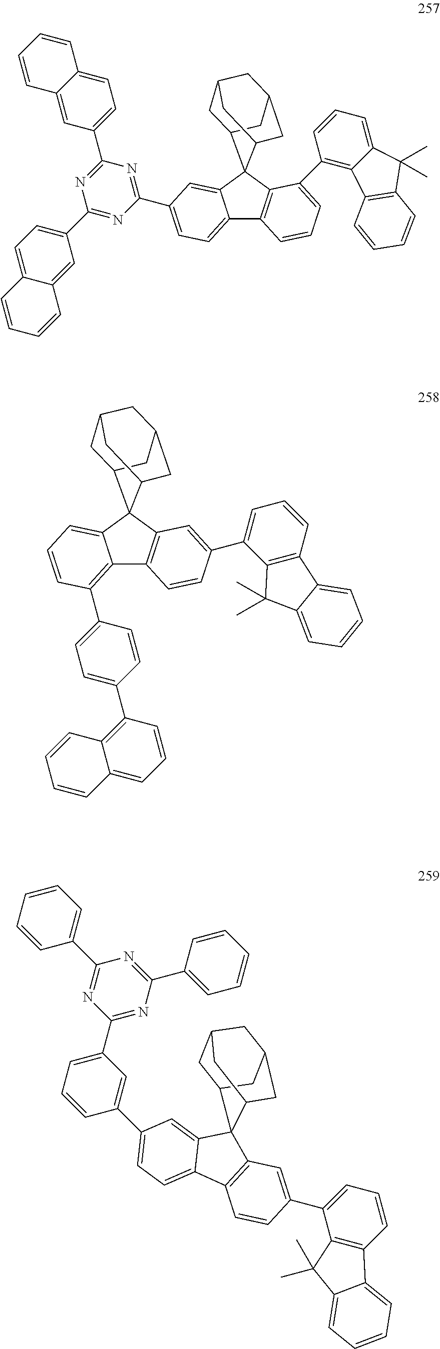

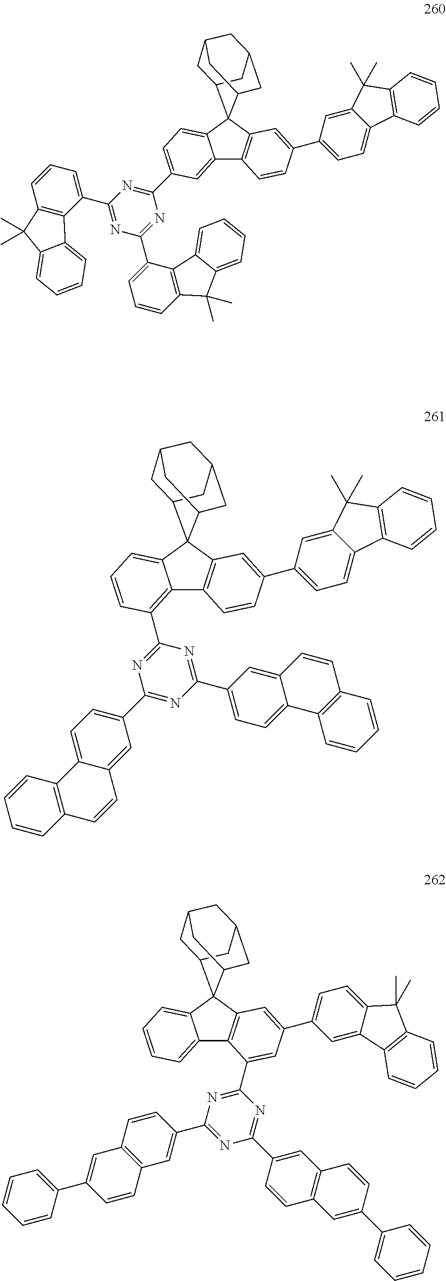 [SMILES: CC1(C)c2ccccc2-c2ccc(-c3ccc4c(c3)C3(c5ccc(-c6nc(-c7cccc8c7-c7ccccc7C8(C)C)nc(-c7cccc8c7-c7ccccc7C8(C)C)n6)cc5-4)C4CC5CC(C4)CC3C5)cc21 ; CC1(C)c2ccccc2-c2ccc(-c3ccc4c(c3)C3(c5cccc(-c6nc(-c7ccc8c(ccc9ccccc98)c7)nc(-c7ccc8c(ccc9ccccc98)c7)n6)c5-4)C4CC5CC(C4)CC3C5)cc21 ; CC1(C)c2ccccc2-c2cc(-c3cc(-c4nc(-c5ccc6cc(-c7ccccc7)ccc6c5)nc(-c5ccc6cc(-c7ccccc7)ccc6c5)n4)c4c(c3)C3(c5ccccc5-4)C4CC5CC(C4)CC3C5)ccc21]

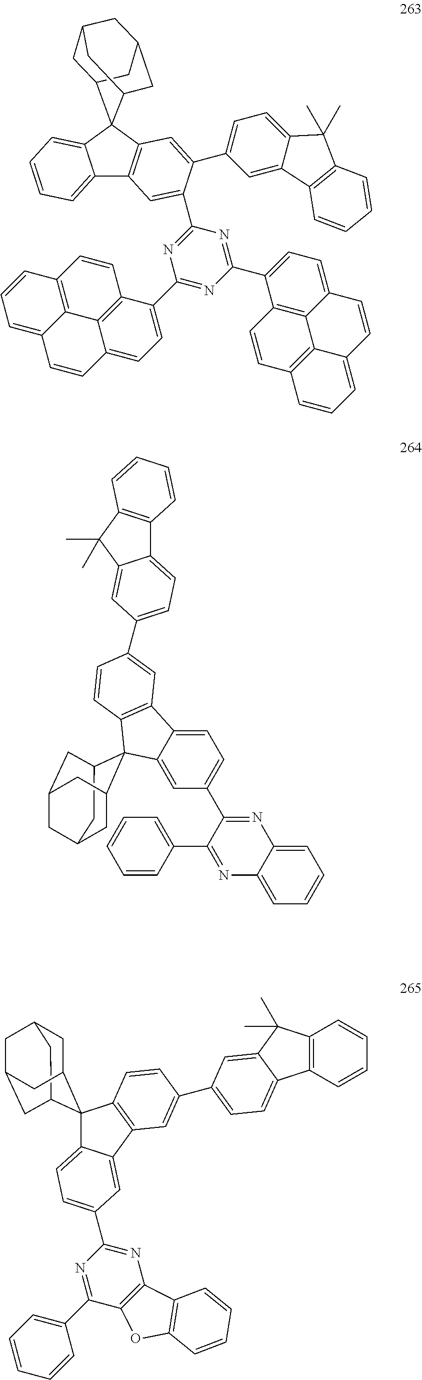 [SMILES: CC1(C)c2ccccc2-c2cc(-c3cc4c(cc3-c3nc(-c5ccc6ccc7cccc8ccc5c6c78)nc(-c5ccc6ccc7cccc8ccc5c6c78)n3)-c3ccccc3C43C4CC5CC(C4)CC3C5)ccc21 ; CC1(C)c2ccccc2-c2ccc(-c3ccc4c(c3)-c3ccc(-c5nc6ccccc6nc5-c5ccccc5)cc3C43C4CC5CC(C4)CC3C5)cc21 ; CC1(C)c2ccccc2-c2ccc(-c3ccc4c(c3)-c3cc(-c5nc(-c6ccccc6)c6oc7ccccc7c6n5)ccc3C43C4CC5CC(C4)CC3C5)cc21]

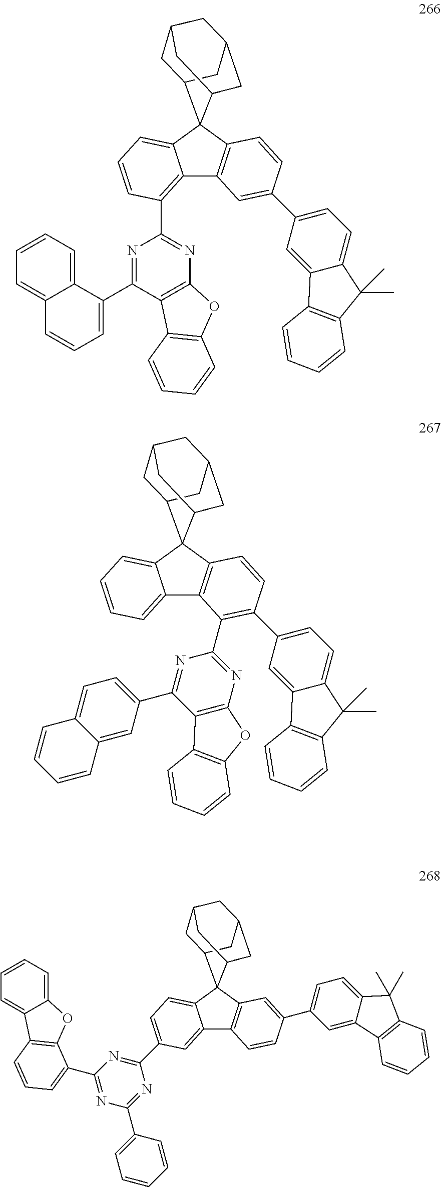

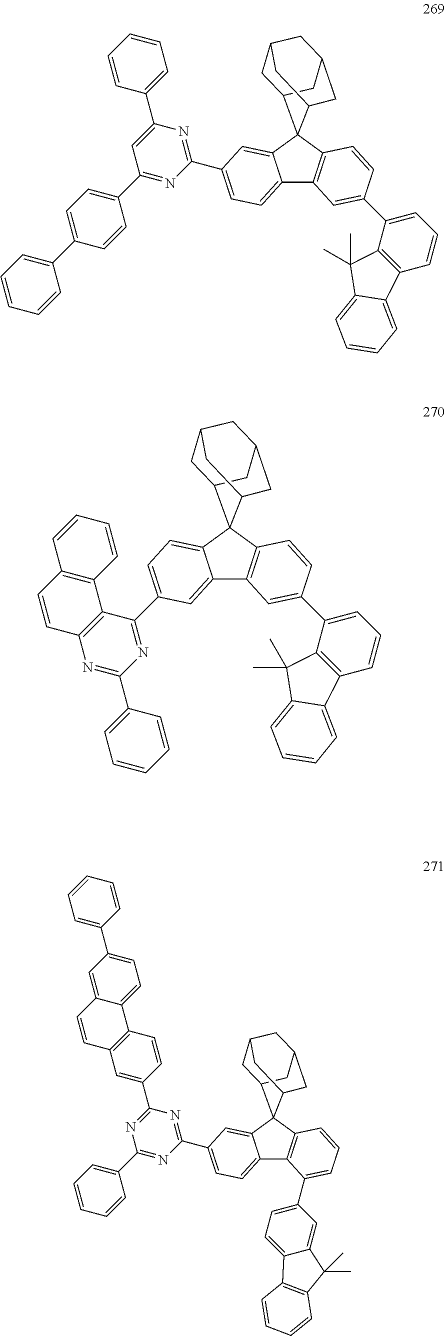 [SMILES: CC1(C)c2ccccc2-c2cccc(-c3ccc4c(c3)-c3ccc(-c5nc(-c6ccccc6)cc(-c6ccc(-c7ccccc7)cc6)n5)cc3C43C4CC5CC(C4)CC3C5)c21 ; CC1(C)c2ccccc2-c2cccc(-c3ccc4c(c3)-c3cc(-c5nc(-c6ccccc6)nc6ccc7ccccc7c56)ccc3C43C4CC5CC(C4)CC3C5)c21 ; CC1(C)c2ccccc2-c2ccc(-c3cccc4c3-c3ccc(-c5nc(-c6ccccc6)nc(-c6ccc7c(ccc8cc(-c9ccccc9)ccc87)c6)n5)cc3C43C4CC5CC(C4)CC3C5)cc21]

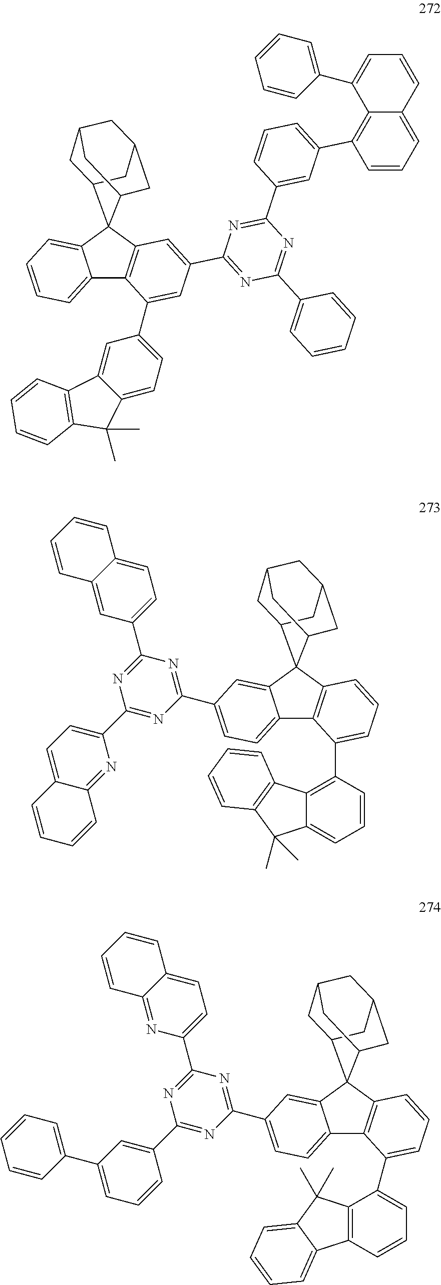 [SMILES: CC1(C)c2ccccc2-c2cc(-c3cc(-c4nc(-c5ccccc5)nc(-c5cccc(-c6cccc7cccc(-c8ccccc8)c67)c5)n4)cc4c3-c3ccccc3C43C4CC5CC(C4)CC3C5)ccc21 ; CC1(C)c2ccccc2-c2c(-c3cccc4c3-c3ccc(-c5nc(-c6ccc7ccccc7c6)nc(-c6ccc7ccccc7n6)n5)cc3C43C4CC5CC(C4)CC3C5)cccc21 ; CC1(C)c2ccccc2-c2cccc(-c3cccc4c3-c3ccc(-c5nc(-c6cccc(-c7ccccc7)c6)nc(-c6ccc7ccccc7n6)n5)cc3C43C4CC5CC(C4)CC3C5)c21]

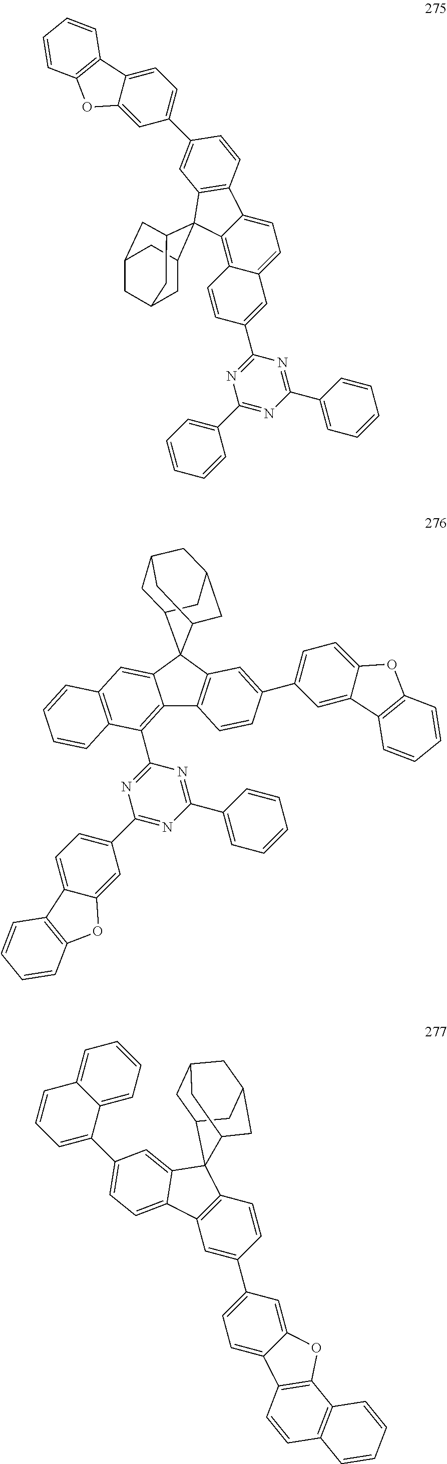 [SMILES: c1ccc(-c2nc(-c3ccccc3)nc(-c3ccc4c5c(ccc4c3)-c3ccc(-c4ccc6c(c4)oc4ccccc46)cc3C53C4CC5CC(C4)CC3C5)n2)cc1 ; c1ccc(-c2nc(-c3ccc4c(c3)oc3ccccc34)nc(-c3c4c(cc5ccccc35)C3(c5cc(-c6ccc7oc8ccccc8c7c6)ccc5-4)C4CC5CC(C4)CC3C5)n2)cc1 ; c1ccc2c(-c3ccc4c(c3)C3(c5ccc(-c6ccc7c(c6)oc6c8ccccc8ccc76)cc5-4)C4CC5CC(C4)CC3C5)cccc2c1]

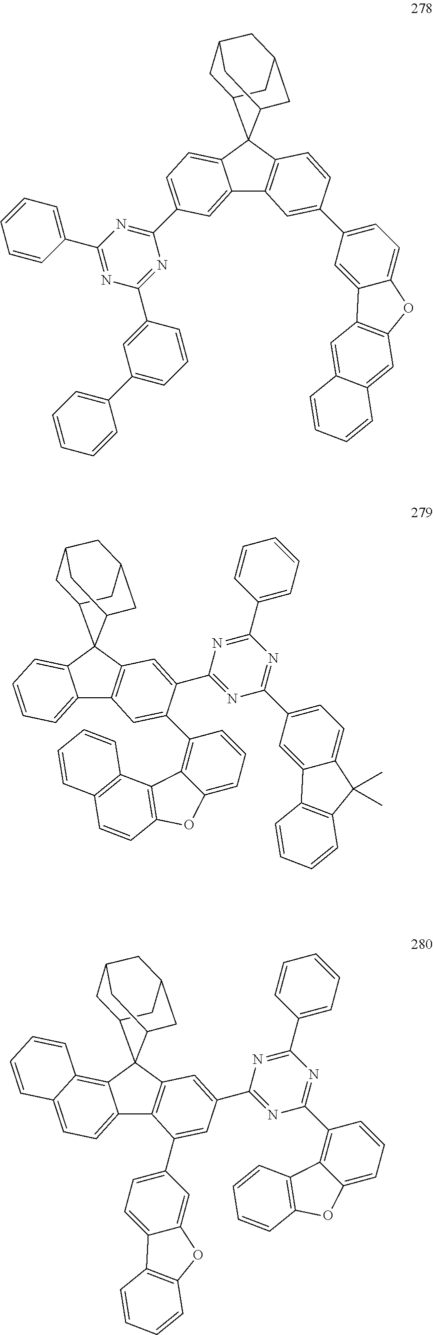 [SMILES: c1ccc(-c2cccc(-c3nc(-c4ccccc4)nc(-c4ccc5c(c4)-c4cc(-c6ccc7oc8cc9ccccc9cc8c7c6)ccc4C54C5CC6CC(C5)CC4C6)n3)c2)cc1 ; CC1(C)c2ccccc2-c2cc(-c3nc(-c4ccccc4)nc(-c4cc5c(cc4-c4cccc6oc7ccc8ccccc8c7c46)-c4ccccc4C54C5CC6CC(C5)CC4C6)n3)ccc21 ; c1ccc(-c2nc(-c3cc(-c4ccc5c(c4)oc4ccccc45)c4c(c3)C3(c5c-4ccc4ccccc54)C4CC5CC(C4)CC3C5)nc(-c3cccc4oc5ccccc5c34)n2)cc1]

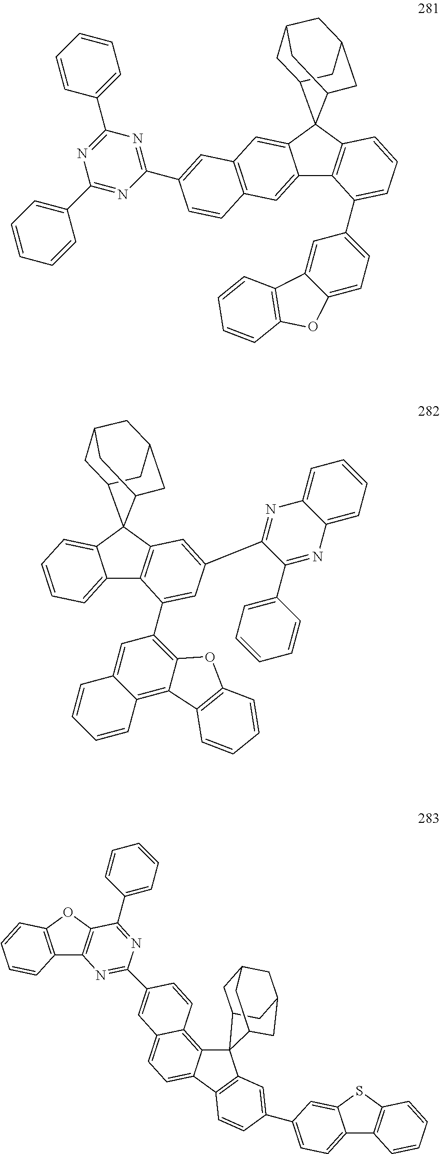 [SMILES: c1ccc(-c2nc(-c3ccccc3)nc(-c3ccc4cc5c(cc4c3)C3(c4cccc(-c6ccc7oc8ccccc8c7c6)c4-5)C4CC5CC(C4)CC3C5)n2)cc1 ; c1ccc(-c2nc3ccccc3nc2-c2cc(-c3cc4ccccc4c4c3oc3ccccc34)c3c(c2)C2(c4ccccc4-3)C3CC4CC(C3)CC2C4)cc1 ; c1ccc(-c2nc(-c3ccc4c5c(ccc4c3)-c3ccc(-c4ccc6c(c4)sc4ccccc46)cc3C53C4CC5CC(C4)CC3C5)nc3c2oc2ccccc23)cc1]

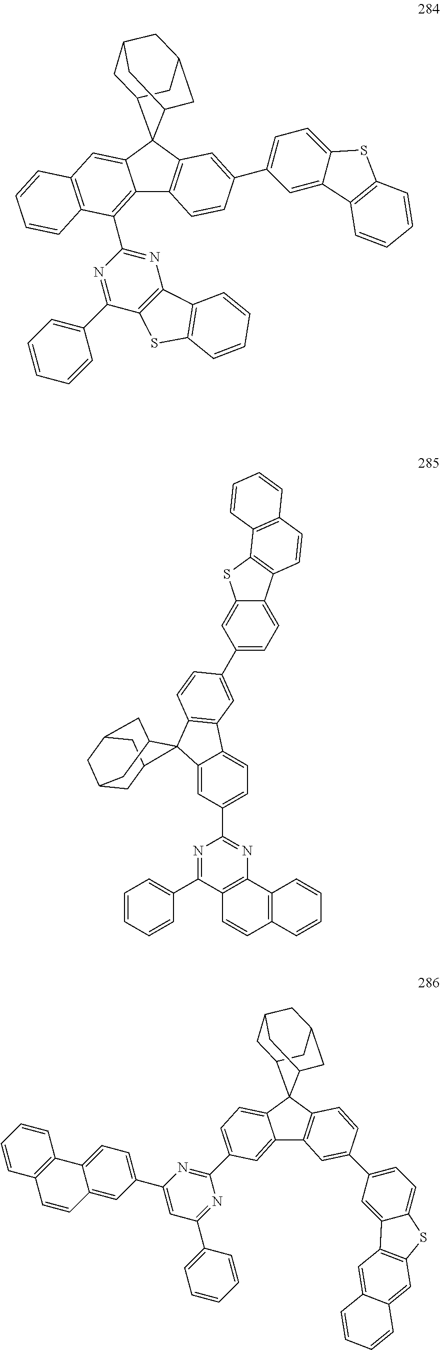 [SMILES: c1ccc(-c2nc(-c3c4c(cc5ccccc35)C3(c5cc(-c6ccc7sc8ccccc8c7c6)ccc5-4)C4CC5CC(C4)CC3C5)nc3c2sc2ccccc23)cc1 ; c1ccc(-c2nc(-c3ccc4c(c3)C3(c5ccc(-c6ccc7c(c6)sc6c8ccccc8ccc76)cc5-4)C4CC5CC(C4)CC3C5)nc3c2ccc2ccccc23)cc1 ; c1ccc(-c2cc(-c3ccc4c(ccc5ccccc54)c3)nc(-c3ccc4c(c3)-c3cc(-c5ccc6sc7cc8ccccc8cc7c6c5)ccc3C43C4CC5CC(C4)CC3C5)n2)cc1]

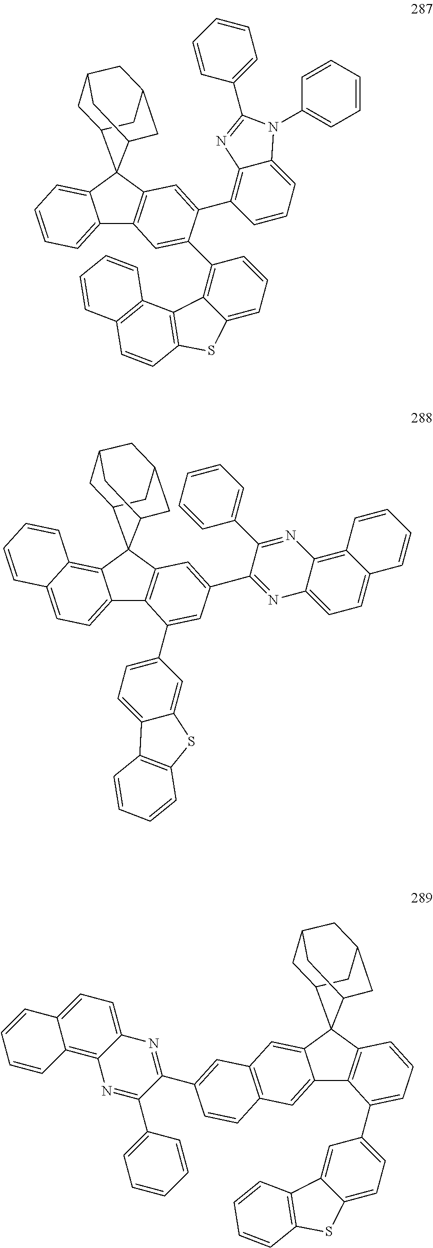 [SMILES: c1ccc(-c2nc3c(-c4cc5c(cc4-c4cccc6sc7ccc8ccccc8c7c46)-c4ccccc4C54C5CC6CC(C5)CC4C6)cccc3n2-c2ccccc2)cc1 ; c1ccc(-c2nc3c(ccc4ccccc43)nc2-c2cc(-c3ccc4c(c3)sc3ccccc34)c3c(c2)C2(c4c-3ccc3ccccc43)C3CC4CC(C3)CC2C4)cc1 ; c1ccc(-c2nc3c(ccc4ccccc43)nc2-c2ccc3cc4c(cc3c2)C2(c3cccc(-c5ccc6sc7ccccc7c6c5)c3-4)C3CC4CC(C3)CC2C4)cc1]

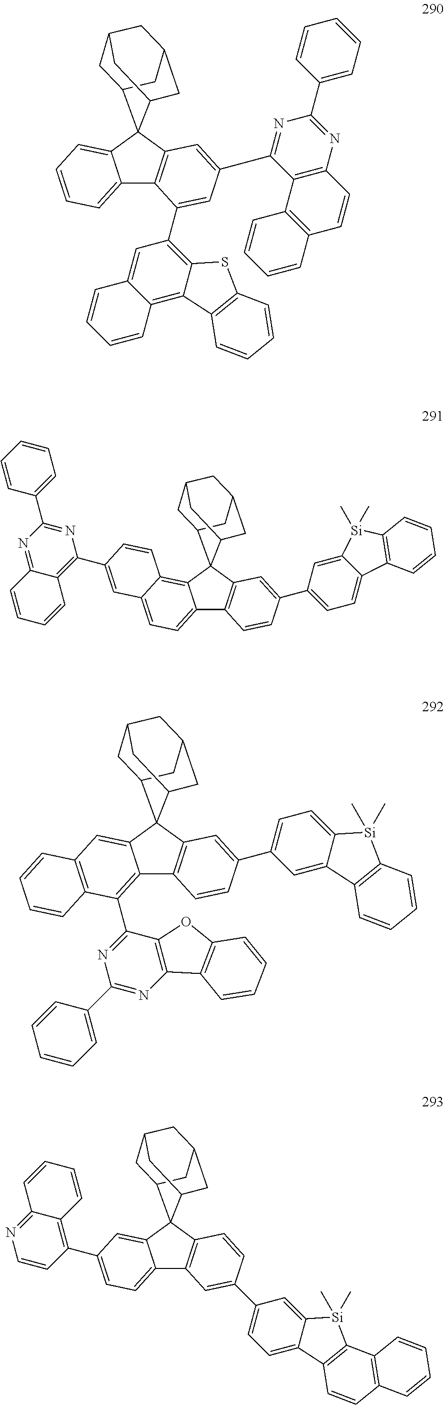 [SMILES: c1ccc(-c2nc(-c3cc(-c4cc5ccccc5c5c4sc4ccccc45)c4c(c3)C3(c5ccccc5-4)C4CC5CC(C4)CC3C5)c3c(ccc4ccccc43)n2)cc1 ; C[Si]1(C)c2ccccc2-c2ccc(-c3ccc4c(c3)C3(c5c-4ccc4cc(-c6nc(-c7ccccc7)nc7ccccc67)ccc54)C4CC5CC(C4)CC3C5)cc21 ; C[Si]1(C)c2ccccc2-c2cc(-c3ccc4c(c3)C3(c5cc6ccccc6c(-c6nc(-c7ccccc7)nc7c6oc6ccccc67)c5-4)C4CC5CC(C4)CC3C5)ccc21 ; C[Si]1(C)c2cc(-c3ccc4c(c3)-c3ccc(-c5ccnc6ccccc56)cc3C43C4CC5CC(C4)CC3C5)ccc2-c2ccc3ccccc3c21]

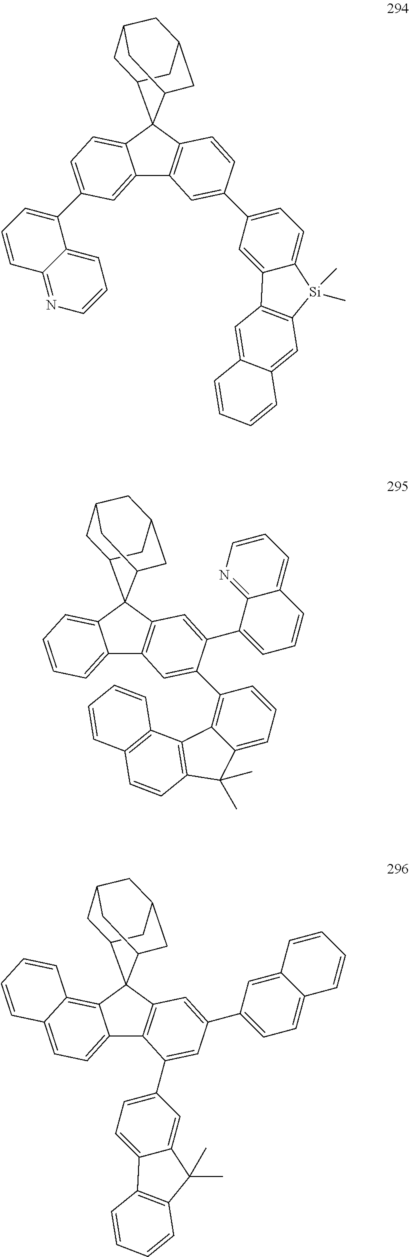

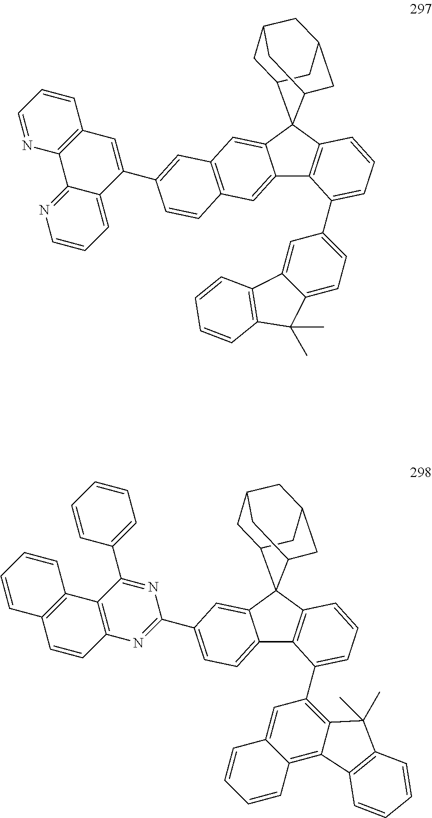 [SMILES: CC1(C)c2ccccc2-c2cc(-c3cccc4c3-c3cc5ccc(-c6cc7cccnc7c7ncccc67)cc5cc3C43C4CC5CC(C4)CC3C5)ccc21 ; CC1(C)c2ccccc2-c2c1c(-c1cccc3c1-c1ccc(-c4nc(-c5ccccc5)c5c(ccc6ccccc65)n4)cc1C31C3CC4CC(C3)CC1C4)cc1ccccc21]

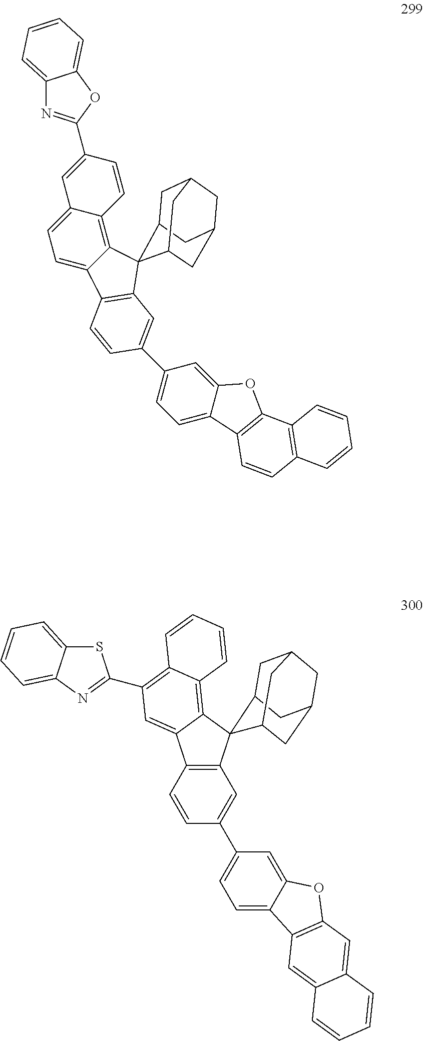 [SMILES: c1ccc2c(c1)ccc1c3ccc(-c4ccc5c(c4)C4(c6c-5ccc5cc(-c7nc8ccccc8o7)ccc65)C5CC6CC(C5)CC4C6)cc3oc21 ; c1ccc2cc3c(cc2c1)oc1cc(-c2ccc4c(c2)C2(c5c-4cc(-c4nc6ccccc6s4)c4ccccc54)C4CC5CC(C4)CC2C5)ccc13]

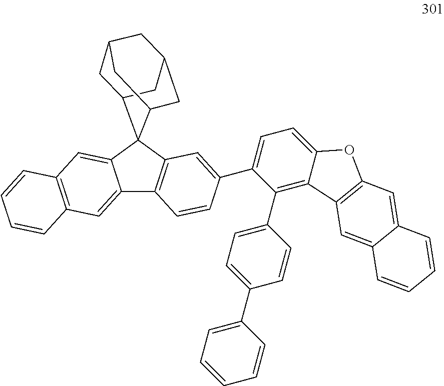 [SMILES: c1ccc(-c2ccc(-c3c(-c4ccc5c(c4)C4(c6cc7ccccc7cc6-5)C5CC6CC(C5)CC4C6)ccc4oc5cc6ccccc6cc5c34)cc2)cc1]

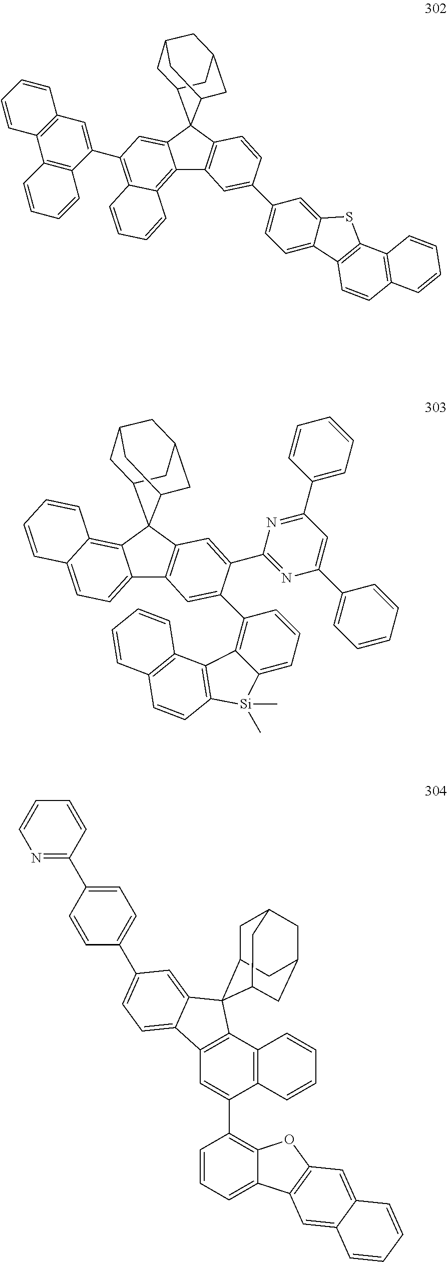 [SMILES: c1ccc2c(c1)cc(-c1cc3c(c4ccccc14)-c1cc(-c4ccc5c(c4)sc4c6ccccc6ccc54)ccc1C31C3CC4CC(C3)CC1C4)c1ccccc12 ; C[Si]1(C)c2cccc(-c3cc4c(cc3-c3nc(-c5ccccc5)cc(-c5ccccc5)n3)C3(c5c-4ccc4ccccc54)C4CC5CC(C4)CC3C5)c2-c2c1ccc1ccccc21 ; c1ccc(-c2ccc(-c3ccc4c(c3)C3(c5c-4cc(-c4cccc6c4oc4cc7ccccc7cc46)c4ccccc54)C4CC5CC(C4)CC3C5)cc2)nc1]

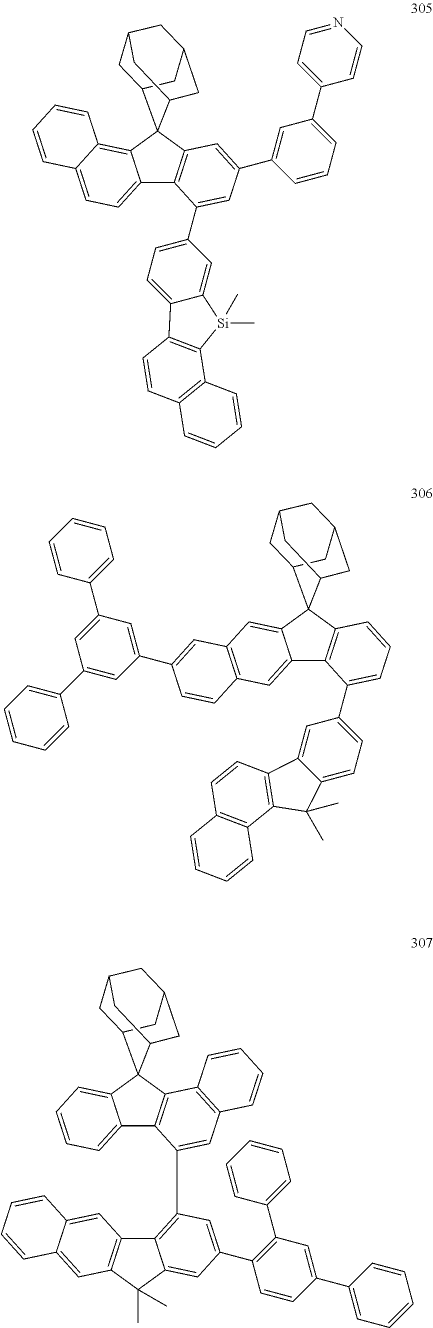

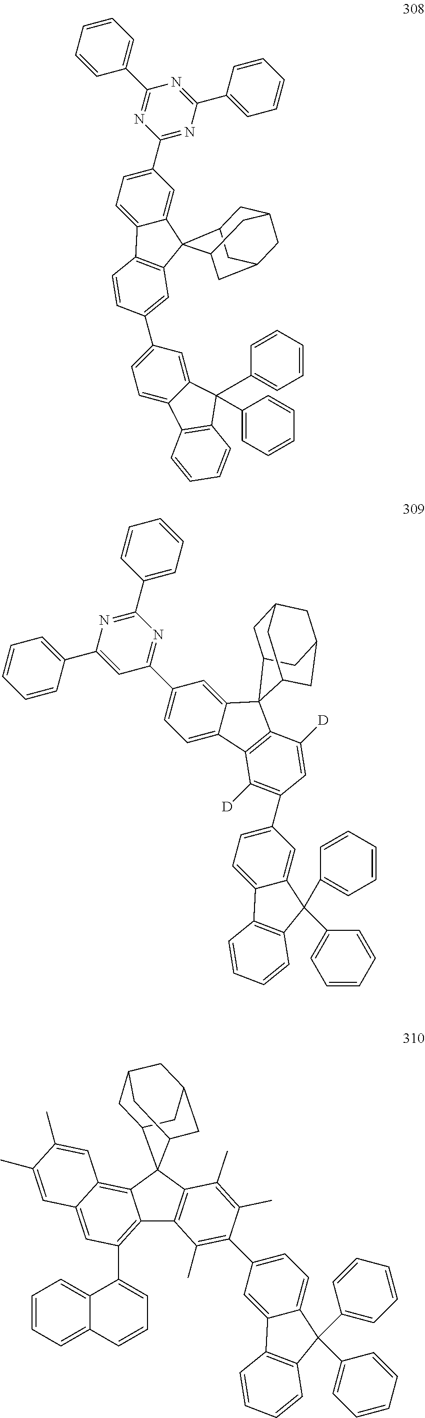

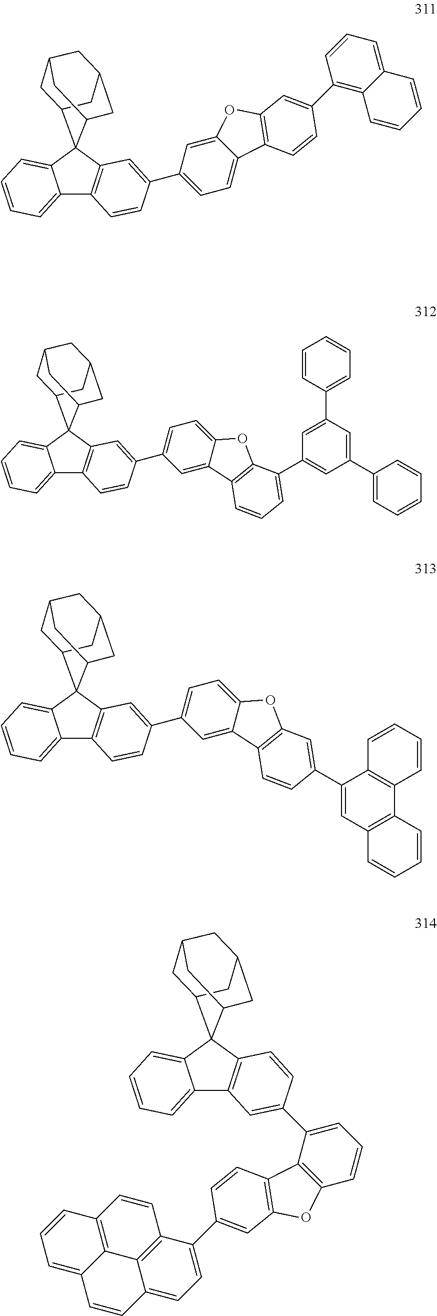 [SMILES: c1ccc2c(c1)-c1ccc(-c3ccc4c(c3)oc3cc(-c5cccc6ccccc56)ccc34)cc1C21C2CC3CC(C2)CC1C3 ; c1ccc(-c2cc(-c3ccccc3)cc(-c3cccc4c3oc3ccc(-c5ccc6c(c5)C5(c7ccccc7-6)C6CC7CC(C6)CC5C7)cc34)c2)cc1 ; c1ccc2c(c1)-c1ccc(-c3ccc4oc5cc(-c6cc7ccccc7c7ccccc67)ccc5c4c3)cc1C21C2CC3CC(C2)CC1C3 ; c1ccc2c(c1)-c1cc(-c3cccc4oc5cc(-c6ccc7ccc8cccc9ccc6c7c89)ccc5c34)ccc1C21C2CC3CC(C2)CC1C3]

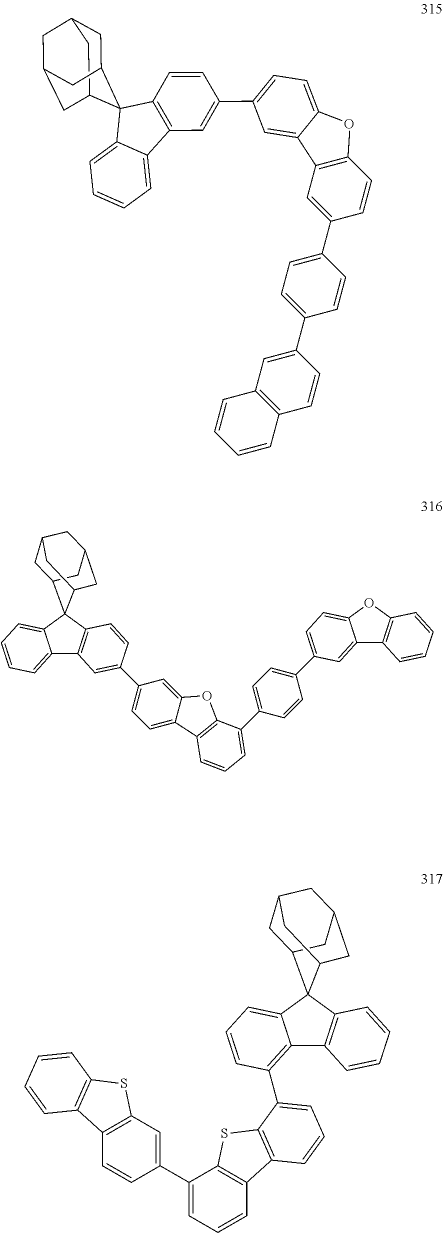 [SMILES: c1ccc2c(c1)-c1cc(-c3ccc4oc5ccc(-c6ccc(-c7ccc8ccccc8c7)cc6)cc5c4c3)ccc1C21C2CC3CC(C2)CC1C3 ; c1ccc2c(c1)-c1cc(-c3ccc4c(c3)oc3c(-c5ccc(-c6ccc7oc8ccccc8c7c6)cc5)cccc34)ccc1C21C2CC3CC(C2)CC1C3 ; c1ccc2c(c1)-c1c(-c3cccc4c3sc3c(-c5ccc6c(c5)sc5ccccc56)cccc34)cccc1C21C2CC3CC(C2)CC1C3]

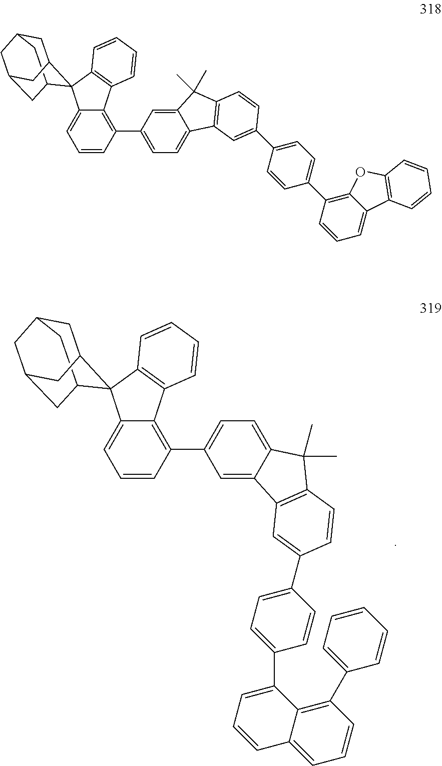

16. An organic electroluminescence device, comprising an anode and a cathode which are disposed oppositely, and a functional layer disposed between the anode and the cathode; wherein the functional layer contains the compound according to claim 1.

17. The organic electroluminescence device according to claim 16, wherein the functional layer comprises a hole injection layer, a hole transport layer, an organic electroluminescence layer, an electron transport layer and an electron injection layer, and the organic electroluminescence layer contains the compound.

18. An electronic apparatus, comprising the organic electroluminescence device according to claim 16.
